(19) United States
(12) Patent Application Publication
(10) Pub. No.: US 2026/0146041 A1
(43) Pub. Date: May 28, 2026

(54) PANNEXIN-1 MODULATORS AND METHODS OF TREATING DISORDERS IN WHICH PANNEXIN-1 IS IMPLICATED

(71) Applicant: Pannex Therapeutics Inc., New York, NY (US)

(72) Inventors: David Alexandre Bravo López, New York City, NY (US); Gerhard Max Gross, Fuldabruck (DE)

(21) Appl. No.: 19/107,299

(22) PCT Filed: Aug. 30, 2023
(86) PCT No.: PCT/US2023/031577

§ 371 Date: Feb. 27, 2025

Related U.S. Application Data
(60) Provisional application No. 63/402,336, filed on Aug. 30, 2022.

(51) Int. Cl. C07D 473/40; A61K 31/52; C07D 209/16; C07D 215/06; C07D 401/14; C07D 403/10; C07D 405/04; C07D 417/12; C07D 471/04; C07D 473/32; C07D 487/04; C07D 487/14; C07D 491/04; C07D 491/14; C07D 513/04

(52) U.S. Cl. CPC C07D 473/40; A61K 31/52; C07D 209/16; C07D 215/06; C07D 401/14; C07D 403/10; C07D 405/04; C07D 417/12; C07D 471/04; C07D 473/32; C07D 487/04; C07D 487/14; C07D 491/04; C07D 491/14; C07D 513/04

(57) ABSTRACT

Disclosed herein are small molecule modulators of pannexin. Also disclosed herein are methods of treating disorders associated with exacerbated activation of ATP-induced signal pathways mediated by pannexin, such as pain and opioid addiction, by administering small molecule modulators of pannexin.

### CROSS-REFERENCE TO RELATED APPLICATIONS

This application claims the benefit of U.S. Provisional Application No. 63/402,336, filed on Aug. 30, 2022. The entire teachings of the above application are incorporated herein by reference.

### FIELD OF THE INVENTION

The present invention relates to small molecule modulators of pannexin and correlated channels. It is also related to the treatment of disorders associated with exacerbated activation of ATP-induced signal pathways mediated by pannexin and correlated channels, such as pain and opioid addiction.

### BACKGROUND OF THE INVENTION

Cell-cell and cell-matrix interactions are fundamental properties of multicellular organisms. Gap junctions allow direct passage of ions and small molecules (<2,000 Da) from cell to cell. The vertebrate homologues of innexins, called “pannexins”, form mostly hemichannels, or pannexons due to the high level of glycosylation in their extracellular domains. Pannexins have a cytosolic N-terminal domain, four transmembrane domains with two extracellular loops, and a cytosolic C-terminal domain (1).

The pannexin (PANX) family consists of three proteins, PANX1, PANX2, and PANX3, all of which have been shown to form a single-membrane channel. Panx1 is ubiquitously expressed in almost all cell types, including those in the nervous and immune systems, eye, muscle, olfactory epithelium, blood vessels, exocrine glands (e.g., lacrimal and salivary glands), thyroid, prostate, kidney, and liver. Panx1 proteins are localized primarily at the plasma membrane.

Pannexins are ATP-release channels that can be activated by caspase cleavage of their pore-associated C-terminal tail, the autoregulatory region controlling channel permeability. The regulated ATP (nucleotide) release through pannexin HCs is implicated in a number of normal physiological functions and in response to stressors or pathological states in cells and tissue. Functions of pannexins include regulation of cell differentiation and migration, tissue development and regeneration, inflammation, wound healing and cell death. PANX1 was shown to exhibit different conformations with different conductance and permeability properties depending on which type of stimulus resulted in channel activation. The pore-associated C-termini inhibits the PANX1 channel function effectively. Panx1 has a low voltage channel opening for Cl—and a high voltage opening for molecules like ATP and others. The opening can be gradual and can be permanent (caspase cleavage) or temporary (1).

In the resting state PANX1 is closed. Panx1 has different activation modes. Permeability and conductances of pannexons vary with the activation mode. The pannexon has high conductance and is permeable to ATP, when activated by various physiological stimuli or by elevated extracellular [K⁺]. Activation of pannexons only by stepping the membrane potential to positive values results in a small channel conductance and selective permeability to Cl⁻.

Panx1 is expressed in most cell types and tissues in the somatic and nervous system in particular heart, skeletal muscle, skin, ovary, testes placenta, prostate, thymus, lung, liver, small intestine, pancreas, spleen, colon, blood epithelium and erythrocytes. In the CNS, Panx1 is found in cerebellum, cortex lens, retina, pyramidal cells, hippocampus, amygdala, substantia negra, olfactory bulb, neurons and glial cells. Therefore, there are numerous pathologies associated with PANX1. The PANX1 channel is an integral component of the P2X/P2Y purinergic signalling pathway, and it is the key contributor to pathophysiological ATP release. For example, the PANX1 channel, along with ATP, purinergic receptors, and ectonucleotidases, contributes to several feedback loops during the inflammatory response (2).

The three major important processes where extracellular ATP and therefore Panx1 are involved are as follows:

Extracellular ATP is an important signalling molecule throughout the inflammatory cascade, serving as a danger signal that causes activation of the inflammasome, enhancement of immune cell infiltration, and fine-tuning of several signalling cascades including those important for the resolution of inflammation. Panx1-mediated ATP release is involved in inflammasome activation and neutrophil/macrophage chemotaxis, and activation of T cells. A key role for Panx1 in inducing and propagating inflammation has been demonstrated in various organs, including lung and the central and peripheral nervous system.

Moreover, extracellular ATP can be broken down by ectonucleotidases into ADP, AMP, and adenosine, which is critical in the resolution of inflammation. Therefore, PANX1 contributes to important feedback loops during the inflammatory response and is thus representing promising candidates for new therapies (2).

In extracellular spaces within the nervous system, ATP acts as a signalling molecule that can play many different roles. It can act as a fast neurotransmitter, as a trophic factor promoting growth and development, as well as a damage-associated molecular pattern (DAMP; any molecule that can elicit a non-infectious inflammatory response) that regulates communication with phagocytic cells including acting as an activator of microglia in the injured cortex. PANX1 is expressed on both neurons and glia and it is thought that Panx1 mediates the crosstalk between those cells. Pathological activity of Panx1 is believed to be strongly contributing to several disease processes including seizure, stroke, migraine headache and chronic pain. Newer evidence also revealed a physiological function of Panx1 in regulating neural stem cell survival, neuronal maturation and synaptic plasticity, with possible relevance to normal cognitive functioning (3)(4). I

In the tumor microenvironment extracellular ATP (released by Panx1) at high levels triggers cell death. For cancer cells, high levels of ATP are associated with enhanced cancer cell survival, proliferation and metastatic potential (5).

### SUMMARY OF THE INVENTION

The invention relates to compounds with PANX1 modulatory activity. In some embodiments, said compounds are a compound of formula I or a pharmaceutically acceptable salt, hydrate, solvate, or polymorph thereof:

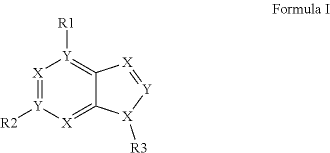

wherein,
X and Y are individually C, CH or N, or preferably wherein Y is C, or CH and X is N;
R¹, R² and R³ are independently hydrogen; lower alkyl, optionally substituted with hydroxy; lower cycloalkyl; lower alkenyl; lower alkoxy; lower alkynyl; phenyl; halogenated phenyl; halogen; a substituted or non-substituted aromatic or non-aromatic heterocycle, preferably diazine, pyrazole, diazole, triazole, or their alkyl derivatives; 2-hydroxyisopropyl; or have one of the following structures:

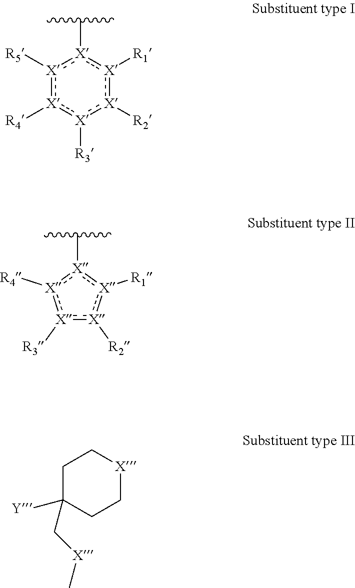

wherein,
X′ is C, CH, CH₂, N, S, or O, or preferably can be CH₂, N, or O;
X″ is independently C, CH, CH₂, N, S, or O, preferably CH₂, N, or O;
X′″ is NH, N-lower alkyl or O;
R1′-R5′ are independently absent, hydrogen, lower alkyl, lower cycloalkyl, halogen, OH, NH₂, hydroxy-lower alkyl, or NH-lower alkyl, O-lower alkyl, O—PO—OR₆′, O-Lower alkyl or alkyloxy-O—PO—O—R₆′, or O—PO—O-lower alkyl-O—PO—R₆′, preferably OH, NH₂, NH-lower alkyl, wherein vicinal R1′ and R5′ may form a 5-7 membered ring moiety;
R₆′ is H or O-lower alkyl;
R1″-R4″ are independently absent, hydrogen, lower alkyl, lower cycloalkyl, OH, NH₂, hydroxy-lower alkyl, NH-lower alkyl, O-lower alkyl, O—PO—OR₆′, O-lower alkyl or alkyloxy-O—PO—O—R₆′, or O—PO—O-lower alkyl-O—PO—R₆′, preferably OH, NH₂, NH-lower alkyl, or halogen;
Y′″ is halogen; and
wherein the bonds between adjacent ring substituents X′ or adjacent ring substituents X″ may constitute single or double bonds.

In particular embodiments, the compound of Formula I may be selected from the group consisting of:

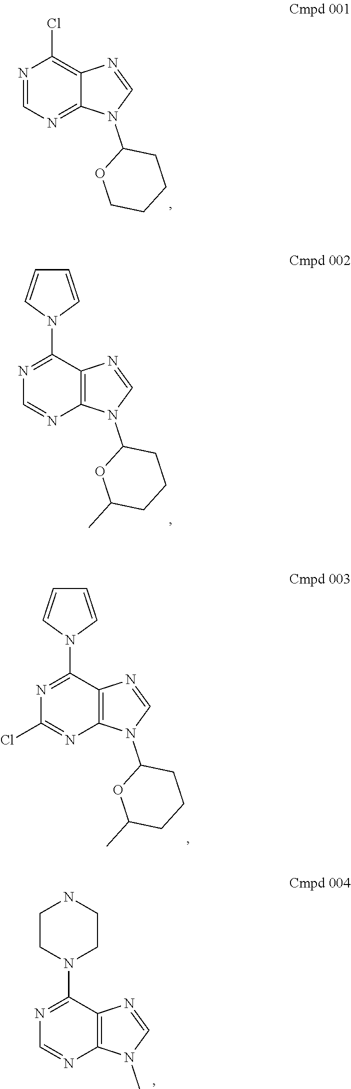 [SMILES: Clc1ncnc2c1ncn2C1CCCCO1 ; CC1CCCC(n2cnc3c(-n4cccc4)ncnc32)O1 ; CC1CCCC(n2cnc3c(-n4cccc4)nc(Cl)nc32)O1 ; Cn1cnc2c(N3CCNCC3)ncnc21]

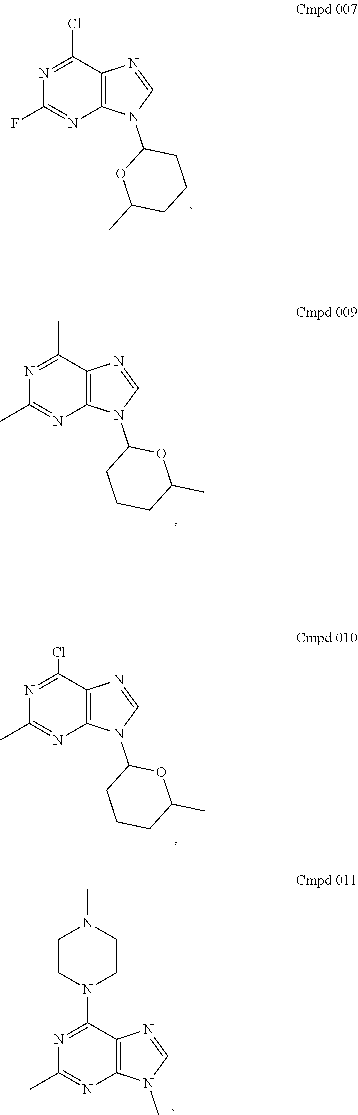

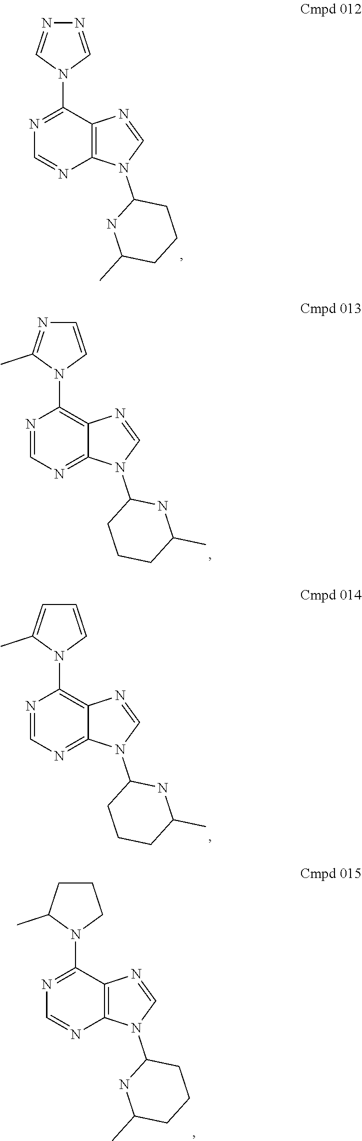

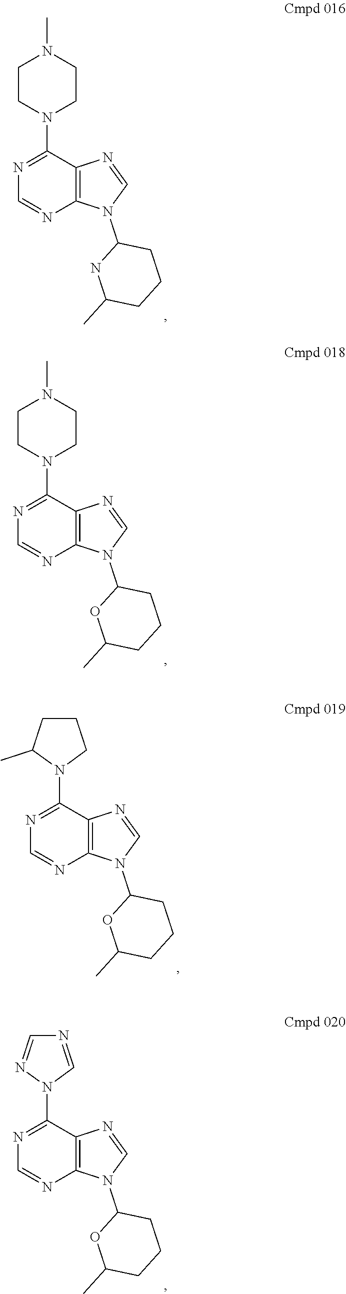 [SMILES: CC1CCCC(n2cnc3c(N4CCN(C)CC4)ncnc32)N1 ; CC1CCCC(n2cnc3c(N4CCN(C)CC4)ncnc32)O1 ; CC1CCCC(n2cnc3c(N4CCCC4C)ncnc32)O1 ; CC1CCCC(n2cnc3c(-n4cncn4)ncnc32)O1]

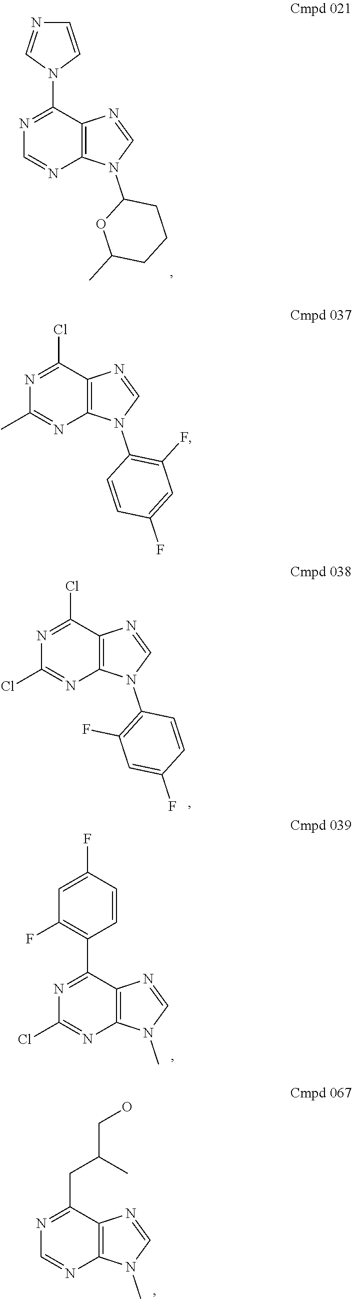 [SMILES: CC1CCCC(n2cnc3c(-n4ccnc4)ncnc32)O1 ; Cc1nc(Cl)c2ncn(-c3ccc(F)cc3F)c2n1 ; Fc1ccc(-n2cnc3c(Cl)nc(Cl)nc32)c(F)c1 ; Cn1cnc2c(-c3ccc(F)cc3F)nc(Cl)nc21 ; CC(CO)Cc1ncnc2c1ncn2C]

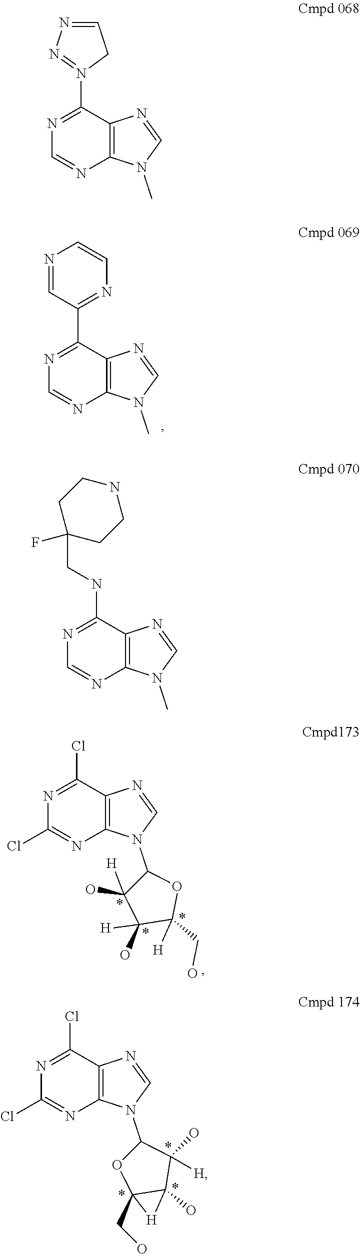

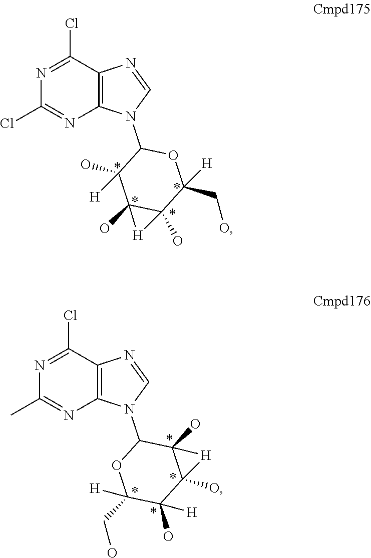

and a pharmaceutically acceptable salt, hydrate, solvate, or polymorph thereof.

In another aspect, the invention relates to compounds of formula I′ or a pharmaceutically acceptable salt, hydrate, solvate, or polymorph thereof:

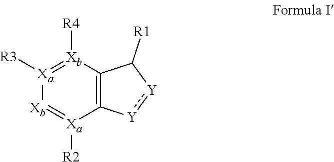

wherein,
Xₐ or X_{b} is C, CH or N, or preferably Xₐ is C or CH, and X_{b} is N; Y is CH, CH₂, C-lower alkyl, CH-lower alkyl, C-halogen, CH-halogen or C-dihalogen; Z is CH or N, preferably CH; wherein the bond between adjacent ring substituents Y may constitute a single or double bond;
R¹, R², R³, and R⁴ are independently absent or are hydrogen; lower alkyl; lower cycloalkyl; lower alkenyl; lower alkoxy; lower alkynyl; phenyl; halogenated phenyl; halogen; a substituted or non-substituted aromatic or non-aromatic heterocycle, preferably triazole, or diazine; 2-hydroxyisopropyl, or have one of the following structures:

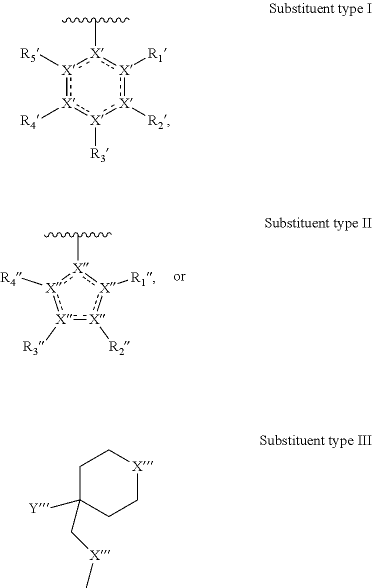

wherein,
X′ is C, CH, CH₂, N, S or O, preferably CH₂, N, or O;
X″ is C, CH, CH₂, N, S or O, preferably CH₂, N, or O;
X′″ is NH, N-lower alkyl or O;
R1′-R5′ are independently absent, or are H, lower alkyl, lower cycloalkyl, OH, NH₂, NH-lower alkyl, halogen, O-lower alkyl, O—PO—OR₆′, O-Lower alkyl or alkyloxy-O—PO—O—R₆′, or O—PO—O-lower alkyl-O—PO—R₆′, preferably OH, NH₂, NH-lower alkyl, wherein vicinal R1′ and R5′ may form a 5-7 membered ring moiety;
R₆′ is H or O-lower alkyl;
R1″-R4″ are independently absent, or are hydrogen, lower alkyl, lower cycloalkyl, or halogen;
Y′″ is halogen, and
wherein the bonds between adjacent ring substituents X′ or adjacent ring substituents X″ may constitute single or double bonds, forming thereby a non-aromatic or aromatic ring.

In certain embodiments, a compound of Formula I′ may be selected from the group consisting of:

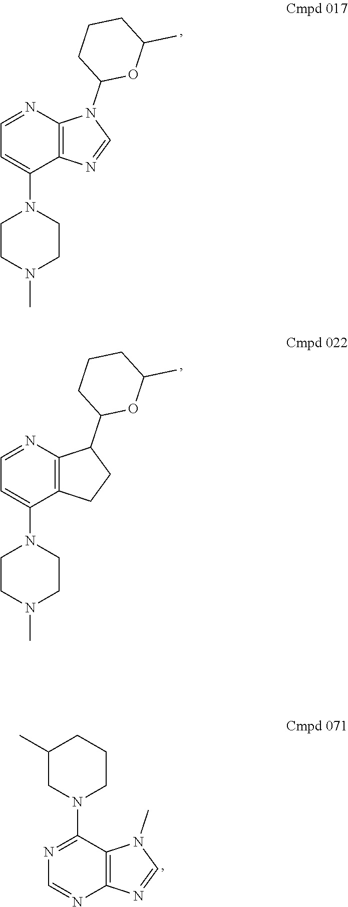

and a pharmaceutically acceptable salt, hydrate, solvate, or polymorph thereof.

In another particular embodiment, the invention relates to compounds of Formula II or a pharmaceutically acceptable salt, hydrate, solvate, or polymorph thereof:

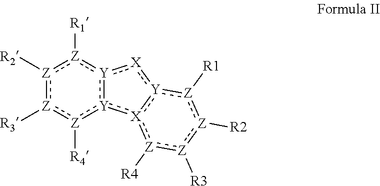

wherein,
X is N; Y is C; and Z is N, C or CH
R¹-R⁴ are independently absent when Z is N, or,
one or more of R¹-R⁴ or R¹′-R⁴′ are lower to medium chain oxo- or keto fatty acids, optionally substituted with oxo or lower alkyl; H; halogen; lower alkyl; lower cycloalkyl; or COOH; or
one or more of R¹-R⁴ or R¹′-R⁴′ comprise the substituent of Formula X:

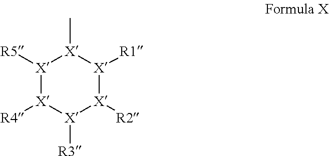

wherein,
X′ is CH, CO, N or O, preferably wherein X′ comprises up to two N or O;
R¹″-R⁵″ are independently absent, or are H lower alkyl; lower cycloalkyl; halogen; NH₂; NH-lower alkyl; hydroxy; oxo; COOH, SO₂NH₂; SO₂NH-loweralkyl; a substituted or non-substituted aromatic or non-aromatic heterocycle, preferably tetrazole; or comprises the substituent of Formula XI:

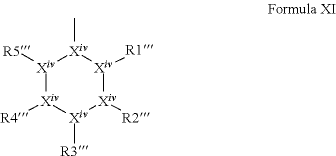

wherein,
X^{iv} is CH, CH₂, CO, N or O, or preferably wherein X^{iv} comprises up to two N or O;
R¹′″-R⁵′″ are independently absent, or are H, halogen, NH₂, lower alkyl, lower cycloalkyl, NH-lower alkyl, hydroxy, COOH, SO₂NH₂, SO₂NH-loweralkyl; a substituted or non-substituted aromatic or non-aromatic heterocycle, preferably tetrazole; or preferably wherein only one of R¹′″-R⁵′″ comprises a non-substituted aromatic or non-aromatic heterocycle; or
one or more of R¹-R⁴ comprise the substituent of formula XII:

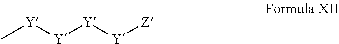

wherein,
Y′ is absent or can each be, independently, CH₂, CH₂(CH₃), CO, SO, SO₂, CHOH or NH; and Z′ is COOH; SO₂NH₂, a substituted or non-substituted aromatic or non-aromatic heterocycle, preferably tetrazole; forming a C₃-C₈ linear or branched chain; or
one or more of R¹-R⁴ comprise the substituent of formula XIII:

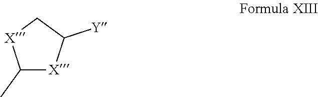

wherein,
Y″ is COOH, SO₂NH₂, or tetrazole; and X′″ is N or O.

In a particular embodiment, the compound of Formula II may be selected from the group consisting of:

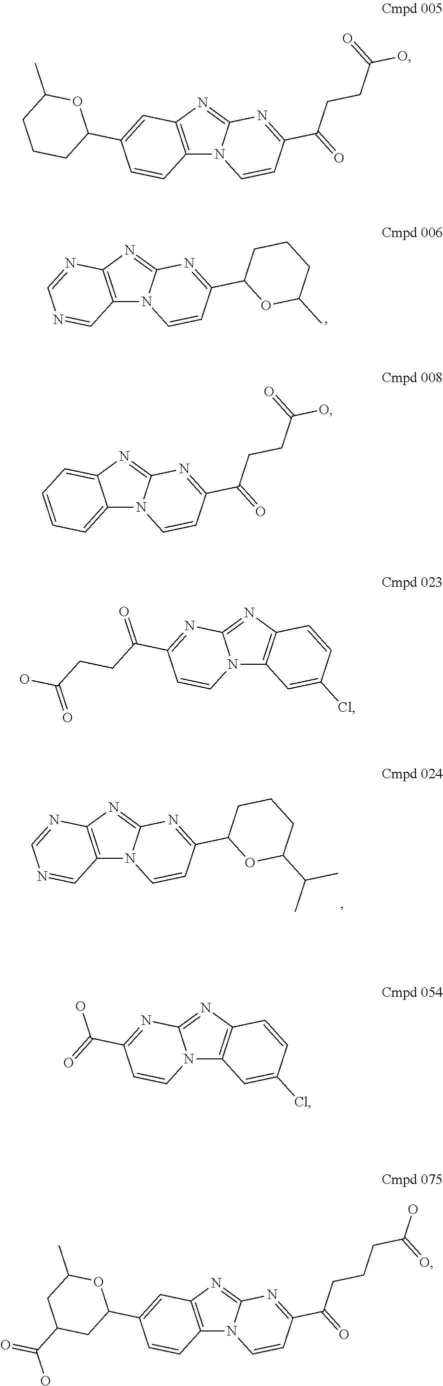

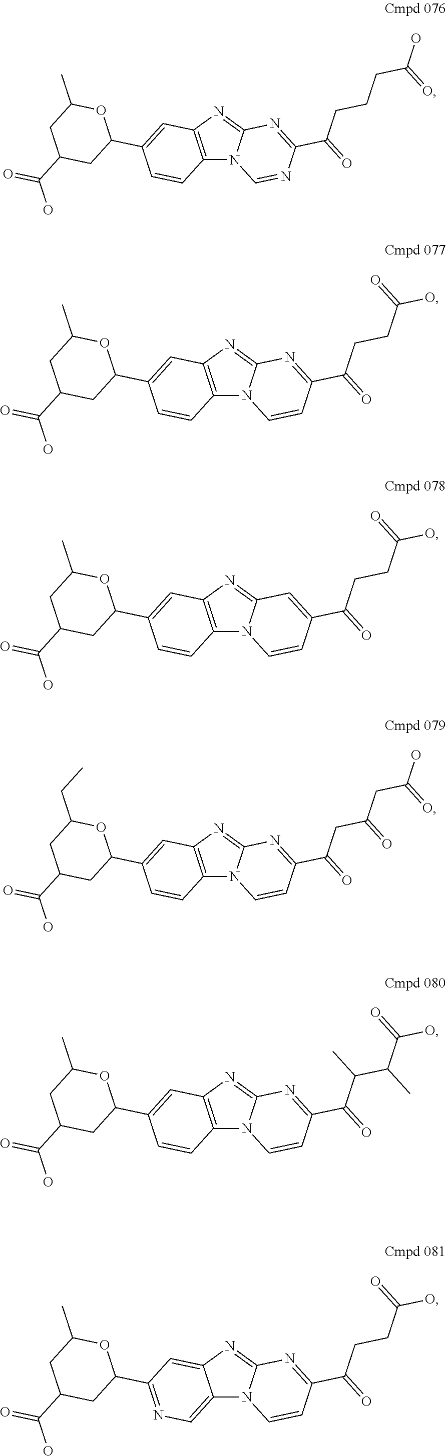

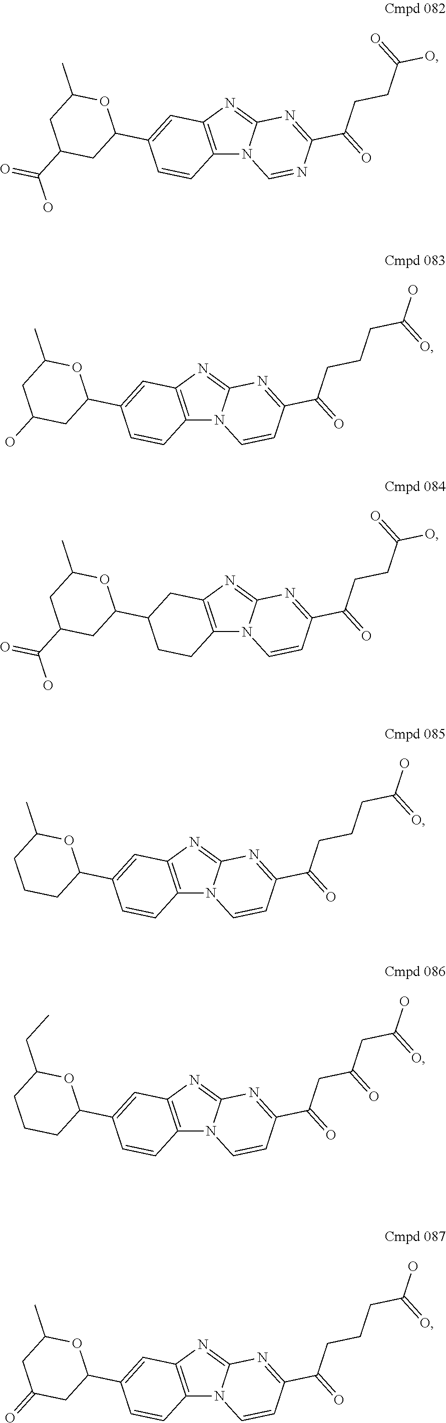

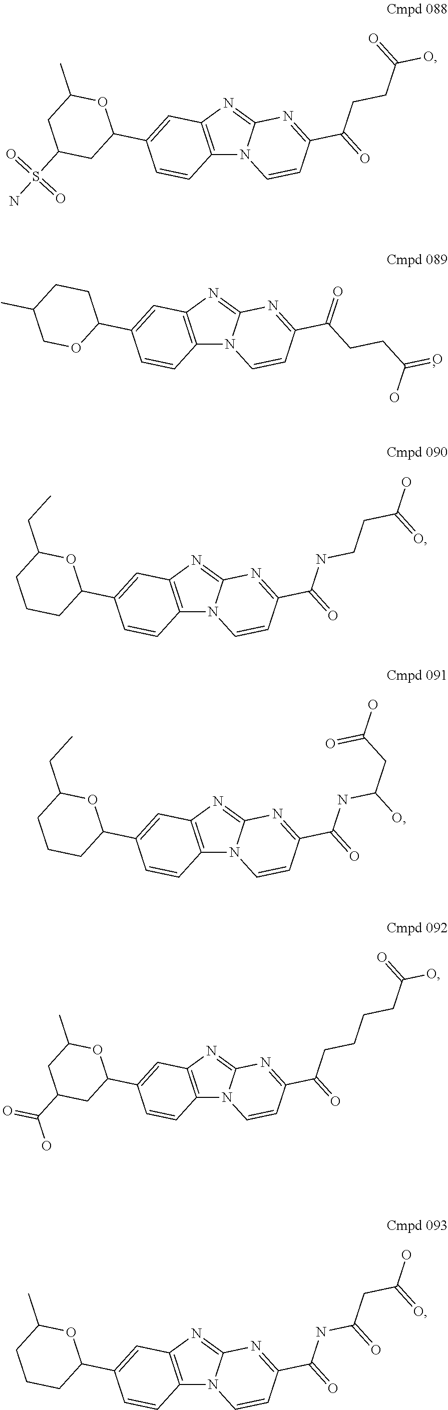

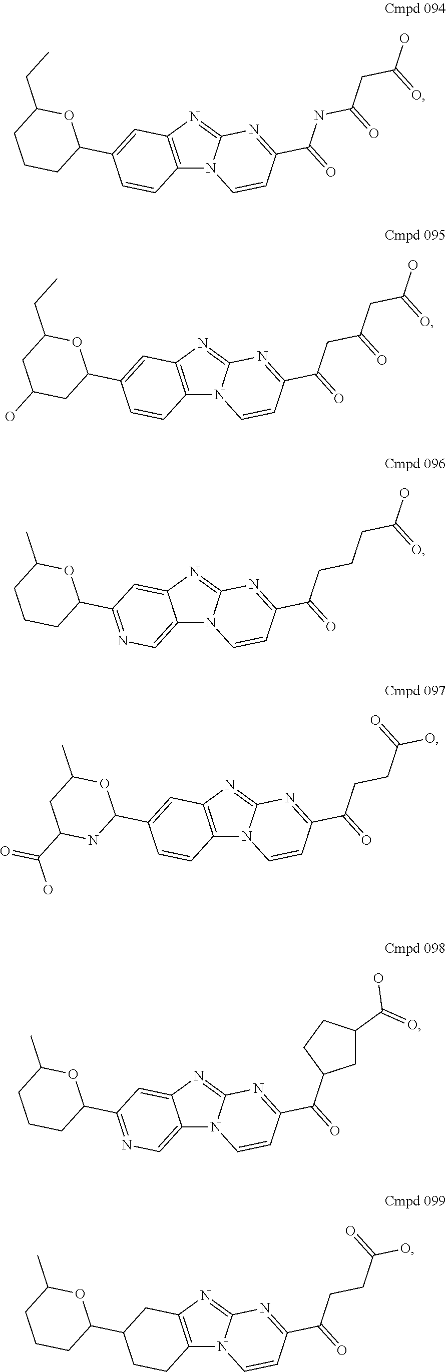

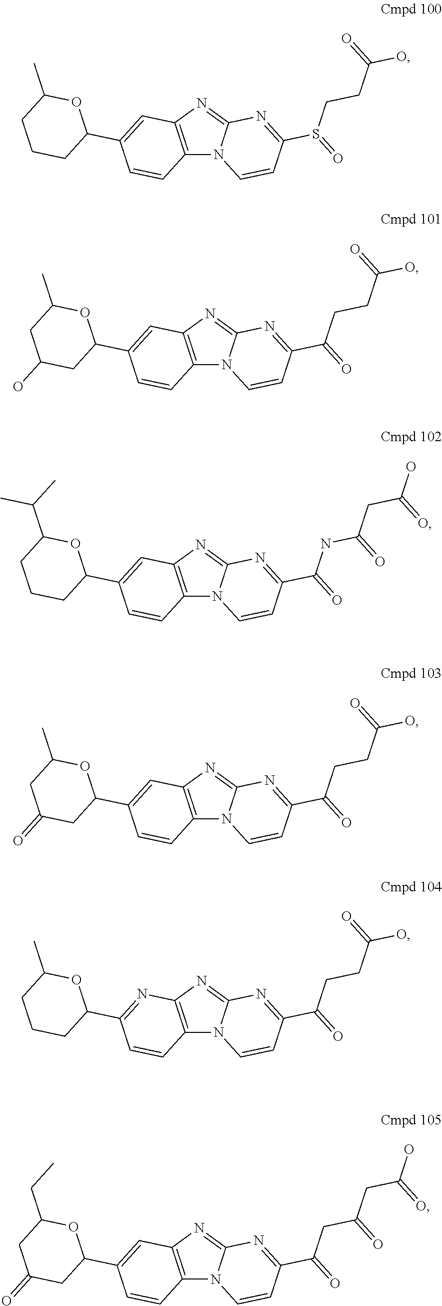

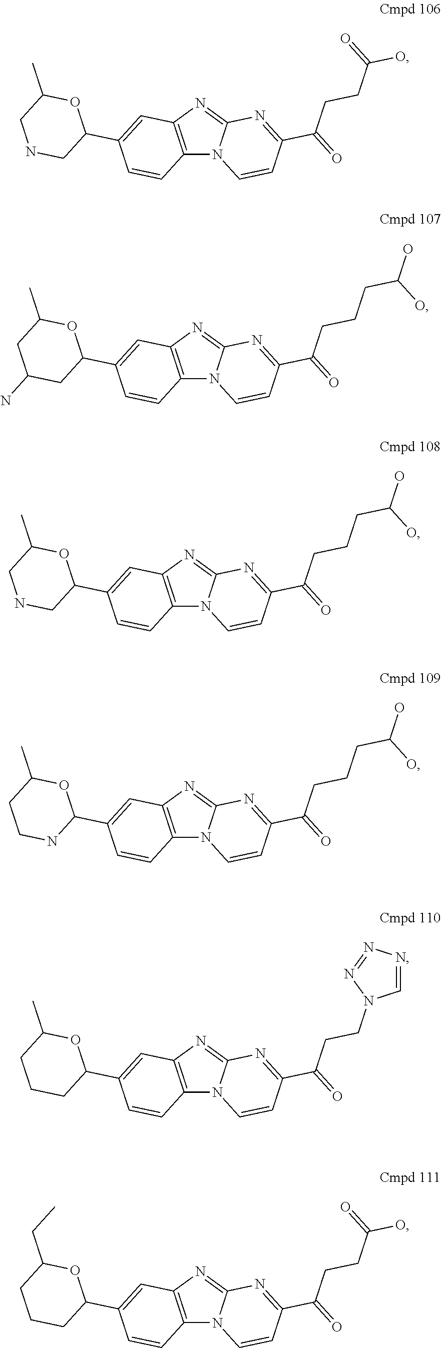

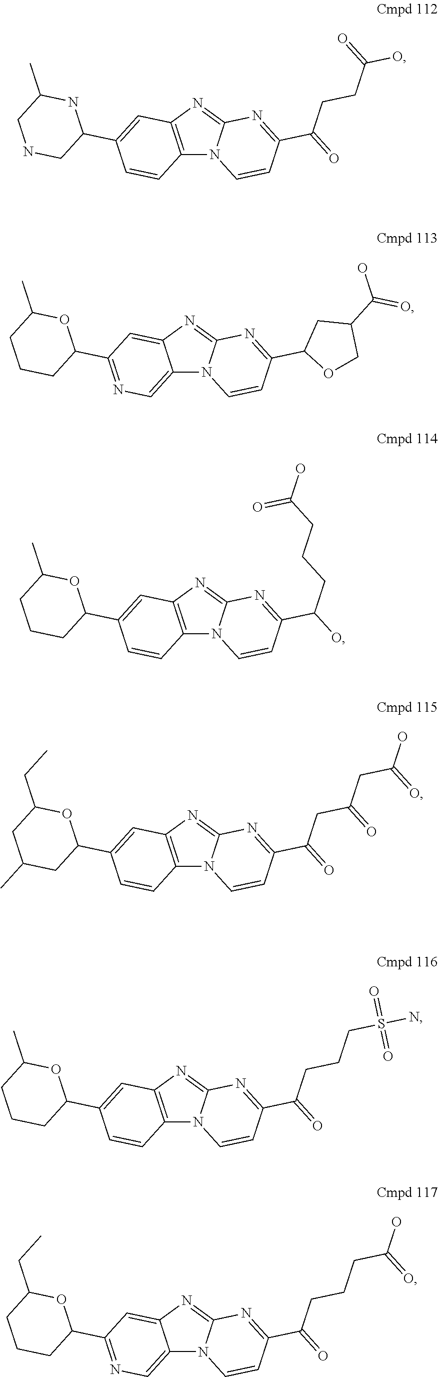

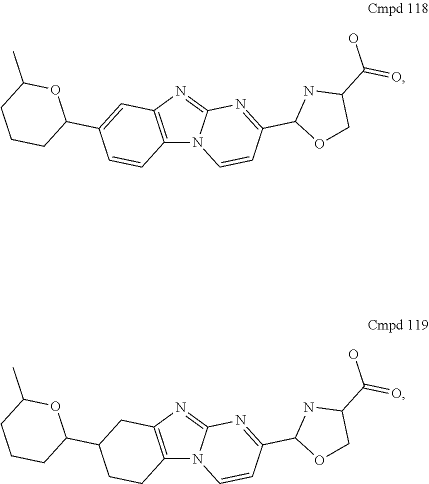 [SMILES: CC1CCCC(c2ccc3c(c2)nc2nc(C4NC(C(=O)O)CO4)ccn23)O1 ; CC1CCCC(C2CCc3c(nc4nc(C5NC(C(=O)O)CO5)ccn34)C2)O1]

and a pharmaceutically acceptable salt, hydrate, solvate, or polymorph thereof.

In another aspect, the invention relates to compounds of Formula III or a pharmaceutically acceptable salt, hydrate, solvate, or polymorph thereof:

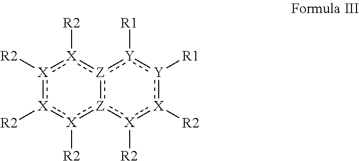

wherein,
each Z is independently selected from CH or C; each X is independently selected from CH, C, CH₂, O, N, S, SO, or SO₂; each Y is independently selected from N, C, CH, CH₂, CO, SO, S, or N-alkyl; wherein the bonds between adjacent ring substituents X, Z and/or Y constitute single or double bonds;
each R¹ is either absent or is H, halogen, O, NH₂, N-lower cycloalkyl, a phenyl group, alkyl group, optionally substituted with one or both of oxo and carboxy, OH, SO₂N(CH₃)₂, C(O)N(CH₃)₂, a five membered heterocycle, preferably tetrazole, —CO—(CH₂)ₙ—COOH, wherein n is 0-4, wherein R₁ optionally may form together with neighboring substituents, an aromatic or aliphatic 5, 6, or 7-membered ring system, which may contain an heteroatom, optionally substituted with one or more COOH, carboxamide, alkoxy, oxo, halogen, or triazole groups, or R₁ optionally comprises the substituent of Formula XIV:

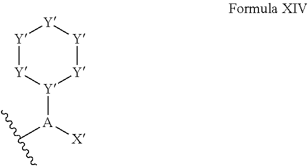

wherein,
X′ is absent or is O, N, or N-lower cycloalkyl; A is CH or may be a direct bond when X′ is absent, Y′ is O, N, N-lower cycloalkyl, CH₂, CH-lower alkyl, or CH—OH, or preferably X′ is absent or is O and the Y′-ring contains 1 or 2 N or O atoms; wherein the bonds between adjacent substituents Y′ and other atoms in the ring constitute single or double bonds, thereby forming a non-aromatic or aromatic ring; and
R² is absent, or is halogen; H; lower alkyl, optionally substituted with carboxy or oxo; hydroxy; alkoxy, COOH; SO₂NH₂; SO₂NH—O—CH₃, SO₂NH—C(O)—CH₃, SO₂N(CH₃)₂, SO₂-lower alkyl; C(O)N(CH₃)₂, a substituted or non-substituted aromatic or non-aromatic heterocycle, preferably triazole or tetrazole; or comprises the substituent of formula XV:

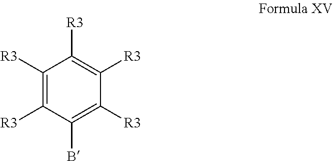

wherein,
B′ is attachment side, wherein B′ may comprise a linker or a direct bond, wherein the linker is —N—; and R³ is independently H, COOH, SO₂NH₂, SO₂NH— loweralkyl, halogen, or a substituted or non-substituted aromatic or non-aromatic heterocycle, preferably triazole or tetrazole.

In particular aspects of the invention, the compound of Formula III may be selected from the group consisting of:

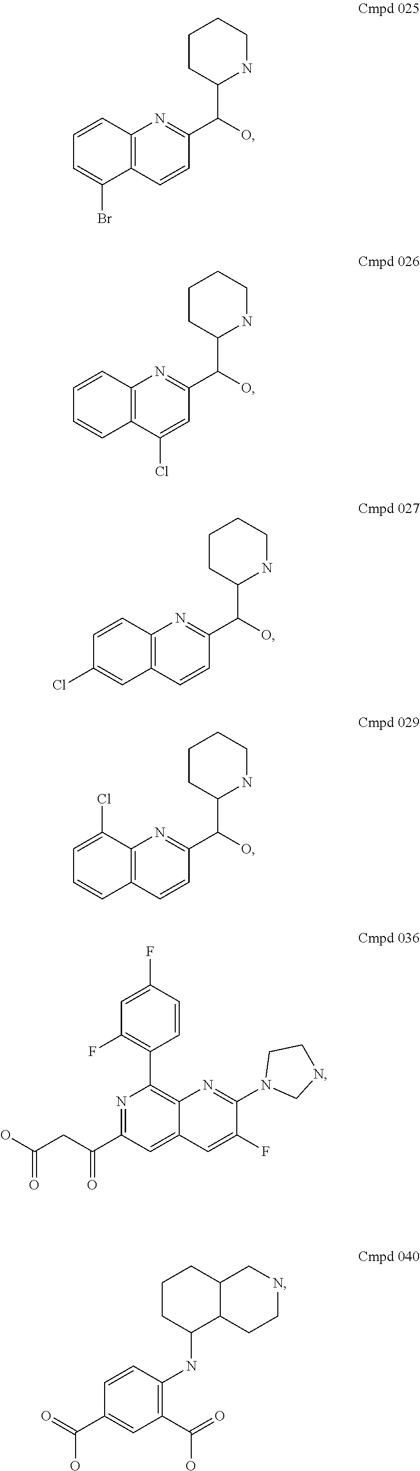

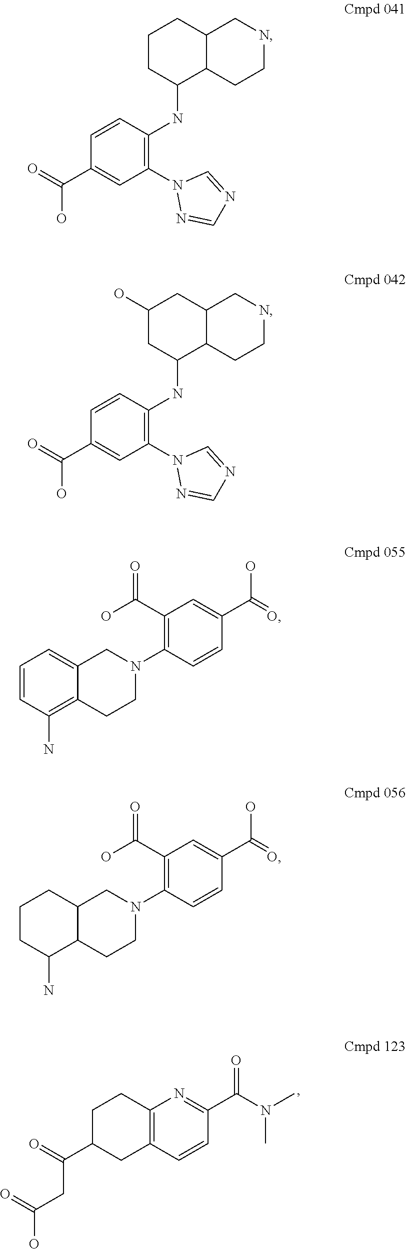

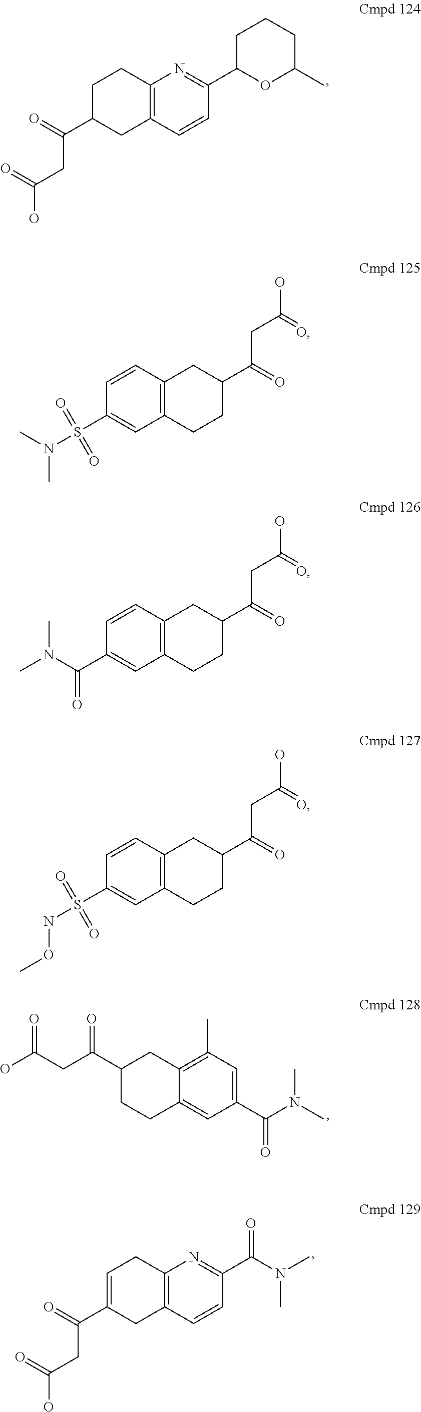

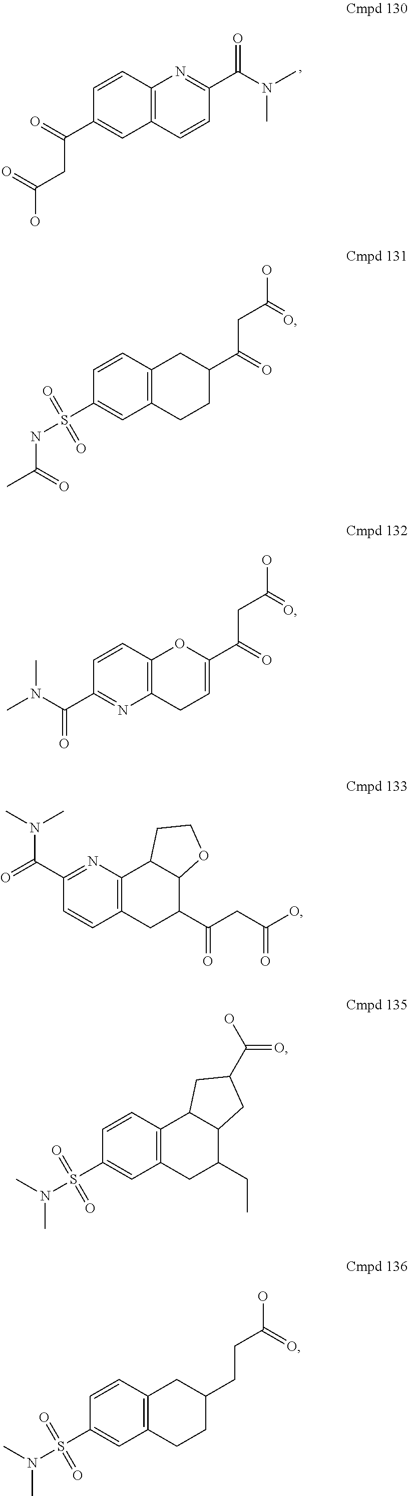

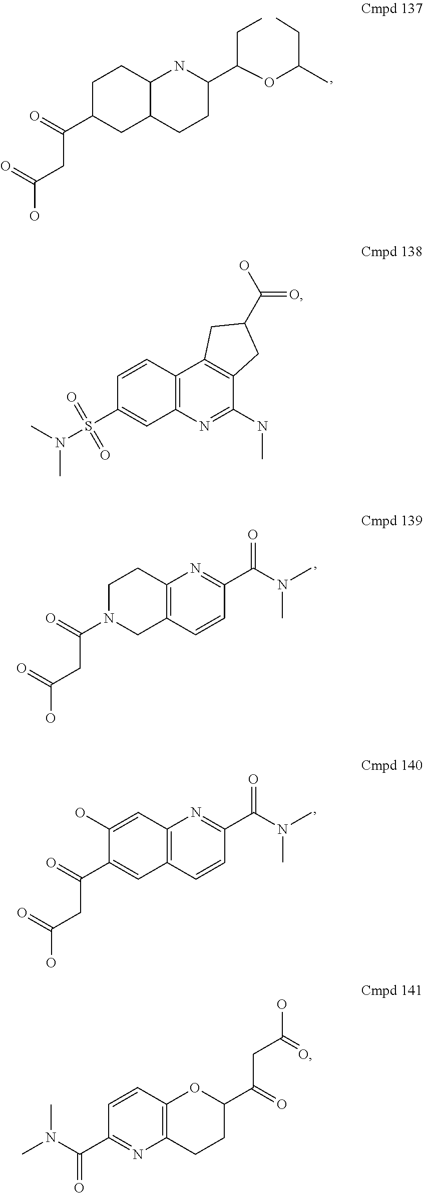

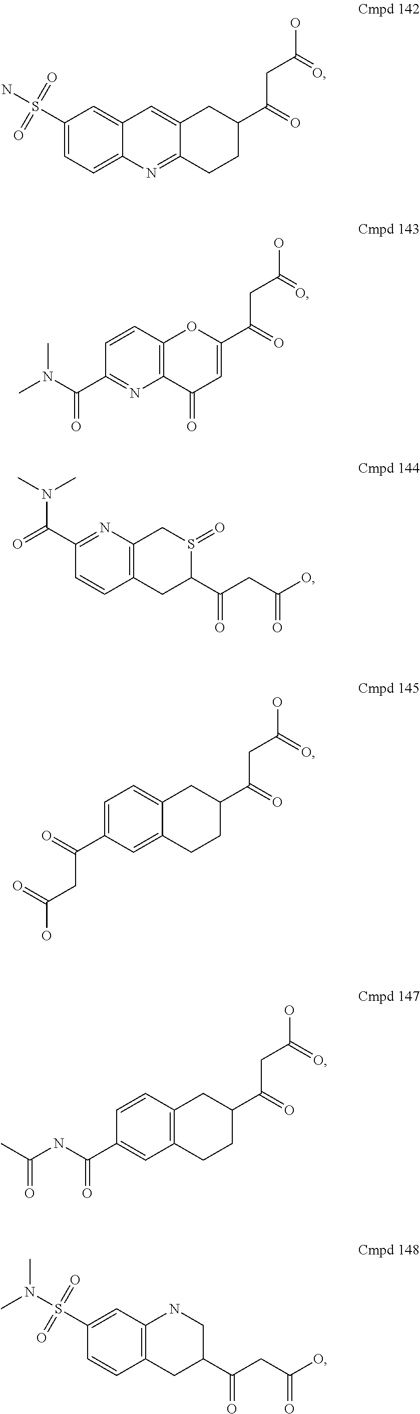

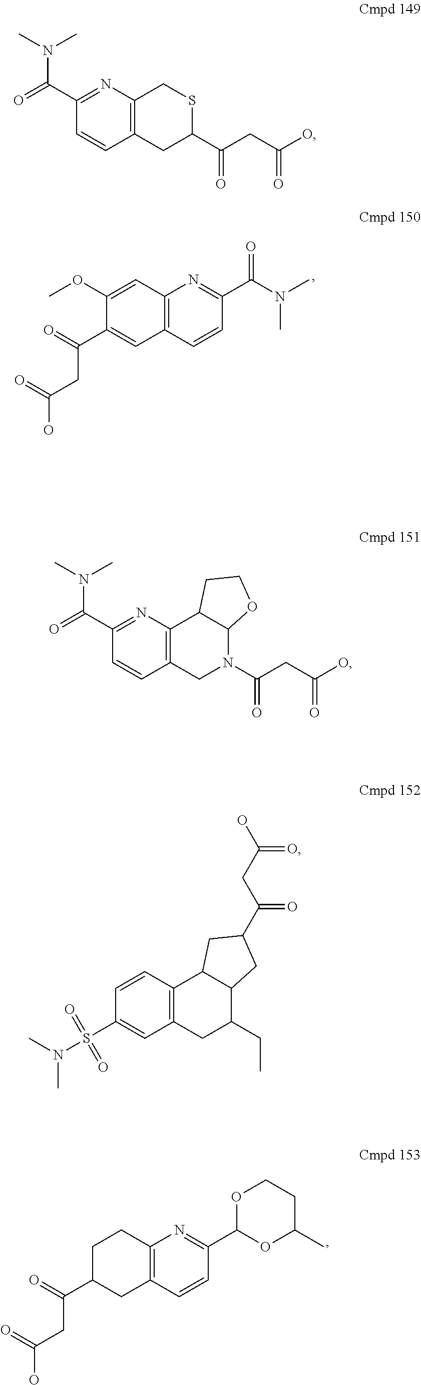

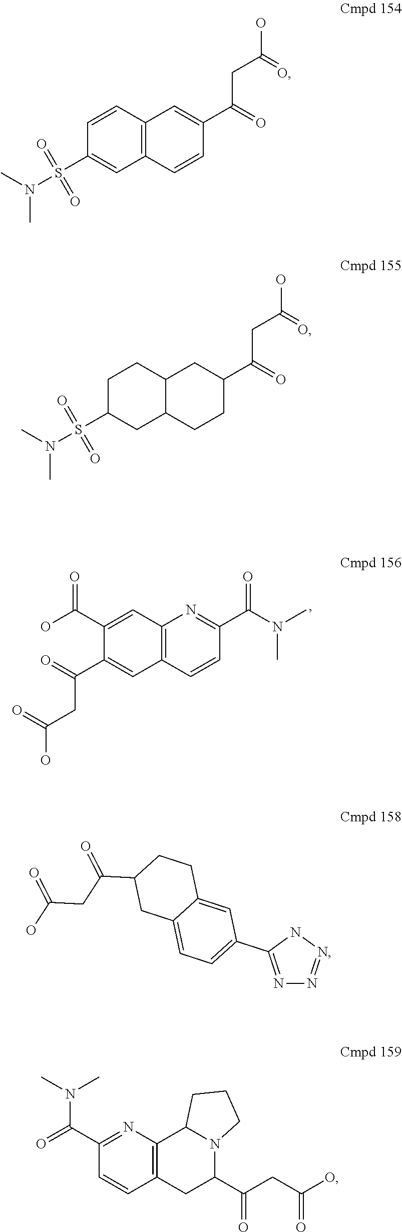

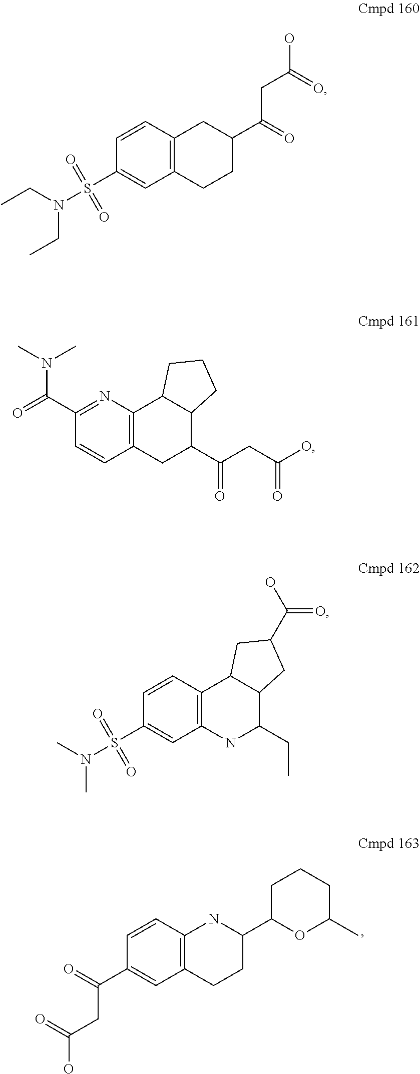

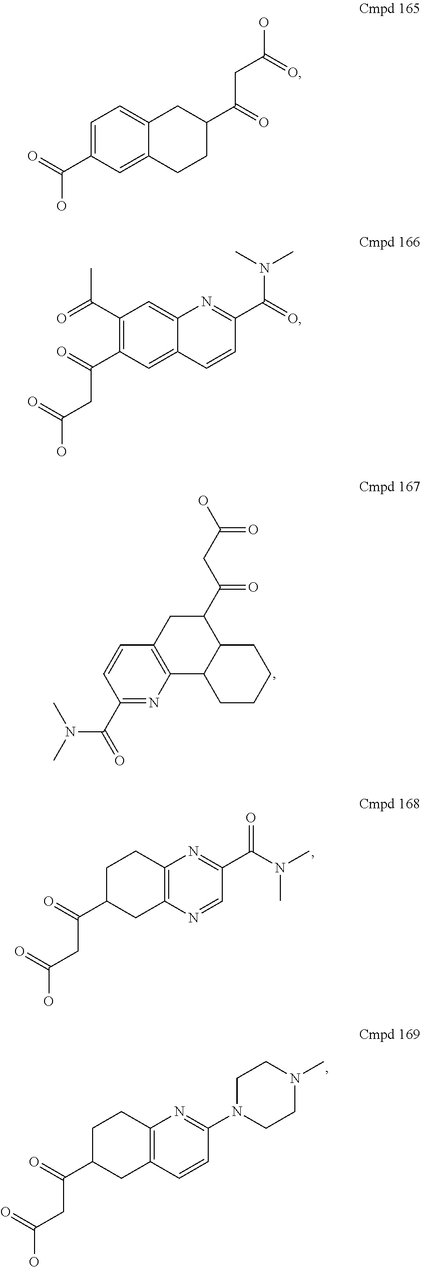

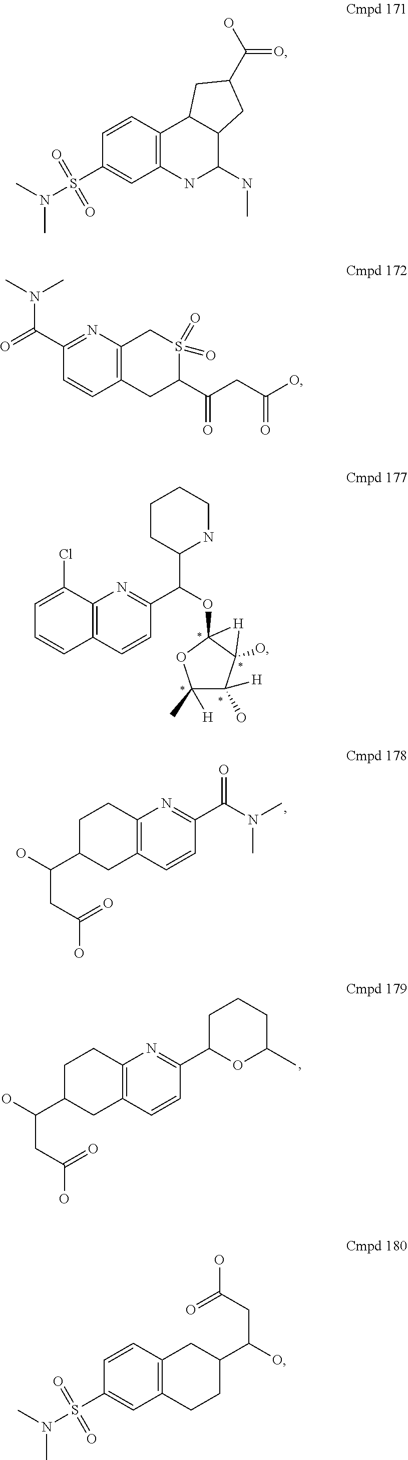

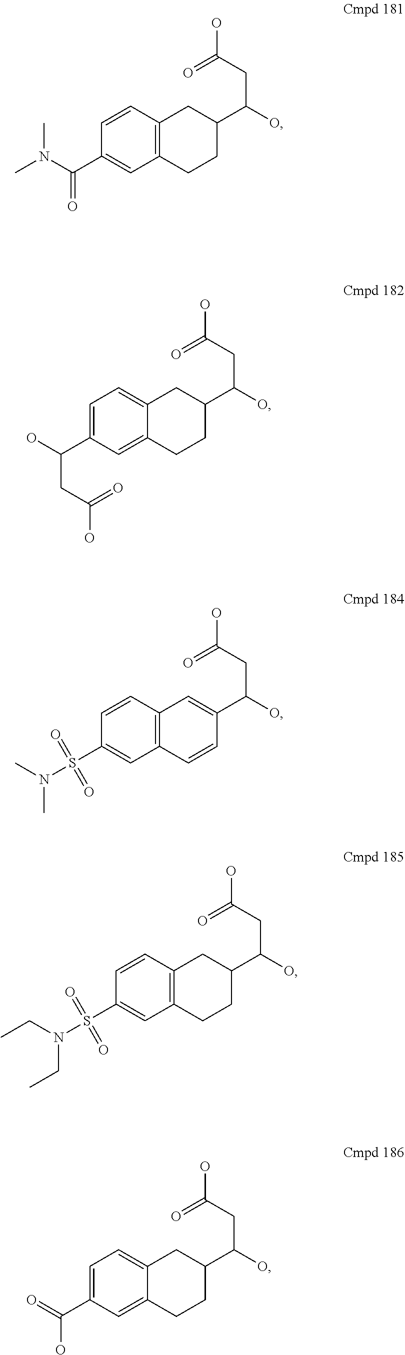

and a pharmaceutically acceptable salt, hydrate, solvate, or polymorph thereof.

In another aspect, the invention relates to compounds of formula IV or a pharmaceutically acceptable salt, hydrate, solvate, or polymorph thereof:

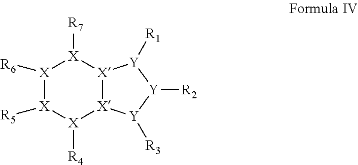

wherein,
each X is independently selected from CH or C;
X′ is C;
each Y is independently selected from N, CH, C, SO₂ or CO;
R¹ and R² are each independently H, COOH, O—CH3, lower cycloalkyl, or join together to form a five- or six-membered heterocyclic ring, which is optionally substituted;
R³-R⁷ is each independently H, COOH, O—CH3, lower cycloalkyl, or comprises the substituent of formula XVI:

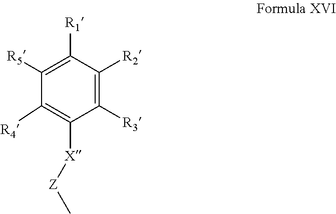

wherein,
Z is —N—CO—N or lower alkyl-CO—N;
X″ is lower alkyl or partly halogenated lower alkyl; and
R1′-R5′ are independently H; CHF₂; COOH; SO₂NH₂; NH—CH₂—COOH; lower alkyl, lower alkyl-COOH, lower alkyl-CO—, lower alkyl-COOH, —O—CO—CH₃, OCH3, N—CO—N—O-lower alkyl, N—CO—N—O—CF₂, —SO₂—N (lower alkyl)₂, lower alkyl-O-lower alkyl-COOH, SO₂—N-lower alkyl-phenylcarboxylic acid; or a substituted or non-substituted aromatic or non-aromatic heterocycle, preferably tetrazole, which may optionally be connected to the bicyclic ring of formula IV with a lower alkyl linker.

In a particular embodiment, the compound of formula IV is selected from the group consisting of:

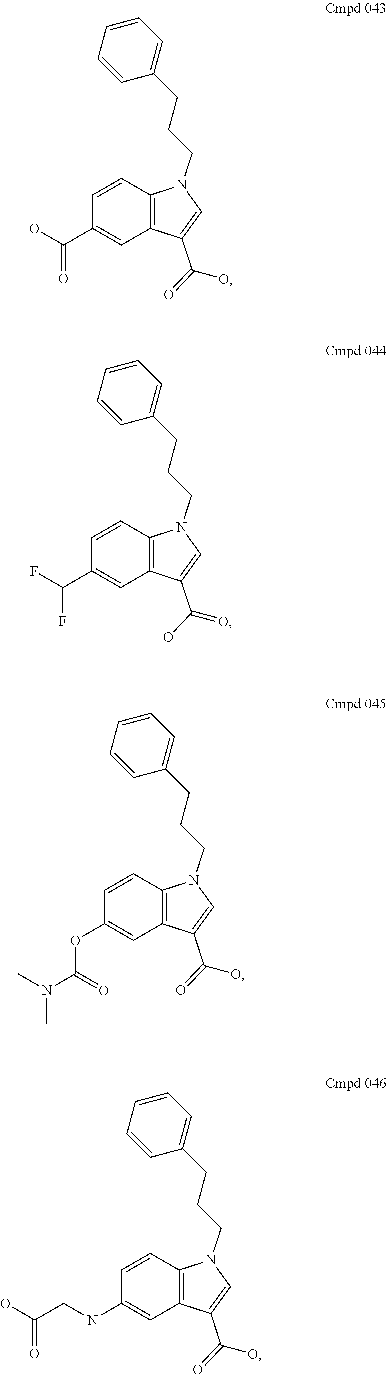

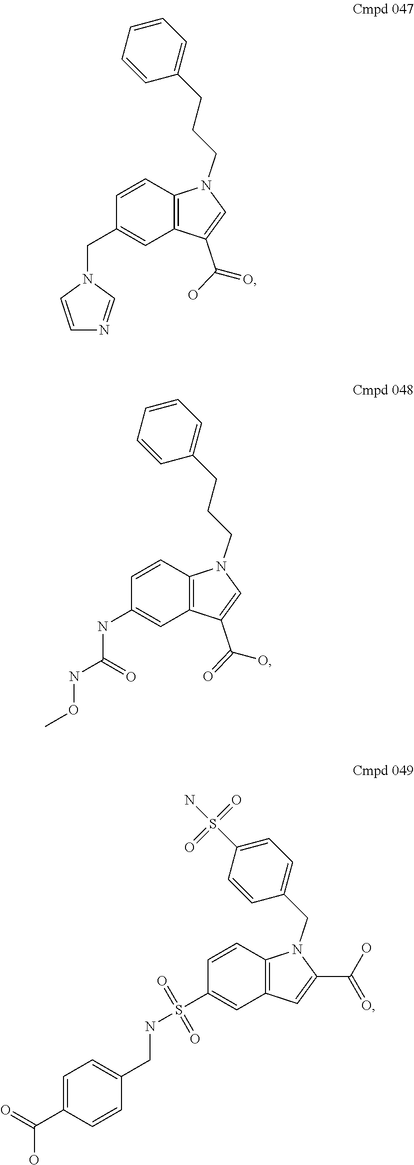

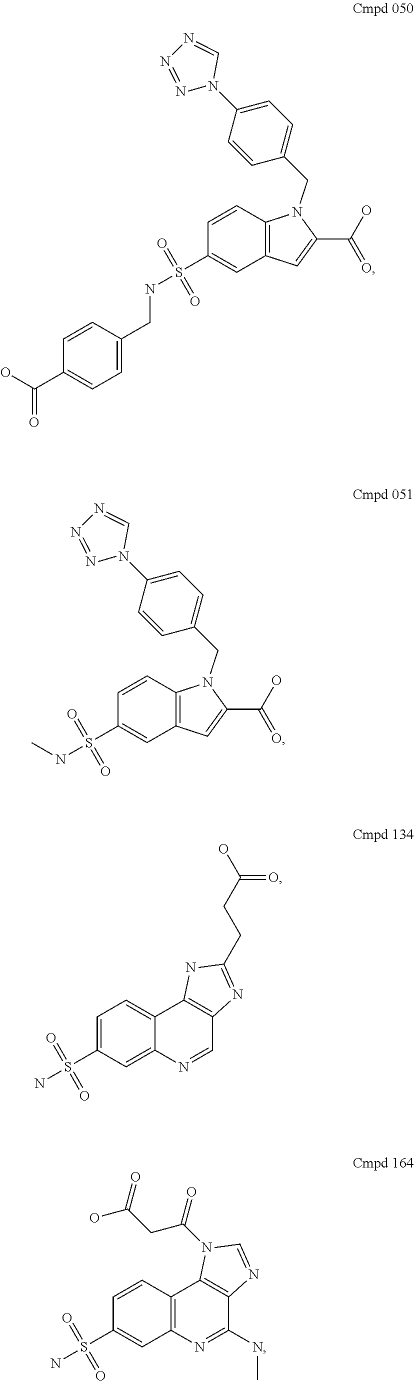

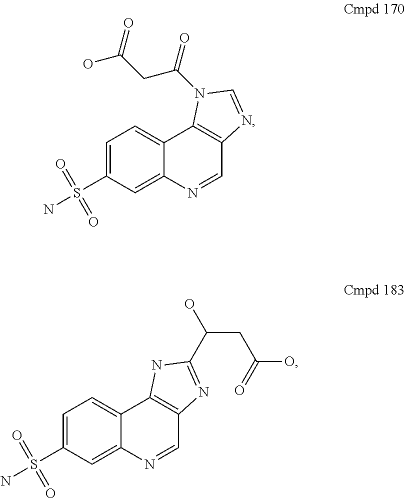

and a pharmaceutically acceptable salt, hydrate, solvate, or polymorph thereof.

In a further aspect, the invention pertains to compounds of formula V or a pharmaceutically acceptable salt, hydrate, solvate, or polymorph thereof:

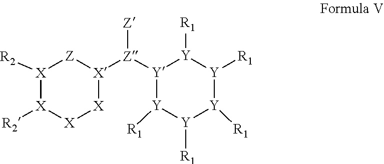

wherein,
X is CH₂, C, or CH; X′ is either C or CH; Y is C or CH; Y′ is C; Z is independently N, O, or N-lower alkyl; Z′ is O; R₂ is H, halogen, lower alkyl, ketobutyric acid, cyclohexanonecarboxylic acid or hydroxymethyl(cyclohexenone); wherein the bonds between adjacent ring substituents X, X′ and/or Z constitute single or double bonds, and wherein the bonds between adjacent Y and/or Y′ constitute single or double bonds; Z″ can be CH or N, or lower alkyl, lower cycloalkyl, alkyl ether or cycloalkyl; and R₁ can be H, lower alkyl, halogen, O-lower alkyl, COOH, N—CO—N—O-lower alkyl or cycloalkyl, partly halogenated lower alkyl or cycloalkyl, —CO—CH₃, -lower alkyl-O-phenyl carboxylic acid with a lower alkyl substituent, or

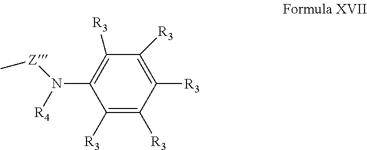

wherein,
Z′″ is lower alkyl or cycloalkyl, R₃ is each independently H, COOH, NH—CO—NH—OCH₃ or partly halogenated NH—CO—NH—CH₃, O-lower alkyl, CO—CH₃ or lower alkyl, and R₄ is lower alkyl.

In a particular embodiment, the compounds of formula V are selected from the group consisting of:

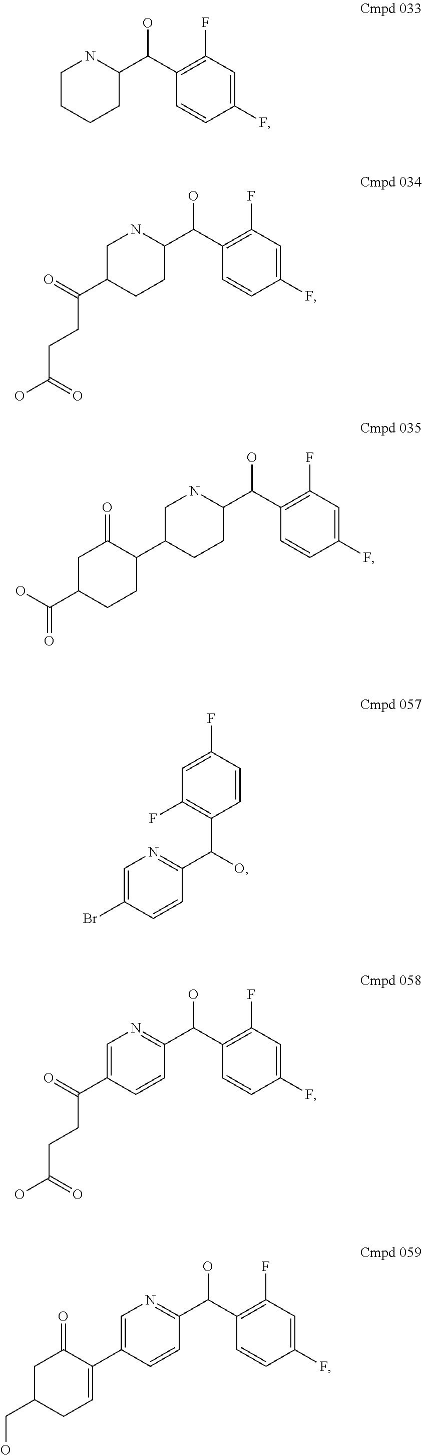

and a pharmaceutically acceptable salt, hydrate, solvate, or polymorph thereof.

In a further aspect, the invention is directed to compounds of formula VI or a pharmaceutically acceptable salt, hydrate, solvate, or polymorph thereof:

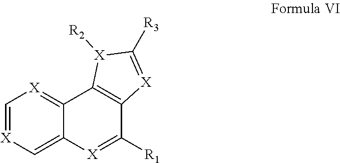

wherein,
independently X is N or CH; R¹ is NH₂, NH-lower alkyl, OH, or O-lower cycloalkyl; R² and R³ can be H, lower alkyl, SO₂NH₂ or comprise the substituent of formula XVIII:

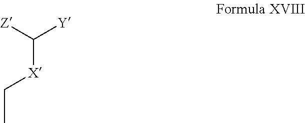

wherein,
X′ is a direct bond, NH, N—CH₃ or CH₂; Y′ is H or lower alkyl; and Z′ is H, lower alkyl, COOH, SO₂NH₂, SO₂NH—CH₃, or a substituted or non-substituted aromatic or non-aromatic heterocycle, preferably tetrazole, wherein X′ is N—CH₃ when both Y′ and Z′ are H.

In some embodiments, the compounds of formula VI are selected from the group consisting of:

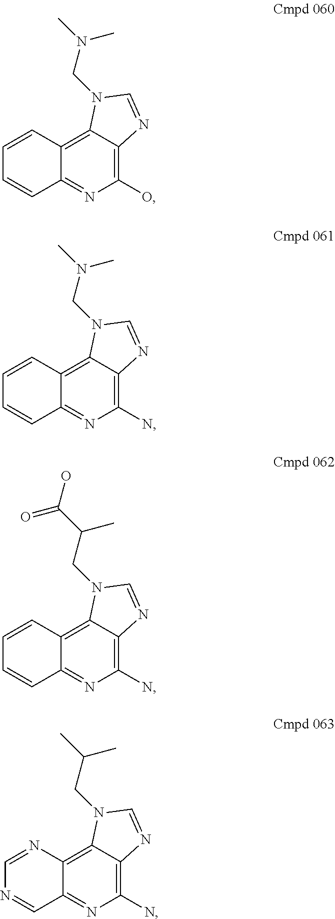

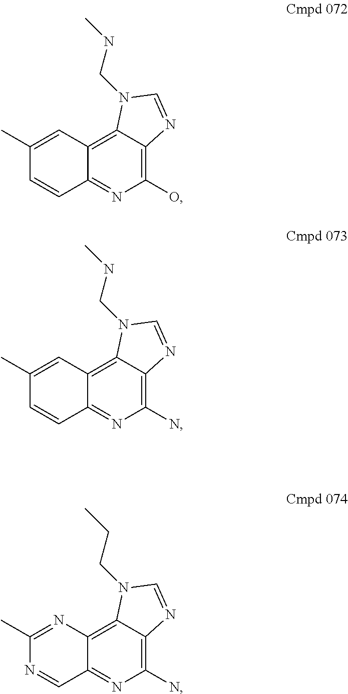

and a pharmaceutically acceptable salt, hydrate, solvate, or polymorph thereof.

In a further embodiment, the invention is directed to compounds of formula VII or a pharmaceutically acceptable salt, hydrate, solvate, or polymorph thereof:

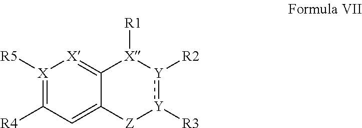

wherein,
X is N or C, X′ is CH or N; X″ is N, or CH; Y is CH or C; Z is CH₂— or C═O; wherein the bond between adjacent ring substituents Y constitute a single or double bond;
R¹ comprises a substituent of formula XIX:

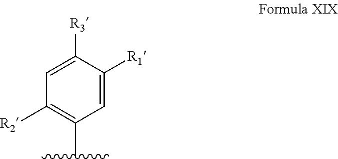

wherein,
R1′-R3′ can be independently halogen, carboxylic acid, SO₂NH₂, SO₂—NH— lower alkyl, COOH, or NO₂, or a substituted or non-substituted aromatic or non-aromatic heterocycle, preferably tetrazole; or
R1 can comprise a substituent of formula XX:

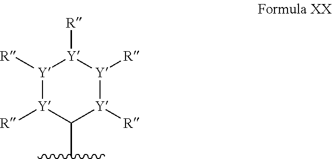

wherein,
Y′ is N, CH, or C; R″ is independently H, halogen, dihalogen or lower alkyl;
R² is H, lower cycloalkyl or a substituted or non-substituted aromatic or non-aromatic heterocycle, preferably 1,3-diazolidine;
R³ is halogen, carboxylic acid, SO₂NH₂, or SO₂—NH-lower alkyl, or a substituted or non-substituted aromatic or non-aromatic heterocycle, preferably tetrazole;
R⁴ is H, halogen, or low alkyl ketoacid, preferably ketopropionic acid;
R⁵ is absent if X is N or CH; if X is C, R5 is a substituted or non-substituted aromatic or non-aromatic heterocycle, preferably pyrrolidine, a piperidine, pyrazine, or a bicyclic structure of formula XXI:

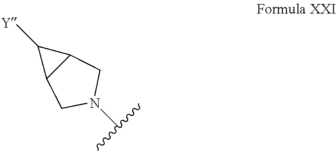

wherein
Y″ is N, N-lower alkyl, or COOH.

In a particular embodiment, the compounds of formula VII are selected from the group consisting of:

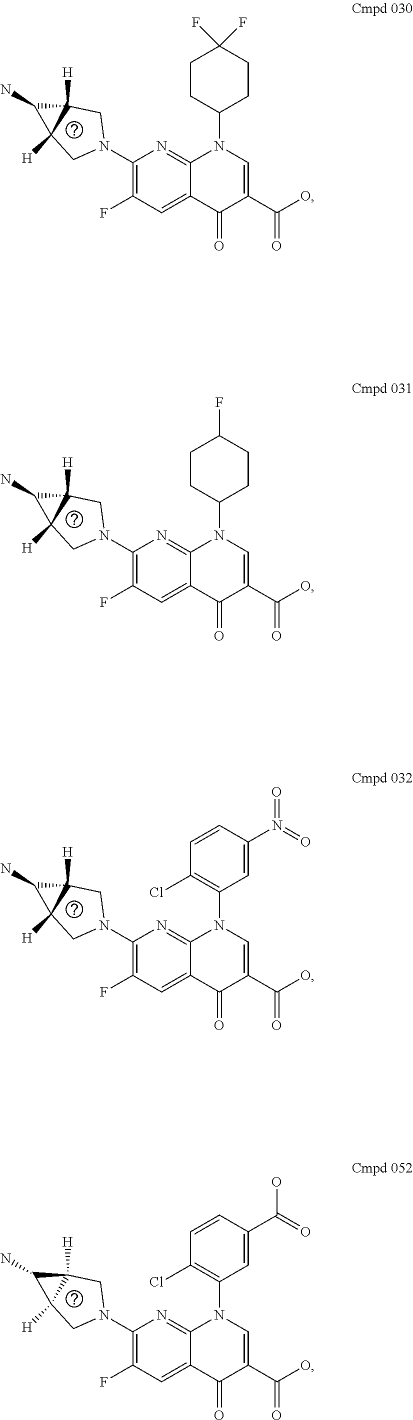

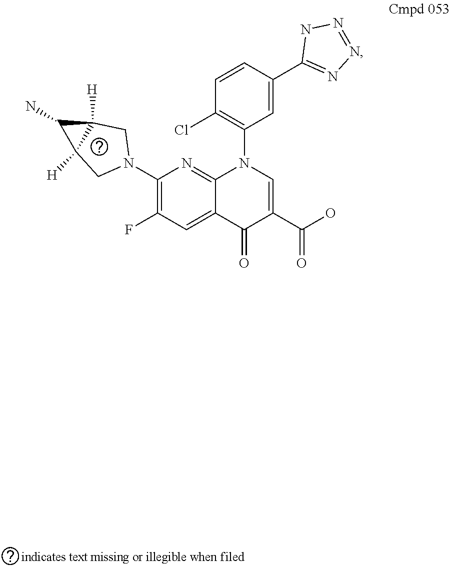 [SMILES: N[C@@H]1[C@H]2CN(c3nc4c(cc3F)c(=O)c(C(=O)O)cn4-c3cc(-c4nnn[nH]4)ccc3Cl)C[C@@H]12]

and a pharmaceutically acceptable salt, hydrate, solvate, or polymorph thereof.

In other aspects, the invention is directed to compounds of formula VIII or a pharmaceutically acceptable salt, hydrate, solvate, or polymorph thereof:

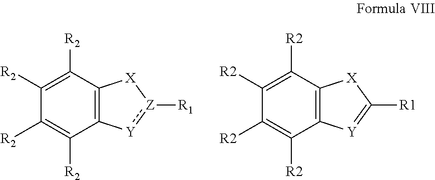

wherein,
X is SO₂, NR₄ or O; Z is C or N; Y is N, CR₃ or CH; R₁-R₄ are each independently H, oxo, halogen, —(CH₂)ₙ—COOH, wherein n=0 to 6, —NH—C(O)—NH—CH₃, —NH—C(O)—NH—CHF₂, alkyl or alkoxy, optionally substituted with COOH or oxo, or wherein one or more of R₁-R₄ comprise a substituent of Formula XXII:

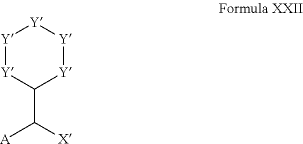

wherein,
A is attachment side, wherein the substituent of Formula XXII is attached directly or through a linker L to the compound of formula VIII, wherein L is alkyl, —CH₂—C(O)—NH—, or —NH—C(O)—NH—;
X′ is H, O, N, or N-lower cycloalkyl;
Y′ is O, N, N-lower cycloalkyl, CH₂, CR₅, CHR₅, wherein R₅ is alkyl, hydroxy, alkoxy, halogen, wherein the alkyl is optionally substituted by one or more of COOH, oxo, alkoxy, halogen, or hydroxylamine, wherein adjacent Y′ are bonded with a single or double bond, and wherein two R₅ attached to two adjacent Y′ are optionally joined to form an aromatic or non-aromatic five- or six-membered ring; preferably X′ is O and the Y′-ring contains 1 or 2 N or O atoms.

In particular embodiments, the compounds of formula VIII are selected from the group consisting of:

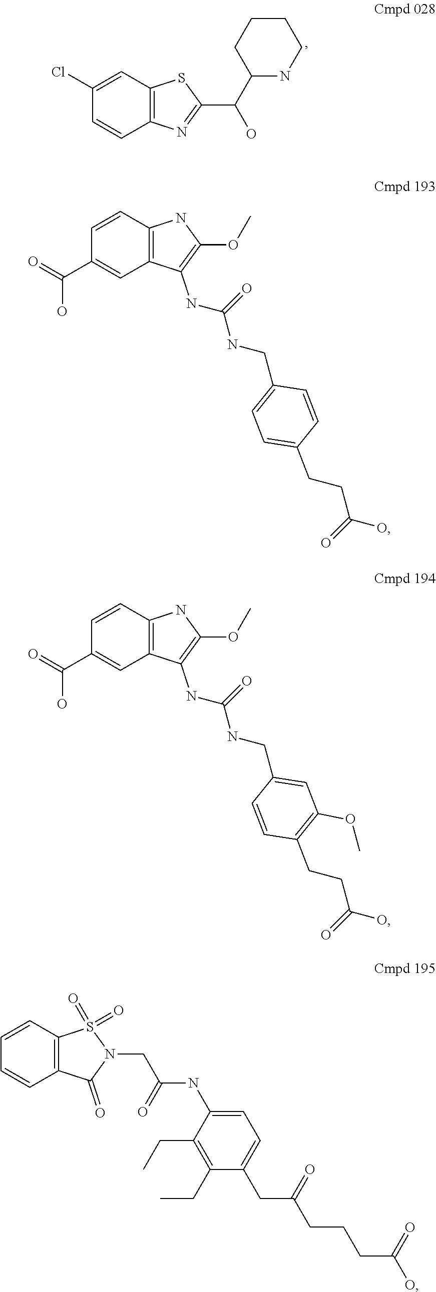

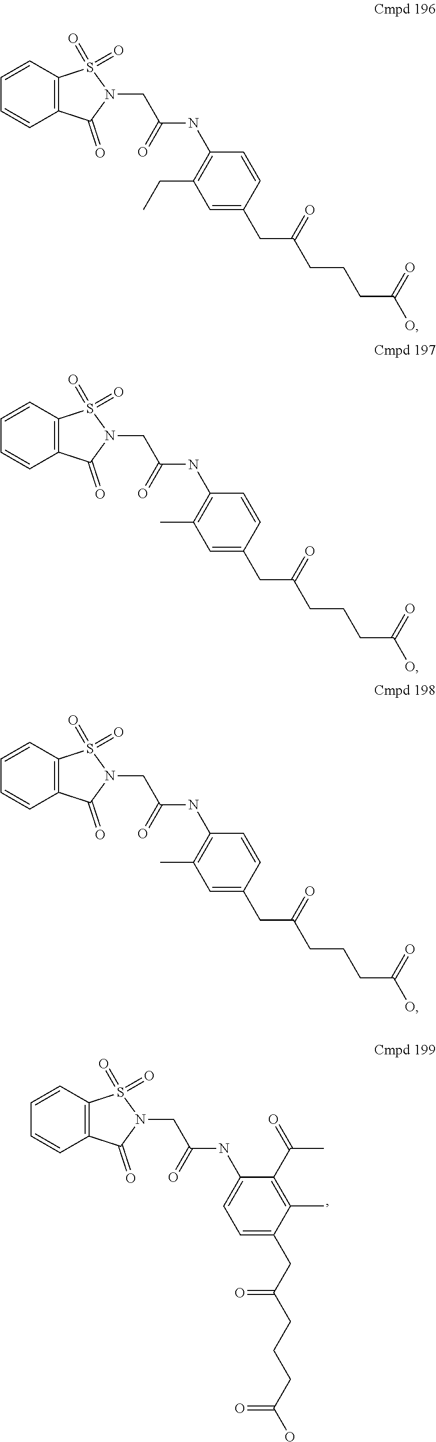

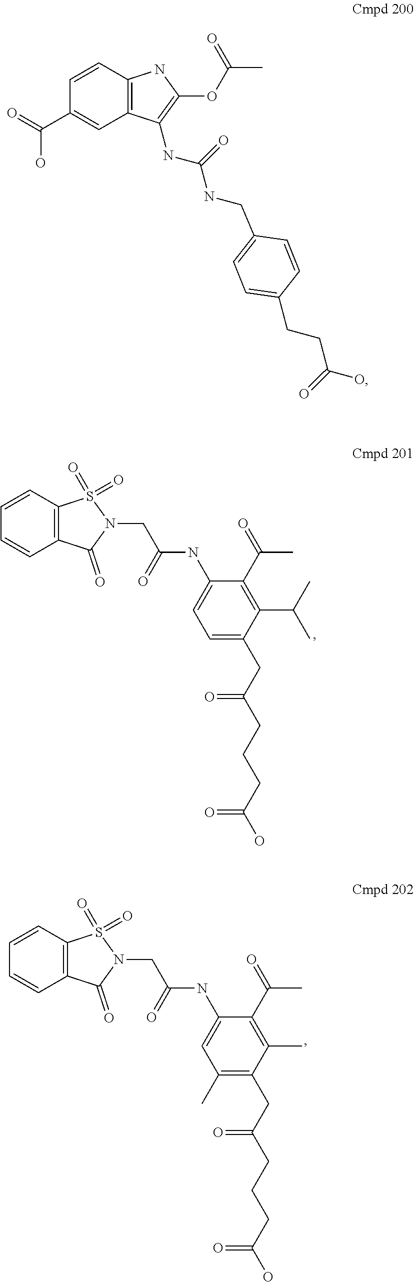

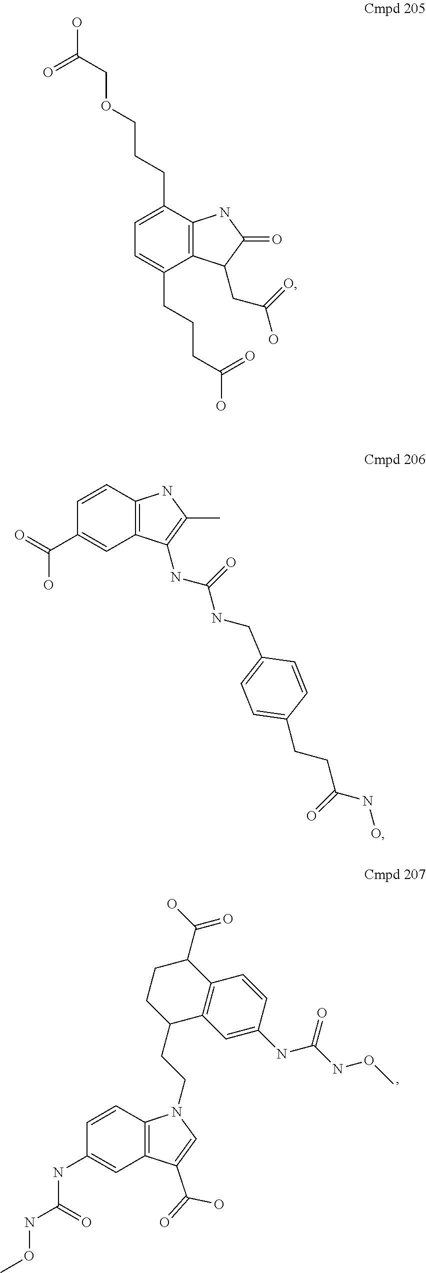

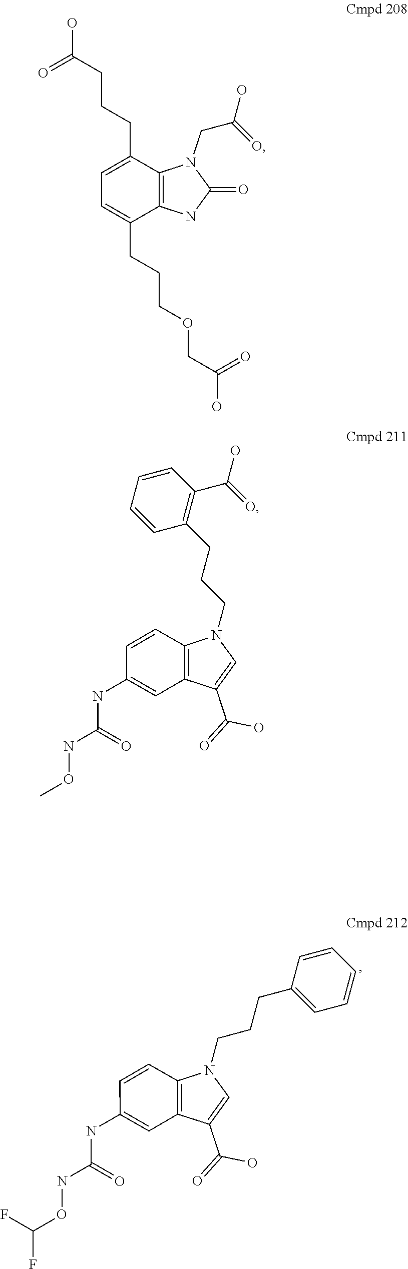 [SMILES: O=C(O)CCCc1ccc(CCCOCC(=O)O)c2[nH]c(=O)n(CC(=O)O)c12 ; CONC(=O)Nc1ccc2c(c1)c(C(=O)O)cn2CCCc1ccccc1C(=O)O ; O=C(NOC(F)F)Nc1ccc2c(c1)c(C(=O)O)cn2CCCc1ccccc1]

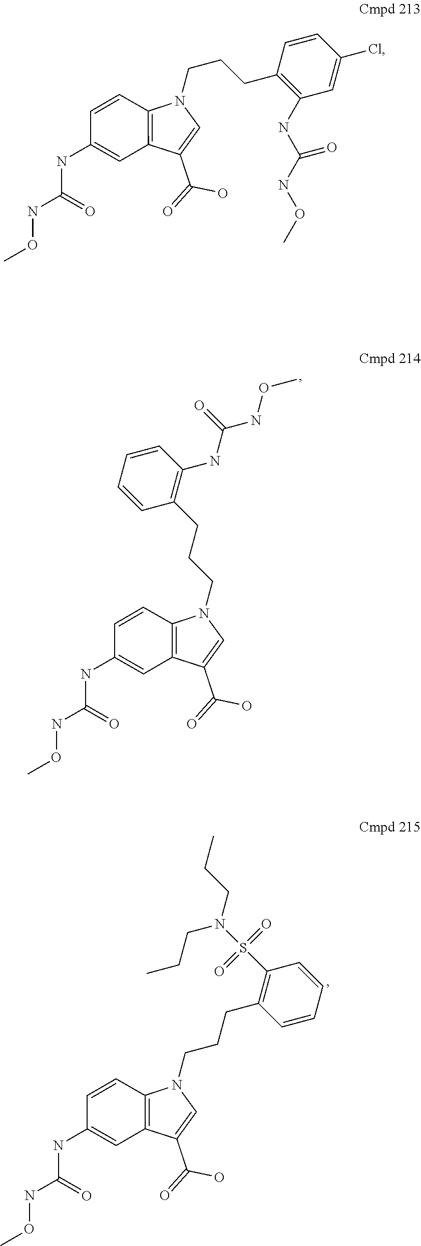

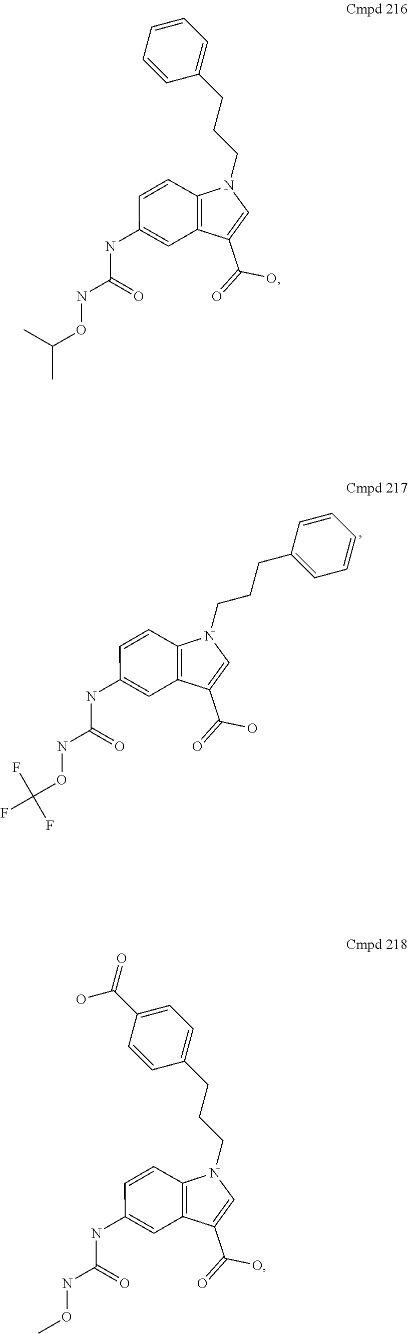

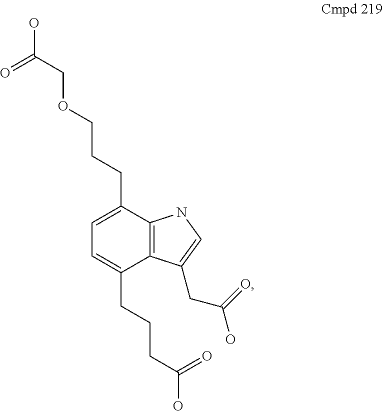 [SMILES: O=C(O)CCCc1ccc(CCCOCC(=O)O)c2[nH]cc(CC(=O)O)c12]

and a pharmaceutically acceptable salt, hydrate, solvate, or polymorph thereof.

In another aspect, the invention comprises a pharmaceutical composition comprising a compound of formula I, I′, II, III, IV, V, VI, VII, or VIII, or a salt or solvate thereof, and one or more pharmaceutically acceptable excipients.

In another aspect, the invention comprises a method of treating a disease or disorder related to PANX1 abnormal signaling comprising administering a compound of formula I, I′, II, III, IV, V, VI, VII, or VIII, or a salt or solvate thereof, to a subject in need thereof.

In a particular embodiment, the disease or disorder is selected from the group comprising chronic pain; chemotherapy-associated pain; addiction, particularly opioid addiction; epilepsy; Parkinson's Disease; Alzheimer's Disease; multiple sclerosis; traumatic brain injury; migraine; stroke; cancer, particularly melanoma, hepatocellular carcinoma, breast cancer, colorectal cancer, pancreatic cancer, and leukemia; cardiovascular diseases, particularly arrythmia, vascular inflammation, elevated blood pressure, and pulmonary arterial hypertension; inflammatory diseases, particularly joint inflammation and wound healing inflammatory disorders; pulmonary diseases, particularly Covid-19, asthma, and primary and secondary ciliary dyskinesia; fibrosis; diabetes; eye diseases; and skin diseases.

In a preferred embodiment, the disease or disorder is chronic pain. In another preferred embodiment, the disease or disorder is opioid addiction.

### DESCRIPTION OF THE FIGURES

FIG. 1 shows β-catenin protein expression in MDA-MB-231 breast cancer cells treated or untreated with the PANX1 blockers of the invention.

FIG. 2 shows E-cadherin protein expression in MDA-MB-231 breast cancer cells treated or untreated with the PANX1 blockers of the invention.

FIG. 3 shows Matrix Metalloproteinase 2 (MMP2) protein expression in MDA-MB-231 breast cancer cells treated or untreated with the PANX1 blockers of the invention.

FIG. 4 shows Matrix Metalloproteinase 9 (MMP9) protein expression in MDA-MB-231 breast cancer cells treated or untreated with the PANX1 blockers of the invention.

FIG. 5 shows tissue invasiveness potential of MDA-MB-231 cells cultured in Matrigel treated or untreated with the PANX1 blockers of the invention.

FIG. 6 shows the dissected biceps femoris muscle of a rat with the exposed sciatic nerve (1) and its further division into the sural (4), peroneal (2) common, and tibial (3) nerves.

FIG. 7 shows the effect of intrathecal administration of Cmpd 004, carbenoxolone (Cbx) or Saline on the withdrawal threshold of neuropathic rats.

FIG. 8 shows the effect of intrathecal administration of Cmpd 011, Cbx, or Saline on the withdrawal threshold of neuropathic rats.

FIG. 9 shows the effect of intrathecal administration of Cmpd 054, Cbx, or Saline on the withdrawal threshold of neuropathic rats.

FIG. 10 shows the effect of intrathecal administration of Cmpd 055, Cbx, or Saline on the withdrawal threshold of neuropathic rats.

FIG. 11 shows the effect of intrathecal administration of Cmpd 019, Cbx, or Saline on the withdrawal threshold of neuropathic rats.

FIG. 12 shows the effect of intrathecal administration of Cmpd 027, Cbx, or Saline on the withdrawal threshold of neuropathic rats.

FIG. 13 shows the effect of intrathecal administration of Cmpd 043, Cbx, or Saline on the withdrawal threshold of neuropathic rats.

FIG. 14 shows the effect of either Cmpd 004 or Cmpd 011 over the withdrawal score of rats in model of opioid-induced addiction.

FIGS. 15A-15B show ATP release by astrocytes (FIG. 15A) and microglial cells (FIG. 15B) in vitro upon stimulation with lipopolysaccharide in the presence or absence of the PANX1 blockers of the invention.

### DETAILED DESCRIPTION OF THE INVENTION

The present invention provides purine, indole, piperidine, pyrido(1,2-a)benzimidazole, napthyridine, imidazoquinolines, imidazo-triazanaphthalene and isoquinoline derivatives, their salts, hydrates, solvates, and/or polymorphs, which are PANX1 modulators. Pharmaceutical compositions comprising said compounds or their salts, hydrates, solvates, and/or polymorphs and pharmaceutically acceptable excipients are also provided. Said compounds salts, hydrates, solvates, and/or polymorphs may be for use in the treatment of diseases wherein panx-1 overactivity is implicated. Therefore, the invention also provides methods of treating patients suffering from diseases related to PANX1 overactivity, wherein a compound of the present invention or one of its salts, hydrates, solvates, and/or polymorphs is administered to the patient in need thereof. Finally, the invention also provides the use of the panx-1 modulators described herein, or their salts, hydrates, solvates, and/or polymorphs, to prepare a medicament for preventing or treating a disease wherein PANX1 overactivity is implicated.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise.

It will be appreciated that the same thing can be said in more than one way. Consequently, alternative language and synonyms may be used for any one or more of the terms discussed herein. No special significance is to be placed upon whether a term is elaborated or discussed herein. Synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any exemplified term. Likewise, the disclosure is not limited to various embodiments given in this specification.

As used herein, modulators of PANX1 denote low molecular weight molecules that bind to PANX1 and interfere with its activity. A modulator may be classified according to its effect on PANX1 as antagonist or agonist, which can be further classified as full, partial or inverse agonist. Agonists are modulators that binds to a receptor and alters the receptor state resulting in a biological response. If the induced response is maximal, the modulator is said to be a full agonist; if the response is lower than maximal at the agonist highest concentrations, the modulator is a partial agonist. If the modulator binds to the PANX1 reducing its fraction in an active conformation, then the ligand is an inverse agonist. Lastly, an antagonist is a modulator that prevents an agonist of a receptor from binding to it or, if bound, is prevented from inducing conformational changes on the receptor that can result in signal transduction, thus inhibiting signal transduction.

In the context of the present invention, the PANX1 modulators can prevent the ATP outflow from cells upon PANX1 activation. The prevention of ATP outflow through PANX1 channels promoted by the compounds of the present invention may be further referred to as blockage. Therefore, the PANX-1 modulators of the present invention may be PANX-1 blockers.

As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, an alkyl group may be a lower alkyl group, wherein a lower alkyl group comprises 10 or fewer carbon atoms in its backbone. Likewise, lower cycloalkyls have from about 3 to about 10 carbon atoms in their ring structure, and alternatively about 5, 6 or 7 carbons in the ring structure.

As used herein, the term “alkenyl” is given its ordinary meaning in the art and may include monounsaturated or polyunsaturated aliphatic groups containing one or more carbon-carbon double bonds, including straight-chain alkenyl groups, branched chain alkenyl groups, which may be unsubstituted or substituted with alkyl, alkenyl, alkynyl, hydroxy, carboxy, alkoxy, heteroalkyl, heterocyclic, aryl, heteroaryl, oxo, and amino groups. In some embodiments, the carbon-carbon double may be internal or terminal. In some embodiments, an alkenyl group may be a lower alkenyl group, wherein a lower alkenyl group comprises 10 or fewer carbon atoms in its backbone, 6 or fewer carbon atoms, or alternatively 5 or fewer carbon atoms.

As used herein, the term “alkynyl” is given its ordinary meaning in the art and may include unsaturated aliphatic groups containing one or more carbon-carbon triple bonds, and may be unsubstituted or substituted with alkyl, alkenyl, alkynyl, hydroxy, carboxy, alkoxy, heteroalkyl, heterocyclic, aryl, heteroaryl, oxo, and amino groups. In some embodiments, the carbon-carbon triple bond may be internal or terminal. In some embodiments, an alkynyl group may be a lower alkynyl group, wherein a lower alkenyl group comprises 10 or fewer carbon atoms in its backbone, 6 or fewer carbon atoms, or alternatively 5 or fewer carbon atoms.

The term “heteroalkyl”, as used herein, is given its ordinary meaning in the art and refers to alkyl groups as described herein in which one or more atoms is a heteroatom (e.g., oxygen, nitrogen, sulfur, and the like). Examples of heteroalkyl groups include, but are not limited to, alkoxy, poly(ethylene glycol)-, alkyl-substituted amino, tetrahydrofuranyl, piperidinyl, morpholinyl, etc.

In the context of the present invention, the term “aryl” refers to aromatic groups, optionally substituted, having a single ring (e.g., phenyl), multiple rings (e.g., biphenyl), or multiple fused rings in which at least one is aromatic (e.g., 1,2,3,4-tetrahydronaphthyl, naphthyl, anthryl, or phenanthryl). That is, at least one ring may have a conjugated pi electron system, while other, adjoining rings can be cycloalkyls, cycloalkenyls, cycloalkynyls, aryls, and/or heterocyclyls. The aryl group may be optionally substituted, as described herein.

The term “carbocyclic aryl groups” refers to aryl groups wherein the ring atoms on the aromatic ring are carbon atoms. Carbocyclic aryl groups include monocyclic carbocyclic aryl groups and polycyclic or fused compounds (e.g., two or more adjacent ring atoms are common to two adjoining rings) such as naphthyl groups. In some cases, the aryl groups may include monocyclic carbocyclic aryl groups and polycyclic or fused compounds (e.g., two or more adjacent ring atoms are common to two adjoining rings) such as naphthyl group. Non-limiting examples of aryl groups include phenyl, quinolinyl, naphthyl, tetrahydronaphthyl, indanyl, indenyl, pyrido(1,2-a)benzimidazole, imidazoquinolines and the like.

The term “heteroaryl” is given its ordinary meaning in the art and refers to aryl groups as described herein in which one or more atoms is a heteroatom (e.g., oxygen, nitrogen, sulfur, and the like), optionally substituted. Examples of aryl and heteroaryl groups include, but are not limited to, phenyl, pyrrolyl, furanyl, thiophenyl, benzothiazolyl, imidazolyl, benzoimidazolyl, imidazoquinolinyl, napthyridinyl, oxazolyl, thiazolyl, triazolyl, pyrazolyl, pyridinyl, pyrazinyl, pyridazinyl and pyrimidinyl, and the like.

Atoms making up the compounds of the present disclosure are intended to include all isotopic forms of such atoms. “Isotopes,” as used herein, include those atoms having the same atomic number but different mass numbers. By way of general example and without limitation, isotopes of hydrogen include tritium and deuterium, and isotopes of carbon include ¹³C and ¹⁴C. In addition, it is contemplated that one or more carbon atoms of a compound may be replaced by a silicon atom. Furthermore, it is contemplated that one or more oxygen atoms of a compound may be replaced by a sulfur or selenium atoms.

A compound having a formula that is represented with a dashed bond is intended to include the formula optionally having zero, one or more double bonds. Any undefined valency on an atom of a structure shown in this application implicitly represents a hydrogen atom bonded to the atom.

The term “pharmaceutically acceptable” is meant that the ingredients of the pharmaceutical composition must be compatible with each other and not deleterious to the recipient thereof. In this sense, “pharmaceutically acceptable salt” refers to a salt which possesses the effectiveness of the parent compound and which is not biologically or otherwise undesirable (e.g., is neither toxic nor otherwise deleterious to the recipient thereof).

As used herein, “diseases wherein PANX1 is implicated” refers to a disease or disorder characterized by inappropriate PANX1 activity. Inappropriate PANX1 activity refers to either an increase or decrease in PANX1 activity as measured by cellular assays, for example, compared to the activity in a healthy cell or subject. Inappropriate activity could also be due to overexpression of PANX1 in diseased tissue compared with healthy adjacent tissue.

The term “therapeutic activity” as used herein refers to a demonstrated or potential biological activity whose effect is consistent with a desirable therapeutic outcome in humans, or to desired effects in non-human mammals or in other species or organisms. A given PANX1 modulator may have one or more therapeutic activities, however, the term “therapeutic activities’ as used herein may refer to a single therapeutic activity or multiple therapeutic activities. “Therapeutic activity” includes the ability to induce the desired response and may be measured in vivo or in vitro. For example, a desirable effect may be assayed in cell culture, isolated tissues, animal models, clinical evaluation, EC₅₀ assays, IC₅₀ assays, or dose-response curves. The term therapeutic activity includes preventive or curative treatment of a disease, disorder, or condition. Treatment of a disease, disorder or condition can include improvement of a disease, disorder or condition by any amount, including the elimination of a disease, disorder or condition.

As used herein, the acronyms IC and EC stand for, respectively, “Inhibitory Concentration” and “Effective Concentration” and the notation IC₅₀ and EC₅₀ denote the half-maximal inhibition or activation of a particular biological phenomenon (e.g. ATP release) promoted by a compound in an in vitro assay.

The term “therapeutically effective” as used herein depends on the condition of a subject and the specific compound administered. The term refers to an amount effective to achieve a desired clinical effect. A therapeutically effective amount varies with the nature of the condition being treated, the length of time that activity is desired, and the age and the condition of the subject, and ultimately is determined by the health care provider.

Herein, the term “treating” includes abrogating, substantially inhibiting, slowing or reversing the progression of a disease or disorder, substantially ameliorating clinical symptoms of a disease or disorder or substantially preventing the appearance of clinical symptoms of a disease or disorder.

### Pannexin-1 Modulators

The PANX1 modulators of the present invention may be compounds of Formula I, II, III, IV, V, VI, or VII, which are, respectively, purine, indole, naphthyridine, piperidine, pyrido(1,2-a)benzimidazole, quinoline and isoquinoline derivatives.

The invention relates to compounds with PANX1 modulatory activity. In some embodiments, said compounds are a compound of formula I or a pharmaceutically acceptable salt, hydrate, solvate, or polymorph thereof:

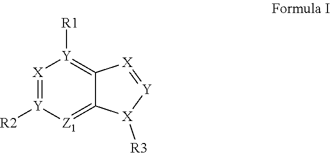

wherein,
X and Y are individually C, CH or N, or preferably wherein Y is C, or CH and X is N;
R¹, R² and R³ are independently hydrogen; lower alkyl, optionally substituted with hydroxy; lower cycloalkyl; lower alkenyl; lower alkoxy; lower alkynyl; phenyl; halogenated phenyl; halogen; a substituted or non-substituted aromatic or non-aromatic heterocycle, preferably diazine, pyrazole, diazole, triazole, or their alkyl derivatives; 2-hydroxyisopropyl; or have one of the following structures:

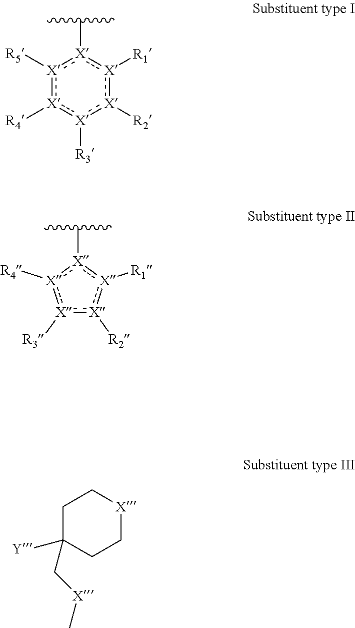

wherein,
X′ is C, CH, CH₂, N, S, or O, or preferably can be CH₂, N, or O;
X″ is independently C, CH, CH₂, N, S, or O, preferably CH₂, N, or O;
X′″ is NH, N-lower alkyl or O;
R1′-R5′ are independently absent, hydrogen, lower alkyl, lower cycloalkyl, halogen, OH, NH₂, hydroxy-lower alkyl, or NH-lower alkyl, O-lower alkyl, O—PO—OR₆′, O-Lower alkyl or alkyloxy-O—PO—O—R₆′, or O—PO—O-lower alkyl-O—PO—R₆′, preferably OH, NH₂, NH-lower alkyl, wherein vicinal R1′ and R5′ may form a 5-7 membered ring moiety;
R₆′ is H or O-lower alkyl;
R1″-R4″ are independently absent, hydrogen, lower alkyl, lower cycloalkyl, OH, NH₂, hydroxy-lower alkyl, NH-lower alkyl, O-lower alkyl, O—PO—OR₆′, O-lower alkyl or alkyloxy-O—PO—O—R₆′, or O—PO—O-lower alkyl-O—PO—R₆′, preferably OH, NH₂, NH-lower alkyl, or halogen;
Y′″ is halogen; and
wherein the bonds between adjacent ring substituents X′ or adjacent ring substituents X″ may constitute single or double bonds.

In particular embodiments, the compound of Formula I may be selected from the group consisting of:

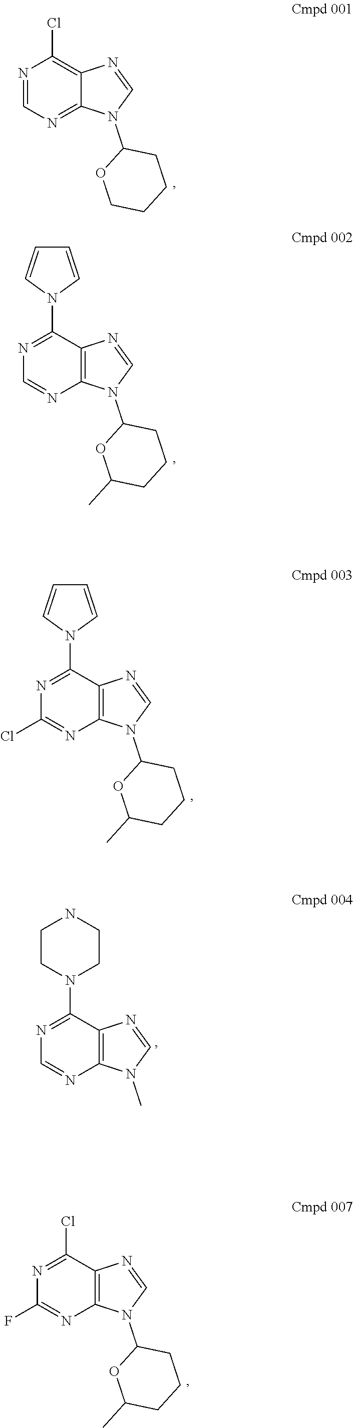 [SMILES: Clc1ncnc2c1ncn2C1CCCCO1 ; CC1CCCC(n2cnc3c(-n4cccc4)ncnc32)O1 ; CC1CCCC(n2cnc3c(-n4cccc4)nc(Cl)nc32)O1 ; Cn1cnc2c(N3CCNCC3)ncnc21 ; CC1CCCC(n2cnc3c(Cl)nc(F)nc32)O1]

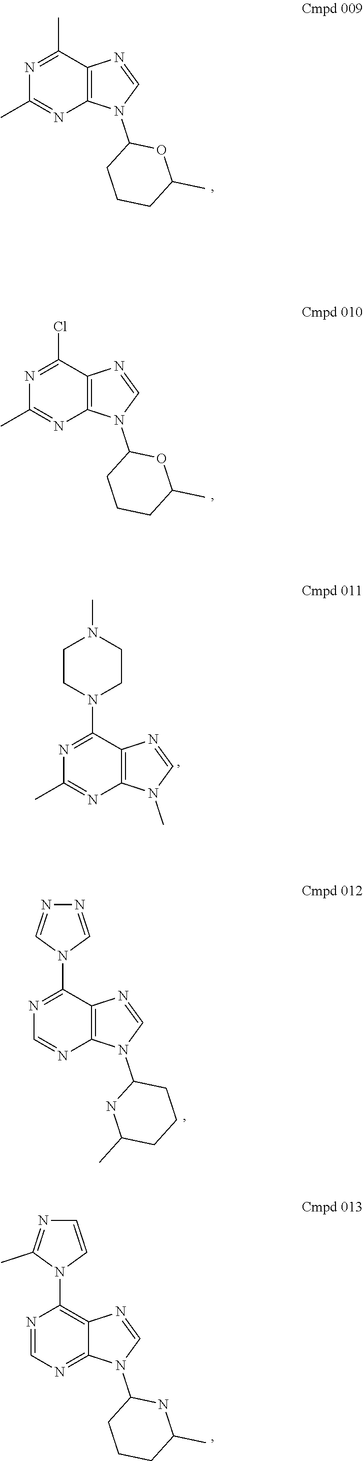

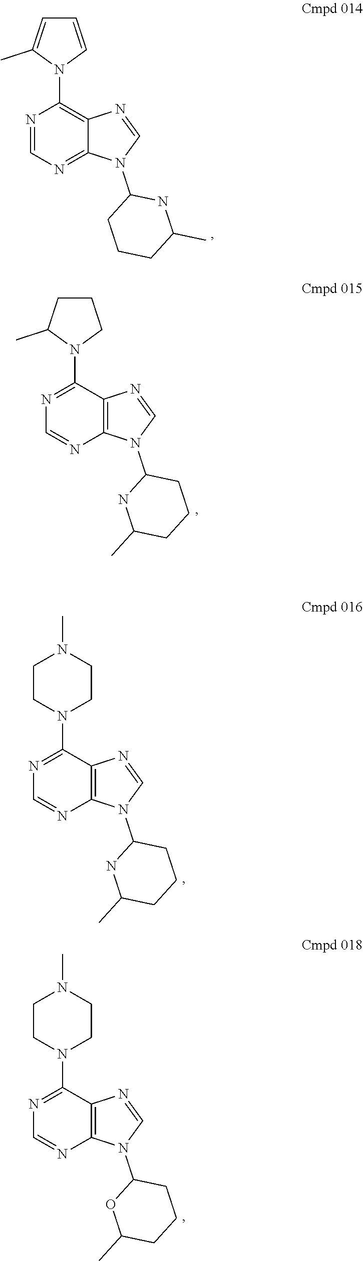

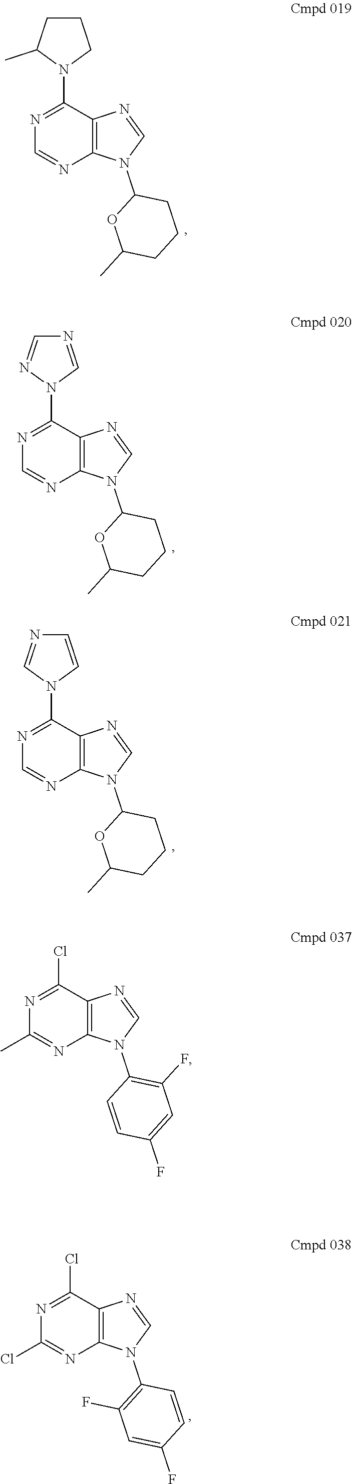

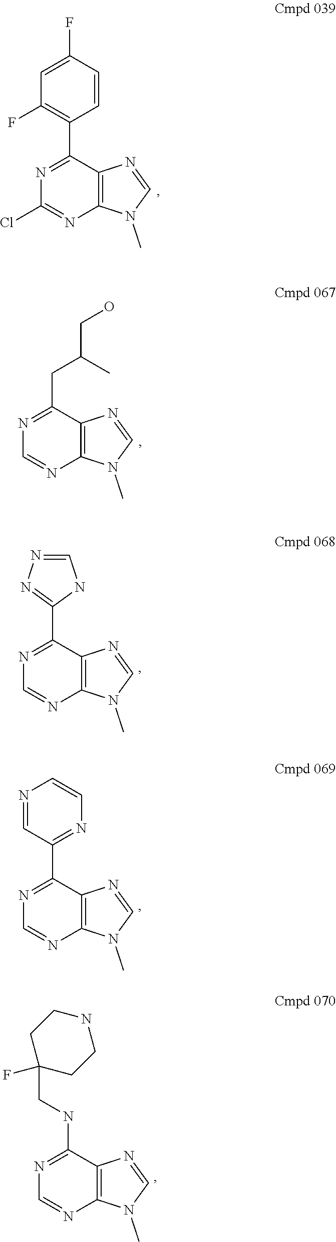 [SMILES: Cn1cnc2c(-c3ccc(F)cc3F)nc(Cl)nc21 ; CC(CO)Cc1ncnc2c1ncn2C ; Cn1cnc2c(-c3nnc[nH]3)ncnc21 ; Cn1cnc2c(-c3cnccn3)ncnc21 ; Cn1cnc2c(NCC3(F)CCNCC3)ncnc21]

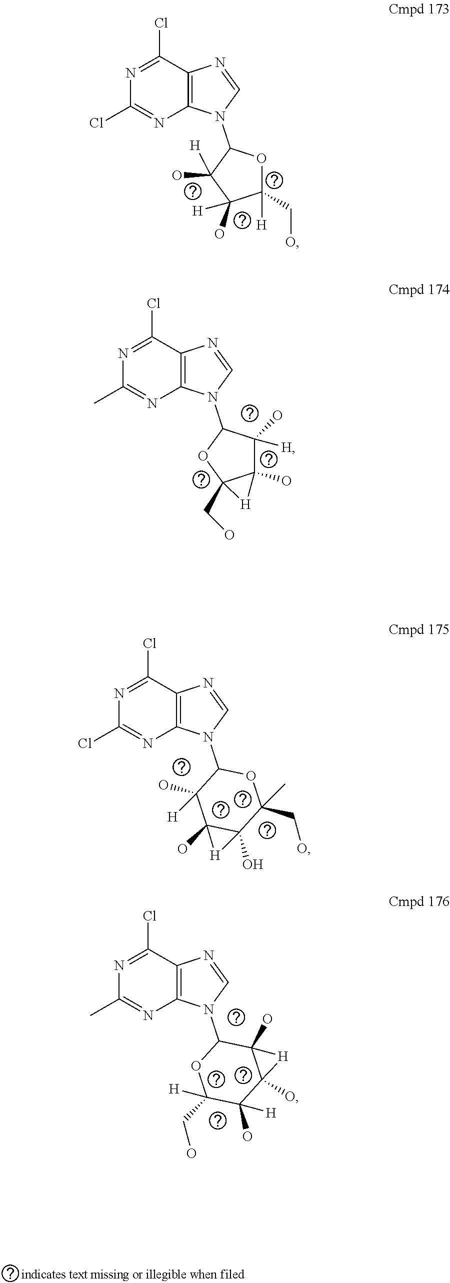

and a pharmaceutically acceptable salt, hydrate, solvate, or polymorph thereof.

In some aspects of the invention, as subclass of the preceding class of compounds may further assume Formula I′, or a pharmaceutically acceptable salt, hydrate, solvate, or polymorph thereof:

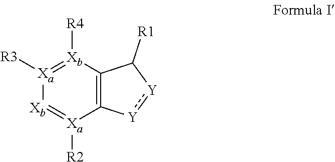

wherein,
Xₐ or X_{b} is C, CH or N, or preferably Xₐ is C or CH, and X_{b} is N; Y is CH, CH₂, C-lower alkyl, CH-lower alkyl, C-halogen, CH-halogen or C-dihalogen; Z is CH or N, preferably CH; wherein the bond between adjacent ring substituents Y may constitute a single or double bond;
R¹, R², R³, and R⁴ are independently absent, hydrogen; lower alkyl; lower cycloalkyl; lower alkenyl; lower alkoxy; lower alkynyl; phenyl; halogenated phenyl; halogen; a substituted or non-substituted aromatic or non-aromatic heterocycle, preferably triazole, or diazine; 2-hydroxyisopropyl, or have one of the following structures:

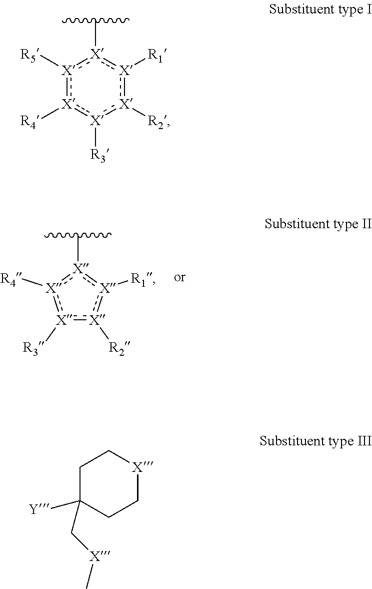

wherein,
X′ is C, CH, CH₂, N, S or O, preferably CH₂, N, or O;
X″ is C, CH, CH₂, N, S or O, preferably CH₂, N, or O;
X′″ is NH, N-lower alkyl or O;
R1′-R5′ are independently absent, or are H, lower alkyl, lower cycloalkyl, OH, NH₂, NH-lower alkyl, halogen, O-lower alkyl, O—PO—OR₆′, O-Lower alkyl or alkyloxy-O—PO—O—R₆′, or O—PO—O-lower alkyl-O—PO—R₆′, preferably OH, NH₂, NH-lower alkyl, wherein vicinal R1′ and R5′ may form a 5-7 membered ring moiety;
R₆′ is H or O-lower alkyl;
R1″-R4″ are independently absent, or are hydrogen, lower alkyl, lower cycloalkyl, or halogen;
Y′″ is halogen, and
wherein the bonds between adjacent ring substituents X′ or adjacent ring substituents X″ may constitute single or double bonds, forming thereby a non-aromatic or aromatic ring.

In particular embodiments, compounds of Formula I′ may be selected from the group consisting of:

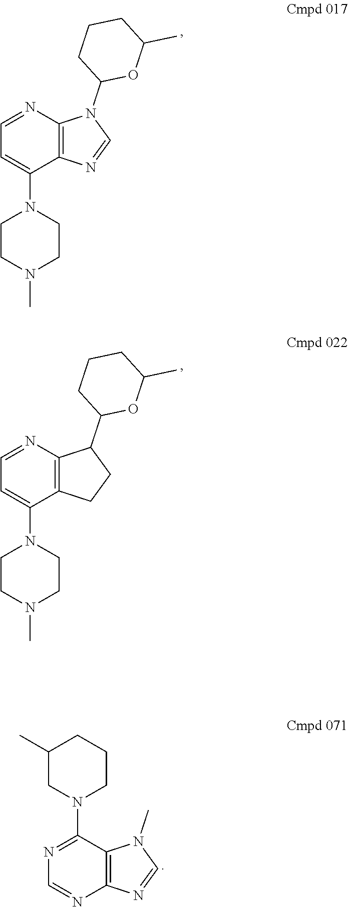

and a pharmaceutically acceptable salt, hydrate, solvate, or polymorph thereof.

Another aspect of the invention comprises compounds of Formula II or a pharmaceutically acceptable salt, hydrate, solvate, or polymorph thereof:

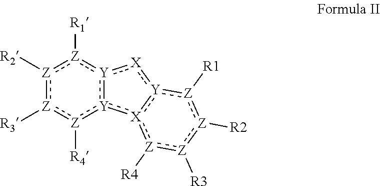

wherein,
X is N; Y is C; and Z is N, C or CH;
R¹-R⁴ are independently absent when Z is N, or,
one or more of R¹-R⁴ or R¹′-R⁴′ are lower to medium chain oxo- or keto fatty acids, optionally substituted with oxo or lower alkyl; H; halogen; lower alkyl; lower cycloalkyl; or COOH; or
one or more of R¹-R⁴ or R¹′-R⁴′ comprise the substituent of Formula X:

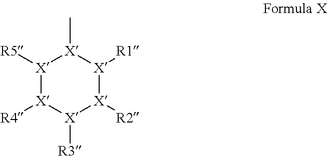

wherein,
X′ is CH, CO, N or O, preferably wherein X′ comprises up to two N or O; R¹″-R⁵″ are independently absent, or are H; lower alkyl; lower cycloalkyl; halogen; NH₂; NH-lower alkyl; hydroxy; oxo; COOH, SO₂NH₂; SO₂NH-loweralkyl; a substituted or non-substituted aromatic or non-aromatic heterocycle, preferably tetrazole; or comprises the substituent of Formula XI:

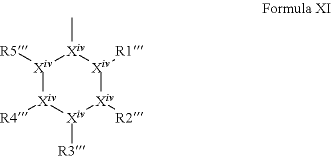

wherein,
X″ is CH, CH₂, CO, N or O, or preferably wherein X^{iv} comprises up to two N or O; R¹′″-R⁵′″ are independently absent, H, halogen, NH₂, lower alkyl, lower cycloalkyl, NH-lower alkyl, hydroxy, COOH, SO₂NH₂, SO₂NH-loweralkyl; a substituted or non-substituted aromatic or non-aromatic heterocycle, preferably tetrazole; or preferably wherein only one of R¹′″-R⁵′″ comprises a non-substituted aromatic or non-aromatic heterocycle; wherein the bonds between adjacent ring substituents X^{iv} may constitute single or double bonds, forming thereby a non-aromatic or aromatic ring; or
one or more of R¹-R⁴ comprise the substituent of formula XII:

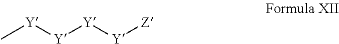

wherein,
Y′ is absent or can each be, independently, CH₂, CH₂(CH₃), CO, SO, SO₂, CHOH or NH; and Z′ is COOH; SO₂NH₂; a substituted or non-substituted aromatic or non-aromatic heterocycle, preferably tetrazole; forming a C₃-C₈ linear or branched chain; or
one or more of R₁-R₄ comprise the substituent of formula XIII:

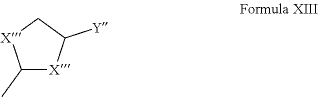

wherein,
Y″ is COOH, SO₂NH₂, or tetrazole; and X′″ is N or O.

In particular embodiments, compounds of Formula II may be selected from the group consisting of:

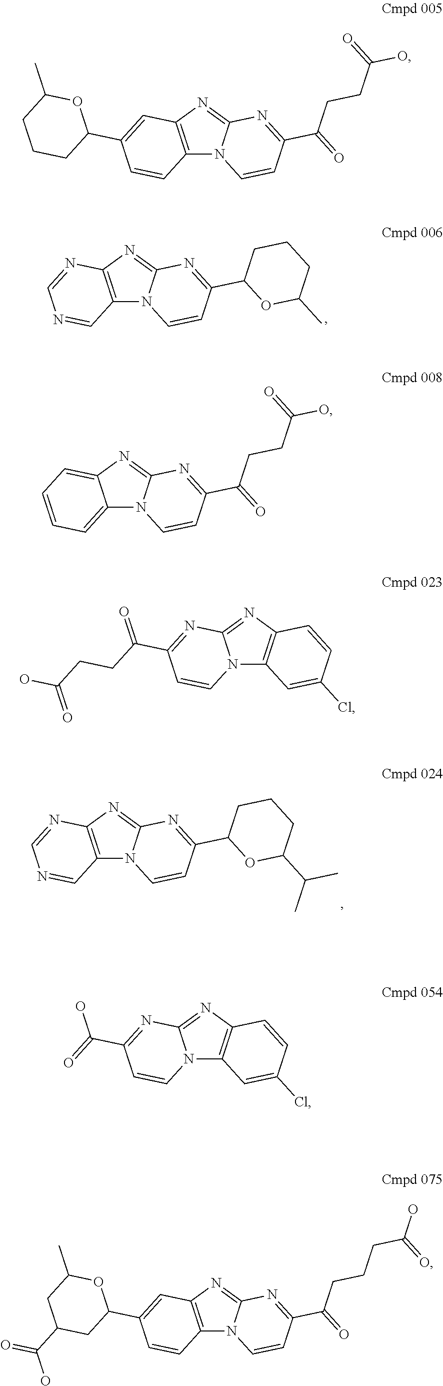

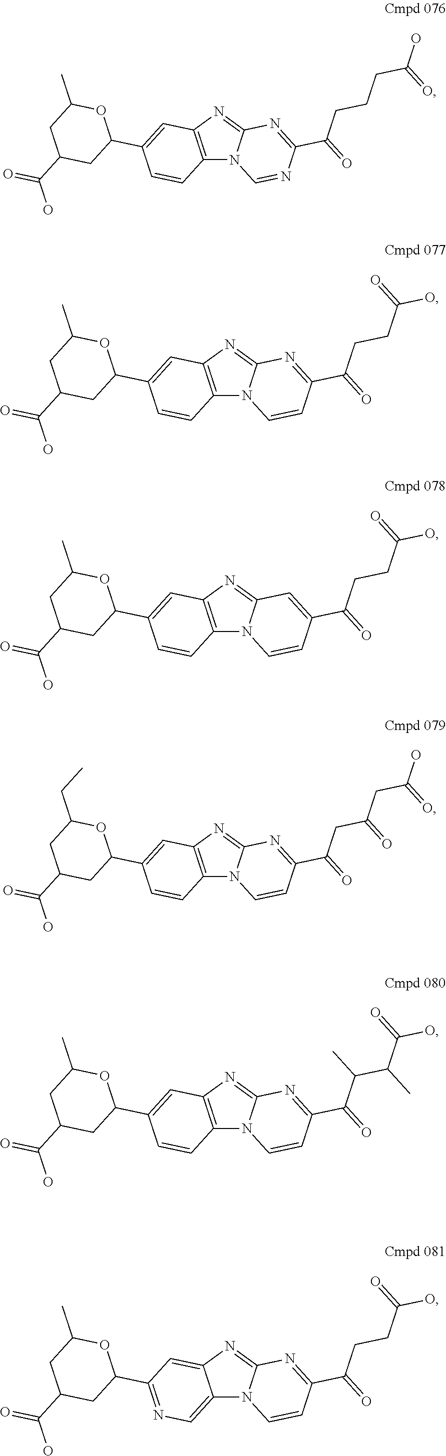

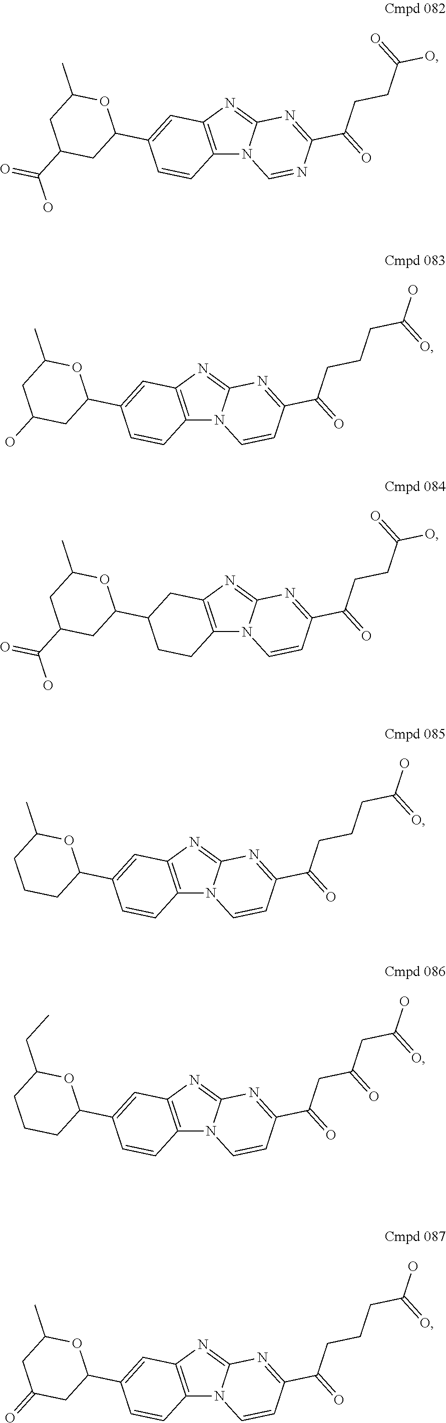

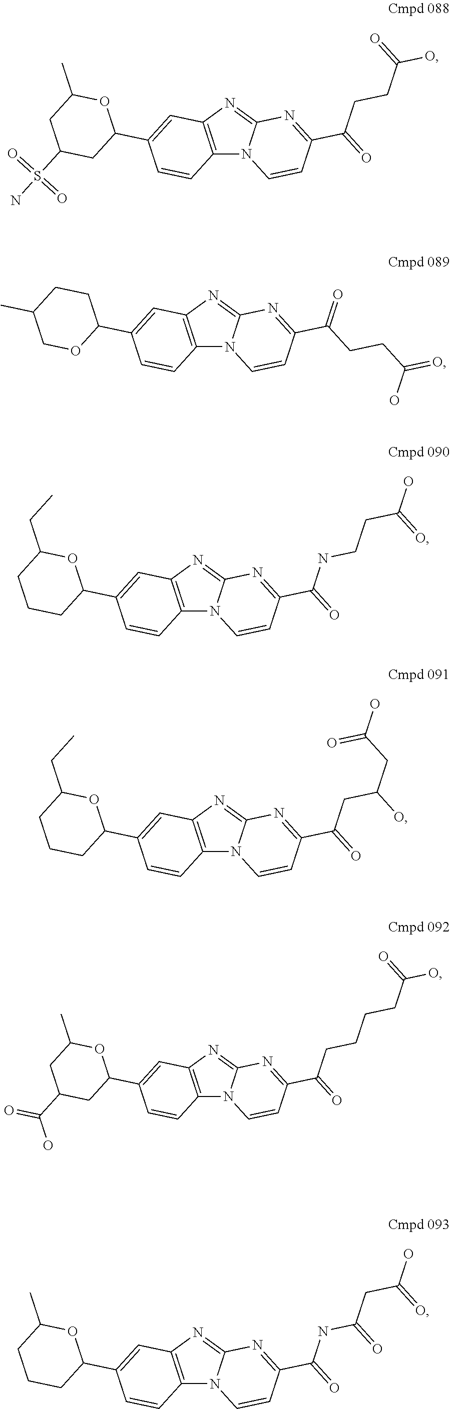

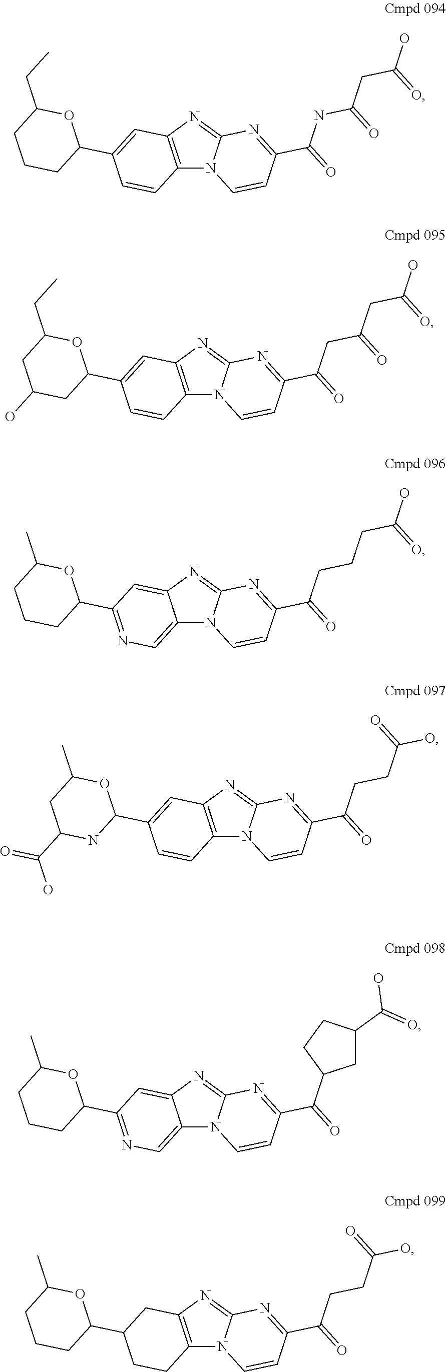

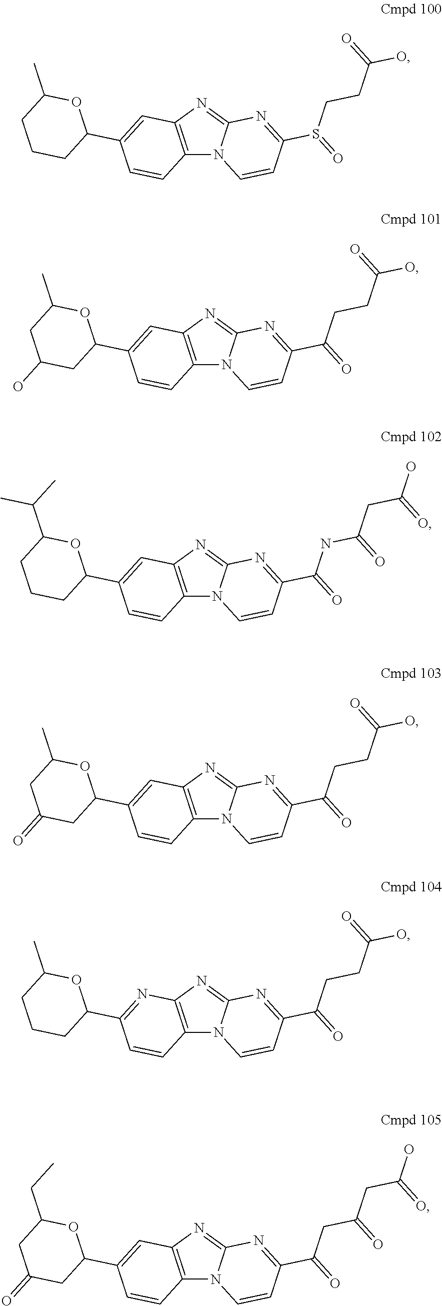

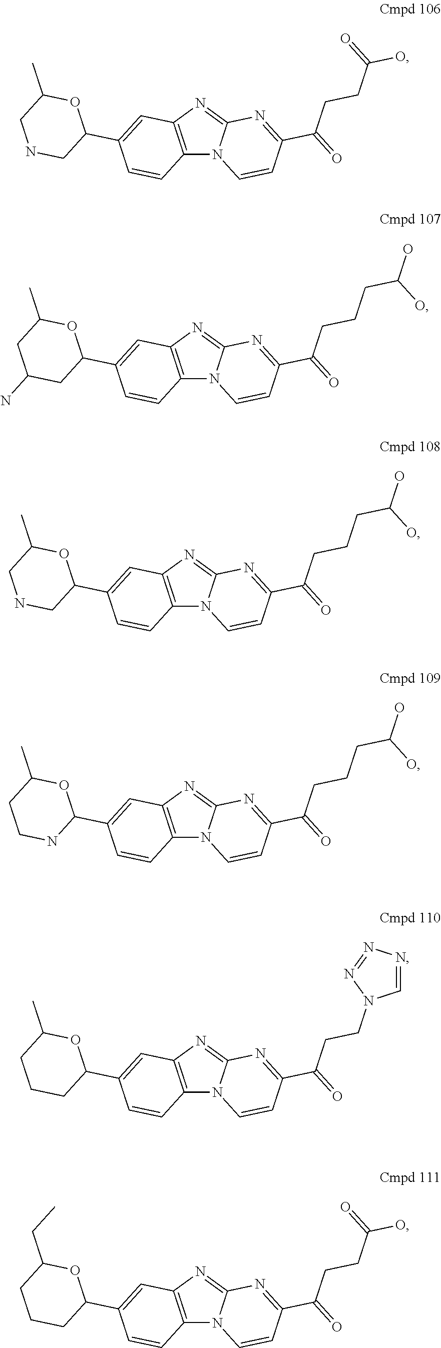

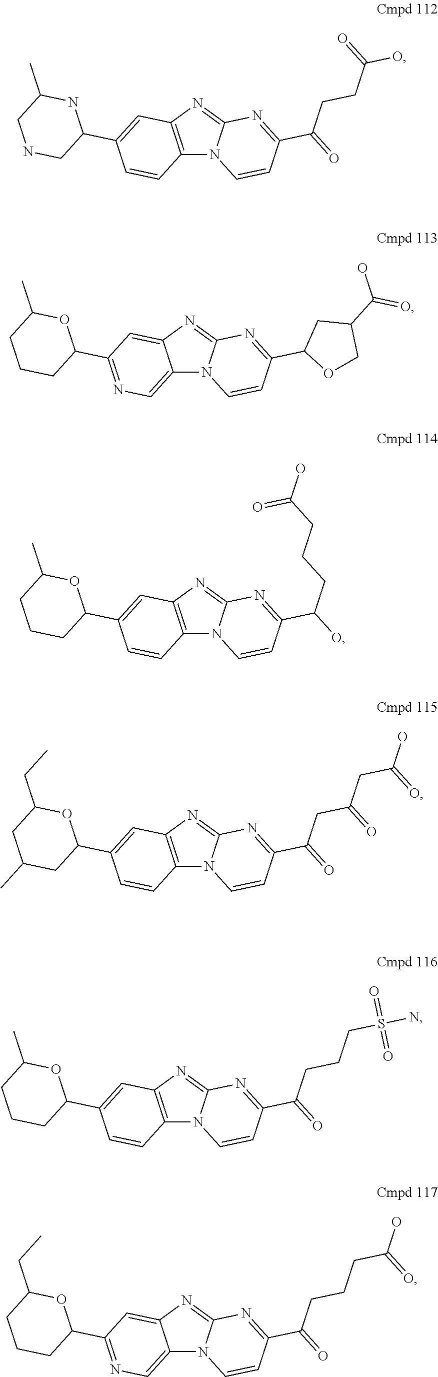

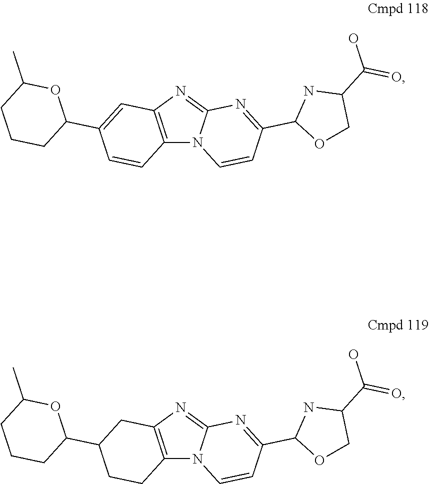 [SMILES: CC1CCCC(c2ccc3c(c2)nc2nc(C4NC(C(=O)O)CO4)ccn23)O1 ; CC1CCCC(C2CCc3c(nc4nc(C5NC(C(=O)O)CO5)ccn34)C2)O1]

and a pharmaceutically acceptable salt, hydrate, solvate, or polymorph thereof.

In a further embodiment, the invention comprises compounds of Formula III or a pharmaceutically acceptable salt, hydrate, solvate, or polymorph thereof:

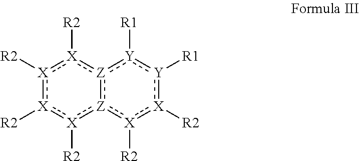

wherein,
each Z is independently selected from CH or C; each X is independently selected from CH, C, CH₂, O, N, S, SO, or SO₂; each Y is independently selected from N, C, CH, CH₂, CO, SO, S, or N-alkyl; wherein the bonds between adjacent ring substituents X, Z and/or Y constitute single or double bonds;
each R¹ is either absent or is H, halogen, O, NH₂, N-lower cycloalkyl, a phenyl group, alkyl group, optionally substituted with one or both of oxo and carboxy, OH, SO₂N(CH₃)₂, C(O)N(CH₃)₂, a five membered heterocycle, preferably tetrazole, —CO—(CH₂)ₙ—COOH, wherein n is 0-4, wherein R₁ optionally may form together with neighboring substituents, an aromatic or aliphatic 5, 6, or 7-membered ring system, which may contain an heteroatom, optionally substituted with one or more COOH, carboxamide, alkoxy, oxo, halogen, or triazole groups, or R₁ optionally comprises the substituent of Formula XIV:

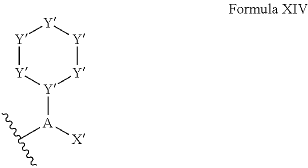

wherein,
X′ is absent or is O, N, or N-lower cycloalkyl; A is CH or may be a direct bond when X′ is absent; Y′ is O, N, N-lower cycloalkyl, CH₂, CH-lower alkyl, or CH—OH, or preferably X′ is absent or is O and the Y′-ring contains 1 or 2 N or O atoms; wherein the bonds between adjacent substituents Y′ and other atoms in the ring constitute single or double bonds, thereby forming a non-aromatic or aromatic ring; and
R² is absent, or is halogen; H; lower alkyl, optionally substituted with carboxy or oxo; hydroxy; alkoxy, COOH; SO₂NH₂; SO₂NH—O—CH₃, SO₂NH—C(O)—CH₃, SO₂N(CH₃)₂, SO₂-lower alkyl; C(O)N(CH₃)₂, a substituted or non-substituted aromatic or non-aromatic heterocycle, preferably triazole or tetrazole; or comprises the substituent of formula XV:

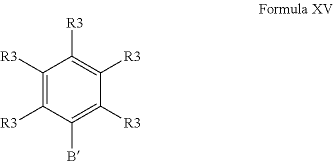

wherein,
B′ is attachment side, wherein B′ may comprise a linker or a direct bond, wherein the linker is —N—; and R³ is independently H, COOH, SO₂NH₂, SO₂NH— loweralkyl, halogen, or a substituted or non-substituted aromatic or non-aromatic heterocycle, preferably triazole or tetrazole.

In particular aspects of the invention, compounds of Formula III can be selected from the group consisting of:

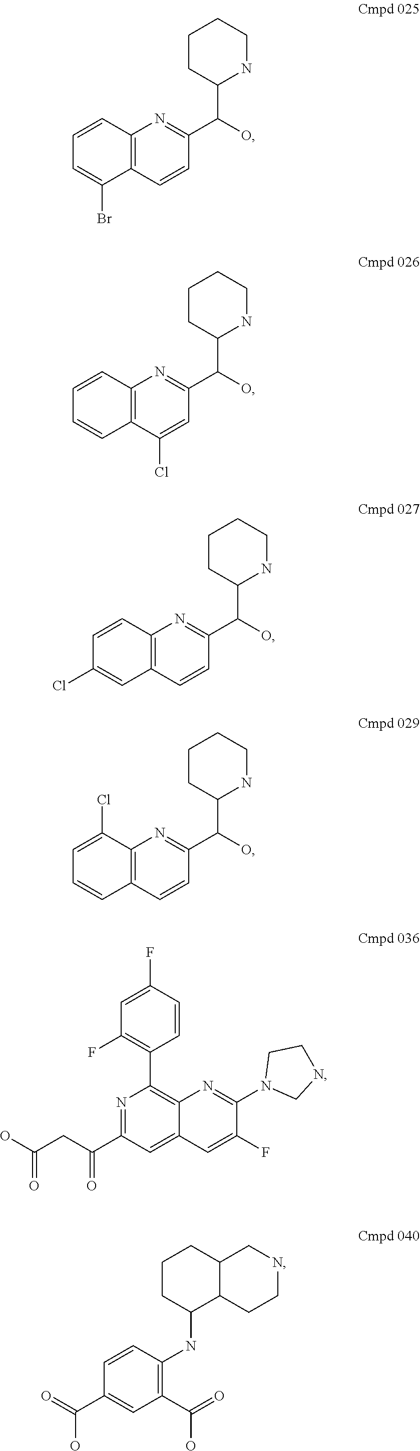

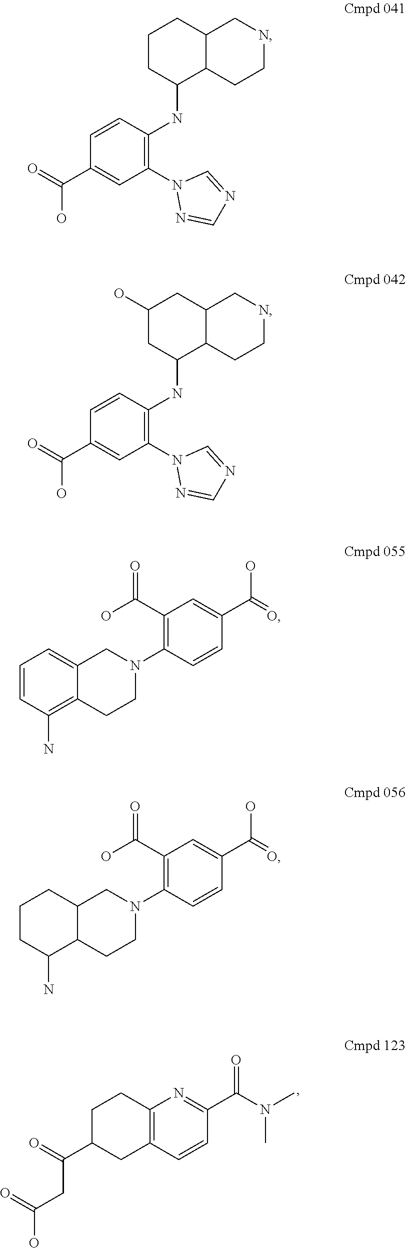

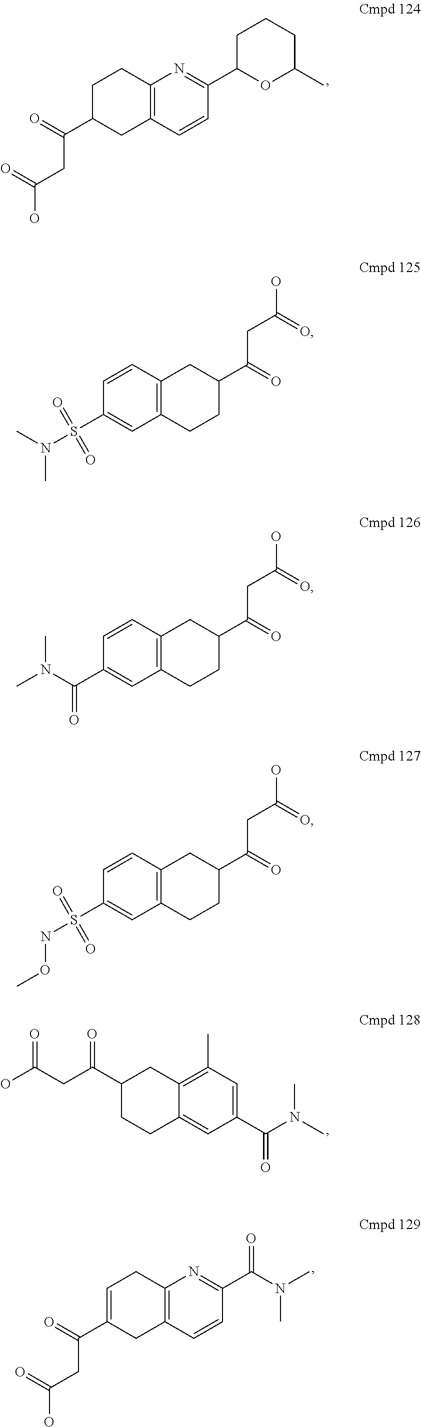

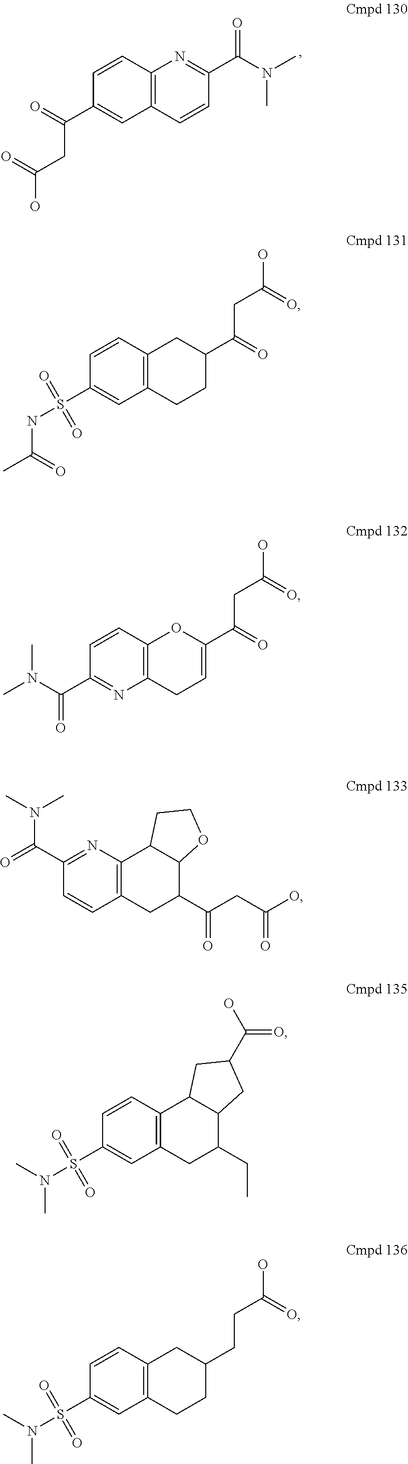

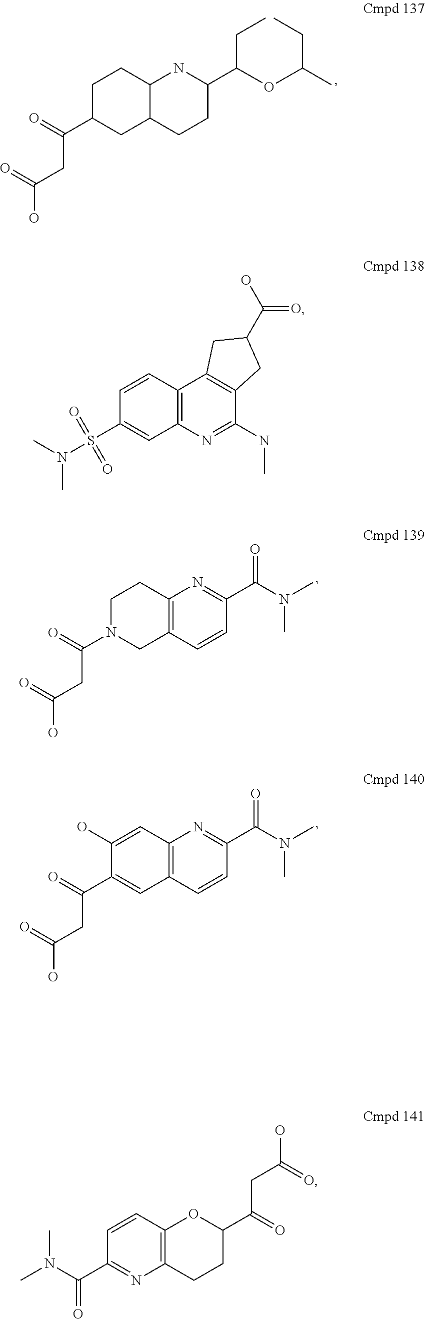

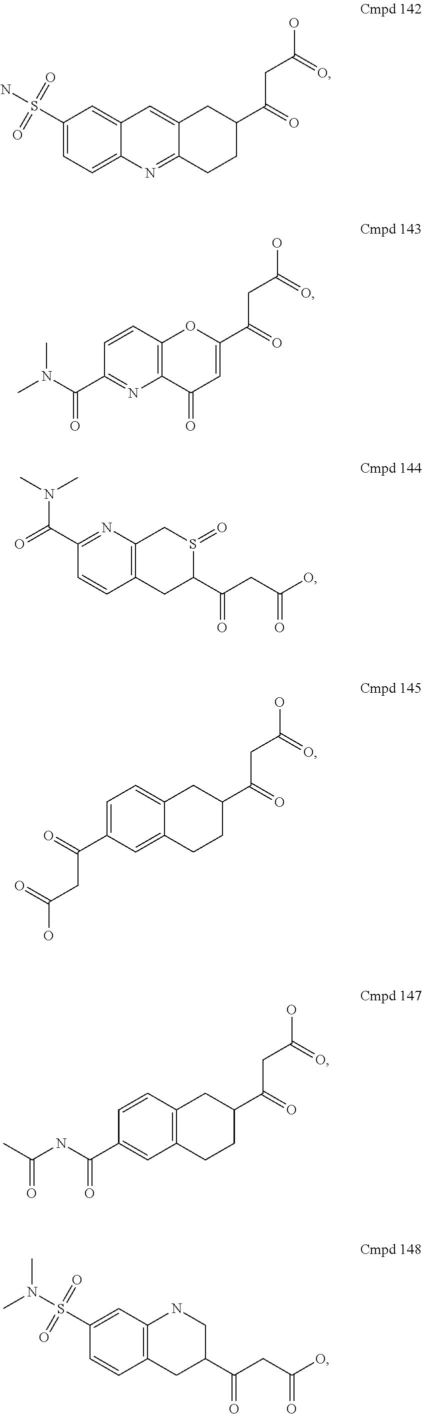

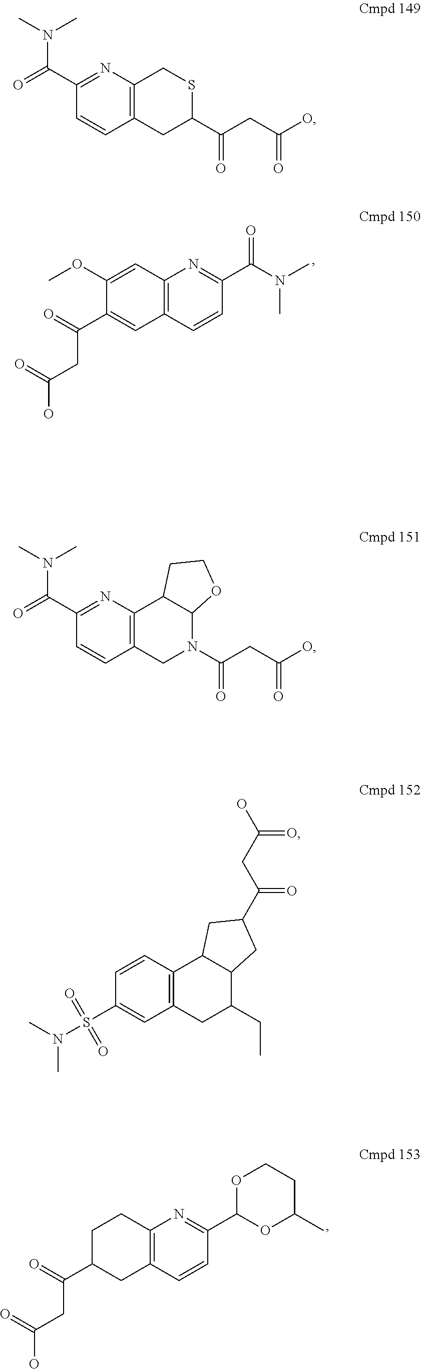

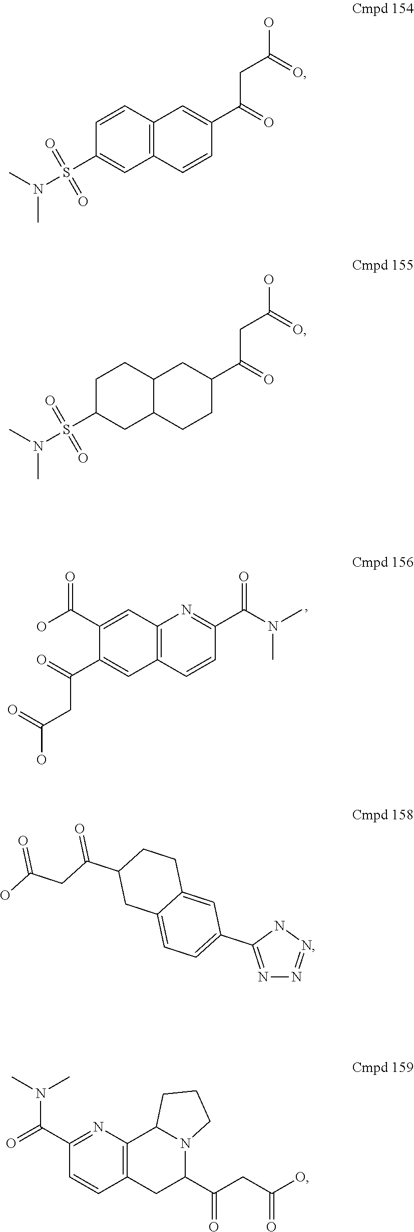

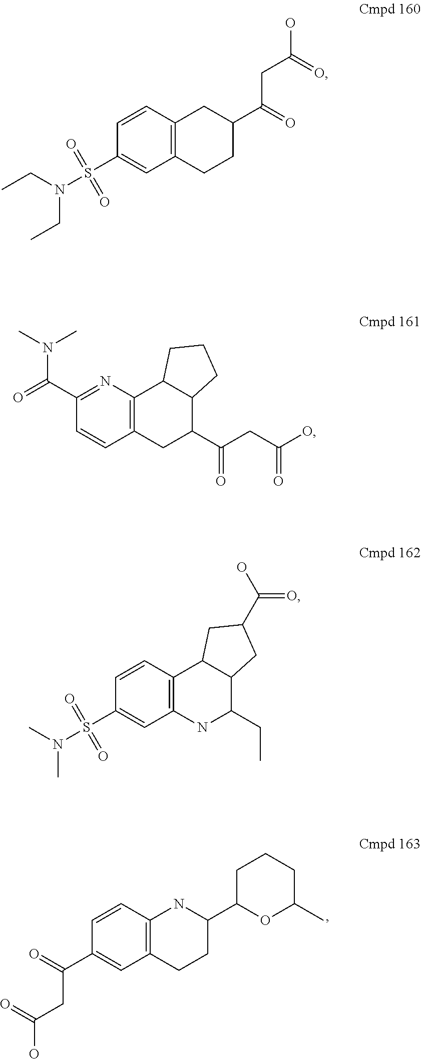

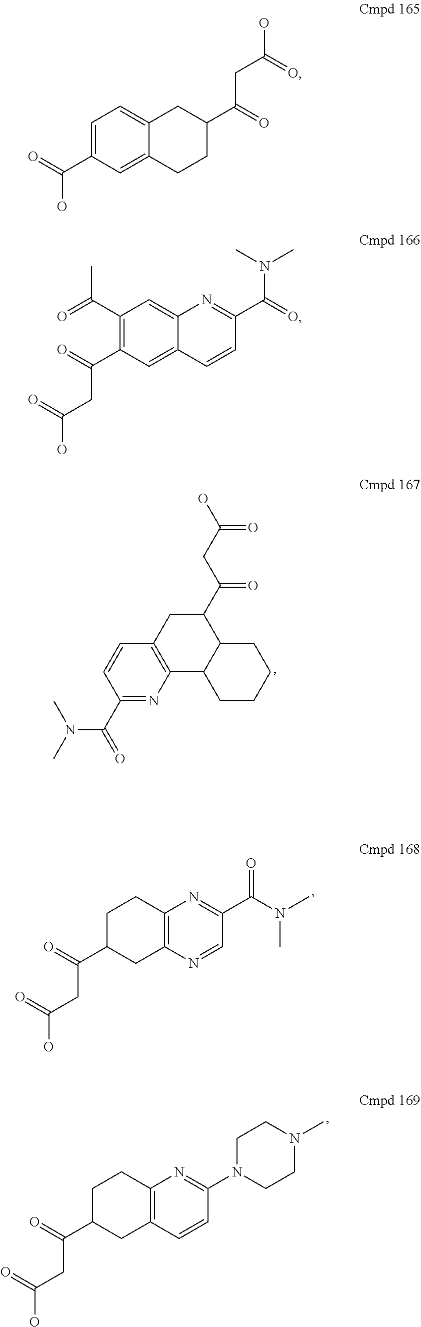

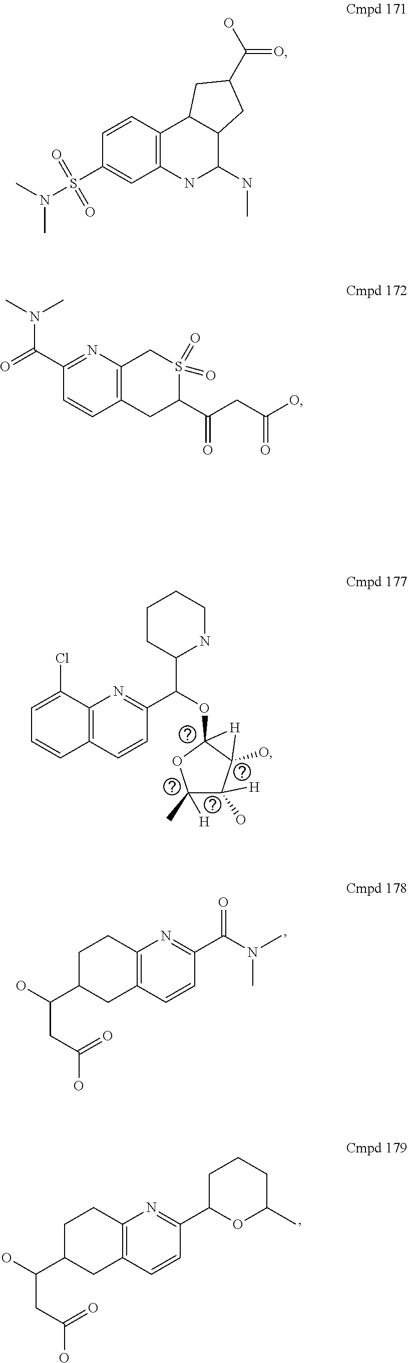

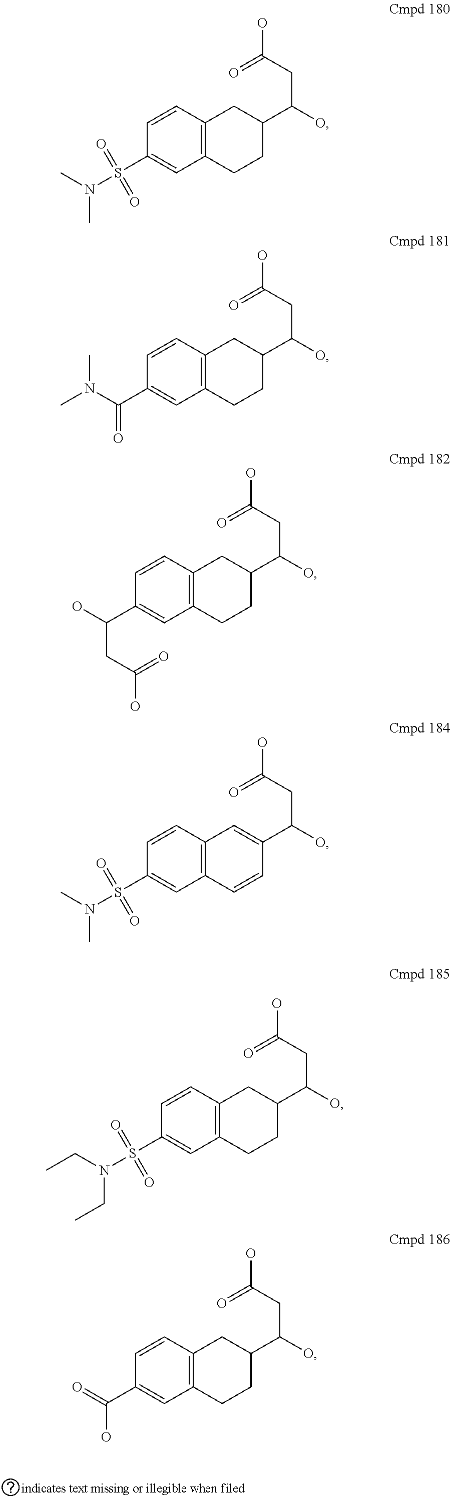

and a pharmaceutically acceptable salt, hydrate, solvate, or polymorph thereof.

In further embodiments, the invention relates to compounds of formula IV or a pharmaceutically acceptable salt, hydrate, solvate, or polymorph thereof:

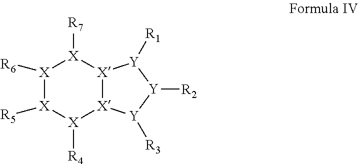

wherein,
each X is independently selected from CH or C;
X′ is C;
each Y is independently selected from N, CH, C, SO₂ or CO;
R¹ and R² are each independently H, COOH, O—CH3, lower cycloalkyl, or join together to form a five- or six-membered heterocyclic ring, which is optionally substituted;
R³-R⁷ are each independently H, COOH, O—CH3, lower cycloalkyl, or comprises the substituent of formula XVI:

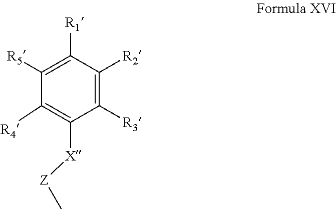

wherein,
Z is —N—CO—N or lower alkyl-CO—N,
X″ is lower alkyl or partly halogenated lower alkyl; and
R1′-R5′ are independently H; CHF₂; COOH; SO₂NH₂; NH—CH₂—COOH; lower alkyl; lower alkyl-COOH; lower alkyl-CO—; lower alkyl-COOH; —O—CO—CH₃; OCH₃; N—CO—N—O-lower alkyl; N—CO—N—O—CF₂; —SO₂—N (lower alkyl)₂; lower alkyl-O-lower alkyl-COOH; SO₂—N-lower alkyl-phenylcarboxylic acid; or a substituted or non-substituted aromatic or non-aromatic heterocycle, preferably tetrazole, which may optionally be connected to the bicyclic ring of formula IV with a lower alkyl linker.

In a particular embodiment, the compound of formula IV is selected from the group consisting of:

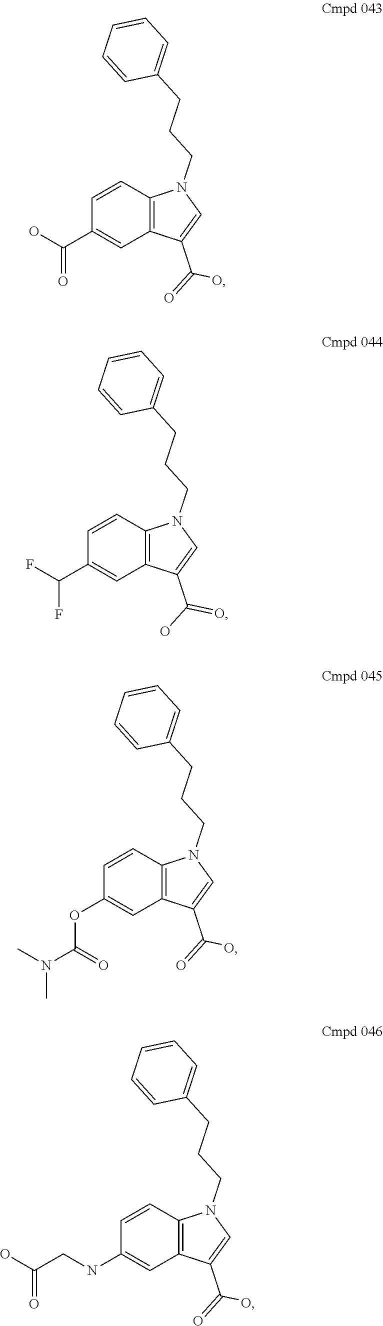

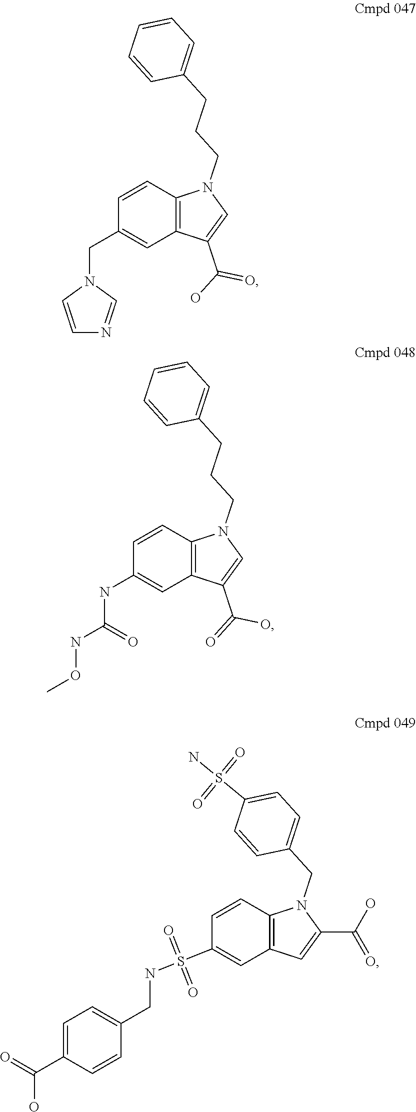

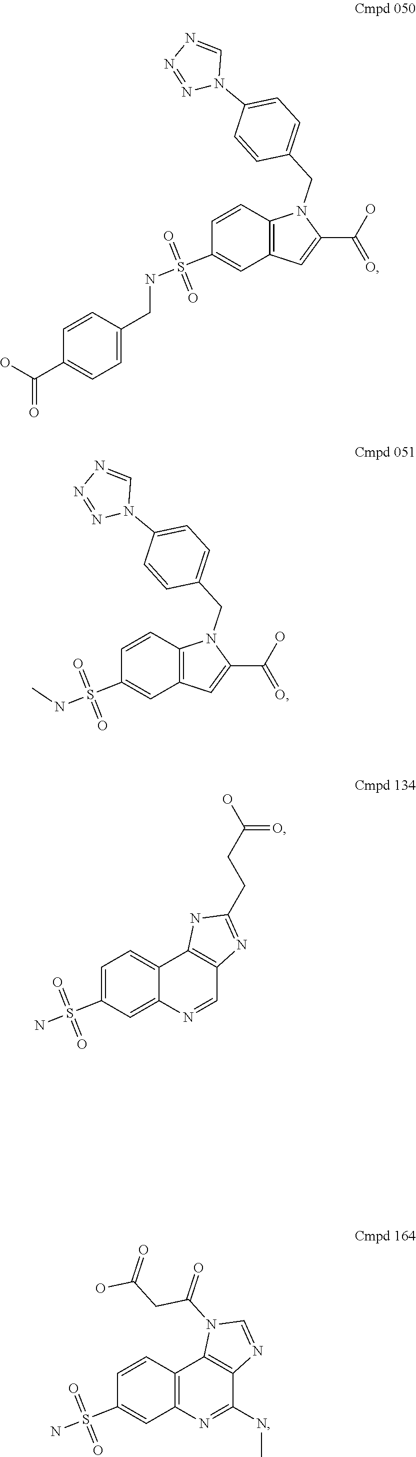

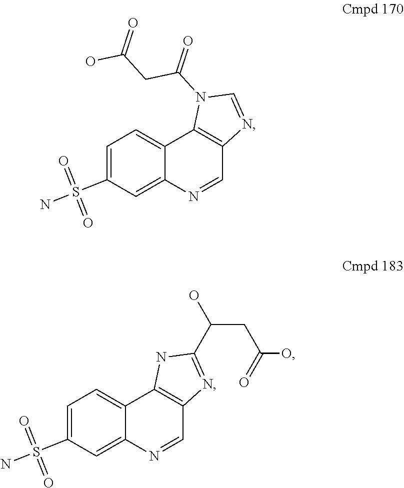

and a pharmaceutically acceptable salt, hydrate, solvate, or polymorph thereof.

In another embodiment, the invention comprises compounds of formula V or a pharmaceutically acceptable salt, hydrate, solvate, or polymorph thereof:

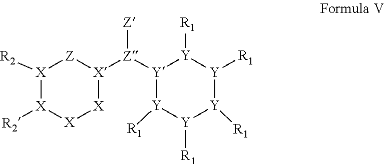

wherein, X is CH₂, C, or CH; X′ is either C or CH; Y is C or CH; Y′ is C; Z is independently N, O, or N-lower alkyl; Z′ is O; R₂ is H, halogen, lower alkyl, ketobutyric acid, cyclohexanonecarboxylic acid or hydroxymethyl(cyclohexenone); wherein the bonds between adjacent ring substituents X, X′ and/or Z constitute single or double bonds, and wherein the bonds between adjacent Y and/or Y′ constitute single or double bonds; Z″ can be CH or N, or lower alkyl, lower cycloalkyl, alkyl ether or cycloalkyl; and R₁ can be H, lower alkyl, halogen, O-lower alkyl, COOH, N—CO—N—O— lower alkyl or cycloalkyl, partly halogenated lower alkyl or cycloalkyl, —CO—CH₃, -lower alkyl-O-phenyl carboxylic acid with a lower alkyl substituent, or

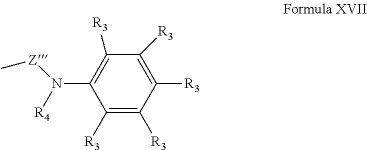

wherein,
Z′″ is lower alkyl or cycloalkyl, R3 is each independently H, COOH, NH—CO—NH—OCH₃ or partly halogenated NH—CO—NH—CH₃, O-lower alkyl, CO—CH₃ or lower alkyl, and R4 is lower alkyl.

In particular embodiments, compounds of formula V can be selected from the group consisting of:

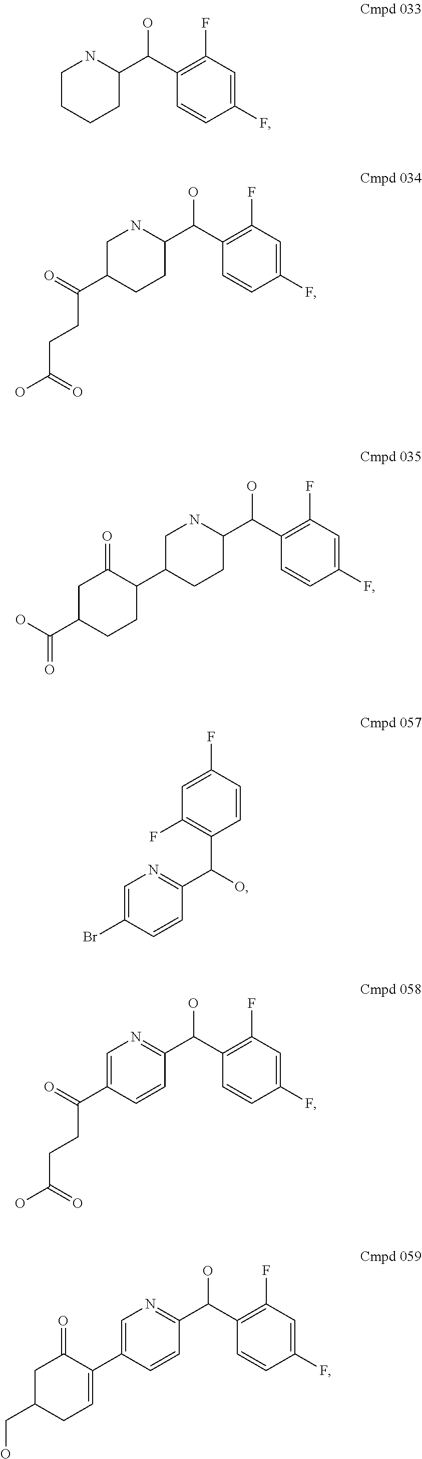

and a pharmaceutically acceptable salt, hydrate, solvate, or polymorph thereof.

In another embodiment, the invention comprises compounds of formula VI or a pharmaceutically acceptable salt, hydrate, solvate, or polymorph thereof:

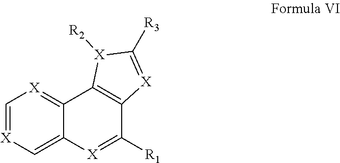

wherein,
independently X is N or CH; R¹ is NH₂, NH-lower alkyl, OH, or O-lower cycloalkyl; R² and R³ can be H, lower alkyl, SO₂NH₂ or comprise the substituent of formula XVIII:

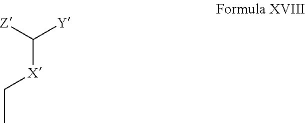

wherein,
X′ is a direct bond, NH, N—CH₃ or CH₂; Y′ is H or lower alkyl; and Z′ is H, lower alkyl, COOH, SO₂NH₂, SO₂NH—CH₃, or a substituted or non-substituted aromatic or non-aromatic heterocycle, preferably tetrazole, wherein X′ is N—CH₃ when both Y′ and Z′ are H.

In particular embodiments, the compounds of formula VI can be selected from the group consisting of:

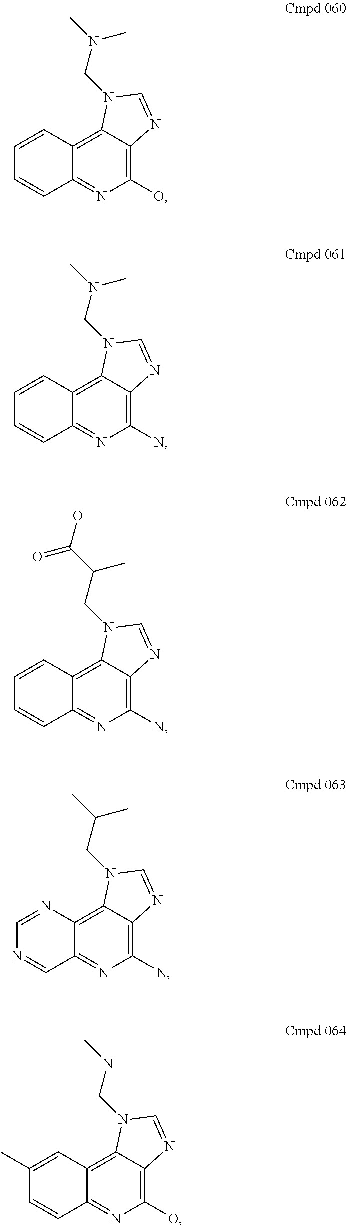

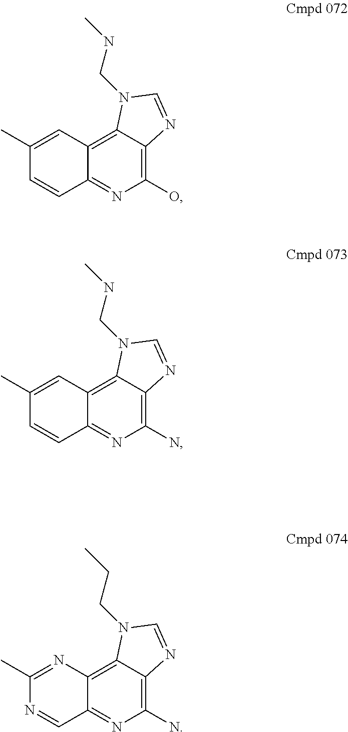

and a pharmaceutically acceptable salt, hydrate, solvate, or polymorph thereof.

In a further embodiment, the invention comprises compounds of formula VII or a pharmaceutically acceptable salt, hydrate, solvate, or polymorph thereof:

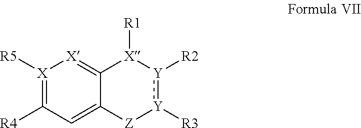

wherein,
X is N or C; X′ is CH or N; X″ is N or CH; Y is CH or C; Z is CH₂— or C═O; wherein the bond between adjacent ring substituents Y constitute a single or double bond;
R¹ comprises a substituent of formula XIX:

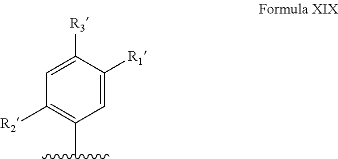

wherein,
R1′-R3′ can be independently halogen, carboxylic acid, SO₂NH₂, SO₂—NH— lower alkyl, COOH, or NO₂, or a substituted or non-substituted aromatic or non-aromatic heterocycle, preferably tetrazole; or
R1 can comprise a substituent of formula XX:

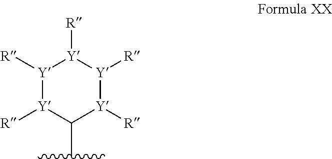

wherein,
Y′ is N, CH, or C; R″ is independently H, halogen, dihalogen or lower alkyl;
R² is H, lower cycloalkyl or a substituted or non-substituted aromatic or non-aromatic heterocycle, preferably 1,3-diazolidine;
R³ is halogen, carboxylic acid, SO₂NH₂, SO₂—NH-lower alkyl, or a substituted or non-substituted aromatic or non-aromatic heterocycle, preferably tetrazole;
R⁴ is H, halogen, or low alkyl ketoacid, preferably ketopropionic acid;
R⁵ is absent if X is N or CH; if X is C, R5 is a substituted or non-substituted aromatic or non-aromatic heterocycle, preferably pyrrolidine, a piperidine, pyrazine, or a bicyclic structure of formula XXI:

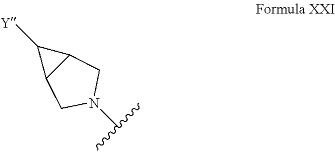

wherein,
Y″ is N, N-lower alkyl, or COOH.

In particular embodiments, compounds of formula VII can be selected from the group consisting of:

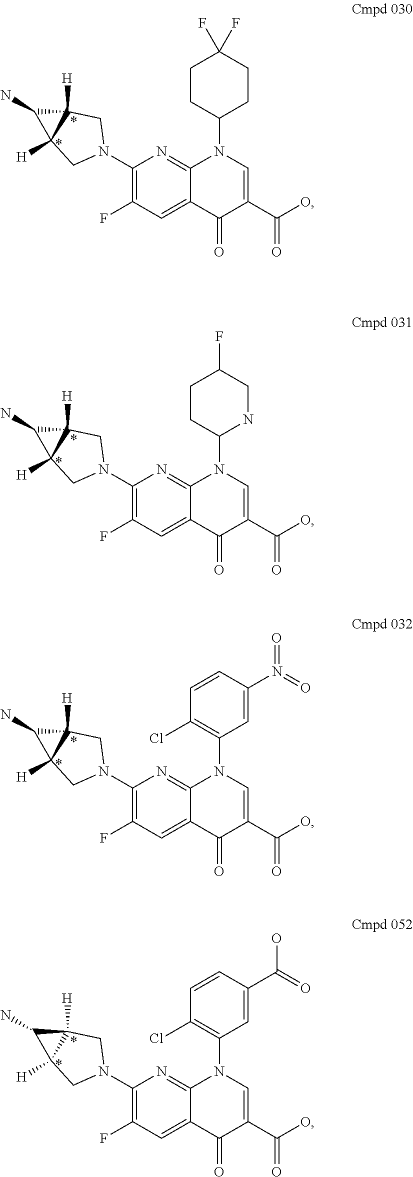

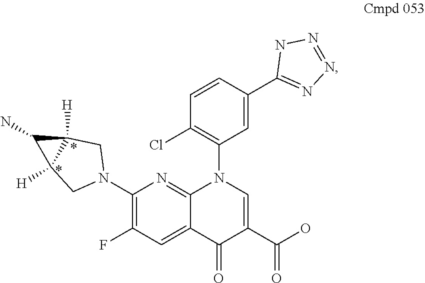 [SMILES: N[C@@H]1[C@H]2CN(c3nc4c(cc3F)c(=O)c(C(=O)O)cn4-c3cc(-c4nnn[nH]4)ccc3Cl)C[C@@H]12]

and a pharmaceutically acceptable salt, hydrate, solvate, or polymorph thereof.

In another embodiment, the invention comprises compounds of formula VIII or a pharmaceutically acceptable salt, hydrate, solvate, or polymorph thereof:

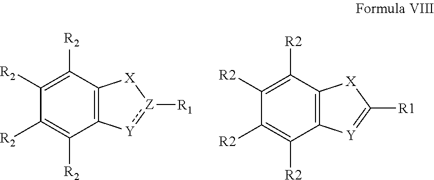

wherein,
X is SO₂, NR₄ or O; Z is C or N; Y is N, CR₃ or CH; R₁-R₄ are each independently H, oxo, halogen, —(CH₂)ₙ—COOH, wherein n=0 to 6, —NH—C(O)—NH—CH₃, —NH—C(O)—NH—CHF₂, alkyl or alkoxy, optionally substituted with COOH or oxo, or wherein one or more of R₁-R₄ comprise a substituent of Formula XXII:

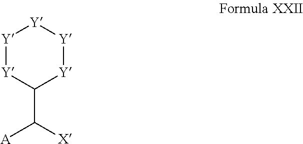

wherein,
A is attachment side, wherein the substituent of Formula XXII is attached directly or through a linker L to the compound of formula VIII, wherein L is alkyl, —CH₂—C(O)—NH—, or —NH—C(O)—NH—;
X′ is H, O, N, or N-lower cycloalkyl;
Y′ is O, N, N-lower cycloalkyl, CH₂, CR₅, CHR₅, wherein R₅ is alkyl, hydroxy, alkoxy, halogen, wherein the alkyl is optionally substituted by one or more of COOH, oxo, alkoxy, halogen, or hydroxylamine, wherein adjacent Y′ are bonded with a single or double bond, and wherein two R₅ attached to two adjacent Y′ are optionally joined to form an aromatic or non-aromatic five- or six-membered ring; preferably X′ is O and the Y′-ring contains 1 or 2 N or O atoms.

In particular embodiments, the compounds of formula VIII can be selected from the group consisting of:

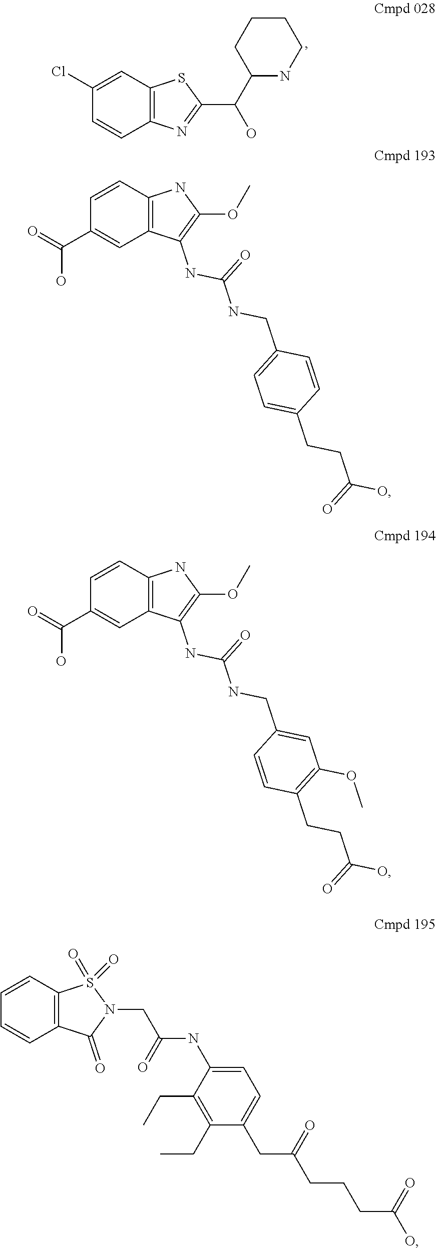

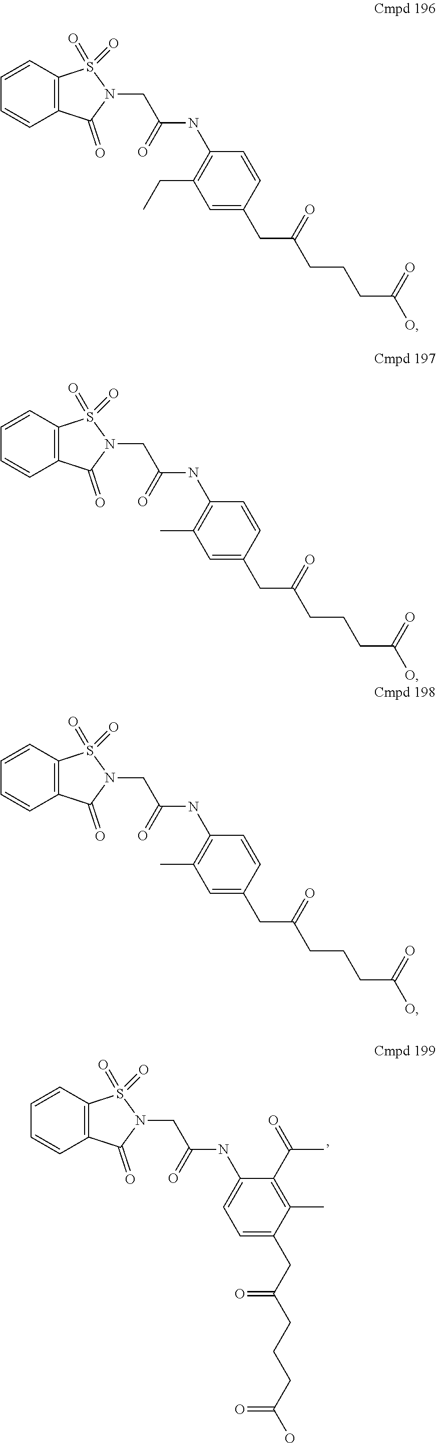

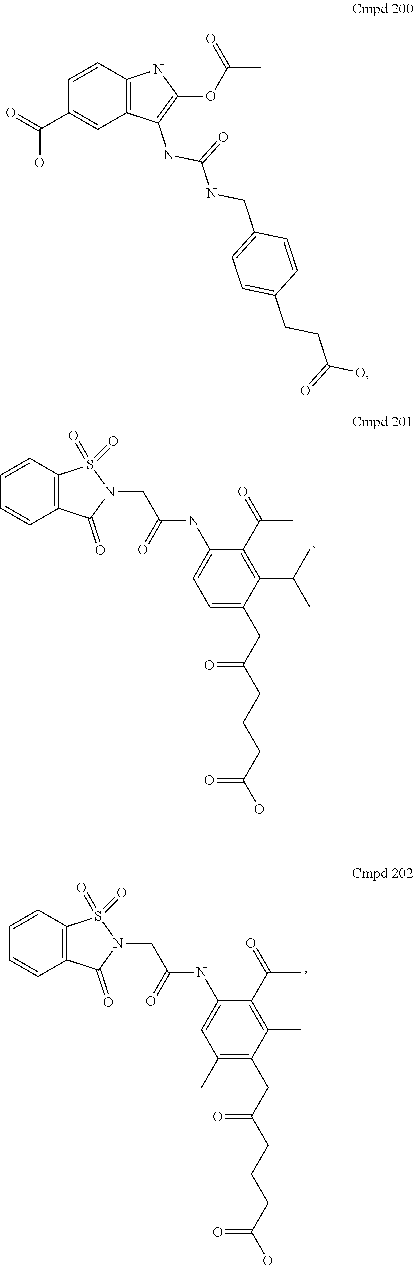

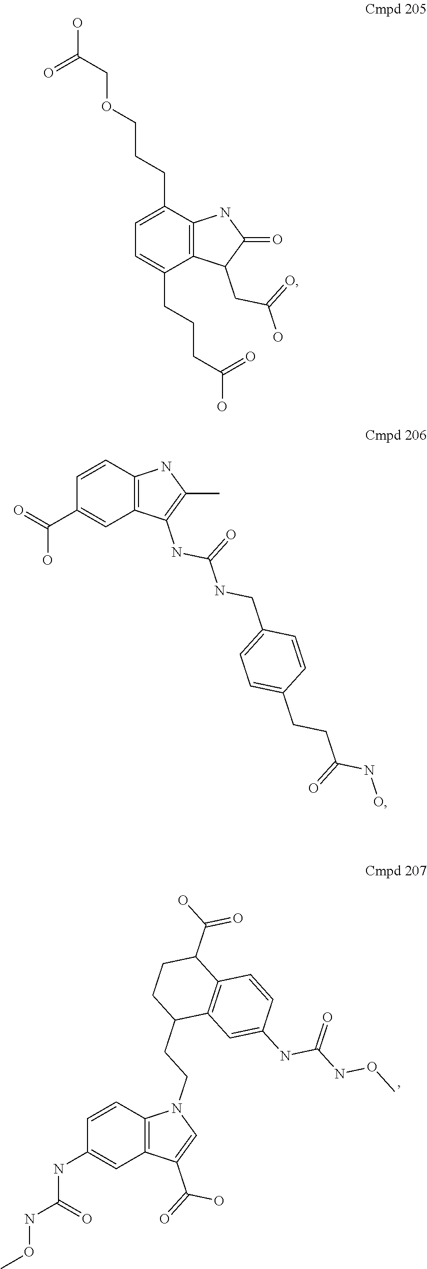

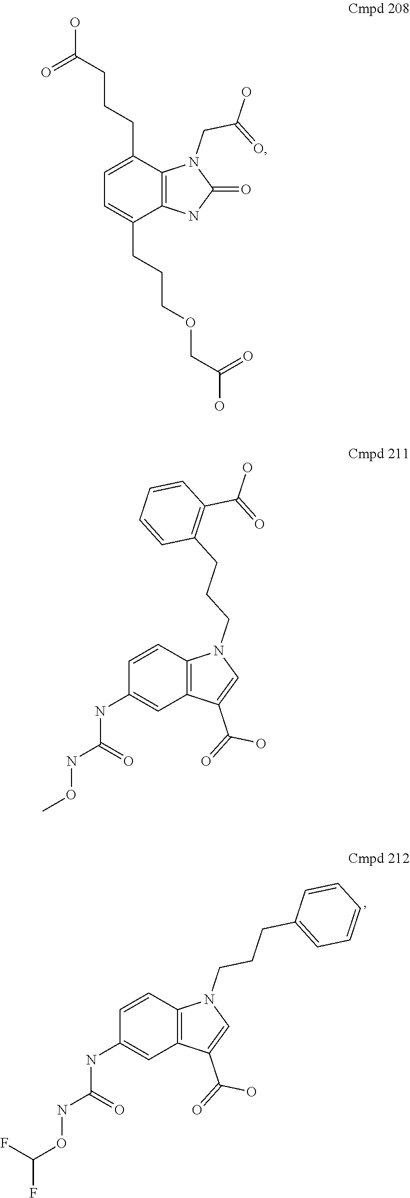 [SMILES: O=C(O)CCCc1ccc(CCCOCC(=O)O)c2[nH]c(=O)n(CC(=O)O)c12 ; CONC(=O)Nc1ccc2c(c1)c(C(=O)O)cn2CCCc1ccccc1C(=O)O ; O=C(NOC(F)F)Nc1ccc2c(c1)c(C(=O)O)cn2CCCc1ccccc1]

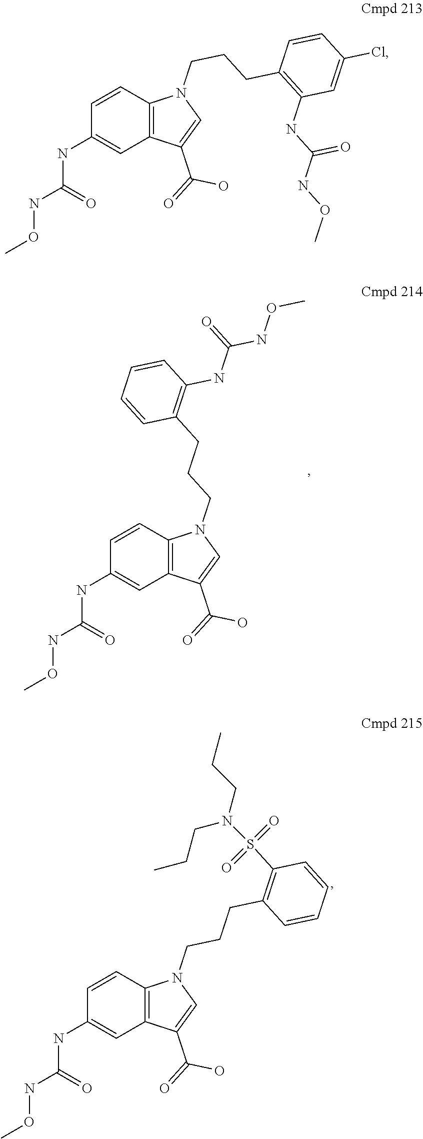

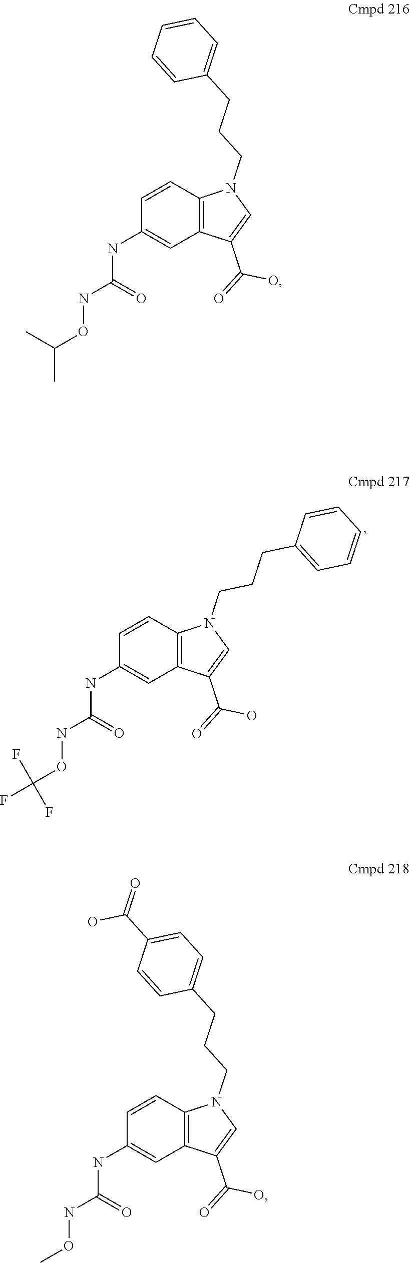

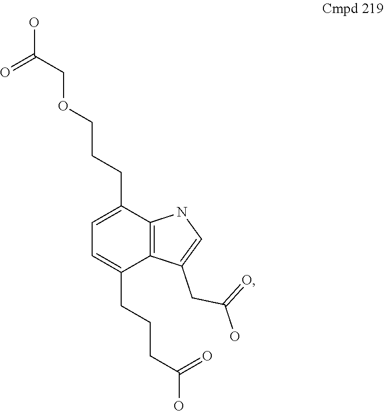 [SMILES: O=C(O)CCCc1ccc(CCCOCC(=O)O)c2[nH]cc(CC(=O)O)c12]

and a pharmaceutically acceptable salt, hydrate, solvate, or polymorph thereof.

The compounds of the present invention can be in the form of pharmaceutically acceptable salts. Suitable salts include acid addition salts which may, for example, be formed by mixing a solution of the compound of the present invention with a solution of a pharmaceutically acceptable acid such as hydrochloric acid, sulfuric acid, acetic acid, or benzoic acid. Acceptable salts may also include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

### Biologic Activity and Therapeutic Application of PANX-1 Modulators

In some embodiments, the PANX1 modulators of the present invention are applicable in the treatment of diseases. The PANX1 modulatory activity of a given compound can be measured using a variety of in vitro models known to a skilled artisan. For instance, models to evaluate ATP release by Human Embryonic Kidney (HEK)-293 cells or Xenopus Oocytes genetically transformed to overexpress PANX1 hemichannels are available (6)(7). Models based on cells naturally expressing PANX1 are also known. For instance, the skilled artisan would know that human subcutaneous fibroblasts release ATP via PANX1 upon mechanical or histamine stimulation (8)(9).

It is also possible to investigate the potential preventive or therapeutic effects of PANX1 modulators on several disease conditions using both in vitro and in vivo models. For instance, the antitumor effect of PANX1 blockers over colorectal cancer, breast cancer, and melanoma can be assessed using corresponding cell lines (10-12). Animal models to evaluate the ability of PANX1 modulators to prevent the advance or manage the symptoms of Alzheimer's disease or Multiple Sclerosis have been described as well (13)(14).

In some embodiments of the present invention, the PANX1 modulators or their pharmaceutically acceptable salts, hydrates, solvates, and/or polymorphs may be for use in the treatment of diseases wherein PANX1 overactivity is implicated.

In another aspect, the invention provides methods of treating patients suffering from diseases related to PANX1 overactivity, wherein a compound of the present invention or one of its pharmaceutically acceptable salts, hydrates, solvates, and/or polymorphs is administered to the patient in need thereof.

In another embodiment, the invention provides the use of the PANX1 modulators described herein, or their salts, hydrates, solvates, and/or polymorphs, to prepare a medicament for preventing or treating a disease wherein panx-1 overactivity is implicated.

In some embodiments, the therapeutic methods and the therapeutic uses of the PANX1 modulators described herein are related to the treatment of diseases that benefit from the modulation of pannexin-1 activity, but not limited to: chronic pain, opioid addiction, epilepsy, Parkinson's disease (PD), multiple sclerosis (MS), Alzheimer's disease (AD), traumatic brain injury, migraine, stroke, neoplastic diseases and/or symptoms, cardiovascular diseases, inflammatory or autoimmune diseases, and pulmonary diseases.

In some embodiments of the invention, the neoplastic diseases and/or symptoms treatable with PANX1 modulators of the invention may be selected from hepatocellular carcinoma (HCC), breast cancer, colorectal cancer, pancreatic cancer, leukemia, chemotherapy-associated pain, and others known to the skilled person.

In other embodiments of the invention, the PANX1 modulators are applicable to the treatment of cardiovascular diseases, which may be selected from arrythmia, vascular inflammation, pulmonary arterial hypertension (PAH), elevated blood pressure, and others known to the skilled person.

In other embodiments of the invention, the PANX1 modulators are applicable to the treatment of inflammatory or autoimmune diseases, which may be selected from joint inflammation, wound healing disorders, and others known to the skilled person.

In other embodiments of the invention, the PANX1 modulators can be applied to the treatment of pulmonary diseases, which may be selected from asthma, COPD, primary and secondary ciliary dyskinesia (PCD and SCD), coronavirus-mediated pulmonary diseases (such as Covid-19), and others known to the skilled person.

As already pointed out earlier PANX1 is involved in an impressive number of pathologies in different organs. Having designed and elaborated several different chemical classes of compounds with distinctly different physicochemical properties, this will enable assigning the PANX1 inhibitors towards specific organs and their respective underlying pathologies. The following outlines the most investigated pathologies and their relationship/role in different pathologies.

### Chronic Pain

Neuropathic pain is an incapacitating consequence of cancer treatment with cytotoxic chemotherapeutics, such as paclitaxel. The economic cost of chronic pain, including neuropathic pain as a large component, has been estimated to be over $500 billion in the US alone. PANX1-deleted mice developed acute mechanical hypersensitivity after an initial bout of paclitaxel, but unlike wild type mice, the neuropathic pain was not maintained and resolved after a second bout of paclitaxel. (16-18)

PANX1 in hematopoietic cells is required for pain-like responses following nerve injury in mice, and a potential therapeutic target. PANX1 knockout mice (PANX1^{-/-}) were protected from hypersensitivity in two sciatic nerve injury models. Bone marrow transplantation studies show that expression of functional PANX1 in hematopoietic cells is necessary for mechanical hypersensitivity following nerve injury (16-18).

### Opioid Addiction, General Addiction

In mice studies PANX1 is activated during opioid withdrawal. Therefore, the P₂X₇-Panx1 signalling cascade is likely to function as a feed forward loop, amplifying the cellular response to withdrawal. Mice lacking PANX1 on microglia showed decreased withdrawal behaviours as compared to controls, yet morphine analgesia was not affected. When the PANX1 inhibitors probenecid and mefloquine, were administered before the opioid antagonist naloxone in mice, withdrawal behaviours were markedly reduced (19).

Ethanol-induced Cx43 hemichannel and Panx1 channel activity were correlated with increased levels of inflammatory messengers IL-1β, TNF-α, IL-6 in the hippocampus, as well as with profound alterations in astrocyte arbor complexity. Thus uncontrolled opening of astrocyte hemichannels and pannexons may contribute to the pathogenesis of alcohol use disorders in the adulthood (20).

### Epilepsy

As PANX1 expression is raised in several animal seizure models and in resected human epileptic brain tissue, it is suggested to be of relevance to epilepsy. Seizure activity was suppressed in PANX1 knockouts and by application of PANX1 channel modulators in epilepsy animal models. Following electrical stimulation of the hippocampal CA3 region, PANX1 knockouts had significantly shorter evoked after discharges and were resistant to kindling. Activation or inhibition of Panx channel has been shown to regulate the release of adenosine triphosphate (ATP) and other signals, which is very important for the onset and control of nervous system diseases including epilepsy. Postoperative human tissue samples from patients with epilepsy showed that Pannexin-1 channel activation promoted seizure generation and maintenance through adenosine triphosphate signaling via purinergic 2 receptors. Pharmacological inhibition of PANX1 channels with probenecid or mefloquine respectively-blocked ictal discharges in human cortical brain tissue slices. Genetic deletion of PANX1 channels in mice had anticonvulsant effects when the mice were exposed to kainic acid, a model of temporal lobe epilepsy. This suggests a proepileptic role of PANX1 channels in chronic epilepsy in human patients and that pannexin-1 channel inhibition might represent an alternative therapeutic strategy for treating lesional and drug-resistant epilepsies (20-26).

### Parkinson's Disease

Nod-like-receptor pyrin domain-containing 3 plays a key role in the pathogenesis of Parkinson's disease. PANX1 channels might therefore contribute importantly to the inflammatory cascade underlying this neurodegenerative disease.

It was found that α-synuclein enhances the opening of connexin 43 (Cx43) hemichannels and PANX1 channels in mouse cortical astrocytes. Therefore, it has been proposed that α-synuclein-mediated opening of astroglial Cx43 hemichannels and Panx1 channels might constitute a novel mechanism involved in the pathogenesis and progression of α-synucleinopathies (27)(28).

### Multiple Sclerosis

Probenecid (a PANX1 inhibitor) reduced clinical symptoms (disease score) in the experimental autoimmune encephalomyelitis MS model in mice by reducing inflammation, the number of T lymphocytes infiltrating the spinal cord, and the loss of oligodendroglia lineage cells (14)(29)(30).

### Alzheimer's Disease

Age-dependent increase in PANX1 expression correlates with increased Aβ levels in hippocampal tissue from Tg mice together with an exacerbated PANX1 activity upon basal conditions and in response to glutamate receptor activation. The acute inhibition of Panx1 activity with the drug probenecid (a Panx1 inhibitor) significantly reduced excitatory synaptic defects in the AD model by normalizing long-term potentiation (LTP) and depression and improving dendritic arborization and spine density in hippocampal neurons of the Tg mice. This strongly suggests a major contribution of Panx1 in the early mechanisms leading to the synaptopathy in AD (31).

### Traumatic Brain Injury

A murine-controlled cortical impact (CCI) model with myeloid-specific PANX1 conditional knockout (Cx3cr1-Cre::Panx1fl/fl) mice showed that myeloid PANX1 mediates neuroinflammation and brain damage. CCI-related outcomes correlated well with PANX1 channel function in myeloid cells, thus indicating that activation of PANX1 channels in myeloid cells is a major contributor to acute brain inflammation following TBI (32).

### Migraine

Cortical spreading depression (CSD) is the putative cause of migraine aura and headache. CSD causes neuronal Pannexin1 (Panx1) megachannel opening and caspase-1 activation followed by high-mobility group box 1 (HMGB1) release from neurons and nuclear factor κB activation in astrocytes. Suppression of this cascade abolished CSD-induced trigeminovascular activation, dural mast cell degranulation, and headache. Thus, inhibition of Panx1 looks like a logical step to ameliorate this disease (33)(34).

### Stroke

Wild type and Panx1 KO mice were subjected to permanent middle cerebral artery (MCA) occlusion, and infarct size and astrocyte and microglia activation were assessed. The sexually dimorphic nature of Panx1 deletion was also explored tested by analyzing the effect of probenecid to alter stroke volume. Panx1 KO females displayed significantly smaller infarct volumes (˜50% reduction) compared to their wild-type counterparts, whereas no such KO effect occurred in males (35).

### Cancer

Adenosine triphosphate (ATP) is one of the main biochemical components of the tumour microenvironment (TME), where it can promote tumour progression or tumour suppression depending on its concentration and on the specific ecto-nucleotidases and receptors expressed by immune and cancer cells (36). Most literature refers to the microtumor environment, stressing an overexpression of Panx1 in some cancers:

### Melanoma

Panx1 forms large pore channels capable of passing ions and metabolites such as ATP for cellular communication. Panx1 has been implicated in many diseases including breast cancer and melanoma, where inhibition or deletion of PANX1 reduced the tumorigenic and metastatic properties of the cancer cells. Potential mechanism: direct interaction between the C-terminal region of PANX1 and the N-terminal portion of β-catenin, a key transcription factor in the Wnt pathway (11).

### Hepatocellular Carcinoma (HCC)

The expressions of Panx1 in 126 cases of HCC were analysed by immunohistochemistry (IHC). The effects of Panx1 on HCC cell metastasis and invasion were observed by investigation of the expression levels of Panx1 and epithelial-mesenchymal transition (EMT) related proteins in HCC cells and tissues. The tumour metastatic abilities were compared between PANX1 knockout mice and nude mice.

A higher expression of PANX1 in HCC was positively correlated with tumour lymph node metastasis. In conclusion, overexpression of Panx1 seems to promote invasion and migration of HCC cells through modulation of EMT in vitro and in vivo (37).

### Breast Cancer

PANX1 overexpression in breast cancer is associated with a shift towards an EMT phenotype, in silico and in vitro, thus attributing to a tumor-promoting effect, with poorer clinical outcomes in breast cancer patients. This association offers a novel target for breast cancer therapy (10).

### Colorectal Cancer

Nuclear factor kappa B (NF-κB) signaling pathway is activated in many colorectal cancer (CRC) cells and in the tumor microenvironment. It was shown found that mefloquine was an NF-κB inhibitor and subsequently it induced growth arrest and apoptosis of CRC cells harboring phosphorylated p65 in culture and in mice. Thus mefloquine could exert antitumor action through inhibiting the NF-κB signaling pathway (12).

### Pancreatic Cancer

Analysis of the expression of PANX1 in human pan-cancer in the Oncomine and GEPIA2.0 databases (Kaplan-Meier plotter and OncoLnc tools). It was shown that PANX1 was overexpressed in most cancers compared to normal tissues. The high expression of PANX1 was associated with poor prognosis in multiple tumors, especially in pancreatic adenocarcinoma (PAAD) (38).

### Leukaemia

Anti-tumour immune responses have been linked to the regulated release of ATP from apoptotic cancer cells to engage P2 purinergic receptor signalling cascades in nearby leukocytes. Comparison of PANX1 levels indicated much higher expression in leukemic T lymphocytes than in normal, untransformed T lymphoblasts. This suggests that signalling roles for PANX1 may be amplified in leukemic leukocytes (39).

### Chemotherapy and Pain

Neuropathic pain is an incapacitating consequence of cancer treatment with cytotoxic chemotherapeutics, such as paclitaxel. The economic cost of chronic pain, including neuropathic pain as a large component, has been estimated to be over $500 billion in the US alone (40).

### Cardiovascular Diseases

### Arrythmia

Crosstalk between GJCs and HCs/PANX1 channels could be crucial in the development of arrhythmogenic substrates, including fibrosis. Current evidence indicates that HCs and PANX1 channel activation can enhance the risk of cardiac arrhythmias. This field may contribute to novel therapeutic approaches for patients prone to develop atrial or ventricular fibrillation (41)(42).

### Vascular Inflammation

Ischemia-reperfusion (I/R) injury (IRI) imposes a significant threat to graft and recipient survival leading to increased morbidity and mortality among patients undergoing lung transplantation.

In wild-type (WT) mice, pharmacological antagonism of PANX1 attenuated lung IRI. Endothelial-specific Panx1 inducible knockout mice demonstrated a protective phenotype after I/R with reduced endothelial permeability, edema, and inflammation. A mechanism of Panx1-mediated protection involves release of ATP by endothelial cells, thereby identifying a potentially effective therapeutic target for the prevention of lung I/R injury.

PANX1 channels on endothelial cells mediate vascular inflammation during lung ischemia-reperfusion injury (43).

### PAH

Hypoxic pulmonary vasoconstriction (HPV) is a physiological response to alveolar hypoxia that diverts blood flow from poorly ventilated to better aerated lung areas to optimize ventilation-perfusion matching. ATP release via PANX1 and subsequent signalling via purinergic P2Y receptors have been identified as regulator of vasoconstriction in systemic arterioles. Pharmaceutical inhibition as well as genetic deletion of the hemichannel PANX1 in pulmonary artery smooth muscle cells attenuates the physiological HPV response (44).

### Blood Pressure

Spironolactone interferes with α1AR (α1 adrenoceptor)-mediated vasoconstriction of resistance vessels and acutely lowers blood pressure in mice. These effects require PANX1 channel expression in vascular smooth muscle cells but are independent of the MR (mineralocorticoid receptor)—the traditional target of spironolactone. It has been proposed that PANX1 is a novel target of spironolactone that, in combination with MR-dependent actions, may contribute to the beneficial blood pressure-lowering effects of spironolactone that are especially relevant for treatment of resistant hypertensive patients (45)(46).

### Inflammatory Diseases

### Joint Inflammation

All joint tissues express one or more connexins and pannexins, and their expression is altered in some pathological conditions, such as osteoarthritis (OA) and rheumatoid arthritis (RA), indicating that they may be involved in the onset and progression of these pathologies. The aging of the global population, along with increases in the prevalence of obesity and metabolic dysfunction, is associated with a rising frequency of joint diseases along with the increased costs and burden of related illness. The modulation of connexins and pannexins represents an attractive therapeutic target in joint disease (47).

### Wound Healing Inflammatory Disorders

Following dorsal skin punch biopsies in Panx1 knockout (KO) mouse, these mutant mice exhibited a significant delay in wound healing. Scratch wound and proliferation assays revealed that cultured keratinocytes from KO mice were more migratory, whereas dermal fibroblasts were more proliferative compared with controls. Also, collagen gels populated with fibroblasts from KO mice exhibited significantly reduced contraction, comparable to WT fibroblasts treated with the Panx1 modulator, probenecid. KO fibroblasts did not increase α-smooth muscle actin expression in response to TGF-β, as is the case for differentiating WT myofibroblasts during wound contraction. PANX1 seems to control cellular properties of keratinocytes and dermal fibroblasts during early stages of skin development and modulates wound repair upon injury (48).

### Pulmonary Diseases

### Covid 19

PANX1 channels regulate inflammation and host responses to several pathogens, including viruses. Several lines of evidence demonstrated that PANX1 channel opening (and release of ATP) enhances inflammatory responses, including in the systemic endothelium (lung microvasculature), lung epithelium, olfactory epithelium, and the parenchyma of several tissues throughout the body. Targeting the hyperinflammation and cytokine storm especially the early phase that occurs in severe cases of COVID-19, could be a key application for PANX1 inhibitors (49).

### Primary and Secondary Ciliary Dyskinesia (PCD and SCD)

Pannexin 1 contributes to release of ATP, an important paracrine regulator of mucociliary function, in airway epithelia. Given the regulation of pannexins, this might have important implications for the availability of ATP in the airway surface liquid in airway homeostasis and disease (50).

### Asthma

Stressed or injured cells release ATP into the extracellular milieu via PANX1 channels, which is the basis of inflammation in a variety of conditions, including allergic lung inflammation. Blockade of PANX1 significantly attenuated goblet cell hyperplasia and inflammatory cell infiltration into the lungs of OVA-sensitized mice. Inhibition of PANX1 also reduced the total and eosinophil cell numbers in the bronchoalveolar lavage fluid (BALF) and reduced expression of CCL11 and CCL2 in lung tissues from mice (51).

### Other Diseases

### Fibrosis

Liver fibrosis is the final common pathway for almost all causes of chronic liver injury. This chronic disease is characterized by excessive deposition of extracellular matrix components mainly due to transdifferentiation of quiescent hepatic stellate cell into myofibroblasts-like cells, which in turn is driven by cell death and inflammation. Gene expression profiling revealed a downregulation of fibrotic and immune responses in pannexin1 knock-out mice treated with carbon tetrachloride, whereas bile duct ligated pannexin1-deficient animals showed a pronounced inflammatory profile (52).

### Diabetes and Related Diseases

Insulin has been identified as a novel activator of PANX1 channels. In obese humans PANX1 expression in adipose tissue is increased and correlates with the degree of insulin resistance. PANX1 seems to be involved in the regulation of β-cells as well. PANX1 is further implicated in glucose resistance and defective glucose uptake in adipocytes leads to impaired metabolic homeostasis and insulin resistance, both hallmarks of type 2 diabetes.

Adipocytes expressed functional Panx1 channels that can be activated to release ATP. Pharmacologic inhibition or selective genetic deletion of PANX1 from adipocytes decreased insulin-induced glucose uptake in vitro and in vivo and exacerbated diet-induced insulin resistance in mice.

Fructose exposure reduced intracellular ATP levels and favoured ATP release from the β-cells upon acute glucose stimulation. The resulting increase in extracellular ATP, mediated by PANX1 channels, activated the calcium-mobilizer P2Y purinergic receptors. Immunodetection revealed the presence of both Panx1 channels and P2Y1 receptors in β-cells.

Type 1 diabetes (T1D) causes a range of skeletal problems, including reduced bone density and increased risk for bone fractures. High glucose levels in T1D alters expression and function of purinergic receptors (P2Rs) and PANX1 channels, and thereby impairs ATP signalling that is essential for proper bone response to mechanical loading and maintenance of skeletal integrity (53-54).

### Eye Diseases

Potential changes in the corneal nerve terminals in non-insulin-dependent diabetes mellitus of moderate duration were investigated in mice. The dissected corneas were subjected to a protocol of ultracentrifugation to obtain synaptosomes of sensory nerve terminals. Within these nerve varicosities, 2 major mechanisms were examined, viz., alterations of the mechanosensitive channel pannexin1 and ATP release on stimulation of these terminals. Thus, altered cellular location and function of the PANX1 channel may contribute to altered mechanosensitivity of the cornea, which in turn may affect wound healing and primary visual function of the cornea (55-56).

### Skin Diseases

### Psoriasis

Psoriasis is a chronic inflammatory disease of the skin associated with systemic and joint manifestations and accompanied by comorbidities, such as metabolic syndrome and increased risk of cardiovascular diseases. Since psoriasis is likely triggered by skin-damaging events and trauma, it is highly likely that intracellular ATP, released by damaged cells, may play a role in triggering the inflammatory response underlying the pathogenesis of the disease by activating the inflammasome. Therefore, purinergic signalling in the skin could represent a new and early step of psoriasis; thus, opening the possibility to target single molecular actors of the purinome to develop new psoriasis treatments. Therefore, a prevention of excessive ATP release may prevent or ameliorate this disease (57)(58).

### Pharmaceutical Compositions

The pharmaceutical compositions of the invention can be prepared and formulated in accordance with the conventional methods such as disclosed, for example, in the British, European and United States Pharmacopeias (59)(60)(61), Remington's Pharmaceutical Sciences (62), Martindale: The Extra Pharmacopoeia (63), and Harry's Cosmeticology (64).

The pharmaceutical forms may comprise, for example, one or more parts of water, buffers (for example, sodium bicarbonate, buffered neutral saline solution of saline solution buffered with phosphate), ethanol, mineral oil, vegetable oil, dimethyl sulfoxide, carbohydrates (for example, lactose, sorbitol, trehalose, glucose, mannose, sucrose, amide, glycerol, mannitol or dextrans), proteins, adjuvants (such as stabilizers like polymers and cyclodextrins), polypeptides or amino acids (such as His, Gly, Lys, Asp, Glu and Arg), antioxidants (such as ascorbic acid, alpha-tocopherol, sulfites, BHA (butylhydroxyanisole), BHT (butylhydroxytoluene), surfactant agents (such as non-ionic detergents—Triton X-100, polysorbate 20, polysorbate 80, Pluronic F68, Pluronic F88, Pluronic F127, Brij 35), chelating agents (such as EDTA and/or glutathione) and/or preservatives (such as parabens, sorbic acid, imidazole urea, ammonia quaternarium compounds hydantoin, phenolic derivatives, acidic derivatives halogenated compounds).

Pharmaceutical forms can be formulated for any route of administration including, for example, topical, oral nasal, rectal or parenteral administration. The term parenteral, as used herein, includes subcutaneous injection, intradermic injection, intravascular injection (for example, intravenous), intramuscular injection, spinal injection, intracranial injection, intrathecal injection, and intraperitoneal injection, as well as any similar technique of injection or infusion. In certain modalities, compositions for oral use are preferred. Such compositions include, for example, pills, tablets, solutions, aqueous or oily suspensions, dispersible powders or granules, emulsions, hard or soft capsules or syrups or elixirs. Among other modalities, pharmaceutical compositions may be formulated with a freeze-dried powder.

Pharmaceutical forms intended for oral use may further comprise other components, such as sweetening agents, flavoring agents, coloring agents and/or preservative agents in order to provide attractive and palatable preparations.

Pills have the active ingredient mixed with physiologically compatible excipients that are adequate for the manufacture of pills. Such excipients include, for example, inert diluents (for example, calcium carbonate, sodium carbonate, lactose, calcium phosphate or sodium phosphate), granulation and disintegration agents (for example, corn starch or alginic acid), bonding agents (for example, starch, gelatin or acacia), and lubricating agents (for example, magnesium stearate, stearic acid or talcum). Pills may be formed using standard techniques, including dry granulation, direct compression, and wet granulation. The pills may not be coated, or they may be coated using known techniques.

Formulations for oral use may also be presented as hard gelatinous capsules wherein the active ingredient is mixed with an inert solid diluent (for example, calcium carbonate, calcium phosphate, kaolin, talcum, monohydrated lactose, colloidal silicon dioxide, microcrystalline cellulose, sodium lauryl sulfate, sodium amide glycolate) or as soft gelatinous capsules, wherein the active ingredient is mixed with water or an oily medium (for example, peanut oil, liquid vaseline or olive).

Aqueous suspensions contain the active material(s) mixed with adequate excipients, such as suspension agents (for example, sodium cellulose carboxymethyl, methylcellulose, hydroxypropyl methylcellulose, sodium alginate, polyvinylpyrrolidone, tragacanth gum, and acacia gum); and dispersion or wetting agents (for example, naturally occurring phosphatides, such as lecithin, products of condensation of an alkylene oxide with fatty acids, such as polyoxyethylene stearate, products of condensation of ethylene oxide with long-chain aliphatic alcohols, such as heptadeca-ethyleneoxy-cetanol, products of the condensation of ethylene oxide with partial esters derived from fatty acids and one hexitol, such as sorbitol polyoxyethylene mono-oleate or products of the condensation of ethylene oxide with partial esters derived from fatty acids and hexitol anhydrides, such as mono-oleate of polyethylene sorbitan). Aqueous suspensions may also comprise one or more preservatives, such as ethyl p-hydroxybenzoate or n-propyl, one or more coloring agents, one or more flavoring agents and/or one or more sweetening agents, such as sucrose or saccharine.

Oily suspensions can be formulated by means of the suspension of the active ingredient(s) in vegetable oil (for example, peanut oil, olive oil, sesame oil or coconut oil) or in mineral oil, such as liquid paraffin. The oily suspensions may contain a thickening agent, such as bee wax, hard paraffin or cetyl alcohol. Sweetening agents, such as those presented above and/or flavoring agents may be added to provide palatable oral preparations. Such suspensions may be preserved by means of the addition of an antioxidant, such as ascorbic acid.

Dispersible powders and granules adequate for the preparation of an aqueous suspension by means of the addition of water provide the active ingredient in a mixture with a dispersion agent or wetting agent, a dispersion agent and one or more preservatives. Adequate dispersion agents or wetting agents are exemplified by those already mentioned above. Additional excipients, such as sweetening agents, flavoring agents, and coloring agents may also be present.

Pharmaceutical forms may also be formulated as water-in-oil emulsions. The oily phase may be a vegetable oil (for example, coconut oil, almonds oil, grape seed oil, olive oil or peanut oil), a mineral oil (for example, liquid Vaseline), or a mixture thereof. Adequate emulsifying agents include naturally occurring gums (for example, acacia gum or tragacanth gum), naturally occurring phospholipids (for example, phosphatidylserine), anhydrides (for example, monooleate of sorbitan) and products of condensation of partial esters derived from fatty acids and hexitol with ethylene oxide (for example, mono-oleate of polyoxyethylene sorbitan). An emulsion can also comprise one or more sweetening agents and/or flavourizers.

Syrups and elixirs may be formulated with sweetening agents, such as glycerol, propylene glycol, sorbitol or sucrose. Such formulations may also comprise one or more preservatives, flavoring agents and/or coloring agents.

Formulations for topical administration typically comprise a topical vehicle combined with the active agent(s), with or without additional optional components. Adequate additional components and topical vehicles are well known in the art and it will be obvious that the choice of a vehicle will depend on the physical form and mode of administration in particular. Topical vehicles include water; organic solvents, such as alcohols (for example, ethanol or isopropyl alcohol) or glycerin; glycols (for example, butylene, isoprene or propylene glycol); aliphatic alcohols (for example, lanoline); mixtures of water and organic solvents and mixtures of organic solvents, such as glycerin alcohol; lipid-based materials, such as fatty acids, acylglycerols (including oils, such as mineral oil and animal or synthetic fats), phosphoglycerides, sphingolipids and waxes; protein-based materials, such as collagen and gelatin; silicone-based materials (volatile and nonvolatile); and hydrocarbon-based materials, such as microsponges and polymeric matrixes.

A composition may further include one or more components adapted to improve the stability or efficacy of the formulation that is applied, such as stabilizing agents, suspension agents, emulsifying agents, viscosity adjusters, gelling agents, preservatives, antioxidants, skin penetration enhancers, humectants, and sustained release materials. Examples of such components are described in the art (65-70). Formulations may comprise microcapsules, such as microcapsules of hydroxymethyl cellulose or gelatin, liposomes, microspheres of albumin, microemulsions, nanoparticles or nanocapsules.

A topical formulation can be prepared through any one a variety of physical forms including, for example, solids, pastes, creams, foams, lotions, gels, powders, aqueous liquids, and emulsions. The physical appearance and viscosity of such pharmaceutically acceptable forms can be oriented by the presence and quantity of emulsifier(s) and viscosity adjuster(s) present in the formulation.

Solids are in general firm and non-pourable and are commonly formulated as bars or clubs or in the form of particles; solids may be opaque or transparent and may optionally contain solvents, emulsifiers, humectants, emollients, fragrances, colorants/dyes, preservatives and other active ingredients that enhance or intensify the effectiveness of the final product.

Creams and lotions are frequently similar to one another, differing mainly in terms of their viscosity; lotions and creams may be opaque, translucid or transparent, and frequently contain emulsifiers, solvents, and agents for adjustment of viscosity, as well as humectants, emollients, fragrances, colorants/dyes, preservatives and other active ingredients that enhance or increase the effectiveness of the final product.

Gels may be prepared with a series of viscosities, from thick with high viscosity to thin with low viscosity. Those formulations, as well as those of lotions and creams, can also contain solvents, emulsifiers, humectants, emollients, fragrances, colorants/dyes, preservatives and other active ingredients that enhance or increase the effectiveness of the final product.

Liquids are thinner than creams, lotions or gels and frequently do not contain emulsifiers. Liquid topical products frequently contain solvents, emulsifiers, humectants, emollients, fragrances, colorants/dyes, preservatives and other active ingredients that enhance or increase the effectiveness of the final product.

Emulsifiers adequate for use in topical formulations include, without limitations, ionic emulsifiers, ceteralylic alcohol, non-ionic emulsifiers, such as polyoxyethylene oleyl ether, PEG-40 stearate, cetearyl alcohol such as ceteareth-12, ceteareth-20, ceteareth-30, PEG-100 stearate, and glyceryl stearate. Adequate agents for the adjustment of viscosity include, without limitation, protective colloids of non-ionic gums, such as hydroxyethyl cellulose, xanthan gum, aluminum magnesium silicate, silica, microcrystalline wax, bee wax, paraffin, and cetyl palmitate. A gel composition may be formed by means of the addition of a gelling agent, such as chitosan, methylcellulose, ethyl cellulose, polyvinyl alcohol, polyquaterniums, hydroxyethyl cellulose, hydroxypropyl cellulose, hydroxypropyl methylcellulose, carbomer or glycyrrhizinate with ammonia. Adequate surfactants include, without limitations, non-ionic surfactants, amphoteric surfactants, ionic surfactants, and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, chloride of cocamidopropyl phosphatidyl PG-diammonium and ammonium laureth sulfate can be used in topical formulations. Adequate preservatives include, without limitations, antimicrobials, such as methylparaben, propylparaben, sorbic acid, benzoic acid, and formaldehyde, as well as physical stabilizers and antioxidants, such as vitamin-E, ascorbic acid, and propyl gallate. Adequate humectants include, without limitations, lactic acid and other hydroxy acids and their salts, glycerin, propylene glycol, and butylene glycol. Adequate emollients include derivatives of lanolin, petrolatum, isostearyl neopentanoate, and mineral oils. Adequate fragrances and colorants include, without limitations, FD&C Red No. 40 and FD&C Yellow No. 5. Other adequate additional ingredients that may be used topically include, without limitations, abrasives, absorbents, anti-foaming agents, anti-static agents, astringents (for example, hamamelis, alcohol and herbal extracts, such as chamomile extract), binders/excipients, buffering agents, chelating agents, film-forming agents, conditioning agents, propellants, opacifying agents, pH regulators and protectors.

Among the formulations for topical use one may further point out cutaneous permeation promoter excipients which may function is to enhance the release of the compound on the surface of the skin, through the stratum corneum, in a transdermic system. The main promoters of permeation used in the release of pharmaceuticals include alcohols, glycols and glycerides, such as ethanol, propylene glycol, ethoxy diglycol, 1-decanol, 2-(2-ethoxyethoxy)ethanol; fatty acids and esters, such as palmitic acid, capric acid, oleic acid, myristic acid, or lauric acid (71-73); sulfoxides, such as dimethylsulfoxide and dimethylformamide (74); phospholipids, such as phosphatidylglycerol, phosphatidylcholine and phosphatidylethanolamine; cyclodextrins (α-cyclodextrin, β-cyclodextrin and γ-cyclodextrin); dodecyl-N,N-dimethylamino acetate (DDAA); polymers such as already cited previously herein.

Other pathways for permeation enhancers comprise physical methods such as iontophoresis (75), electroporation (76), and phonophoresis (77).

Typical modes of administration for topical compositions for external use include direct application of the product using the hands with the use of glove; or indirect application using a physical applicator, such as a spatula, a dosing syringe, a dosing rule, adhesive or stick; spraying (including mist spraying, aerosol or foam); use of single-dose sachets of 1 ml; application with a drop counter; dispersion and rinsing. One other form of indication for topical use is inhalation, or application in other different tissues of the skin, such as eyedrops applied in the conjunctive tissue or otological solutions for auricular application.

These inhalator formulations, in an exemplary form, include gaseous forms in aerosol (using a conventional propellant, for example, dichlorofluoromethane or trichlorofluoromethane), or particulates in the form of spray drying and emulsions, solutions or suspensions for liquids inhaled by nebulization. Further, we may exemplify a pharmaceutical form by ophthalmic or conjunctival pathway, cold creams, post-reconstituted, eye drops in isotonic suspensions or sterile suspensions dispensed by an eye dropper, and by otological pathway, cold creams or liquid isotonic pharmaceutical forms also dispensed with a drop dispenser.

A pharmaceutical form may be prepared as a sterile injectable aqueous or oily suspension. The compound(s) provided herein, depending on the vehicle and the concentration used, may be suspended or dissolved in such composition may be formulated in accordance with the known technique using adequate dispersion agents, wetting agents and/or suspension agents, such as those mentioned hereinabove. Among the acceptable vehicles and solvents that may be employed are water, 1,3-butanediol, Ringer's solution and isotonic solution of sodium chloride, sodium citrate and excipients that may include adjuvants such as complexes of inclusion with cyclodextrins, or releasing systems such as nanoemulsions, nanosuspensions, microemulsions, polymeric micelles, liposomes, niosomes, transfersomes and ethosomes (78-80).

Furthermore, sterile fixed oils can be employed as a solvent or a suspension medium. For that purpose, any soft fixed oil can be used, including synthetic monoglycerides or synthetic diglycerides. Furthermore, fatty acids, such as oleic acid, are useful in the preparation of injectable compositions and adjuvants, such as local anesthetics, preservatives and/or buffering agents can be dissolved in the vehicle.

Pharmaceutical forms can also be formulated as suppositories (for example, for rectal administration). Such compositions can be prepared by mixing the drug with an adequate non-irritating excipient that is solid at ambient temperatures but becomes liquid at the rectal temperature and therefore will dissolve in the rectum to release the drug.

Pharmaceutical forms may be formulated to be released at a predetermined rate. An instant release may be obtained, for example, via sublingual administration (that is, administration through the mouth in such a manner that the active ingredient(s) is/are rapidly absorbed through the blood vessels of the sublingual plexus).

Formulations with a controlled release (that is, formulations such as a capsule, pill or coated table that diminishes and/or delays the release of the active ingredient(s) after administration) may be administered, for example orally, rectally or subcutaneously or through an implant in a target location. In general, a formulation with controlled release may be obtained by means of the combination of the active ingredient(s) with a matrix material that, in itself, changes the release rate and/or through the use of a coating with controlled release, which delays the disintegration and absorption in the intestinal tract (or location of implant), and thereby provides a delayed or a sustained action during a longer period. One such type of formulation with a controlled release is a formulation with sustained release, in which at least one active ingredient is continuously released during a period of time at a constant rate. Preferably, the therapeutic agent is released at a rate such that the concentrations in the blood (for example, plasma) are maintained within the therapeutic range, however below the toxic levels, during a period of time that is at least 4 hours, preferably at least 8 hours, and more preferably at least 12 hours. Such formulations may, in general, be prepared using well-known technologies. Vehicles for use inside such formulations are biocompatible, and may also be biodegradable. Preferably, a formulation provides a constant level of release of the modulator. The amount of modulator contained in a formulation with sustained release depends, for example, on the location of the implant, the expected release the rate and duration and the nature of the condition to be treated or prevented.

The release rate may be varied using methods well known in the art including (a) variation of thickness of composition of the coating, (b) alteration of the quantity of manner of addition of plasticizer on a coating (c) inclusion of additional ingredients, such as agents that modify the release, (d) alteration of the composition, particle size or format of particle of the matrix and (e) provision of one or more passages through the coating. The amount of modulator contained within a sustained release formulation depends, for example, from the method of administration (for example, the location of the implant), the rate and duration of release that is expected and the nature of the condition to be treated or prevented.

The matrix material, which in itself may or not serve a controlled release function, is generally any material that support(s) the active ingredient(s). For example, a material such as a glyceryl monostearate or glyceryl distearate may be employed. Active ingredient(s) may be combined with the matrix material prior to the formation of the dosage form (for example, a pill). Alternatively, or furthermore, the active ingredient(s) may be coated on the surface of a particle, granule, sphere, microsphere, globule or pellet that comprises the matrix material. Such coating may be obtained via conventional means, such as through dissolution of the active ingredient(s) in other or another adequate solvent and spraying. Optionally, extra ingredients are added prior to the coating (for example, to aid in the binding of the active ingredient(s) to the matrix material). The matrix may then be coated with a barrier agent before the application of the controlled release coating. Multiple coated matrix units may, if desired, be encapsulated to generate the final dosage form.

The controlled release coating may be a film, continuous and uniform, capable of supporting pigments and other additives, non-toxic, inert and devoid of adherence. Coatings that regulate the release of the modulator include pH-independent or dependent coatings, which can be used to release the modulator in the stomach and enteric coatings (which permit the formulation to pass intact through the stomach, and in the small intestine the coating dissolves and the contents are absorbed by the body). pH-dependent coatings include, for example, shellac, cellulose acetate phthalate, polyvinyl acetate phthalate, cellulose methyl hydroxypropyl phthalate, copolymers of an ester of methacrylic acid and zeine.

In certain modalities, the coating is a hydrophobic material, preferably used in an amount effective to reduce the hydration of the gelling agent after administration. Adequate hydrophobic materials include alkyl celluloses (for example, ethyl cellulose or carboxymethyl cellulose ethers), cellulose ethers, cellulose esters, acrylic polymers (for example, (poly)acrylic acid, (poly)methacrylic acid, copolymers of acrylic acid and methacrylic acid, copolymers of methyl methacrylate, ethoxy ethyl methacrylate, copolymer of alkamide/methacrylic acid, (poly)methyl methacrylate, polyacrylamide, ammonium methacrylate copolymer, aminoalkyl methacrylate copolymer, (poly)methacrylic acid anhydride and glycidyl methacrylate copolymers) and mixtures thereof.

Aqueous dispersions representative of ethyl cellulose includes, for example, AQUACOAT® (FMC Corp., Philadelphia, Pa.) and SURELEASE® (Colorcon, Inc., West Point, Pa.), both being applicable to the substrate according to the manufacturer's instructions. Representative acrylic polymers include, for example, the various polymers EUDRAGIT® (Rohm America, Piscataway, N.J.), which can be alone or in combination, depending on the desired release profile.

The physical properties of coatings that comprise an aqueous dispersion of hydrophobic material may be improved by means of the addition of one or more plasticizers. Plasticizers adequate for alkyl celluloses include, for example, dibutyl sebacate, diethyl phthalate, triethyl citrate, tributyl citrate, and triacetin. Plasticizers adequate for acrylic polymers include, for example, citric acid esters, such as triethyl citrate and tributyl citrate, dibutyl phthalate, polyethylene glycols, propylene glycol, diethyl phthalate, castor-oil plant, and triacetin.

Controlled release coatings are in general applied using conventional techniques, such as by means of spraying in the form of an aqueous dispersion. If so desired, the coating may comprise pores or channels to facilitate the release of the active ingredient. Pores and channels may be generated using well-known methods, including the addition of an organic or inorganic material that is dissolved, extracted or released from the coating in the environment of use. Some of such pore-formation materials include hydrophilic polymers, such as hydroxyalkyl celluloses (for example, hydroxypropyl methylcellulose), cellulose ethers, water-soluble synthetic polymers (for example, polyvinylpyrrolidone, cross-linked polyvinylpyrrolidone, and polyethylene oxide), water-soluble polydextrose, saccharides and polysaccharides, and alkaline metal salts.

The amount of active ingredient that can be combined with the materials of the vehicle to produce a unit dose will vary depending, for example, from the patient that is being treated, from the mode of administration in particular and any other co-administered drugs. Dosage units generally contain between about 5 pg to about 2 g of the active ingredient. Optimal dosages may be established using tests and routine procedures that are well known in the art.

In one aspect of the present invention, the compositions can comprise, in addition to the one or more panx-1 modulators of the present invention, one or more additional active ingredients that include, without limitations, for example, analgesics, anti-inflammatory agents, anti-arrhythmia agents, anticoagulants, thrombolytic agents, diuretics, anti-depressives, anti-diabetic agents, anti-epileptic agents, anti-histaminic agents, anti-hypertensive agents, anti-muscarinic agents, anti-mycobacterial agents, anti-neoplastic agents, immunomodulators, antiviral agents, anxiolytic sedatives (hypnotics and neuroleptics), beta-adrenoreceptor blockers, inotropic agents cardiac agents, corticosteroids, diuretics, dopaminergics (antiparkinsonian agents), immunologic agents, muscle relaxants, parasympathomimetics, prostaglandins, bronchodilators, anti-allergic agents, sympathomimetic agents, anti-emetic agents, chemotherapic agents, and xanthines.

The invention will thus hereinafter be described by means of examples, which illustrate additionally the present invention, without it being intended, however, that such examples limit the scope of the present invention.

### EXAMPLES

### Example 1—In Silico Docking

The first virtual screening study (docking analyses performed with AUTODOCK on the identified ‘CBX binding site’ in Panx-1 channel (PDB code 7DWG), using known inhibitors (difloxacine, trovafloxacine, Brilliant Blue G-FCF, Levofloxacine, compound 14c (9), compound 5b (9), Imiquimod var 1, compound 15b (9), Baicalein, compound 61 (9), compound 6e (9), plus mefloquine, Carbenoxolone, Quinine, minus mefloquine, Probenecid, 5-nitro-2-(3-phenylpropylamino)benzoic acid, Cholesterol) and some of our suggested Panx1 blockers.

The later approach was done by using the module 3D sim of the software Stardrop 7.1 (https://optibrium.com/stardrop/). In this assay the candidate compounds were compared to the structure of Carbenoxolone, a known PANX1 inhibitor with a well-described binding site. The software computes a similarity index of a candidate compound in view a comparator (i.e., carbenoxolone) based on steric and Coulombic configurations, number of H-bond donors and number of H-bond acceptors. Other known PANX1 inhibitors were introduced in the study as standard comparators, namely: quinine, plus and minus mefloquine, imiquimod var 1, baicalein, levofloxacin, trovafloxacin, difloxacin, and Probenecid) as well as compounds 14c and 6e from Crocetti et al (2021).

This approach followed largely the method published in reference (1). As comparator the 3D structure of human Panx1 with the inhibitor Carbenoxolone was used. The structure was downloaded from the PDB database as 6WBI and 6WBL-Cryo-EM structure of human Pannexin 1 channel with or without deletion of N-terminal helix and C-terminal tail, in complex with CBX (Ruan et al., 2020).

The following molecules have been designed and their binding energy was calculated as outlined with Autodock (PDB database: 7DWB). As a reference the calculated binding energy of one particular conformation of carbenoxolone was used,

The binding energies for known PANX1 ligands are shown in Table 1, while the results for compounds of the invention are described in Table 2. A skilled artisan will recognize that the docking score in silico may predict the in vitro and in vivo affinity of the compounds of the invention for PANX1.

TABLE 1
Binding energies of Carbenoxolone, Quinine,
minus mefloquine, and Probenecid for PANX1.

| Known PANX1 | calculated binding |
|---|---|
| ligand | energy (kcal/mol) |
| Carbenoxolone | −4.469 |
| Quinine | −4.438 |
| minus mefloquine | −4.265 |
| Probenecid | −3.994 |

TABLE 2
Binding energies of the PANX1 modulators
of the present invention for PANX1.

| | Calculated | | Calculated |
|---|---|---|---|
| | binding energy | | binding energy |
| Name | (kcal/mol) | Name | (kcal/mol) |
| CMPD 040 | −6.441 | CMPD 036 | −4.822 |
| CMPD 042 | −6.351 | CMPD 038 | −4.78 |
| CMPD 041 | −6.277 | CMPD 037 | −4.769 |
| CMPD 048 | −5.836 | CMPD 028 | −4.671 |
| CMPD 046 | −5.761 | CMPD 049 | −4.43 |
| CMPD 034 | −5.748 | CMPD 026 | −4.418 |
| CMPD 035 | −5.748 | CMPD 051 | −4.404 |
| CMPD 043 | −5.628 | CMPD 029 | −4.388 |
| CMPD 030 | −5.6 | CMPD 001 | −4.376 |
| CMPD 023 | −5.211 | CMPD 032 | −4.272 |
| CMPD 031 | −5.173 | CMPD 025 | −4.265 |
| CMPD 044 | −5.123 | CMPD 017 | −4.214 |
| CMPD 019 | −5.056 | CMPD 018 | −4.096 |
| CMPD 047 | −5.006 | CMPD 033 | −4.071 |
| CMPD 010 | −4.979 | CMPD 039 | −3.95 |
| CMPD 027 | −4.925 | CMPD 045 | −3.878 |
| CMPD 052 | −4.92 | CMPD 004 | −3.727 |
| CMPD 050 | −4.907 | CMPD 011 | −3.661 |
| CMPD 053 | −4.824 | | |

### Example 2—Preparation of Cmpd 004

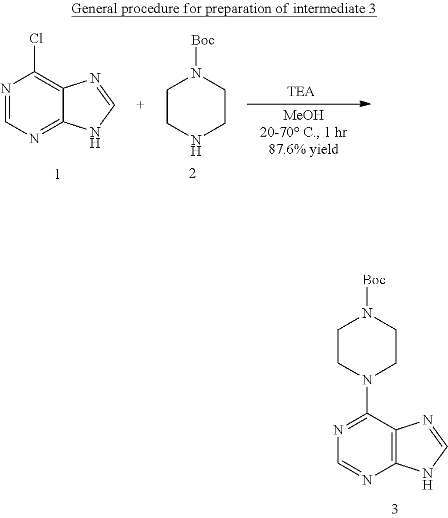 [SMILES: Clc1ncnc2[nH]cnc12 ; CC(C)(C)OC(=O)N1CCNCC1 ; CC(C)(C)OC(=O)N1CCN(c2ncnc3[nH]cnc23)CC1]

Charged Intermediate 1 (2.00 g, 12.9 mmol, 1.00 eq), MeOH (30.0 mL) and Intermediate 2 (2.91 g, 15.6 mmol, 1.21 eq) in a 100 mL flask 1 at 20° C.

Charged TEA (1.58 g, 15.6 mmol, 2.17 mL, 1.21 eq) in a flask 1 at 20° C.

Stirred at 70° C. for 1 hr.

HPLC (EC3602-25-P1A3) indicated Intermediate 1 was consumed completely and a new main peak (Rt=1.920 min) was formed.

The reaction mixture was cooled to 20° C.

Poured the mixture into H₂O (150 mL) stir at 20° C. for 0.50 h and filtered dry the filter cake under vacuum to obtain Intermediate 3 (3.45 g, 11.3 mmol, 87.6% yield, 100% purity) as gray solid, confirmed via LCMS and ¹H NMR.

HPLC: Rt=1.920 min, 23.2% purity under 220 nm

LCMS: Rt=0.406 min, m/z=305.2 (M+H)⁺

¹H-NMR (400 MHz, DMSO-d₆): δ 13.07 (s, 1H), 8.23 (s, 1H), 8.15 (s, 1H), 4.20 (s, 4H), 3.45 (t, J=4.8 Hz, 4H), 1.43 (s, 9H).

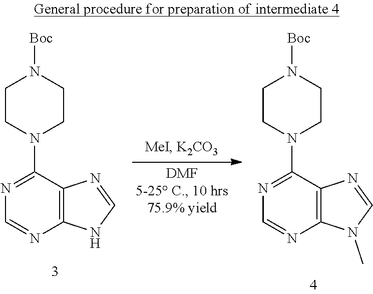 [SMILES: CC(C)(C)OC(=O)N1CCN(c2ncnc3[nH]cnc23)CC1 ; Cn1cnc2c(N3CCN(C(=O)OC(C)(C)C)CC3)ncnc21]

Equipped a 250 mL of three flask with overhead stirrer, addition funnel and thermometer.

Charged DMF (100 mL) to the flask.

Charged intermediate 3 (3.40 g, 11.1 mmol, 1.00 eq) into the flask at 20-25° C.

Charged K₂CO₃ (1.73 g, 12.5 mmol, 1.12 eq) into the flask at 5-10° C.

Charged drop-wise MeI (2.38 g, 16.7 mmol, 1.04 mL, 1.50 eq) into the flask at 5-10° C.

Stirred at 20-25° C. for 10 h.

Took a sample for LCMS (EC378-385-P1E1): intermediate 3 was consumed completely and one main peak with desired mass (Rt=0.421 min) was detected.

Poured the mixture into H₂O (350 mL) and stirred at 20-25° C.

Charged AcOK (10.0 g) into the mixture.

Extracted the mixture with ethyl acetate (100 mL*3).

Washed the organic phase with brine (100 mL*1) and separate.

Dried the organic phase with Na₂SO₄ and filtered, the filtrate was concentrated under vacuum.

The residue was triturated with petroleum ether/ethyl acetate=3/1 (10.0 mL) at 15° C. for 1 h to obtain intermediate 4 (2.70 g, 8.48 mmol, 75.9% yield) as yellow solid, confirmed via ¹H NMR and HSQC.

LCMS: Rt=0.421 min, m/z=319.1 (M+H)⁺.

¹H NMR (400 MHz, CDCl₃): δ 8.40 (s, 1H), 7.74 (s, 1H), 4.31 (s, 4H), 3.84 (s, 3H), 3.59 (t, J=5.2 Hz, 4H), 1.50 (s, 9H).

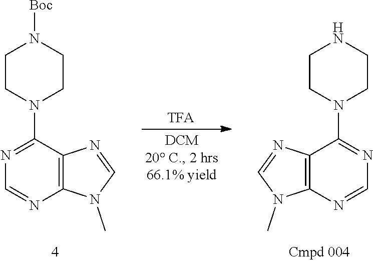 [SMILES: Cn1cnc2c(N3CCN(C(=O)OC(C)(C)C)CC3)ncnc21 ; Cn1cnc2c(N3CCNCC3)ncnc21]

Intermediate 4 (1.20 g, 3.77 mmol, 1.00 eq) was mixed with trifluoroacetic acid (TFA) (5.00 mL) in Dichloromethane (DCM) (20.0 mL) to R1 (100 mL three-necked flask) at 20° C.

The mixture was stirred at 20° C. for 2 h.

A sample was taken for HPLC (EC4923-3-P1A2): intermediate 4 was consumed completely and one main peak with desired mass (Rt=0.922 min) was detected.

The reaction mixture was poured into NaOH solution (1 mmol/mL) then NaOH solution (1 mmol/mL) was added until pH=13.

The mixture was extracted with DCM/MeOH=5/1 (60.0 mL*5), the combined organic layers were dried over Na₂SO₄, filtered and concentrated at 45° C.

Triturate the residue with the solvent (ethyl acetate, 5 mL) at 20° C. for 2 h and filtered to obtain Cmpd 004 (572 mg, 2.49 mmol, 66.1% yield, 98.9% purity) as yellow solid, confirmed by ¹H NMR, HPLC, and LCMS.

HPLC: Rt=0.922 min, 97.3% purity under 220 nm.

LCMS: Rt=0.227 min. m/z=219.0 (M+H)⁺.

HPLC: Rt=0.932 min, 98.9% purity under 220 nm.

¹H NMR (400 MHz, MeOD): δ 8.25 (s, 1H), 7.99 (s, 1H), 4.26 (s, 4H), 3.80 (s, 3H), 2.94 (t, J=5.2 Hz, 4H).

### Example 3—Preparation of Cmpd 010

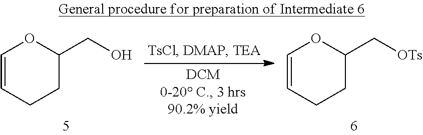 [SMILES: OCC1CCC=CO1 ; Cc1ccc(S(=O)(=O)OCC2CCC=CO2)cc1]

Charged Intermediate 5 (10.0 g, 87.6 mmol, 9.35 mL, 1.00 eq), TsCl (25.0 g, 131 mmol, 1.50 eq), TEA (13.3 g, 131 mmol, 18.2 mL, 1.50 eq), DMAP (1.07 g, 8.76 mmol, 0.10 eq) in DCM (100 mL) to R1 (500 mL three-necked flask) at 0° C.

The mixture was stirred at 20° C. for 3 h.

TLC (petroleum ether/ethyl acetate=3/1) indicated Intermediate 5 (R_{f}=0.40) was consumed completely and one new spot (R_{f}=0.65) formed.

The reaction mixture was poured into H₂O (300 mL) and extracted with DCM (200 mL*2).

The reaction ether phases were washed with brine (200 mL*3), dried over anhydrous Na₂SO₄, filtered and concentrated in vacuum at 45° C.

The residue was purified by column chromatography (SiO₂, petroleum ether/ethyl acetate=20/1 to 1/1, TLC: petroleum ether/ethyl acetate=3/1, R_{f}=0.65) to obtain Intermediate 6 (21.4 g, 79.0 mmol, 90.2% yield, 99.1% purity) as a white solid, confirmed by ¹H NMR, LCMS, and HPLC.

LCMS: Rt=0.530 min, m/z=269.0 (M+H)⁺.

HPLC: Rt=2.345 min, 99.1% purity under 220 nm.

¹H NMR (400 MHz, DMSO-d₆): δ 7.79 (d, J=8.4 Hz, 2H), 7.48 (d, J=8.0 Hz, 2H), 6.29 (d, J=6.0 Hz, 1H), 4.67-4.65 (m, 1H), 4.15-4.11 (m, 1H), 4.08-4.04 (m, 1H), 3.98-3.94 (m, 1H), 2.43 (s, 3H), 1.98-1.87 (m, 3H), 1.53-1.45 (m, 1H).

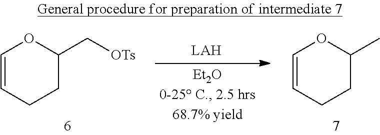

To a solution of compound 6 (70.0 g, 260 mmol, 1.00 eq) in dry Et₂O (600 mL). Then LAH (19.8 g, 521 mmol, 2.00 eq) was added to the mixture in portions (10 times) at 0° C. under N₂. The reaction mixture was stirred at 0° C. for 30 min under N₂, then the mixture was stirred at 25° C. for 2 h.

Took a sample for ¹H NMR (EC378-428-P1D2): Intermediate 6 was consumed completely and desire peak was detected.

Cooled the mixture to −5° C., 100 g Na₂SO₄·10H₂O was added to the mixture in portions (30 times) at −5˜0° C. The mixture was warmed to 20° C. and stirred 15 min. Filtered the mixture, washed the filter cake with Et₂O (250 mL). Wash the organic phase with brine (300 mL*2) and separated. Dried the organic phase with Na₂SO₄ and filtered. The filtrate was distilled in vacuum (35° C., 0.05 Mpa) to remove the Et₂O and unknown impurities.

The filtrate was distilled in vacuum (35° C., 0.05 Mpa) to remove the Et₂O and unknown impurities, thus obtaining Intermediate 7 (17.6 g, 179 mmol, 68.7% yield) as colorless oil, confirmed by ¹H NMR.

¹H NMR (400 MHz, CDCl₃): δ 6.36 (d, J=6.0 Hz, 1H), 4.67-4.65 (m, 1H), 3.95-3.91 (m, 1H), 2.09-2.04 (m, 1H), 1.98-1.97 (m, 1H), 1.84-1.82 (m, 1H), 1.60-1.56 (m, 1H), 1.27 (d, J=6.4 Hz, 3H).

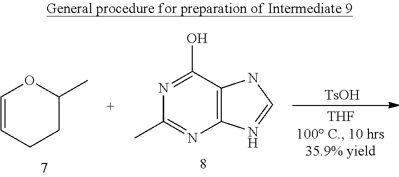

A mixture of Intermediate 7 (686 mg, 6.99 mmol, 3.00 eq), Intermediate 8 (350 mg, 2.33 mmol, 1.00 eq) and TsOH (602 mg, 3.50 mmol, 1.50 eq) in THF (5.00 mL) was stirred at 100° C. for 10 h.

TLC analysis (dichloromethane/methanol=5/1) showed that Intermediate 7 (R_{f}=0.10) was consumed, and Intermediate 9 (R_{f}=0.30) was detected.

Combined six batches to work up. Poured the mixture in 40.0 mL H₂O, extracted the mixture with DCM (50.0 mL*3), washed the organic phase with brine (100 mL*1) and separated. Dried the organic phase with Na₂SO₄ and filtered, the filtrate was concentrated under vacuum.

The residue was purified by MPLC (SiO₂, R_{f}=0.30, dichloromethane/methanol=5/1, petroleum ether/ethyl acetate=10/1 to 0/1) to obtain Intermediate 9 (1.30 g, 5.03 mmol, 35.9% yield, 96.0% purity) as yellow solid, confirmed via HPLC, LCMS, and ¹H NMR.

LCMS: Rt=0.364 min, m/z=497.3 (2M+H)⁺.

HPLC: Rt=1.126 min, 96.0% purity under 220 nm.

¹H NMR (400 MHz, MeOD): δ 8.15 (s, 1H), 5.75-5.71 (m, 1H), 5.49 (s, 1H), 3.87-3.82 (m, 1H), 2.48 (s, 3H), 2.06-2.00 (m, 3H), 1.73-1.69 (m, 2H), 1.38-1.37 (m, 1H), 1.23 (d, J=6.0 Hz, 3H).

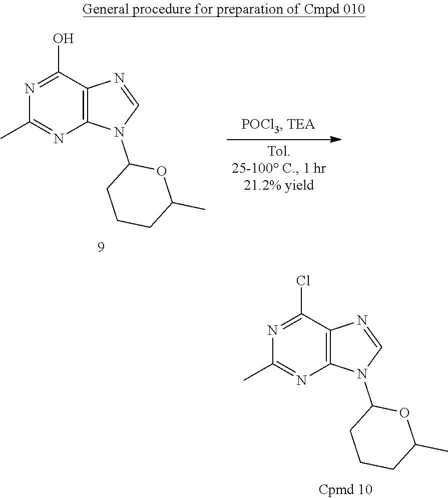

Charged compound 9 (200 mg, 805 μmol, 1.00 eq) and Tol. (5.00 mL) in R1 (40.0 mL flask) at 25° C.

Charged POCl₃ (185 mg, 1.21 mmol, 112 μL, 1.50 eq) in R1 at 25° C.

Drop-wise TEA (122 mg, 1.21 mmol, 168 μL, 1.50 eq) in R1 at 25° C.

Stirred R1 at 100° C. for 1 hr.

Took a sample for TLC (dichloromethane/methanol=5/1): showed compound 9 (R_{f}=0.30) was consumed, and one new point (R_{f}=0.90) was detected.

Cooled R1 to 25° C.

Combined five batches to work up.

The reaction mixture was poured into warm water (30.0 mL, 30-40° C.).

Then extracted with ethyl acetate (50.0 mL*2).

The combined organic layer was washed with brine (10.0 mL*1) dried over Na₂SO₄, filtered and the filtrate was concentrated to give the crude product.

The crude product was purified by prep-TLC (petroleum ether/ethyl acetate=1/1, R_{f}=0.30) to obtain Intermediate 9 (243 mg, 856 μmol, 21.2% yield, 95.2% purity) as yellow solid, confirmed via HPLC, LCMS, and ¹H NMR.

LCMS: Rt=0.364 min, m/z=497.3 (2M+H)⁺.

HPLC: Rt=1.126 min, 96.0% purity under 220 nm.

¹H NMR (400 MHz, MeOD): δ 8.63 (s, 1H), 5.89-5.86 (m, 1H), 3.91-3.86 (m, 1H), 2.74 (s, 3H), 2.15-2.05 (m, 3H), 1.85-1.72 (m, 2H), 1.42-1.25 (m, 1H), 1.24 (d, J=6.4 Hz, 3H).

### Example 4—Preparation of Cmpd 011

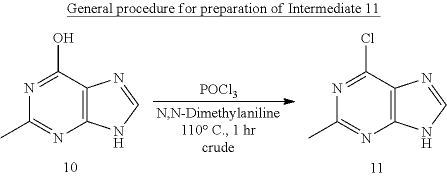

A mixture of Intermediate 10 (2.00 g, 13.3 mmol, 1.00 eq) in N, N-Dimethylaniline (3.30 mL).

Charged POCl₃ (15.0 mL) in the mixture.

The reaction mixture was stirred at 110° C. for 1 hr.

Took a sample for TLC (petroleum ether/ethyl acetate=0/1): Intermediate 10 (R_{f}=0.20) was consumed, and one spot (R_{f}=0.90) was detected.

Cooled R1 to 25° C.

The reaction mixture was poured into finely crushed ice (100 mL).

Then extracted with ethyl acetate (100 mL*2).

The combined organic layer was washed with cold water (50.0 mL*3) dried over Na₂SO₄, filtered and the filtrate was concentrated to obtain Intermediate 11 (2.00 g, crude) as a yellow solid.

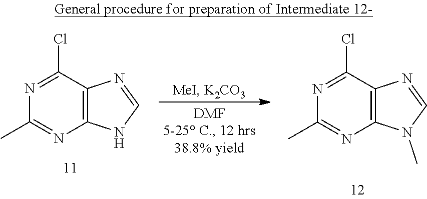

Equipped a 100 mL of three flask with overhead stirrer, addition funnel and thermometer.

Charged DMF (20.0 mL) to the flask.

Charged Intermediate 11 (2.00 g, 11.8 mmol, 1.00 eq) to the flask at 20-25° C.

Charged K₂CO₃ (4.92 g, 35.5 mmol, 3.00 eq) into the flask at 5-10° C.

Charged drop-wise MeI (3.37 g, 23.7 mmol, 1.48 mL, 2.00 eq) into the flask at 5-10° C.

Stirred at 20-25° C. for 12 h.

Took a sample for LCMS (EC378-408-P1D1): Intermediate 11 was consumed completely and one main peak with desired mass (Rt=1.431 min) was detected.

Poured the mixture into H₂O (100 mL) and stir at 20-25° C.

Charged AcOK (10.0 g) into the mixture.

Extracted the mixture with ethyl acetate (30.0 mL*3)

Washed the organic phase with brine (30.0 mL*1) and separated.

Dried the organic phase with Na₂SO₄ and filtered, the filtrate was concentrated under vacuum.

The residue was purified by MPLC (SiO₂, R_{f}=0.20, petroleum ether/ethyl acetate=1/1, petroleum ether/ethyl acetate=10/1 to 1/1) to obtain Intermediate 12 (1.00 g, 4.61 mmol, 38.8% yield, 84.2% purity) as yellow solid, confirmed via LCMS.

LCMS: Rt=1.431 min, m/z=183.1 (M+H)⁺.

LCMS: Rt=1.378 min, m/z=183.2 (M+H)⁺.

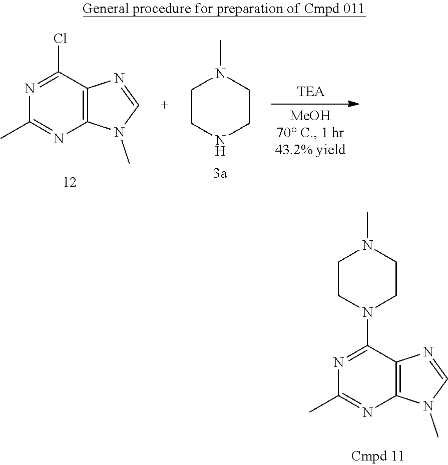

To a mixture of Intermediate 3a (1.10 g, 10.9 mmol, 1.21 mL, 2.00 eq), Intermediate 12 (1.00 g, 5.48 mmol, 1.00 eq) and TEA (1.11 g, 10.9 mmol, 1.52 mL, 2.00 eq) in MeOH (10.0 mL). The mixture was stirred at 70° C. for 1 hr.

Took a sample for LCMS (EC378-409-P1A1): Intermediate 12 was consumed completely and one main peak with desired mass (Rt=1.351 min) was detected.

Poured the mixture in 20.0 mL H₂O, extracted the mixture with DCM (50.0 mL*3), washed the organic phase with brine (100 mL*1) and separated. Dried the organic phase with Na₂SO₄ and filtered, the filtrate was concentrated under vacuum.

The residue was purified by MPLC (SiO₂, R_{f}=0.20, petroleum ether/ethyl acetate=1/1, petroleum ether/ethyl acetate=10/1 to 1/1) to obtain Intermediate 10 (588 mg, 2.37 mmol, 43.2% yield, 99.3% purity) as blue solid, confirmed via LCMS, HPLC, and ¹H NMR.

LCMS: Rt=1.351 min, m/z=247.1 (M+H)⁺.

LCMS: Rt=0.507 min, m/z=247.1 (M+H)⁺.

HPLC: Rt=1.684 min, 99.3% purity under 220 nm.

¹H NMR (400 MHz, MeOD): δ 7.92 (s, 1H), 4.29 (s, 4H), 3.78 (s, 3H), 2.58-2.52 (m, 7H), 2.35 (s, 3H).

### Example 5—Preparation of Cmpd 017

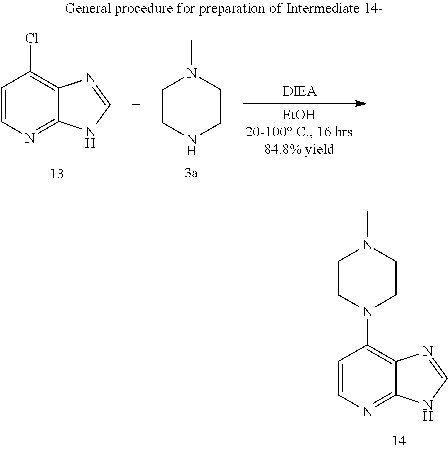

Charged Intermediate 13 (1.50 g, 9.77 mmol, 1.00 eq), Intermediate 3a (3.91 g, 39.0 mmol, 4.33 mL, 4.00 eq), DIEA (6.31 g, 48.8 mmol, 8.51 mL, 5.00 eq) in EtOH (15.0 mL) to bottle at 20° C.

The mixture was stirred at 100° C. for 16 h.

Took a sample for TLC (dichloromethane/methanol=10/1): Intermediate 13 (R_{f}=0.60) was consumed, one main spot (R_{f}=0.40) was detected.

Concentrated the reaction mixture under vacuum at 50° C. to give a residue.

The residue was purified by column chromatography (SiO₂, dichloromethane/methanol=50/1 to 10/1, TLC: dichloromethane/methanol=10/1, R_{f}=0.40) to obtain Intermediate 14 (1.80 g, 8.28 mmol, 84.8% yield, 100% purity) as a yellow solid, confirmed by LCMS, HPLC, and ¹H NMR.

LCMS: Rt=1.136 min, m/z=218.1 (M+H)⁺.

HPLC: Rt=0.862 min. 100% purity under 220 nm

¹H NMR (400 MHz, DMSO-d₆): δ 12.71 (s, 1H), 8.06 (s, 1H), 7.40 (d, J=5.6 Hz, 1H), 6.47 (d, J=5.6 Hz, 1H), 3.86 (s, 4H), 2.46 (s, 4H), 2.22 (s, 3H)

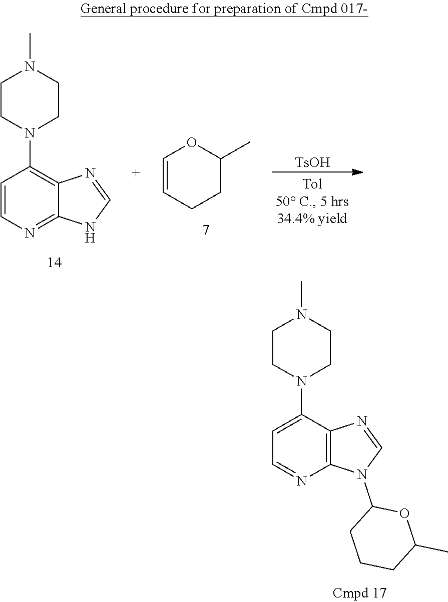

A mixture of Intermediate 7 (135 mg, 1.38 mmol, 3.00 eq), Intermediate 14 (100 mg, 460 μmol, 1.00 eq) and TsOH (103 mg, 598 μmol, 1.30 eq) in Tol. (3.00 mL) was stirred at 50° C. for 5 h.

Took a sample for LCMS (EC378-440-P1W9): Intermediate 14 was consumed, and one peak (Rt=1.556 min) with desired mass was detected.

Combined eight batches to work up. Poured the mixture in 20.0 mL H₂O, extracted the mixture with DCM (50.0 mL*3), washed the organic phase with brine (100 mL*1) and separated. Dried the organic phase with Na₂SO₄ and filtered, the filtrate was concentrated under vacuum.

The residue was purified by MPLC (SiO₂, R_{f}=0.20, dichloromethane/methanol=5/1, petroleum ether/ethyl acetate=10/1 to 0/1), then the product was separated by SFC (column: DAICEL CHIRALCEL OD (250 mm*30 mm, 10 μm; mobile phase: [0.1% NH₃H₂O MeOH]; B %: 15%-15%, 8.0; 160 min) to obtain Intermediate 16 (647 mg, 1.27 mmol, 34.4% yield, 100% purity) as yellow oil, confirmed via HPLC, LCMS, and ¹H NMR.

LCMS: Rt=1.556 min, m/z=316.2 (M+H)⁺.

LCMS: Rt=0.482 min, m/z=316.1 (M+H)⁺.

HPLC: Rt=1.251 min, 100% purity under 220 nm.

¹H NMR (400 MHz, MeOD): δ 8.26 (s, 1H), 8.05 (d, J=6.0 Hz, 1H), 6.65 (d, J=6.0 Hz, 1H), 5.83-5.80 (m, 1H), 3.98 (s, 4H), 3.91-3.88 (m, 1H), 2.83 (t, J=4.8 Hz, 4H), 2.50 (s, 3H), 1.85-1.82 (m, 2H), 1.74-1.71 (m, 2H), 1.40-1.37 (m, 1H), 1.23 (d, J=6.0 Hz, 3H)

### Example 6—Preparation of Cmpd 027

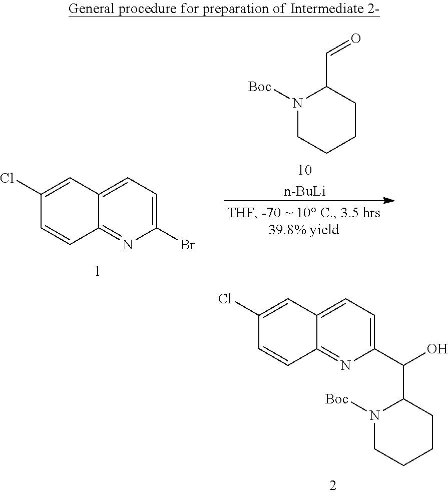

Charged Intermediate 1 (100 mg, 412 μmol, 1.00 eq) and THF (2.00 mL) into flask at 10° C. under nitrogen.

Cooled down to −70° C.

Charged n-BuLi (2.50 M, 247 μL, 1.50 eq) dropwise into flask at −70° C. under nitrogen.

The reaction mixture was stirred at −70° C. for 1.5 h.

Charged a solution of Intermediate 1b (105 mg, 494 μmol, 1.20 eq) in THF (1.00 mL) into flask at −70° C. under nitrogen.

The mixture was stirred at −70° C. for 2 h.

TLC (Petroleum ether:Ethyl acetate=3:1) showed Intermediate 1 (R_{f}=0.8) was consumed completely and the major spot (R_{f}=0.3) was detected.

LCMS showed the desired mass (RT=0.575 min, 0.614 min) was detected.

After the reaction was completed, the mixture was added to sat. NH₄Cl solution (20 mL) at 0° C. and the mixture was extracted with ethyl acetate (2*20.0 mL). The organic phase was separated and washed with brine (25.0 mL). The solution was dried over Na₂SO₄, filtered and then evaporated in vacuum.

The residue was purified by pre-TLC (Petroleum ether:Ethyl acetate=3:1, R_{f}=0.3).

Intermediate 2 (67.0 mg, 164 μmol, 39.8% yield, 92.5% purity) was obtained as yellow oil and confirmed via ¹H NMR (EC361-437-P1B1) and LCMS (EC361-437-P1B1).

LCMS: RT=0.575 min, 0.614 min, m/z=376.9 (M+H)⁺, 377.0 (M+H)⁺.

LCMS: RT=0.572 min, 0.611 min, m/z=377.0 (M+H)⁺, 376.9 (M+H)⁺.

¹H NMR (400 MHz, CDCl₃): δ 7.98-8.07 (m, 2H), 7.79-7.80 (m, 1H), 7.67 (d, J=2.00 Hz, 1H), 7.33-7.64 (m, 1H), 5.09-5.13 (m, 1H), 4.67 (d, J=138 Hz, 1H), 4.19 (s, 1H), 3.14 (t, J=20.8 Hz, 1H), 2.17 (d, J=24.8 Hz, 1H), 1.67-1.83 (m, 1H), 1.41-1.53 (m, 9H), 0.91-1.02 (m, 4H).

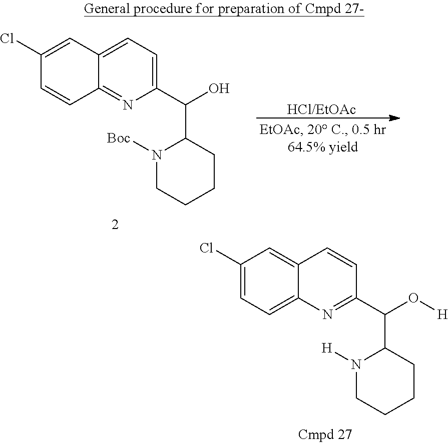 [SMILES: CC(C)(C)OC(=O)N1CCCCC1C(O)c1ccc2cc(Cl)ccc2n1 ; "HCl/EtOAc" ; OC(c1ccc2cc(Cl)ccc2n1)C1CCCCN1]

Charged Intermediate 2 (67.0 mg, 164 μmol, 92.5% purity, 1.00 eq), ethyl acetate (1.00 mL), HCl/EtOAc (4.00 M, 925 μL, 22.5 eq) into 100 mL flask at 20° C.

The mixture was stirred 0.5 h at 20° C.

TLC (Plate 1, Petroleum ether:Ethyl acetate=0:1) indicated Intermediate 2 (R_{f}=0.50) was consumed completely, one new spot (R_{f}=0.20) was detected.

The mixture was filtered and the filtrate cake was washed ethyl acetate (5.00 mL*2). The filter cake was concentrated under vacuum.

The solid was slurried in ethyl acetate (5.00 mL) at 20° C. for 0.5 hr and collected by filtration.

Freeze-dried the product.

Cmpd 027 (31.13 mg, 106 μmol, 64.5% yield, 94.3% purity) was obtained as yellow solid and confirmed via ¹H NMR, LCMS, and HPLC.

LCMS: RT=0.403 min, 0.412 min, m/z=277.2 (M+H)⁺, m/z=277.2 (M+H)⁺.

HPLC: RT=1.333 min, 1.387 min, 94.3% purity under 220 nm.

¹H NMR (400 MHz, MeOD): δ 8.60-8.68 (m, 1H), 8.14-8.22 (m, 2H), 7.93 (t, J=18.8 Hz, 2H), 5.32 (t, 1H), 4.99-5.06 (m, 1H), 3.67-379 (m, 1H), 3.46 (d, J=13.6 Hz, 1H), 3.15 (t, J=14.4 Hz, 1H), 1.82-1.93 (m, 2H), 1.69-1.76 (m, 2H), 1.46 (d, J=23.6 Hz, 1H).

### Example 7—Preparation of Cmpd 029

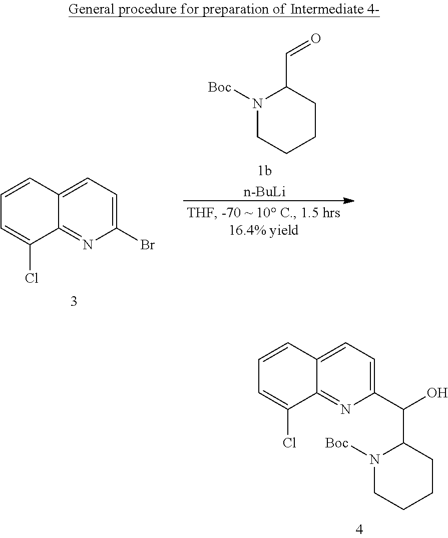

Charged Intermediate 3 (100 mg, 412 μmol, 1.00 eq), THF (3.00 mL), Intermediate 1b (105 mg, 494 μmol, 1.20 eq) into flask at 10° C. under nitrogen.

Cooled down to −70° C.

Charged n-BuLi (2.50 M, 247 μL, 1.50 eq) dropwise into flask at −70° C. under nitrogen.

The mixture was stirred at −70° C. for 1.5 h.

TLC (Plate 1, Petroleum ether:Ethyl acetate=3:1) indicated Intermediate 3 (R_{f}=0.70) was consumed completely, three new spots (R_{f}=0.15, R_{f}=0.30, R_{f}=0.80) was detected.

After the reaction was completed, the mixture was added to sat.NH₄Cl solution (20.0 mL) at 0° C. and the mixture was extracted with ethyl acetate (2*20.0 mL). The organic phase was separated and washed with brine (25.0 mL). The solution was dried over Na₂SO₄, filtered and then evaporated in vacuum.

The mixture was purified by pre-TLC (Petroleum ether:Ethyl acetate=3:1, R_{f}=0.3).

Intermediate 4 (26.0 mg, 67.7 μmol, 16.4% yield, 98.2% purity) was obtained as yellow solid and confirmed via ¹H NMR (EC3360-132-p1), LCMS (EC3360-132-P1C1).

LCMS: RT=0.608 min, 0.639 min, m/z=377.1 (M+H)⁺, m/z=377.1 (M+H)⁺.

¹H NMR (400 MHz, CDCl₃): δ 8.12 (d, J=8.40 Hz, 1H), 7.75 (d, J=8.00 Hz, 2H), 7.43-7.48 (m, 2H), 5.06 (d, J=6.00 Hz, 1H), 4.03-4.28 (m, 3H), 3.14-3.19 (m, 2H), 1.88 (d, J=13.2 Hz, 2H), 1.73 (t, J=23.2 Hz, 3H), 1.40-1.60 (m, 9H).

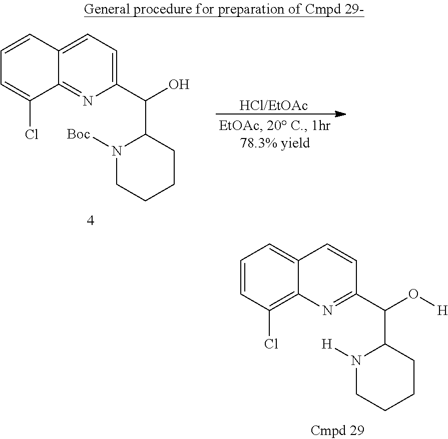 [SMILES: CC(C)(C)OC(=O)N1CCCCC1C(O)c1ccc2cccc(Cl)c2n1 ; "HCl/EtOAc" ; OC(c1ccc2cccc(Cl)c2n1)C1CCCCN1]

Charged Intermediate 4 (50.0 mg, 130 μmol, 98.2% purity, 1.00 eq), ethyl acetate (1.00 mL) into flask at 10° C. under nitrogen.

Charged HCl/EtOAc (4.00 M, 2.00 mL, 60.3 eq) into flask at 10° C.

The mixture was stirred 1 h at 10° C.

TLC (Plate 1, Petroleum ether:Ethyl acetate=0:1) indicated Intermediate 4 (R_{f}=0.50) was consumed completely, one new spot (R_{f}=0.20) was detected.

The mixture was filtered and the filtrate cake was washed ethyl acetate (5.00 mL*2). The filter cake was concentrated under vacuum.

The solid was slurried in ethyl acetate (5.00 mL) at 20° C. for 0.5 h and collected by filtration.

Cmpd 029 (28.79 mg, 102 μmol, 78.3% yield, 98.1% purity) was obtained as yellow solid and confirmed via ¹H NMR, LCMS, and HPLC.

LCMS: RT=0.402 min, m/z=277.2 (M+H)⁺.

HPLC: RT=1.298 min, 1.336 min, 98.1% purity under 220 nm.

¹H NMR (400 MHz, MeOD): δ 8.48-8.51 (m, 1H), 7.93-7.96 (m, 2H), 7.86 (d, J=8.4 Hz, 1H), 7.59 (t, J=15.6 Hz, 1H), 5.12 (d, J=4.8 Hz, 1H), 4.96 (d, J=4.40 Hz, 1H), 3.79 (d, J=4.80 Hz, 1H), 3.51 (d, J=15.2 Hz, 1H), 3.12-3.16 (m, 1H), 1.84 (s, 3H), 1.69-1.74 (m, 2H).

### Example 8—Preparation of Cmpd 038

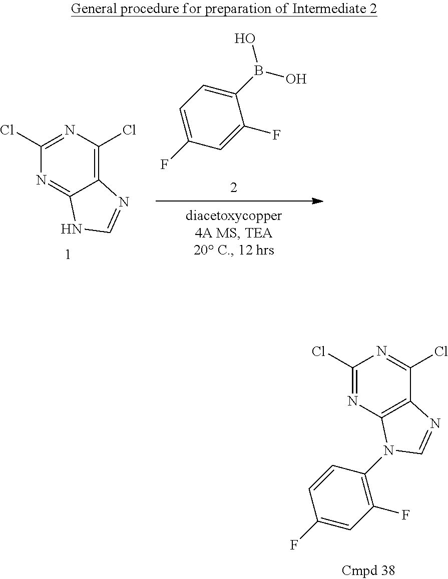 [SMILES: Clc1nc(Cl)c2nc[nH]c2n1 ; OB(O)c1ccc(F)cc1F ; Fc1ccc(-n2cnc3c(Cl)nc(Cl)nc32)c(F)c1]

Added Intermediate 1 (300 mg, 1.59 mmol, 1.00 eq), Intermediate 2 (501 mg, 3.17 mmol, 2.00 eq), diacetoxycopper (577 mg, 3.17 mmol, 2.00 eq), 4A MS (13.2 mmol), TEA (321 mg, 3.17 mmol, 442 μL, 2.00 eq) to DCM (3.00 mL)

Stirred at 20° C. for 12 h under 02 atmosphere.

LCMS showed that Intermediate 1 was consumed and the desired mass (RT=0.570 min) was given.

The mixture was filtered and the filtrate was concentrated.

The crude product was purified by Pre-HPLC (column: Phenomenex luna C18 150*25 mm*10 μm; mobile phase: [water (HCl)-ACN]; B %: 36%-66%, 10 min).

Cmpd 038 (20 mg, 59.07 μmol, 3.72% yield, 99.7% purity, HCl) was obtained as a white solid.

LCMS: RT=0.570 min, m/z=300.9 (M+H)⁺

LCMS: RT=0.565 min. m/z=300.9 (M+H)⁺

HPLC: RT=2.630 min, 99.7% purity under 220 nm.

¹⁹FNMR (400 MHz, CDCl₃)

¹H NMR (400 MHz, CDCl₃): δ 8.29 (d, J=0.88 Hz, 1H) 7.65-7.71 (m, 1H) 7.17 (s, 2H).

### Example 9—Preparation of Cmpd 043

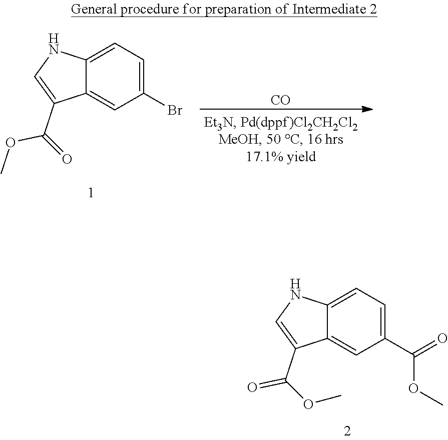 [SMILES: COC(=O)c1c[nH]c2ccc(Br)cc12 ; "CO" ; COC(=O)c1ccc2[nH]cc(C(=O)OC)c2c1]

Intermediate 1 (5.00 g, 19.7 mmol, 1.00 eq) was dissolved in MeOH (50.0 mL) at 15˜25° C.

The solution was degassed and purged with N₂ for 3 times.

Added Et₃N (5.97 g, 59.0 mmol, 8.22 mL, 3.00 eq) and Pd (dppf) Cl₂·CH₂Cl₂ (3.21 g, 3.94 mmol, 0.20 eq) to above solution under N₂ at 15˜25° C.

The mixture was degassed and purged with CO (50 psi) for 3 times.

The mixture was heated to 40˜50° C. and stirred at 40˜50° C. for 16 h under CO (50 psi).

LCMS showed ˜27.1% desired product (RT=1.194 min, m/z=234.2) was detected.

The mixture was filtered. The filtrate was concentrated to remove MeOH.

The residue was diluted with water (20.0 mL) and ethyl acetate (20.0 mL). The aqueous phase was extracted with ethyl acetate (10.0 mL*2). The combined organic layers was washed with brine (10.0 mL*2), dried over Na₂SO₄, filtered and concentrated.

The residue was purified by column chromatography (SiO₂, Petroleum ether/ethyl acetate=50/1 to 1/1, product: Petroleum ether/ethyl acetate=2/1, R_{f}=0.40).

Intermediate 2 (1.10 g, 3.36 mmol, 17.1% yield, 71.3% purity) was obtained as a yellow solid checked by LCMS (EC3718-89-P1LD, RT=1.209 min, m/z=234.2).

LCMS: EC3718-89-P1L4, RT=1.194 min, m/z (M+H⁺)=234.2

LCMS: EC3718-89-P1LD, RT=1.209 min, m/z (M+H⁺)=234.2

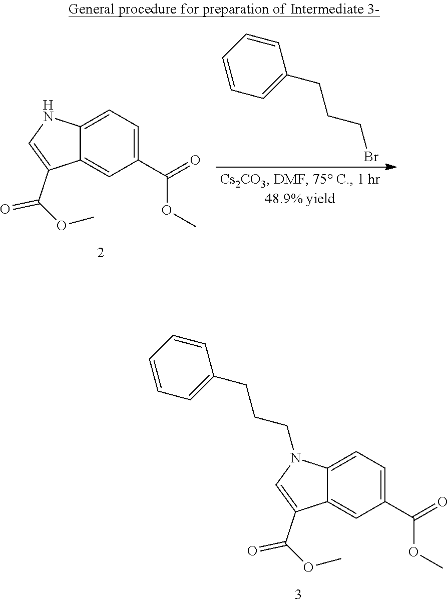 [SMILES: COC(=O)c1ccc2[nH]cc(C(=O)OC)c2c1 ; BrCCCc1ccccc1 ; COC(=O)c1ccc2c(c1)c(C(=O)OC)cn2CCCc1ccccc1]

Intermediate 2 (700 mg, 3.00 mmol, 1.00 eq) and Cs₂CO₃ (1.96 g, 6.00 mmol, 2.00 eq) was dissolved in DMF (7.00 mL) at 15˜25° C.

3-bromopropylbenzene (1.79 g, 9.00 mmol, 1.36 mL, 3.00 eq) was added to above solution.

The mixture was heated to 65˜75° C. and stirred at 65˜75° C. for 1 h.

LCMS (EC3718-97-P1L3) showed Intermediate 2 was consumed and a new peak with desired mass (RT=2.551 min, m/z=352.1) was detected.

The reaction mixture was poured into aq.NH₄Cl (21.0 mL), the aqueous phase was extracted with ethyl acetate (5.00 mL*2). The combined organic layers was washed with brine (5.00 mL*2), dried over Na₂SO₄, filtered and concentrated.

The crude product was purified by prep-HPLC (column: Phenomenex Luna C18 200*40 mm*10 um; mobile phase: [water (TFA)-ACN]; B %: 55%-85%, 10 min)

Intermediate 3 (522 mg, 1.47 mmol, 48.9% yield, 98.7% purity) was obtained as a yellow solid checked by LCMS (EC3718-97-P1L4, RT=1.713 min. m/z=351.9).

LCMS: RT=2.551 min, m/z (M+H⁺)=352.1

LCMS: RT=1.713 min, m/z (M+H⁺)=351.9

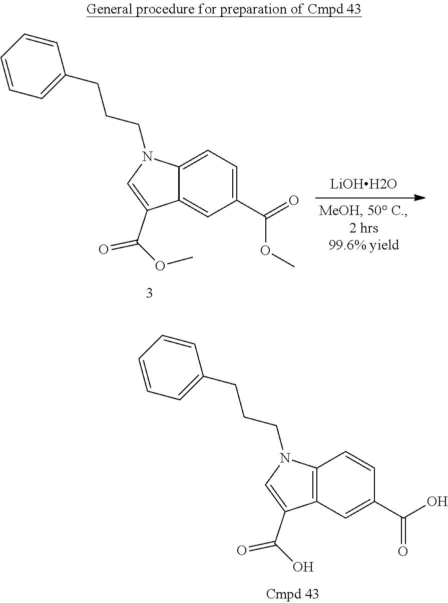 [SMILES: COC(=O)c1ccc2c(c1)c(C(=O)OC)cn2CCCc1ccccc1 ; O=C(O)c1ccc2c(c1)c(C(=O)O)cn2CCCc1ccccc1]

Two batches were carried out in parallel.

Intermediate 3 (200 mg, 569 μmol, 1.00 eq) and LiOH·H2O (71.7 mg, 1.71 mmol, 3.00 eq) was dissolved in MeOH (2.00 mL) at 15˜25° C.

The mixture was heated to 40˜50° C. and stirred at 40˜50° C. for 2 h.

LCMS (EC3718-99-P1L5) showed Intermediate 3 was consumed and a new peak with desired mass (RT=0.525 min, m/z=324.3) was detected.

The reaction mixture was cooled to 15˜25° C. and adjust pH to 3˜5 with 1N HCl (aq). The mixture was filtered and washed with 1N HCl (aq, 10 mL). The filter caked was concentrated to give the product.

PX043 (220.06 mg, 679 μmol, 59.6% yield, 99.7% purity) was obtained as a white solid checked by ¹H NMR, LCMS, and HPLC.

LCMS: RT=0.525 min, m/z (M+H⁺)=324.3.

LCMS: RT=2.043 min, m/z (M+H⁺)=324.0.

HPLC: RT=2.637 min, 99.7% purity under 220 nm.

¹H NMR (400 MHz, DMSO-d6): δ 12.45 (s, 2H) 8.72 (d, J=1.2 Hz, 1H) 8.21 (s, 1H) 7.83 (dd, J=8.6, 1.6 Hz, 1H) 7.62 (d, J=8.8 Hz, 1H) 7.23-7.29 (m, 2H) 7.18 (d, J=6.8 Hz, 3H) 4.31 (t, J=7.0 Hz, 2H) 2.55-2.60 (m, 2H) 2.11 (m, 2H).

### Example 10—Preparation of Cmpd 048

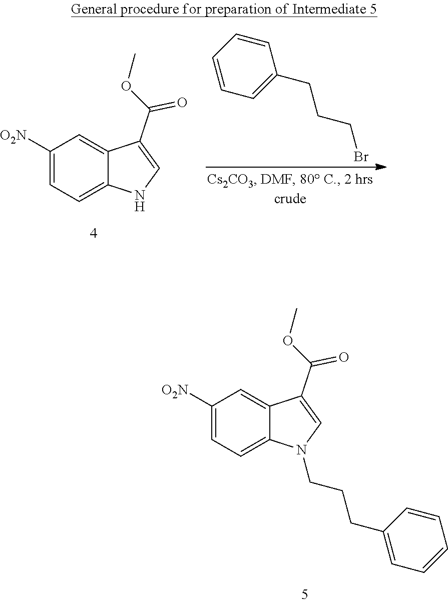

Intermediate 4 (2.00 g, 9.08 mmol, 1.00 eq) and Cs₂CO₃ (5.92 g, 18.2 mmol, 2.00 eq) was dissolved in DMF (20.0 mL) at 15˜25° C.

The mixture was stirred at 15˜25° C. for 0.5 hr.

Then 3-bromopropylbenzene (5.43 g, 27.3 mmol, 4.11 mL, 3.00 eq) was added to above mixture at 15˜25° C.

The mixture was heated to 70˜80° C. and stirred at 70˜80° C. for 1.5 h.

LCMS (EC3718-96-P1L2) showed Intermediate 5 was consumed and a new peak with desired mass (RT=1.876 min, m/z=339.2) was detected.

The reaction mixture was poured into aqueous NH₄Cl (60.0 mL), the aqueous phase was extracted with ethyl acetate (10.0 mL*2). The combined organic layers was washed with brine (10.0 mL*2), dried over Na₂SO₄, filtered and concentrated.

The crude product was used in the next step without purification.

Intermediate 5 (3.50 g, crude) was obtained as a brown oil checked by LCMS.

LCMS: RT=1.876 min, m/z (M+H⁺)=339.2.

LCMS: RT=1.928 min. m/z (M+H⁺)=338.9.

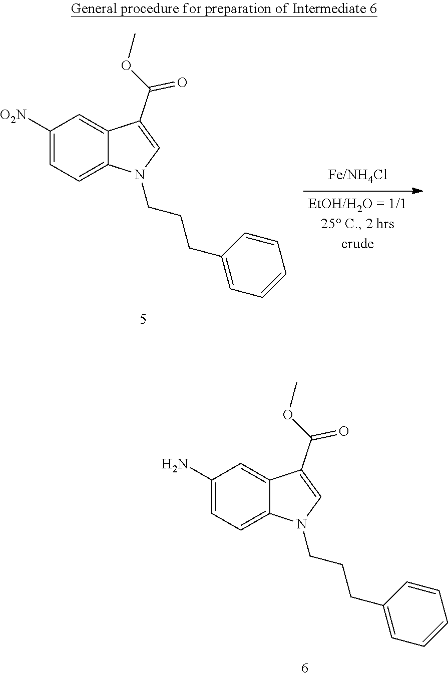 [SMILES: COC(=O)c1cn(CCCc2ccccc2)c2ccc([N+](=O)[O-])cc12 ; COC(=O)c1cn(CCCc2ccccc2)c2ccc(N)cc12]

Intermediate 5 (400 mg, 1.18 mmol, 1.00 eq) was dissolved in EtOH (4.00 mL) at 15˜25° C.

Added the solution of NH₄Cl (632 mg, 11.8 mmol, 10.0 eq) in H₂O (4.00 mL) to above solution.

Fe (330 mg, 5.91 mmol, 5.00 eq) was added to above mixture at 15˜25° C.

The mixture was stirred at 15˜25° C. for 2 h.

LCMS showed Intermediate 5 was consumed and a new peak with desired mass (RT=0.461 min, m/z=309.2) was detected.

The reaction mixture was filtered. The filtrate was concentrated to remove EtOH. The aqueous phase was extracted with ethyl acetate (10.0 mL*2). The combined organic layers were washed with brine (10.0 mL*2), dried over Na₂SO₄, filtered and concentrated.

The crude product was purified by column chromatography (SiO₂, Petroleum ether/Ethyl acetate=100/1 to 2/1, product: Petroleum ether/Ethyl acetate=1/1, R_{f}=0.40).

Intermediate 6 (200 mg, 588 μmol, 49.8% yield, 90.7% purity) was obtained as a yellow solid checked by LCMS.

LCMS: RT=0.461 min, m/z (M+H⁺)=309.2.

LCMS: RT=1.816 min. m/z (M+H⁺)=308.9.

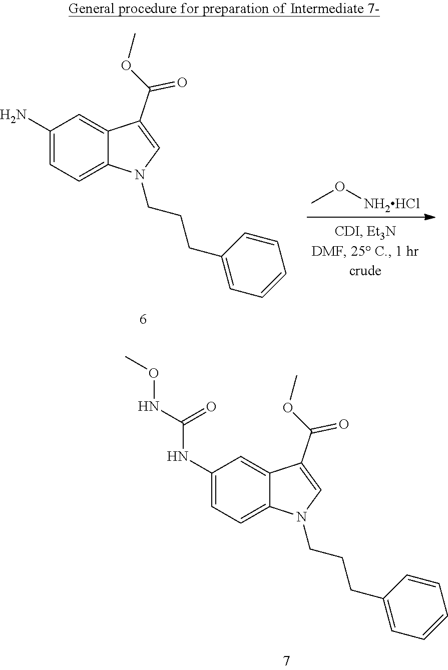

Intermediate 6 (120 mg, 389 μmol, 1.00 eq) and Et₃N (78.8 mg, 778 μmol, 108 μL, 2.00 eq) was dissolved in DMF (3.00 mL) at 15˜25° C.

CDI (69.4 mg, 428 μmol, 1.10 eq) was added to above solution at 15˜25° C.

The mixture was stirred at 15˜25° C. for 0.5 h.

O-methylhydroxylamine (42.3 mg, 506 μmol, 1.30 eq, HCl) was added to above solution at 15˜25° C.

The mixture was stirred at 15˜25° C. for 0.5 h.

LCMS showed Intermediate 6 was consumed and a new peak with desired mass (RT=0.571 min, m/z=382.2) was detected.

The reaction mixture was poured into water (10.0 mL), the aqueous phase was extracted with ethyl acetate (5.00 mL*2). The combined organic layers was washed with brine (5.00 mL*2), dried over Na₂SO₄, filtered and concentrated.

The crude product was used in the next step without purification.

Intermediate 7 (130 mg, crude) was obtained as a brown oil checked by LCMS (RT=0.567 min, m/z=382.2).

LCMS: RT=0.571 min, m/z (M+H⁺)=382.2

LCMS: RT=0.567 min. m/z (M+H⁺)=382.2

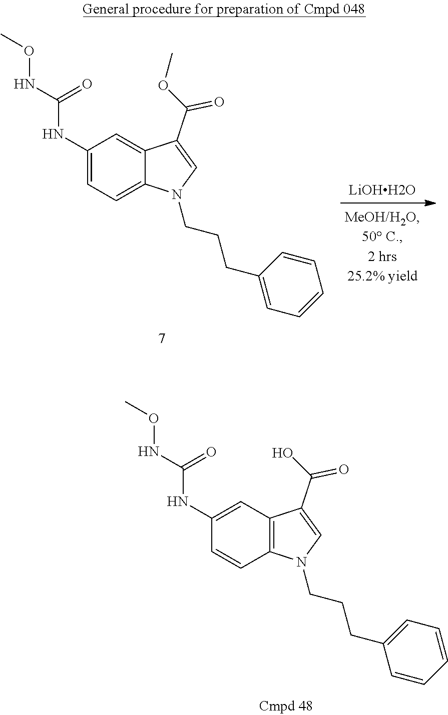 [SMILES: CONC(=O)Nc1ccc2c(c1)c(C(=O)OC)cn2CCCc1ccccc1 ; CONC(=O)Nc1ccc2c(c1)c(C(=O)O)cn2CCCc1ccccc1]

Intermediate 7 (100 mg, 262 μmol, 1.00 eq) and LiOH·H2O (55.0 mg, 1.31 mmol, 5.00 eq) was dissolved in MeOH (3.00 mL) and H₂O (0.30 mL) at 15˜25° C.

The mixture was heated to 40˜50° C. and stirred at 40˜50° C. for 16 h.

LCMS showed Intermediate 7 was consumed and a new peak with desired mass (RT=0.415 min, m/z=368.1) was detected.

The reaction mixture was cooled to 15˜25° C. and adjust pH to 3˜5 with 1N HCl (aq). The mixture was filtered and washed with 1N HCl (aq, 10.0 mL). The filter caked was concentrated to give the crude product.

The crude product was purified by column chromatography (SiO₂, Petroleum ether/Ethyl acetate=10/1 to 0/1, product: Petroleum ether/Ethyl acetate=0/1, R_{f}=0.60)

Cmpd 048 (25.62 mg, 66.0 μmol, 25.2% yield, 94.6% purity) was obtained as a white solid checked by ¹H NMR (EC3718-108-P1N2), LCMS (EC3718-108-P1L1, RT=0.418 min, m/z=368.1) and HPLC (EC3718-108-P1H2).

LCMS: RT=0.415 min, m/z (M+H⁺)=368.1

LCMS: RT=0.418 min, m/z (M+H⁺)=368.1

HPLC: 94.6% purity under 220 nm

¹H NMR (400 MHz, DMSO-d6): δ 11.89 (s, 1H) 9.35 (s, 1H) 8.82-8.87 (m, 1H) 8.19 (s, 1H) 8.03 (s, 1H) 7.40-7.46 (m, 2H) 7.25-7.30 (m, 2H) 7.19 (d, J=7.0 Hz, 3H) 4.24 (t, J=6.8 Hz, 2H) 3.63 (s, 3H) 2.54-2.59 (m, 2H) 2.04-2.13 (m, 2H),

### Example 11—Preparation of Cmpd 54

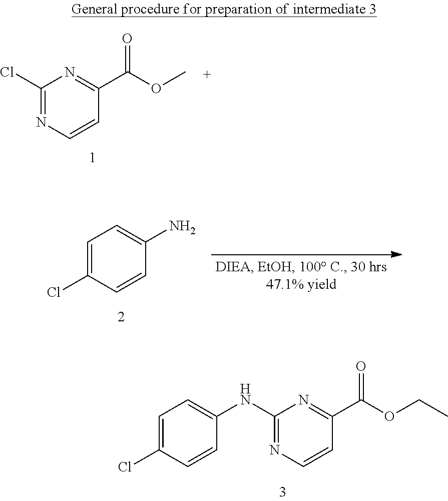 [SMILES: COC(=O)c1ccnc(Cl)n1 ; Nc1ccc(Cl)cc1 ; CCOC(=O)c1ccnc(Nc2ccc(Cl)cc2)n1]

Charged intermediate 1 (23.0 g, 133 mmol, 1.00 eq) and intermediate 2 (25.5 g, 200 mmol, 1.50 eq), DIEA (34.5 g, 267 mmol, 46.4 mL, 2.00 eq) and EtOH (200 mL) into flask at 25° C. under nitrogen.

The mixture was stirred at 100° C. for 30 h.

LCMS showed that intermediate 1 was consumed, and one peak (Rt=0.583 min) with desired mass was detected.

Poured the mixture in 500 mL H₂O, extracted the mixture with DCM (250 mL*7), washed the organic phase with brine (100 mL*1) and separated. Dried the organic phase with Na₂SO₄ and filtered, the filtrate was concentrated under vacuum.

The residue was purified by MPLC (SiO₂, Rf=0.60, Petroleum ether:Ethyl acetate=5/1, Petroleum ether/Ethyl acetate=10/1 to 0/1).

Intermediate 3 (19.0 g, 62.7 mmol, 47.1% yield) was obtained as yellow solid and confirmed via LCMS (EC378-470-P1O1).

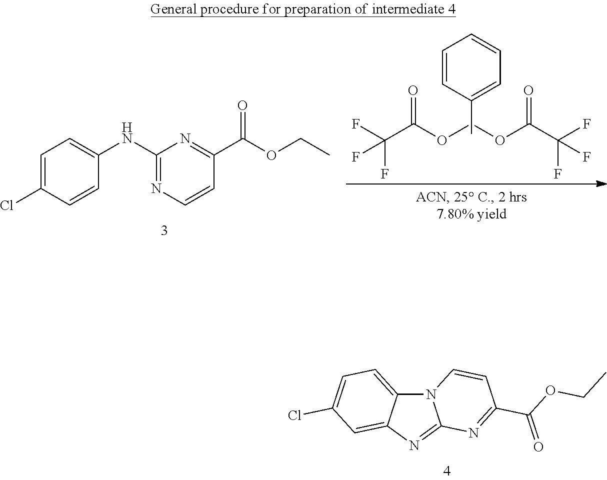

Charged intermediate 3 (19.0 g, 68.4 mmol, 1.00 eq), [phenyl-(2,2,2-trifluoroacetyl)oxy-λ3-iodanyl]2,2,2-trifluoroacetate (38.3 g, 88.9 mmol, 1.30 eq), ACN (200 mL) into 500 mL flask at 20° C.

The reaction was stirred at 25° C. for 2 h under N₂.

Take a sample for LCMS (EC378-476-P1R1): intermediate 3 was consumed, and two peaks (Rt=0.451 &0.494 min) with desire mass was detected.

Poured the mixture in 300 mL H₂O, extracted the mixture with DCM (500 mL*7), washed the organic phase with brine (100 mL*1) and separated. Dried the organic phase with Na₂SO₄ and filtered, the filtrate was concentrated under vacuum.

The crude product was purified by prep-HPLC (0.1% NH₃*H₂O).

Give intermediate 4 (1.60 g, 5.34 mmol, 7.80% yield) as yellow solid, confirmed via LCMS.

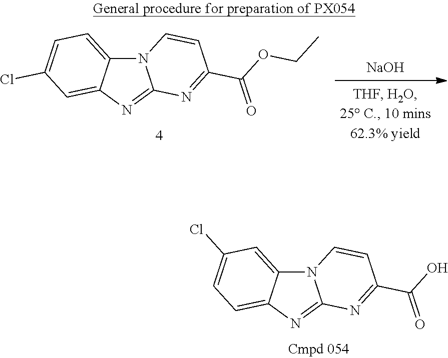 [SMILES: CCOC(=O)c1ccn2c(n1)nc1cc(Cl)ccc12 ; "NaOH" ; O=C(O)c1ccn2c(n1)nc1ccc(Cl)cc12]

A mixture of intermediate 4 (1.60 g, 5.80 mmol, 1.00 eq) and NaOH (464 mg, 11.6 mmol, 2.00 eq) in THF (20.0 mL) and H₂O (5.80 mL).

The mixture was stirred at 25° C. for 10 min.

Took a sample for TLC (Petroleum ether:Ethyl acetate=0:1): intermediate 4 (R_{f}=0.50) was consumed, and a new spot (Rf=0.00) was detected.

The mixture was filtered and concentrated under reduced pressure to give a residue. Then added 50.0 mL HCl—H₂O (1.00 N) to the residue, the aqueous layer was extracted with DCM (30.0 mL*3). The combined organic layer was washed with H₂O (10.0 mL*2), dried over Na₂SO₄, filtered and concentrated.

Give Cmpd 054 (262.8 mg, 3.62 mmol, 62.3% yield, 99.5% purity) as yellow solid, confirmed via LCMS & HPLC & ¹H NMR.

LCMS: RT=0.331 min, m/z=247.0 (M+H)⁺.

HPLC: RT=1.348 mins, 99.5% purity in 254 nm.

¹H NMR (400 MHz, DMSO): δ 9.76 (d, J=6.80 Hz, 1H), 8.48 (d, J=8.80 Hz, 2H), 8.04 (s, 1H), 7.58 (m, 1H), 7.55 (m, 1H).

### Example 12—Preparation of Cmpd 55

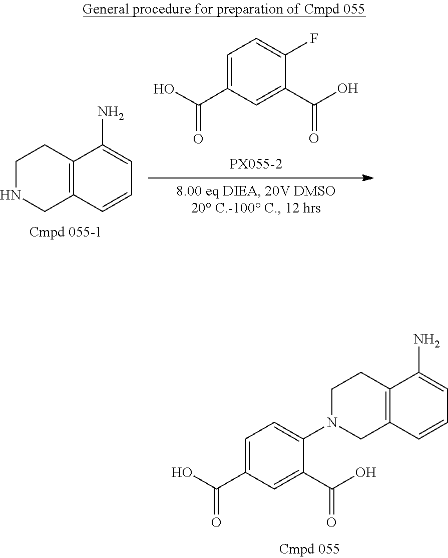 [SMILES: Nc1cccc2c1CCNC2 ; O=C(O)c1ccc(F)c(C(=O)O)c1 ; Nc1cccc2c1CCN(c1ccc(C(=O)O)cc1C(=O)O)C2]

To a solution of intermediate 055-1 (96.0 mg, 648 μmol, 1.00 eq) and intermediate 055-2 (119 mg, 648 μmol, 1.00 eq) in DMSO (2.00 mL) was added DIEA (670 mg, 5.19 mmol, 903 μL, 8.00 eq) at 20° C., then the mixture was heated to 100° C. and stirred at 100° C. for 12 h.

LCMS (EC3963-107-P1A1) showed that ˜14.0% of intermediate 055-2 (RT=0.945 min) was remained and the desired MS (RT=0.994 min) was detected.

The crude product was purified by reversed-phase HPLC (0.1% FA condition, Phenomenex luna C18 150*25 mm*10 μm; mobile phase: [water (FA)-ACN]; B %: 5%-35%, 10 min), concentrated under vacuum to removed ACN and water to give a residue.

The reaction was successful, Cmpd 055 (30.0 mg, 92.9 umol, 14.3% yield, 96.8% purity) was obtained as a yellow solid and confirmed by ¹H NMR (EC3963-107-P1A1), LCMS (EC3963-107-P1D), HPLC (EC3963-107-P1D2).

LCMS: RT=0.994 min, m/z=313.0 (M+H)⁺.

LCMS: RT=0.389 min, m/z=313.1 (M+H)⁺.

HPLC: RT=1.768 mins, 96.8% purity under 220 nm.

¹H NMR: (400 MHz, DMSO-d₆): δ 8.29 (d, J=2.0 Hz, 1H), 8.00 (dd, J=2.4, 2.0 Hz, 1H), 7.36 (d, J=8.8 Hz, 1H), 6.89 (t, J=8.0 Hz, 1H), 6.52 (d, J=8.0 Hz, 1H), 6.38 (d, J=7.2 Hz, 1H), 4.24 (s, 2H), 3.47 (t, J=5.6 Hz, 2H), 2.67-2.62 (m, 2H).

### Example 13—Preparation of Cmpd 56

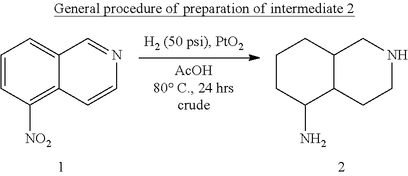 [SMILES: O=[N+]([O-])c1cccc2cnccc12 ; NC1CCCC2CNCCC12]

To a solution of intermediate 1 (1.00 g, 5.74 mmol, 1.00 eq) in AcOH (20.0 mL) was added PtO₂ (500 mg, 2.20 mmol) at 20° C., then the mixture was heated to 80° C. and stirred at 80° C. for 24 hrs under 50 Psi. TLC (Petroleum ether:Ethyl acetate=1:2) showed that intermediate 1 (R_{f}=0.50) was consumed and the desired spot (R_{f}=0.00) was formed. The reaction mixture was filtered and the filtrated cake was washed with MeOH (100 mL), then the filtrate was concentrated under vacuum to give a residue. The residue was combined with EW3695-141 for purification. The crude product was purified by silica gel chromatography (diameter: 100-200 mesh silica gel, Petroleum ether:Ethyl acetate=10:1 to 0:1, Petroleum ether:Ethyl acetate=1:2, R_{f}=0.00) to give a product. Intermediate 2 (200 mg, 1.30 mmol, crude purity) was obtained as a yellow solid, which was confirmed by HNMR.

¹H NMR (400 MHz, CDCl₃): δ 3.58-3.55 (m, 1H), 3.40-3.35 (m, 1H), 3.25-3.24 (m, 1H), 3.15-3.04 (m, 2H), 2.16-1.94 (m, 1H), 1.71-1.44 (m, 9H).

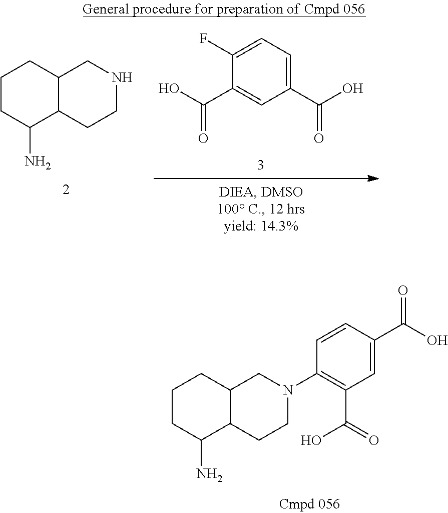 [SMILES: NC1CCCC2CNCCC12 ; O=C(O)c1ccc(F)c(C(=O)O)c1 ; NC1CCCC2CN(c3ccc(C(=O)O)cc3C(=O)O)CCC12]

To a solution of intermediate 2 (200 mg, 0.524 mmol, 1.00 eq,) and intermediate 3 (96.5 mg, 0.524 mmol, 1.00 eq) in DMSO (2.00 mL) was added DIEA (0.338 g, 2.62 mmol, 0.456 mL, 5.00 eq) at 20° C., then the mixture was heated to 100° C. and stirred at 100° C. for 12 h.

LCMS showed intermediate 2 was consumed and desired MS (RT=0.693 min) was detected. The reaction mixture was cooled to 25° C. and poured into H₂O (50 mL), the aqueous phase was extracted with ethyl acetate (30 mL*2). The combined organic phase was washed with brine (50 mL*2), dried with anhydrous Na₂SO₄, filtered, and concentrated in vacuum to give a crude product. The residue was combined with EW3977-133 for purification. The crude product was purified by reverse-phase HPLC (0.1% HCl condition, column: Welch Xtimate C18 150*25 mm*5 um; mobile phase: [water (HCl)-ACN]; B %: 0%-17%, 8 min), the eluent was concentrated in vacuum to remove ACN, the aqueous phase was lyophilized to give a product.

Cmpd 056 (30.0 mg, 90.37 μmol, 14.3% yield, 95.9% purity) was obtained as a white solid, which was confirmed by LCMS, HPLC, 2D HNMR and HNMR.

LCMS: RT=0.693 min, m/z=319.1 (M−H)⁺.

LCMS: RT=1.119 mins, m/z=319.1 (M−H)⁺.

HPLC: RT=1.733 mins, 95.9% purity under 220 nm.

¹H NMR (400 MHz, MeOD): δ 8.83 (s, 1H), 8.45 (d, J=8 Hz, 1H), 8.08 (d, J=7.6 Hz, 1H), 3.80-3.78 (m, 2H), 3.60-3.58 (m, 2H), 3.09 (s, 1H), 2.37-2.34 (m, 1H), 2.16 (s, 1H), 1.96-1.81 (m, 4H), 1.73-1.70 (m, 1H), 1.60-1.58 (m, 2H), 1.29-1.23 (m, 1H).

### Example 14—Preparation of Cmpd 57

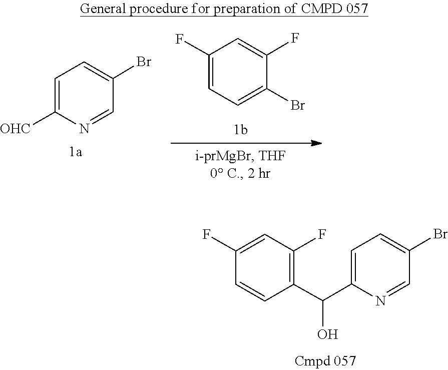

Charged intermediate 1b (9.34 g, 48.4 mmol, 1.80 eq) in THF (90.0 mL) was added i-PrMgBr (2 M, 24.2 mL, 1.80 eq) at 0° C., the mixture was stirred at 0° C. for 1 h.

The mixture was added to a solution of intermediate 1a (5.00 g, 26.9 mmol, 1.00 eq) in THF (50.0 mL) at 0° C. and the mixture was stirred at 0° C. for 1 h.

Took a sample for LCMS: intermediate 1a was consumed completely and one main peak with desired mass (Rt=0.405 min) was detected.

The mixture was poured into saturated aqueous NH₄Cl solution (200 mL) under 0° C., extracted the mixture with DCM (100 mL*3), washed the organic phase with brine (300 mL*1) and separated, dried the organic phase with Na₂SO₄ and filtered, the filtrate was concentrated under vacuum.

The crude product was purified by prep-HPLC: column: Phenomenex Luna C18 150*25 mm*10 um; mobile phase: [water (HCl)-ACN]; B %: 39%-69%, 10 min.

Cmpd 057 (30 mg, 98.87 umol, 3.15% yield, 98.9% purity) was obtained as yellow solid and confirmed via LCMS: EC378-531-P1E2, HPLC: EC378-532-P1E4, HNMR: EC378-532-P1H.

LCMS: RT=0.405 min, m/z=300.1 (M+H)⁺.

LCMS: RT=0.430 min, m/z=300.1 (M+H)⁺.

HPLC: RT=1.833 mins, 98.9% purity in 220 nm.

¹H NMR (400 MHz, DMSO): δ 8.56 (m, 1H), 8.08 (m, 1H), 7.58 (m, 1H), 7.43 (m, 1H), 7.15 (m, 2H), 5.89 (s, 1H).

### Example 15—Preparation of Cmpd 58

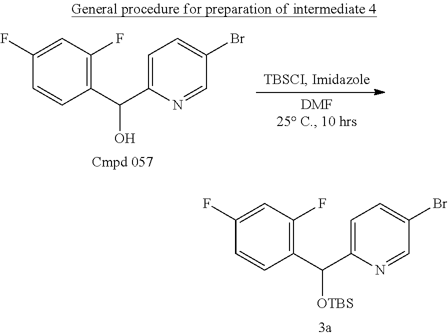 [SMILES: OC(c1ccc(Br)cn1)c1ccc(F)cc1F ; CC(C)(C)[Si](C)(C)OC(c1ccc(Br)cn1)c1ccc(F)cc1F]

To a solution of Cmpd 057 (6.00 g, 20.0 mmol, 1.00 eq) and TBSCl (3.62 g, 24.0 mmol, 2.94 mL, 1.20 eq) in DMF (15.0 mL), then the mixture was added imidazole (3.40 g, 49.9 mmol, 2.50 eq) at 25° C. and the mixture was stirred at 25° C. for 10 h.

Take a sample for LCMS (EC378-451-P1Q1): Cmpd 057 was consumed completely and one main peak with desired mass (Rt=2.351 mins) was detected.

The mixture was poured into H₂O (70.0 mL) under 25° C., extracted the mixture with EA (60.0 mL*3), washed the organic phase with brine (100 mL*2) and separated, dried the organic phase with Na₂SO₄ and filtered, the filtrate was concentrated under vacuum.

The residue was purified by MPLC (SiO₂, Rf=0.80, (Petroleum ether:Ethyl acetate=10/1), Petroleum ether/Ethyl acetate=100/1 to 50/1) to obtain Intermediate 3a (8.00 g, 19.3 mmol, 96.6% yield) was obtained as a white solid, confirmed by LCMS.

LCMS: RT=2.351 mins, m/z=415.9 (M+H)+.

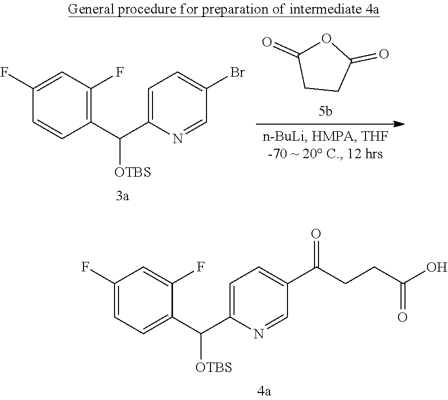

To a solution of intermediate 3a (2.00 g, 4.83 mmol, 1.00 eq) and HMPA (1.21 g, 6.76 mmol, 1.19 mL, 1.40 eq) in THF (30.0 mL), The mixture was cooled to −70° C. A dropwised n-BuLi (2.5 M, 2.70 mL, 1.40 eq) to the mixture at −70° C. under N2. Then the mixture was stirred at −70° C. for 1 hr. intermediate 5b (966 mg, 9.65 mmol, 2.00 eq) was dissolved in THF (10.0 mL) was added dropwise to the mixture at −70° C. under N₂. The mixture was stirred at 20° C. for 12 h under N2.

Take a sample for LCMS (EC378-468-P1E2): intermediate 3a was consumed completely and one main peak with desired mass (Rt=0.481 min) was detected.

The reaction mixture was poured into saturated NH₄Cl aqueous (50.0 mL) at 0° C. The mixture was extracted with ethyl acetate (50.0 mL*3). The combined organic phase was washed with brine (60.0 mL) and concentrated. The residue was purified by prep-HPLC (0.1% HCl) to obtain intermediate 4a (250 mg, 529 μmol, 10.9% yield) as yellow oil, confirmed by LCMS.

LCMS: RT=0.481 min, m/z=436.0 (M+H)+.

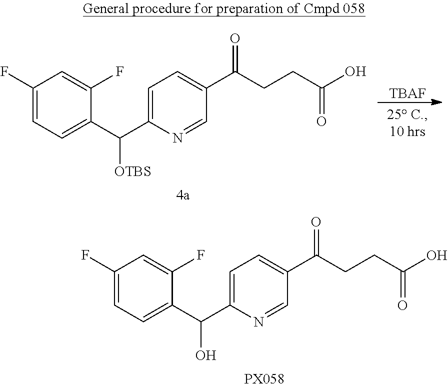

A mixture of intermediate 4a (150 mg, 344 μmol, 1.00 eq) in TBAF (1 M, 5.00 mL, 14.5 eq) stirred at 25° C. for 10 h.

Take a sample for LCMS: showed intermediate 4a was consumed, and one pain peak (RT=0.209 min) with desired mass was detected.

The reaction mixture was poured into H₂O (20.0 mL) and extracted with EA (20.0 mL*3). The combined organic phases were washed with brine (20.0 mL), dried by Na₂SO₄, filtered and concentrated.

The crude product was purified by prep-HPLC: column: Phenomenex luna C18 150*25 mm*10 um; mobile phase: [water (HCl)-ACN]; B %: 7%-37%, 10 min.

The crude product was purified by prep-HPLC (column: Phenomenex Luna C18 150*25 mm*10 um; mobile phase: [water (HCl)-ACN]; B %: 39%-69%, 10 min) to obtain Cmpd 058 (20 mg, 59.76 umol, 17.35% yield, 96.0% purity) as a yellow oil, confirmed by LCMS, HPLC, and ¹H NMR.

LCMS: RT=0.209 min, m/z=322.0 (M+H)⁺.

LCMS: RT=0.430 min, m/z=300.1 (M+H)⁺.

HPLC: RT=1.833 mins, 98.9% purity in 220 nm.

¹H NMR (400 MHz, DMSO): δ 8.57 (m, 1H), 8.06 (m, 1H), 7.63 (m, 1H), 7.61 (m, 1H), 7.49 (m, 2H), 7.25 (m, 1H), 6.08 (s, 1H), 3.10 (m, 2H), 2.58 (m, 2H).

### Example 16—Preparation of Cmpd 59

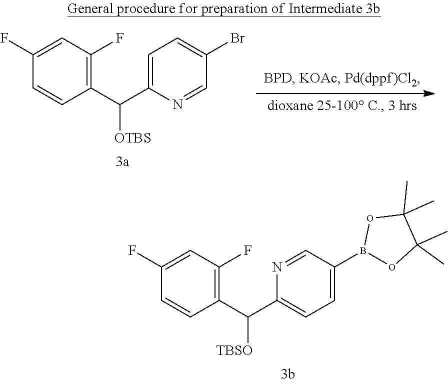 [SMILES: CC(C)(C)[Si](C)(C)OC(c1ccc(Br)cn1)c1ccc(F)cc1F ; CC1(C)OB(c2ccc(C(O[Si](C)(C)C(C)(C)C)c3ccc(F)cc3F)nc2)OC1(C)C]

A mixture of intermediate 3a (1.00 g, 2.41 mmol, 1.00 eq), BPD (1.23 g, 4.83 mmol, 2.00 eq), KOAc (474 mg, 4.83 mmol, 2.00 eq) in dioxane (10.0 mL). Then Pd(dppf)Cl₂ (177 mg, 241 μmol, 0.10 eq) was added to the mixture at 25° C. under N₂. The mixture was stirred at 100° C. for 3 h under N₂ atmosphere.

Took a sample for LCMS (EC378-467-P1D1): intermediate 3a was consumed completely and one main peak with desired mass (Rt=2.328 mins) was detected.

The mixture was filtered, and to the filtrate was added 20.0 mL EtOAc, then washed the organic phase with brine (10.0 mL*1) and separated, dried the organic phase with Na₂SO₄ and filtered, the filtrate was concentrated under vacuum.

The residue was purified by MPLC (SiO₂, R_{f}=0.60, (Petroleum ether:Ethyl acetate=5/1), Petroleum ether/Ethyl acetate=100/1 to 10/1) to obtain intermediate 4a (1.05 g, 2.19 mmol, 90.6% yield,) as a yellow solid, confirmed by LCMS.

LCMS: RT=2.328 min, m/z=380.0 (M+H)⁺.

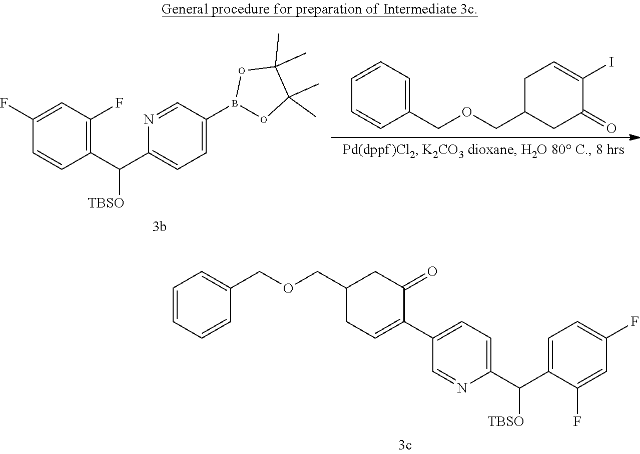

Charged dioxane (15.0 mL), H₂O (3.00 mL) to the bottle.

Charged intermediate 3b (1.50 g, 3.25 mmol, 1.00 eq), 5-(benzyloxymethyl)-2-iodo-cyclohex-2-en-1-one (1.11 g, 3.25 mmol, 1.00 eq), Pd(dppf)Cl₂ (265 mg, 325 μmol, 0.10 eq) to the bottle.

Charged K₂CO₃ (898 mg, 6.50 mmol, 2.00 eq) to the bottle under N₂.

The mixture was stirred at 80° C. for 8 h under N₂.

Took a sample for TLC (Petroleum ether:Ethyl acetate=5:1): intermediate 3b (R_{f}=0.50) was consumed, and a new main spot (R_{f}=0.30) was detected.

The mixture was poured into H₂O (50.0 mL) under 25° C., extracted the mixture with DCM (100 mL*3), washed the organic phase with brine (100 mL*1) and separated, dried the organic phase with Na₂SO₄ and filtered, the filtrate was concentrated under vacuum.

The residue was purified by MPLC (SiO₂, R_{f}=0.30, (Petroleum ether:Ethyl acetate=5/1; Petroleum ether/Ethyl acetate=30/1 to 5/1) to obtain intermediate 3c (1.50 g, 2.73 mmol, 83.9% yield)) as a yellow oil.

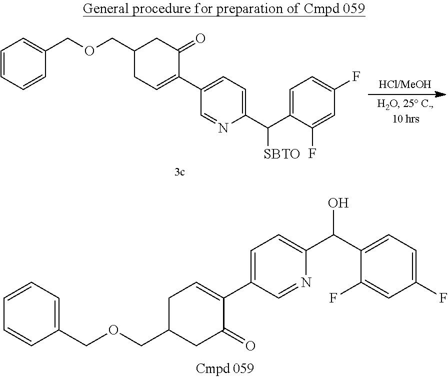

A mixture of intermediate 3c (500 mg, 909 umol, 1.00 eq) in HCl/MeOH (4 M, 2.00 mL, 8.80 eq) and H₂O (1.00 mL) was stirred at 25° C. for 10 h.

Took a sample for LCMS (EC378-535-P1Q1): showed intermediate 3c was consumed, and one pain peak (Rt=0.418 min) with desire mass was detected.

The reaction mixture was poured into H₂O (20 mL) and extracted with EA (20.0 mL*3). The combined organic phases were washed with brine (20.0 mL), dried by Na₂SO₄, filtered and concentrated.

The crude product was purified by prep-HPLC: column: Phenomenex Luna C18 200*40 mm*10 um; mobile phase: [water (HCl)-ACN]; B %: 30%-60%, 10 min.

The crude product was purified by prep-HPLC (column: Phenomenex Luna C18 150*25 mm*10 μm; mobile phase: [water (HCl)-ACN]; B %: 39%-69%, 10 min) to obtain Cmpd 059 (120 mg, 275.57 μmol, 30.30% yield, 100% purity) was obtained as a yellow solid, confirmed via LCMS, HPLC, and ¹H NMR.

LCMS: RT=0.418 min, m/z=435.9 (M+H)⁺.

LCMS: RT=0.588 min, m/z=436.0 (M+H)⁺.

HPLC: RT=2.608 min, 100% purity in 220 nm.

¹H NMR (400 MHz, DMSO): δ 8.45 (s, 1H), 7.95 (m, 1H), 7.66 (m, 1H), 7.36 (m, 1H), 7.33 (m, 6H), 7.30 (m, 1H), 7.19 (m, 1H), 6.05 (s, 1H), 4.50 (s, 2H), 3.50 (s, 2H), 2.64 (m, 3H), 2.40 (m, 2H).

### Example 17—Preparation of Cmpd 62

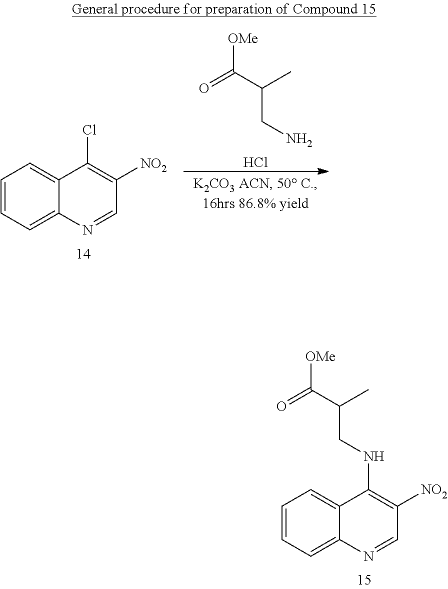

Charged intermediate 14 (5.00 g, 24.0 mmol, 1 eq) into R1 (100 mL) at 15˜25° C.

Charged ACN (50.0 mL) into R1 at 15˜25° C.

Charged intermediate 14_A (3.87 g, 25.2 mmol, 1.05 eq, HCl) into R1 at 15˜25° C.

Charged K₂CO₃ (5.96 g, 43.1 mmol, 1.80 eq) into R1 at 15˜25° C.

Heated the mixture to 50° C.

Stirred the mixture at 50° C. for 20 hrs.

Took a sample for LCMS. Intermediate 14 was consumed and intermediate 15 (RT=0.361 min) was detected.

Cooled the mixture to 20-25° C.

Diluted the mixture with Ethyl acetate (150 mL).

Filtered the mixture under vacuum.

Washed the filter cake with Ethyl acetate (100 mL) and concentrated the filtrate under vacuum to obtain intermediate 15 (6.80 g, 20.8 mmol, 86.8% yield, 88.6% purity) as a yellow solid, confirmed by LCMS and HPLC.

LCMS: RT=0.361 min, m/z=290.1 (M+H)⁺

LCMS: RT=0.352 min, m/z=260.1 (M+H)⁺

HPLC: RT=0.998 min, 88.6% purity under 220 nm

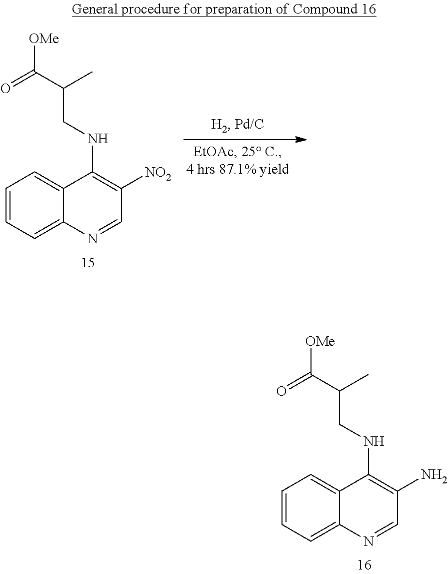 [SMILES: COC(=O)C(C)CNc1c([N+](=O)[O-])cnc2ccccc12 ; COC(=O)C(C)CNc1c(N)cnc2ccccc12]

Charged intermediate 15 (6.80 g, 23.5 mmol, 1.00 eq) into R1 (500 mL).

Charged Pd/C (680 mg, 10% purity) into R1 under N₂.

Charged Na₂SO₄ (3.40 g, 24.0 mmol, 2.43 mL, 1.02 eq) into R1 under N₂.

Charged EtOAc (210 mL) into R1 under N2.

Degas under vacuum and purged with H2 three times.

Stirred at 20-25° C. under H2 (15 psi) for 4 h.

Took a sample for LCMS. The reactant was consumed and Intermediate 16 (RT=0.339 min) was detected.

Filtered the mixture under vacuum.

Washed the filter cake with MeOH (100 mL*2) and concentrated the mixture under vacuum to obtain intermediate 16 (6.00 g, 20.5 mmol, 87.1% yield, 88.5% purity) as a brown solid, confirmed by LCMS and HPLC.

LCMS: RT=0.339 min, m/z=260.1 (M+H)⁺.

LCMS: RT=0.345 min, m/z=260.1 (M+H)⁺.

HPLC: RT=0.959 min, 88.5% purity under 220 nm.

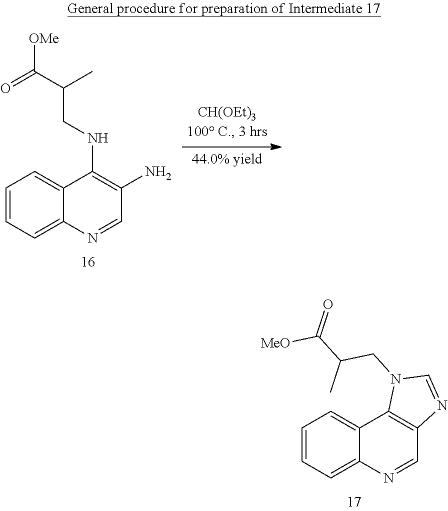 [SMILES: COC(=O)C(C)CNc1c(N)cnc2ccccc12 ; COC(=O)C(C)Cn1cnc2cnc3ccccc3c21]

Charged Intermediate 16 (4.00 g, 15.4 mmol, 1.00 eq) into R1 (35 mL).

Charged CH(OEt)₃ (35.6 g, 240 mmol, 40.0 mL, 15.6 eq) into R1 at 20-25° C.

Stirred at 100° C. for 3 hrs.

Took a sample for LCMS. Intermediate 16 was consumed and intermediate 17 (Rt=0.390 min) was detected.

Cooled the mixture to 20-25° C., added 1M HCl (100 mL) into R1, and stirred at 20-25° C. for 40 min. Adjusted the pH to 8-9 with saturated NaHCO₃ (200 mL), extracted the mixture with Ethyl acetate (200 mL*3), washed the organic layer with brine (300 mL), dried the organic layer over Na₂SO₄, and concentrated the organic layer under vacuum to give the crude product.

The crude product was purified by silica gel chromatography eluted with Petroleum ether/Ethyl acetate=2:1 to 1:3 to obtain intermediate 17 (2.12 g, 6.79 mmol, 44.0% yield, 86.2% purity) as a brown oil, confirmed by LCMS and HPLC.

LCMS: RT=0.390 min, m/z=270.1 (M+H)⁺.

LCMS: RT=0.412 min. m/z=270.1 (M+H)⁺.

HPLC: RT=1.370 mins, 86.2% purity under 220 nm.

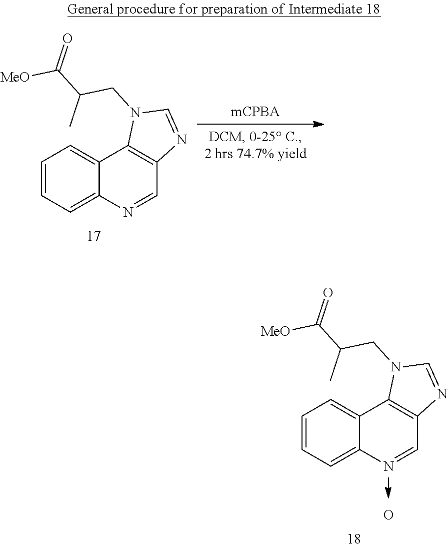 [SMILES: COC(=O)C(C)Cn1cnc2cnc3ccccc3c21 ; COC(=O)C(C)Cn1cnc2cnc3ccccc3c21 ; O]

Charged DCM (21.0 mL) into R1 (50 mL).

Charged intermediate 17 (2.10 g, 7.41 mmol, 95% purity, 1.00 eq) into R1.

Charged mCPBA (2.40 g, 11.11 mmol, 80% purity, 1.50 eq) into R1 at 0-5° C.

Stirred at 20-25° C. for 2 h.

Took a sample for LCMS. Intermediate 17 (Rt=0.414 min) was not consumed completely, but intermediate 18 (Rt=0.465 min) was detected.

Charged saturated Na₂SO₃ (60 mL) into R1 at 0-5° C., adjust the pH of the mixture to 8˜9 with saturated NaHCO₃, extracted the mixture with DCM (30.0 mL*3), washed the organic layer with brine (40.0 mL), dried the organic layer over Na₂SO₄, and concentrated the organic layer under vacuum at 40° C. to give Intermediate 18 (2.00 g, 5.54 mmol, 74.76% yield, 79% purity) as a brown oil without purification, confirmed by LCMS.

LCMS: RT=0.465 min, m/z=286.1 (M+H)⁺.

LCMS: RT=0.478 min, 79.4% purity under 220 nm.

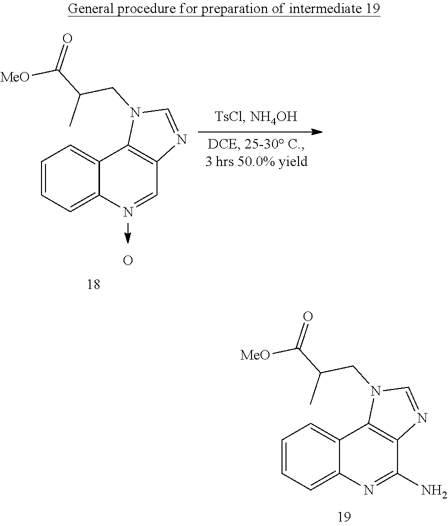 [SMILES: COC(=O)C(C)Cn1cnc2cnc3ccccc3c21 ; O ; COC(=O)C(C)Cn1cnc2c(N)nc3ccccc3c21]

Charged Intermediate 18 (2.00 g, 7.01 mmol, 1 eq) into R1 (100 mL).

Charged DCE (40.0 mL) into R1.

Charged NH₃·H₂O (15.6 g, 111 mmol, 17.1 mL, 25% purity, 15.8 eq) into R1 at 25-30° C.

Charged TosCl (1.60 g, 8.41 mmol, 1.2 eq) in DCE (10 mL) into R1 at 25-30° C.

Stirred at 25-30° C. for 3 hrs.

Took a sample for LCMS. Intermediate 18 was consumed and intermediate 19 (RT=0.436 min) was detected.

Diluted the mixture with DCE (50.0 mL), separated the organic layer between the water layer, washed the organic layer with brine (40.0 mL), dried the organic layer over Na₂SO₄, and concentrated the organic layer under vacuum to obtain Intermediate 19 (1.20 g, 3.51 mmol, 50.03% yield, 83.1% purity) as a brown oil, confirmed by LCMS.

LCMS: RT=0.436 min, m/z=285.1 (M+H)⁺.

LCMS: RT=0.443 min, 83.1% purity under 220 nm.

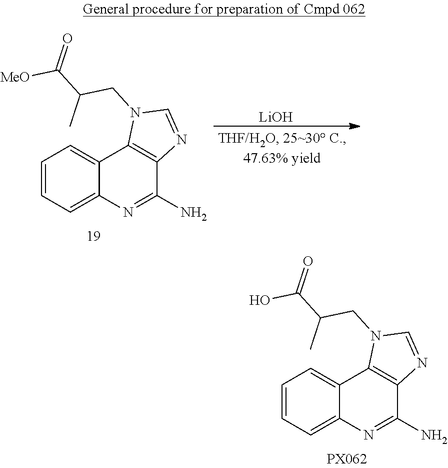 [SMILES: COC(=O)C(C)Cn1cnc2c(N)nc3ccccc3c21 ; "LiOH" ; CC(Cn1cnc2c(N)nc3ccccc3c21)C(=O)O]

Charged intermediate 19 (1.20 g, 4.22 mmol, 1 eq) into R1 (100 mL).

Charged THF (24 mL) into R1.

Charged LiOH·H2O (265.67 mg, 6.33 mmol, 1.5 eq) in H₂O (2.40 mL) into R1 at 25-30° C.

Stirred at 25-30° C. for 3 h.

Took a sample for LCMS. Intermediate 19 was consumed and Target 3 (RT=0.499 min) was detected.

Diluted the mixture with DCE (50 mL), separated the organic layer between the water layer, washed the organic layer with brine (40 mL), dried the organic layer over Na₂SO₄, and concentrated the organic layer under vacuum to obtain Cmpd 062 (0.55 g, 2.01 mmol, 47.63% yield, 98.8% purity) as a gray solid, confirmed by LCMS, HPLC and ¹H NMR.

LCMS: RT=0.499 min, m/z=329.0 (M+H)⁺.

LCMS: RT=0.423 min, m/z=329.0 (M+H)⁺.

HPLC: RT=1.353 mins, 98.8% purity under 220 nm.

¹H NMR: (400 MHz, DMSO-d6): δ 12.0-14.87 (m, 1H), 8.79-8.80 (m, 2H), 8.46 (s, 1H), 8.22-8.24 (m, 1H), 7.82-7.84 (m, 1H), 7.70-7.73 (m, 1H), 7.56-7.59 (m, 1H), 4.89-4.94 (m, 1H), 4.68-4.73 (m, 1H), 2.98-3.04 (m, 1H), 1.18 (d, 7.2 Hz).

### Example 18—Preparation of Cmpd 64

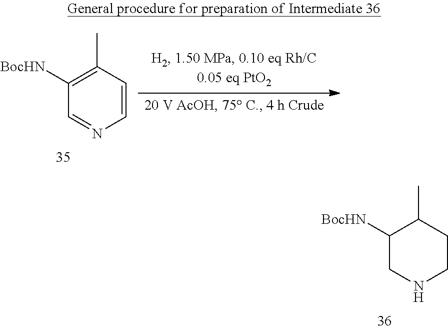

Added Rh/C (2.08 g, 1.01 mmol, 2.03 mL, 5% purity, 0.10 eq) into R1 (100 mL) at 20-25° C. under Ar₂.

Added PtO₂ (109 mg, 480 μmol, 0.05 eq) into R1 at 20-25° C. under Ar₂.

Added AcOH (40.0 mL) into R1 at 20-25° C. under Ar₂.

Added Intermediate 35 (2.00 g, 9.60 mmol, 1.00 eq) at 20-25° C. under Ar₂.

The suspension was degassed under vacuum and purged with H₂ several times.

Stirred under 1.50 MPa at 75° C. for 4 h in a 50 mL of autoclave.

Took a sample for LCMS. The Intermediate 35 was consumed completely and the desired peak (RT=0.395 min) detected.

Cooled down to 20˜25° C.

The reaction mixture was filtered and concentrated in vacuum to obtain Intermediate 36 (3.20 g, crude) as a brown oil, confirmed by LCMS.

LCMS: RT=0.395 min. m/z=215.3 (M+H)⁺.

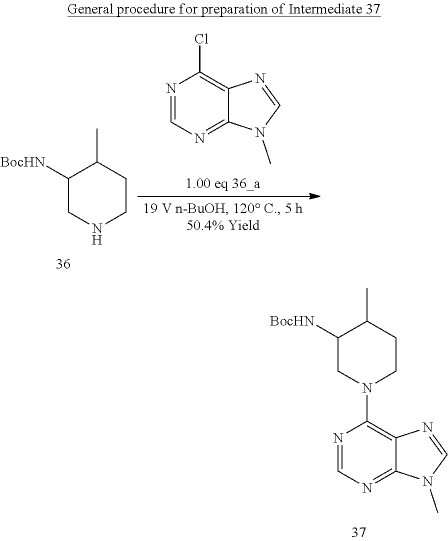 [SMILES: CC1CCNCC1NC(=O)OC(C)(C)C ; Cn1cnc2c(Cl)ncnc21 ; CC1CCN(c2ncnc3c2ncn3C)CC1NC(=O)OC(C)(C)C]

Added n-BuOH (24.0 mL) into R1 (50.0 mL) at 20-25° C.

Added Intermediate 36 (1.27 g, 5.93 mmol, 1.00 eq) into R1 at 20-25° C. Added Intermediate 36_a (999 mg, 5.93 mmol, 1.00 eq) into R1 at 20-25° C.

Stirred at 120° C. for 5 hrs.

Took a sample for LCMS. The intermediate 36 was consumed completely and the desired peak (RT=0.333 min) detected.

Filtered the reaction mixture, washed the filter cake with EtOAc (30.0 mL*2), and concentrated the filter cake under vacuum to obtain Intermediate 37 (1.50 g, 2.99 mmol, 50.4% yield, 69.0% purity) (HPLC: EC8946-1-P1B2) as an off-white solid, confirmed by LCMS and HPLC.

LCMS: RT=0.333 min, m/z=347.2 (M+H)³⁰

HPLC: RT=1.210 min, 69.0% purity under 220 nm.

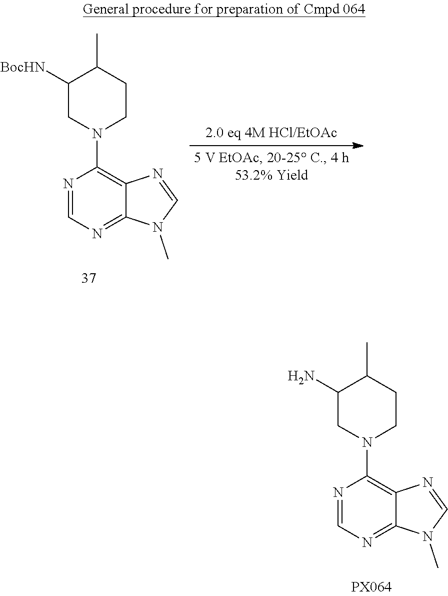 [SMILES: CC1CCN(c2ncnc3c2ncn3C)CC1NC(=O)OC(C)(C)C ; CC1CCN(c2ncnc3c2ncn3C)CC1N]

Added intermediate 37 (1.50 g, 4.33 mmol, 1.00 eq) into R1 (50.0 mL) at 20-25° C.

Added EtOAc (7.00 mL) into R1 at 20-25° C.

Added HCl/EtOAc (4 M, 2.16 mL, 2.00 eq) into R1 at 20-25° C.

Stirred at 20-25° C. for 4 h.

Took a sample for LCMS (EC8946-6-P1A1): The intermediate 37 was consumed completely and the desired peak (RT=1.300 min) detected.

Filtered the reaction mixture and washed the filter cake with EtOAc (10.0 mL*2).

Concentrated the filter cake under vacuum to give crude product.

The crude product on notebook page EC8836-2 (110 mg) was combined to EC8946-6 for further purification.

Added the mixture into R2 (50.0 mL) at 20-25° C.

Added MeOH (7.50 mL) in EtOAc (15.0 mL) into R2 at 20-25° C.

Stirred at 25-30° C. for 0.3 h.

Filtered the reaction mixture, washed the filter cake with EtOAc (10.0 mL*2), and concentrated the filter cake under vacuum to obtain Cmpd 064 (570 mg, 2.31 mmol, 53.2% yield, 99.7% purity) as a white solid, confirmed by LCMS, HPLC, and ¹H NMR.

LCMS: RT=1.300 mins, m/z=247.2 (M+H)⁺.

LCMS: RT=0.315 min, m/z=246.9 (M+H)⁺.

HPLC: RT=1.451 mins, 99.7% purity under 220 nm.

¹H NMR (400 MHz, MeOD): δ 8.52 (s, 1H), 8.32 (s, 1H), 5.50 (d, J=12.8 Hz, 1H), 4.94-5.10 (s, 1H), 4.08 (d, J=7.8 Hz, 1H), 3.94 (s, 3H), 3.67-3.77 (m, 2H), 2.31-2.43 (m, 1H), 1.88-1.99 (m, 1H), 1.72-1.86 (m, 1H), 1.23 (d, J=7.2 Hz, 3H).

### Example 19—Preparation of Cmpd 65

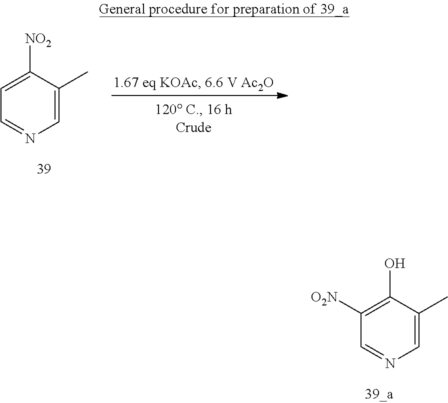

Added intermediate 39 (25.0 g, 181 mmol, 1.00 eq) into R1 (500 mL) at 20-25° C.

Added Ac₂O (166 mL) into R1 at 20-25° C.

Added KOAc (29.6 g, 302 mmol, 1.67 eq) into R1 at 20-25° C.

Stirred at 120° C. for 16 h.

LCMS analysis showed that intermediate 39 was consumed completely and intermediate 39_a (RT=0.127 min) was detected.

Cooled down to 20˜25° C.

Add MTBE (250 mL) into R1 and stir at 20-25° C. for 15 min. Filtered the mixture, washed the filter cake with MTBE (250 mL*2), and concentrated the organic layer under vacuum to obtain intermediate 39_a as a brown oil, confirmed by LCMS.

LCMS: RT=0.127 min, m/z=110.4 (M+H)⁺.

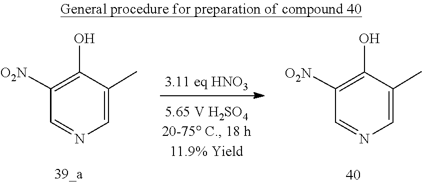

Added H₂SO₄ (90.0 mL) into R1 (500 mL) at 20-25° C.

Added intermediate 39_a (23.0 g, 210 mmol, 1.00 eq) into R1 at 20-25° C.

The solution warmed to 60-65° C.

Added HNO₃ (42.1 g, 656 mmol, 30.1 mL, 98.0% purity, 3.11 eq) in H2SO4 (40 mL) into R2 (250 mL) at 20-25° C.

Slowly added the mixture from R2 to R1 at 60-65° C.

Stirred at 60-65° C. for 2 h.

Stirred at 75° C. for 16 h.

Took a sample for LCMS. Intermediate 39_a (RT=0.639 min) was not completely consumed, but the desired peak (RT=0.588 min) detected.

Cooled down to 10-20° C.

The reaction mixture was slowly poured into H2O (550 mL) at 10-20° C.

Adjusted the mixture pH=5˜6 with NH3·H2O (370 mL) at 10-20° C.

Filtered the mixture and concentrated the filter cake under vacuum to obtain intermediate 40 (3.85 g, 24.9 mmol, 11.9% yield) (HNMR: EC8946-29-P1C3) as a yellow solid, confirmed by LCMS and ¹H NMR.

LCMS: RT=0.588 min, m/z=155.1 (M+H)⁺.

¹H NMR (400 MHz, DMSO): δ 12.18 (s, 1H), 8.73 (s, 1H), 7.73 (s, 1H), 1.92 (s, 3H).

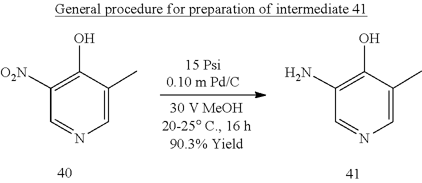

Added Pd/C (0.34 g, 1.70 mmol, 10% purity) into R1 (250 mL) at 20-25° C. under Ar₂.

Added MeOH (100 mL) into R1 at 20-25° C. under Ar2.

Added intermediate 40 (3.40 g, 22.06 mmol, 1.00 eq) at 20-25° C. under N₂.

The suspension was degassed under vacuum and purged with H₂ several times.

The mixture was stirred under H₂ (15 psi) at 25° C. for 16 h.

LCMS analysis showed that Intermediate 40 was consumed completely and intermediate 41 (RT=0.123 min) was detected.

The reaction mixture was filtered and concentrated in vacuum to obtain intermediate 41 (2.66 g, 19.9 mmol, 90.3% yield, 93.0% purity) as a brown solid, confirmed by LCMS and HPLC.

LCMS: RT=0.123 min, m/z=125.3 (M+H)⁺.

HPLC: RT=0.467 min, 96.4% purity under 220 nm.

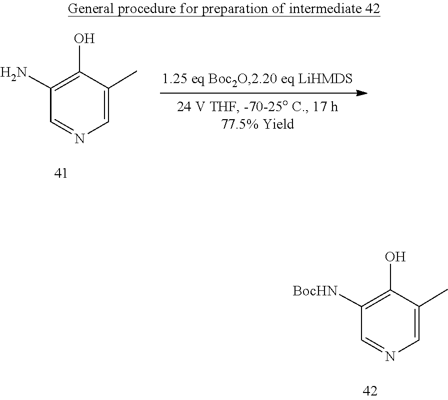

Charged THF (19.0 mL) into R1 (100 mL) at 20-25° C.

Charged intermediate 41 (950 mg, 7.65 mmol, 1.00 eq) into R1.

Degassed with N₂ three times and cooled the mixture to −70˜−60° C.

Charged LiHMDS (1 M, 16.8 mL, 2.20 eq) into R1 at −70˜−60° C. under N₂.

Stirred at −70˜−60° C. for 0.5 hrs.

Charged Boc2O (2.09 g, 9.57 mmol, 2.20 mL, 1.25 eq) in THF (4 mL) into R1 at −70˜−60° C. dropwise under N₂.

Stirred at −70˜−60° C. for 0.5 h, then at 20-25° C. for 16 h.

LCMS analysis showed that Intermediate 41 (RT=0.102 min) was not consumed completely, but Intermediate 42 (RT=0.465 min) detected.

The mixture was poured into H₂O (120 mL) at 20-25° C. and extracted with EtOAc (120 mL*2). The combined organic phase was washed with brine (120 mL), the organic layer was dried over Na₂SO₄, and concentrated under vacuum to obtain intermediate 42 (2.66 g, 11.8 mmol, 77.5% yield, 73.1% purity) as a yellow solid, confirmed by LCMS and HPLC.

LCMS: RT=0.466 min, m/z=169.0 (M−55).

LCMS: RT=0.469 min, m/z=169.0 (M−55).

HPLC: RT=1.672 min, 73.1% purity under 220 nm.

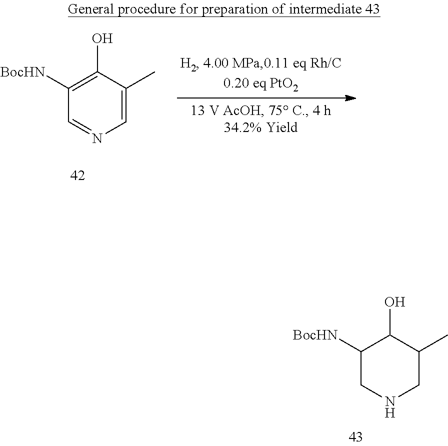 [SMILES: Cc1cncc(NC(=O)OC(C)(C)C)c1O ; CC1CNCC(NC(=O)OC(C)(C)C)C1O]

Added PtO2 (166 mg, 731 μmol, 0.20 eq) into R1 (50.0 mL) at 20-25° C. under Ar2.

Added Rh (827 mg, 402 μmol, 811 μL, 5% purity, 0.11 eq) into R1 at 20-25° C. under Ar2.

Added AcOH (11.0 mL) into R1 at 20-25° C. under Ar2.

Added intermediate 42 (820 mg, 3.66 mmol, 1.00 eq) at 20-25° C. under Ar₂.

The suspension was degassed under vacuum and purged with H₂ several times.

Was stirred under 4.0 MPa at 75° C. for 16 h in a 50 mL of autoclave.

Took a sample for LCMS. Intermediate 42 (RT=0.484 min) was not consumed completely and the desired peak (RT=0.352 min) detected.

Cooled down to 20˜25° C.

The reaction mixture was filtered and concentrated in vacuum to obtain intermediate 43. The crude product was purified by column chromatography (SiO2, Ethyl acetate:Petroleum ether=20/1, R_{f}=0.30). Intermediate 43 (600 mg, 2.50 mmol, 34.2% yield, 96.0% purity) was obtained as a yellow solid, confirmed by LCMS and HPLC.

LCMS: RT=0.353 min, m/z=231.2 (M+H⁺).

LCMS: RT=0.381 min. m/z=231.2 (M+H⁺).

HPLC: RT=0.758 min, 96.6% purity under 220 nm.

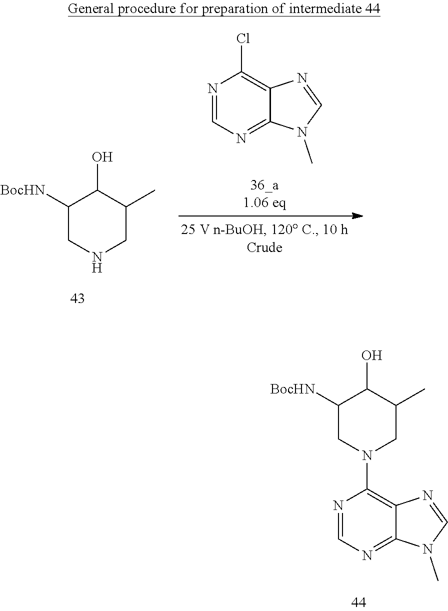 [SMILES: CC1CNCC(NC(=O)OC(C)(C)C)C1O ; Cn1cnc2c(Cl)ncnc21 ; CC1CN(c2ncnc3c2ncn3C)CC(NC(=O)OC(C)(C)C)C1O]

Added n-BuOH (12.0 mL) into R1 (50.0 mL) at 20-25° c.

Added intermediate 43 (470 mg, 2.04 mmol, 1.00 eq) into R at 20-25° C.

Added intermediate 36_a (365 mg, 2.17 mmol, 1.06 eq) into R1 at 20-25° C.

Stirred at 120 CC for 10 h.

LCMS analysis showed that Intermediate 43 was consumed completely and Intermediate 44 (RT=0.413 min) was detected.

Concentrated the reaction mixture under vacuum to obtain intermediate 44 (910 mg, crude) as a yellow solid, confirmed by LCMS.

LCMS: RT=0.413 min m/z=363.1 (M+H⁺).

LCMS: RT=2.126 min m/z (M+H⁺)=363.1 (M+H⁺).

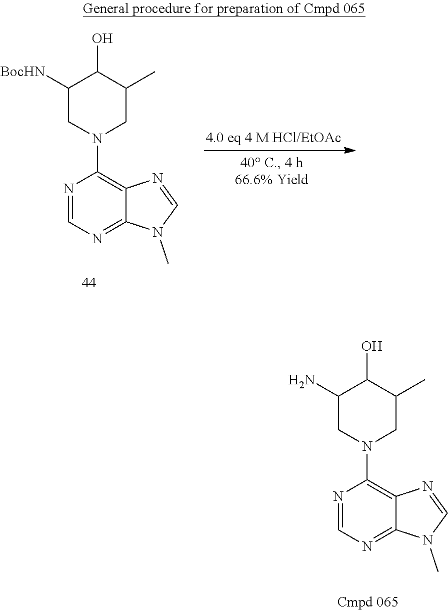 [SMILES: CC1CN(c2ncnc3c2ncn3C)CC(NC(=O)OC(C)(C)C)C1O ; CC1CN(c2ncnc3c2ncn3C)CC(N)C1O]

Added intermediate 44 (1.00 g, 2.76 mmol, 1.00 eq) into R1 (50.0 mL) at 20-25° C.

Added EtOAc (10.0 mL) into R1 at 20-25° C.

Added HCl/EtOAc (4 M, 2.76 mL, 4.00 eq) into R1 at 20-25° C.

Stirred at 35-40° C. for 4 h.

Took a sample for LCMS. The intermediate 44 was consumed completely and the desired peak (RT=0.270 min) detected.

Cooled the reaction mixture to 20-25° C.

The reaction mixture on notebook page EC8946-43 was combined to EC8946-42 for workup.

Filtered the reaction mixture and washed the filter cake with EtOAc (10.0 mL*2).

Concentrated the filter cake under vacuum to give crude product.

The crude product was triturated with MeOH:EtOAc=1:2 (10 V) at 20-25° C. for 0.5 h. The mixture was filtered and concentrated under vacuum to obtain Cmpd 065 (700 mg, 2.25 mmol, 66.6% yield, 95.9% purity, HCl) as a white oil, confirmed as LCMS, HPLC, and ¹H NMR.

LCMS: RT=0.270 min, m/z=263.1 (M+H)⁺.

LCMS: RT=0.285 min, m/z=263.1 (M+H)⁺.

LCMS: RT=6.697 min, m/z=263.3 (M+H)⁺.

HPLC: RT=1.031 mins, 95.9% purity under 220 nm.

¹H NMR (400 MHz, MeOD): δ 8.50 (s, 1H), 8.32 (s, 1H), 4.02 (s, 1H), 3.94 (s, 3H), 3.57-3.72 (m, 3H), 3.33-3.49 (m, 1H), 2.91-3.26 (m, 1H), 2.00-2.10 (m, 1H), 1.13 (d, J=6.8 Hz, 3H).

### Example 20—Preparation of Cmpd 66

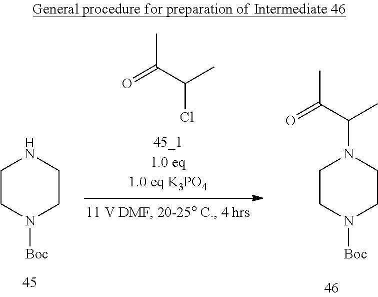

Charged DMF (35.0 mL) into 100-mL three neck round bottomed flask (R1) at 20-25° C.

Charged Intermediate 45_1 (1.44 g, 13.4 mmol, 1.37 mL, 1.00 eq) and Intermediate 45 (3.00 g, 13.4 mmol, 1.00 eq) into R1 at 20-25° C.

Charged K₃PO₄ (2.86 g, 13.4 mmol, 1.00 eq) into R1 at 20-25° C.

The mixture (R1) was heated slowly with stirring and temperature was raised to 20-25° C. for 4 hrs.

LC-MS showed that Intermediate 45 was consumed completely and Intermediate 46 was detected (RT=0.520 min).

The mixture was poured in water (100 mL) at 20-25° C. Extracted the mixture with Ethyl acetate (100 mL*3) and collecting the organic phase at 20-25° C. The organic layer was washed with brine (100 mL*2). The organic layer was dried with anhydrous Na₂SO₄. The filtrate was concentrated at 35-45° C. under vacuum.

The residue was purified by prep-HPLC (neutral condition, 330 g Flash Column Welch Ultimate XB_C18 20-40 μm; 120 A, Gradient B %: 10-60% 25 mins) to obtain Intermediate 46 (1.80 g, 6.10 mmol, 45.2% yield, 86.8% purity) as a yellow oil, confirmed by LCMS and HPLC.

LCMS: RT=0.520 mins, m/z=257.1 (M+H)⁺.

LCMS: RT=0.512 min, m/z=257.1 (M+H)⁺.

HPLC: RT=1.908 mins, 86.8% purity under 220 nm.

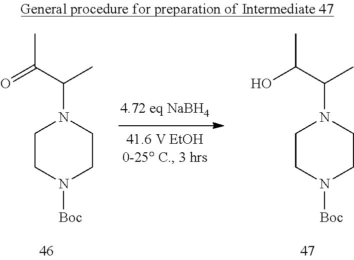 [SMILES: CC(=O)C(C)N1CCN(C(=O)OC(C)(C)C)CC1 ; CC(O)C(C)N1CCN(C(=O)OC(C)(C)C)CC1]

Charged EtOH (75.0 mL) in 250-mL three neck round bottomed flask (R1) under N2 at 20-25° C.

Charged intermediate 46 (1.80 g, 6.10 mmol, 86.8% purity, 1.00 eq) into R1 under N₂ at 20-25° C.

Take the mixture (R1) cooled to 0-5° C.

Charged NaBH₄ (1.09 g, 28.8 mmol, 4.72 eq) into R1 under N2 at 0-5° C.

Stirred the mixture (R1) for 3 hrs at 20-25° C.

TLC (Petroleum ether:Ethyl acetate=0:1) indicated intermediate 46 was consumed completely (R_{f}=0.6) and two new spot formed (R_{f}=0.4 and R_{f}=0.3). The reaction was clean according to TLC.

The mixture poured in saturated aqueous ammonium chloride (300 mL) at 0-10° C. Extracted the mixture with EtOAc (100 mL*3) and collecting the organic phase at 20-25° C. The organic layer was washed with brine (100 mL*2). The organic layer was dried with anhydrous MgSO₄. The filtrate was concentrated at 35-45° C. under vacuum to obtain Intermediate 47 (1.60 g, crude) as a yellow oil, confirmed by LCMS.

LCMS: RT=2.365, 2.683 min. m/z=259.2 (M+H)⁺.

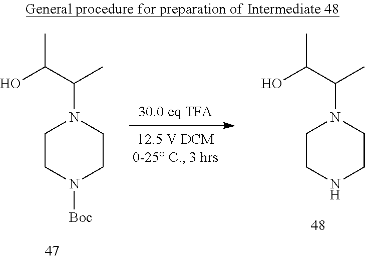 [SMILES: CC(O)C(C)N1CCN(C(=O)OC(C)(C)C)CC1 ; CC(O)C(C)N1CCNCC1]

Charged DCM (20.0 mL) in 100-mL three neck round bottomed flask (R1) at 20-25° C.

Charged intermediate 47 (1.60 g, 6.19 mmol, 1.00 eq) into R1 at 20-25° C.

Take the mixture (R1) cooled to 0-5° C.

Charged TFA (21.1 g, 185 mmol, 13.7 mL, 30.0 eq) into R1 at 0-5° C.

Stirred the mixture (R1) for 3 h at 20-25° C.

TLC analysis (Petroleum ether:Ethyl acetate=0:1) indicated intermediate 47 was consumed completely (R_{f}=0.4 and 0.3) and Intermediate 48 formed (R_{f}=0.25).

The filtrate was concentrated at 35˜45° C. under vacuum to obtain Intermediate 48 (900 mg, crude) as a yellow oil.

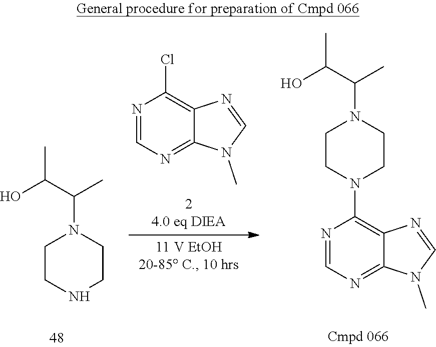 [SMILES: CC(O)C(C)N1CCNCC1 ; Cn1cnc2c(Cl)ncnc21 ; CC(O)C(C)N1CCN(c2ncnc3c2ncn3C)CC1]

Charged intermediate 48 (900 mg, 5.69 mmol, 1.00 eq) and EtOH (10.0 mL) into 100-mL round bottomed flask (R1) at 20-25° C.

Charged intermediate 2 (1.01 g, 5.69 mmol, 95.3% purity, 1.00 eq) and DIEA (2.94 g, 22.7 mmol, 3.96 mL, 4.00 eq) into R1 at 20-25° C.

The mixture (R1) was stirred for 10 h at 80-85° C.

LC-MS showed intermediate 48 was consumed completely and two main peaks with desired mass was detected (RT1=1.820 min and RT2=2.053 min).

The mixture poured in water (100 mL) at 0-10° C. Extracted the mixture with EtOAc (50.0 mL*3) and collecting the organic phase at 20-25° C. The organic layer was washed with brine (50.0 mL*2). The organic layer was dried with anhydrous Na₂SO₄. The filtrate was concentrated at 35-45° C. under vacuum.

The residue was purified by column chromatography (SiO₂, Dichloromethane:Methanol=8/1, R_{f}=0.5 and R_{f}=0.4). The racemic product (1.30 g) was separated by Prep-Chiral-SFC (column: column: DAICEL CHIRALCEL OJ (250 mm*30 mm, 10 um); mobile phase: [0.1% NH₃H₂O IPA]; B %: 15%-15%, 0 min) to obtain Cmpd 066 (500 mg, 1.70 mmol, 37.9% yield, 98.4% purity) as an Off-White solid, confirmed by LCMS, HPLC, and ¹H NMR.

LCMS: RT=1.820, 2.053 min, m/z=291.0 (M+H)+.

LCMS: RT=0.557 min, m/z=291.0 (M+H)⁺.

HPLC: RT=1.943 mins, 98.4% purity under 220 nm.

¹H NMR: (400 MHz, DMSO-d6): δ 8.24 (s, 1H), 8.13 (s, 1H), 4.24-4.23 (m, 5H), 3.72 (s, 3H), 3.54-3.51 (m, 1H), 2.67-2.66 (m, 2H), 2.47-2.43 (m, 2H), 2.35-2.32 (m, 1H), 1.05 (d, J=6.0 Hz, 3H), 0.86 (d, J=6.4 Hz, 3H).

### Example 21—Preparation of Cmpd 68

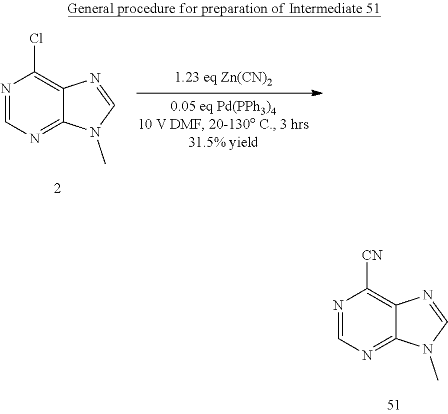

Charged Intermediate 2 (1.50 g, 8.18 mmol, 91.8% purity, 1.00 eq) and DMF (15.0 mL) into 100-mL three neck round bottomed flask (R1) at 20-25° C.

Charged Pd(PPh₃)₄ (472 mg, 408 μmol, 0.05 eq) into R1 under N₂ at 20-25° C.

Charged Zn(CN)₂ (1.18 g, 10.0 mmol, 637 μL, 1.23 eq) into R1 under N₂ at 20-25° C.

The solution was degassed and purged with N₂ for 3 times.

The reaction mixture was stirred at 130° C. for 3 h.

LC-MS showed Intermediate 2 was consumed completely and one main peak with desired mass was detected (RT=1.194 min).

The reaction mixture was cooled to 25° C. The mixture (R1) was poured into H₂O (20.0 mL) and Ethyl acetate (20.0 mL) at 20-25° C. The insoluble material was removed by filtering through a celite pad. The aqueous layer was separated and extracted with Ethyl acetate (30.0 mL*3). The combined organic layers were washed with brine (50.0 mL), dried over Na₂SO₄, filtered and concentrated.

The residue was purified by prep-HPLC (basic condition; 80 g Flash Column Welch Ultimate XB_C18 20-40 μm; 120A, Gradient B %: 33% 22 mins) to obtain Intermediate 51 (490 mg, 2.56 mmol, 31.2% yield, 83.0% purity) as a white solid, confirmed by LCMS and HPLC.

LCMS: RT=1.194 min, m/z=160.1 (M+H)⁺.

LCMS: RT=1.192 min, m/z=160.1 (M+H)⁺.

HPLC: RT=1.255 min, 81.8% purity under 220 nm.

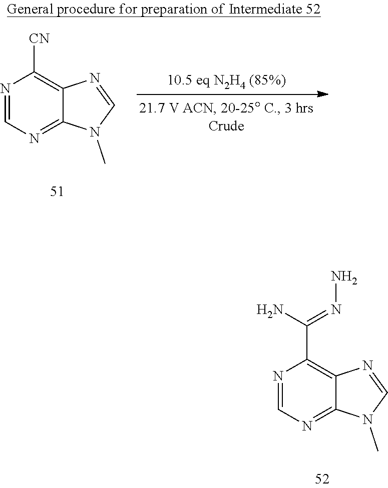

Charged EtOH (75.0 mL) in 250-mL three neck round bottomed flask (R1) under N₂ at 20-25° C.

Charged ACN (20.0 mL) into 100-mL three neck round bottomed flask (R1) at 20-25° C.

Charged Intermediate 51 (920 mg, 4.99 mmol, 86.3% purity, 1.00 eq) into R1 under N₂ at 20-25° C.

Charged N₂H₄·H₂O (3.12 g, 52.9 mmol, 3.03 mL, 85.0% purity, 10.6 eq) into R1 under N₂ at 20-25° C.

The solution was degassed and purged with N₂ for 3 times.

The reaction mixture was stirred at 20° C. for 3 hrs.

LCMS analysis showed that Intermediate 51 was consumed completely and Intermediate 52 was detected (RT=0.591 min)

The solution was filtered and washed with ACN (20.0 mL). The filter cake was concentrated at 30-40° C. under vacuum to obtain Intermediate 52 (800 mg, crude) as a yellow solid, confirmed by LCMS.

LCMS: RT=0.591 min, m/z=192.1 (M+H)⁺.

LCMS: RT=0.590 min, m/z=192.1 (M+H)⁺.

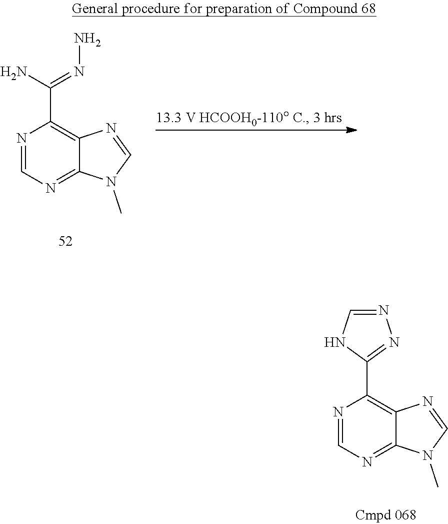

Charged HCOOH (10.0 mL) into 100-mL three neck round bottomed flask (R1) at 20-25° C.

Cooled the mixture to 0-5° C.

Dropped slowly Intermediate 52 (750 mg, 3.92 mmol, 1.00 eq) into R1 at 0-5° C. under N₂.

The solution was degassed and purged with N₂ for 3 times.

The reaction mixture was stirred at 110° C. for 3 h.

LC-MS (EC8860-42-P1A6) showed Intermediate 52 was consumed completely and one main peak with desired mass was detected (RT=0.329 min).

The mixture was poured into 50% NaOH to adjust pH 7˜8. Extracted with ethyl acetate (20.0 mL*4) and saturated salt solution 3 times. The combined organic layers were dried over Na₂SO₄. The filtrate was concentrated at 35-45° C. under vacuum.

The residue was purified by prep-HPLC (column: YMC Triart C18 70*250 mm*7 μm; mobile phase: [water (NH₄HCO₃)-ACN]; B %: 0%-30%, 15 min). The residue was purified by prep-HPLC (column: Phenomenex luna C18 250*50 mm*15 μm; mobile phase: [water (FA)-ACN]; B %: 0%-18%, 20 min) to obtain Cmpd 068 (510 mg, 2.26 mmol, 13.5% yield, 94.1% purity) as a white solid, confirmed by LCMS, HPLC, and ¹H NMR.

LCMS: RT=0.329 min, m/z=202.0 (M+H)+.

LCMS: RT=0.192 min, m/z=201.8 (M+H)⁺.

HPLC: RT=0.928 mins, 94.1% purity under 220 nm.

¹H NMR: (400 MHz, DMSO-d6): δ 15.2-14.4 (m, 1H), 9.02 (s, 1H), 8.81-8.65 (m, 2H), 3.88 (s, 3H).

### Example 22—Preparation of Cmpd 69

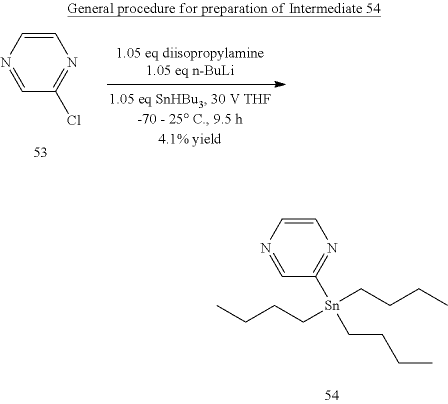 [SMILES: Clc1cnccn1 ; CCC[CH2][Sn]([CH2]CCC)([CH2]CCC)[c]1cnccn1]

Charged THF (200 mL) and DIISOPROPYLAMINE (18.5 g, 183 mmol, 25.9 mL, 1.05 eq) into 2.0 L three-neck-round bottomed flask (R1) at 25° C.

Degassed and purged with N₂ for 3 times.

n-BuLi (2.50 M, 73.3 mL, 1.05 eq) is dropwise added under an ice-water bath under N₂ at 0-5° C., then react for 10 mins under N₂ at 0-5° C.

SnHBu₃ (49.7 g, 171 mmol, 45.2 mL, 9.82e-1 eq) was added dropwise under water bath under N₂ at 0-5° C., continued to react for 20 mins under N₂.

Cooled to −70-−65° C., then compound 53 (20.0 g, 174 mmol, 15.6 mL, 1.00eq) is dissolved in THF (400 mL) then the mixture was slowly added dropwise to R1 at −70-−65° C. under N₂.

Stirred the mixture at −70˜−65° C. for 8 h under N₂.

LCMS analysis showed that 38.6% of Intermediate 53 (RT=0.164 min) remained and the desired compound (RT=0.660 min) was detected.

Temperature was raised to −40° C., the reaction as quenched by adding dropwise aqueous solution of potassium fluoride (400 mL) at −40-−35° C.

Stirred the mixture at 10-20° C. for 0.5 hr.

The reaction solution was filtered and extracted with ethyl acetate (400 mL*3) at 25° C., dried over sodium sulfate, the desiccant was filtered, and the filtrate was concentrated and dried under reduced pressure. The crude product is purified by column chromatography (SiO₂, Petroleum ether/Ethyl acetate=0 to 15%, Petroleum ether/Ethyl acetate=3/1, product: RF=0.35) to obtain Intermediate 54 (3.00 g, 7.17 mmol, 4.10% yield, 88.2% purity) a yellow oil, confirmed by LCMS.

LCMS: RT=0.660 min, m/z=370.9 (M+H)⁺.

LCMS: RT=0.660 min, m/z=370.9 (M+H)⁺.

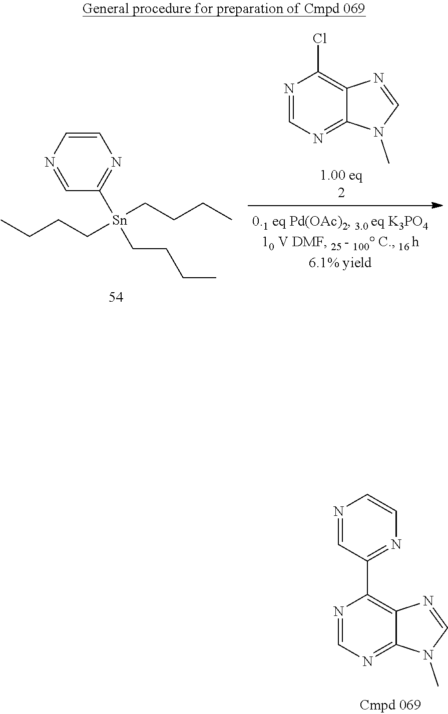 [SMILES: CCC[CH2][Sn]([CH2]CCC)([CH2]CCC)[c]1cnccn1 ; Cn1cnc2c(Cl)ncnc21 ; Cn1cnc2c(-c3cnccn3)ncnc21]

Charged intermediate 54 (3.10 g, 7.41 mmol, 88.2% purity, 1.00 eq) and DMF (31.0 mL) into 100 mL three-neck-round bottomed flask (R1) at 25° C.

Charged intermediate 2 (2.87 g, 8.15 mmol, 73.8% purity, 1.10 eq) and K₃PO₄ (4.72 g, 22.2 mmol, 3.00 eq) into R1 at 25° C.

Charged Pd(OAc)₂ (166 mg, 740 μmol, 0.10 eq) into R1 at 25° C. under N₂.

Reaction was stirred at 95-100° C. for 16 h.

LCMS analysis showed that intermediate 54 was consumed and the desired MS (RT=0.189 min) was detected.

Concentrated under reduced pressure under 45-50° C. to give a residue.

Residue was purified by column chromatography (SiO₂, DCM:MeOH=50/1 to 10/1, product: DCM:MeOH=10/1, product: RF=0.21.) to get crude product.

Crude product was triturated with MeOH at 20-25° C. for 20 mins to obtain Cmpd 069 (100 mg, 454 μmol, 6.13% yield, 96.4% purity) as a yellow solid, confirmed by LCMS, HPLC, and ¹H NMR.

LCMS: RT=0.189 min, m/z=213.1 (M+H)⁺.

LCMS: RT=0.378 min, m/z=213.0 (M+H)⁺.

HPLC: RT=1.865 min, 96.4% purity under 220 nm.

¹H NMR (400 MHz, DMSO): δ 9.79 (d, J=1.2 Hz, 1H), 9.10 (s, 1H), 8.92-8.91 (t, J=2.0 Hz, 1H), 8.81 (d, J=2.4 Hz, 1H), 8.70 (s, 1H), 3.91 (s, 1H).

### Example 23—Preparation of Cmpd 70

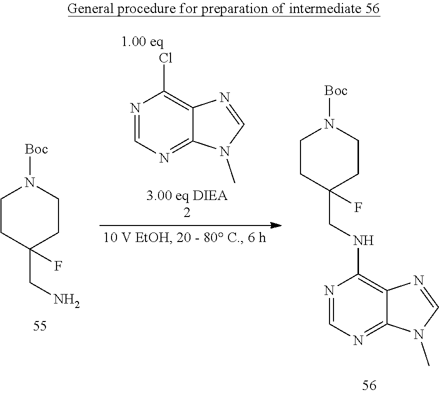 [SMILES: CC(C)(C)OC(=O)N1CCC(F)(CN)CC1 ; Cn1cnc2c(Cl)ncnc21 ; Cn1cnc2c(NCC3(F)CCN(C(=O)OC(C)(C)C)CC3)ncnc21]

Charged intermediate 55 (1.00 g, 4.30 mmol, 1.00 eq) into a 100 mL three-necked round-bottomed flask (R1) at 20-25° C.

Charged intermediate 2 (770 mg, 4.30 mmol, 94.2% purity, 1.00 eq) into R1 at 20-25° C.

Charged DIEA (1.67 g, 12.9 mmol, 2.25 mL, 3.00 eq) into R1 at 20-25° C.

Charged EtOH (10 mL) into R1 at 20-25° C.

The mixture was stirred 6 h at 70-80° C.

LCMS showed that intermediate 55 was consumed and intermediate 56 (RT=0.347 min) was detected.

The mixture was concentrated under vacuum at 45° C. to obtain compound 56 (2.40 g, crude) as a brown oil, confirmed by LCMS.

LCMS: RT=0.347 min, m/z=365.0 (M+H)⁺.

LCMS: RT=0.345 min. m/z=365.4 (M+H)⁺.

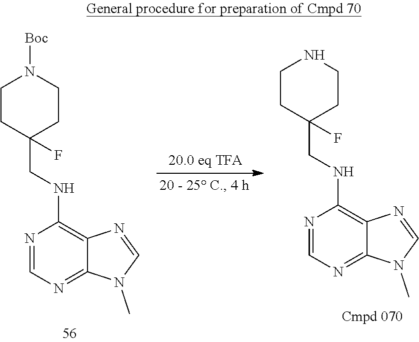 [SMILES: Cn1cnc2c(NCC3(F)CCN(C(=O)OC(C)(C)C)CC3)ncnc21 ; Cn1cnc2c(NCC3(F)CCNCC3)ncnc21]

Charged intermediate 56 (2.40 g, 6.59 mmol, 1.00 eq) into a 100 mL stand-up bottle (R1) at 20-25° C.

Charged TFA (15.0 g, 131 mmol, 9.75 mL, 20.0 eq) into R1 at 20-25° C.

The mixture was stirred 4 h at 20-25° C.

LCMS showed intermediate 56 was consumed and the desired compound (RT=0.610 min) was detected.

The mixture was poured into the saturated of NaOH (10.0 mL).

Filtered and washed the filter cake with H₂O (30.0 mL).

Washed the aqueous phase with DCM:MeOH=10:1 (80.0 ml*4).

Washed the organic phase with saturated salt solution and dried over Na₂SO₄.

The mixture was concentrated under vacuum at 45° C., the crude product was purified by reversed phase HPLC (0.1% NH₃·H₂O), and freeze-dried.

The crude product was triturated with EtOAc (30.0 mL) at 20-25° C. for 30 min and dried over the filter cake under vacuum at 45° C. to obtain Cmpd 070 (631 mg, 2.35 mmol, 35.6% yield, 98.2% purity) white solid, confirmed by LCMS, HPLC, ¹H NMR, and ¹⁹F NMR.

LCMS: RT=0.610 min, m/z=265.4 (M+H)⁺.

LCMS: RT=0.605 min, m/z=265.2 (M+H)⁺.

HPLC: RT=2.829 mins, 98.2% purity under 220 nm.

19F NMR (400 MHz, DMSO-d6)

¹H NMR (400 MHz, DMSO-d6): δ 8.22 (br s, 1H), 8.12 (s, 1H), 7.65 (br s, 1H), 5.75 (s, 1H), 2.81-2.72 (m, 2H), 2.71-2.60 (m, 2H), 1.81-1.40 (m, 4H).

### Example 24—Preparation of Cmpd 71

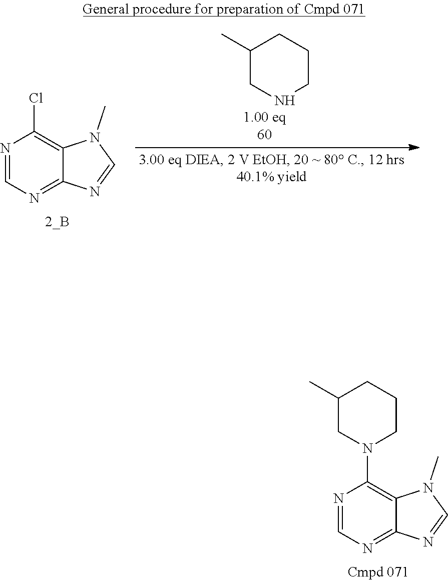

Charged intermediate 2_B (1.50 g, 8.01 mmol, 90.0% purity, 1.00 eq) and intermediate 60 (794 mg, 8.01 mmol, 939 μL, 1.00 eq) into a 10 ml single-necked round-bottomed flask (R1) in EtOH (3 mL) at 20˜25° C.

Charged DIEA (3.10 g, 24.0 mmol, 4.18 mL, 3.00 eq) into R1 at 20˜25° C.

Warmed to 80° C. and stirred at 80° C. for 12 hrs.

LCMS showed intermediate 2_B was consumed completely and the desired product (RT=1.653 and 1.669 mins) was detected.

The mixture was concentrated, purified by prep-HPLC (column: Phenomenex luna C18 250*50 mm*15 μm; mobile phase: [water (NH₄HCO₃)-ACN]; B %: 15%-45%, 20 min), and the eluent was lyophilized to obtain Cmpd 071 (743 mg, 3.21 mmol, 40.1% yield, 100% purity) as a yellow solid, which was confirmed by HPLC, LCMS, ¹H NMR, and Nuclear Overhauser Effect (NOE) NMR.

LCMS: RT=1.653 min, 232.1 (M+H)⁺.

LCMS: RT=0.248 min, 232.1 (M+H)⁺.

¹H NMR/NOE (400 MHz, CDCl₃): δ 8.63 (s, 1H), 7.96 (s, 1H), 4.01 (s, 3H), 3.79-3.64 (m, 2H), 2.94 (dt, J=2.8, 12.4 Hz, 1H), 2.64 (dd, J=10.8, 12.4 Hz, 1H), 2.10 (s, 2H), 1.89-1.81 (m, 2H), 1.78-1.68 (m, 1H), 1.16 (dq, J=4.0, 12.0 Hz, 1H), 0.97 (d, J=6.4 Hz, 3H).

### Example 25—Preparation of Cmpd 158

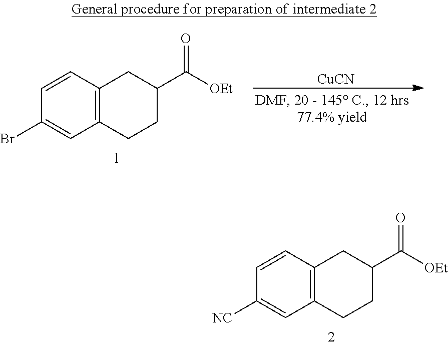 [SMILES: CCOC(=O)C1CCc2cc(Br)ccc2C1 ; "CuCN" ; CCOC(=O)C1CCc2cc(C#N)ccc2C1]

Charged intermediate 1 (3.00 g, 10.5 mmol, 1.00 eq) into R1 at 20° C.

Charged DMF (30.0 mL) into R1 at 20° C.

Charged CuCN (1.99 g, 22.2 mmol, 4.86 mL, 2.10 eq) into R1 at 20° C.

Stirred the mixture at 145° C. for 12 h under N₂.

TLC (Petroleum ether:Ethyl acetate=8:1) showed that intermediate 1 was consumed (R_{f}=0.70) and intermediate 2 was found (R_{f}=0.01, 0.11, 0.21, 0.31).

Cooled down to 20° C. then poured the mixture into NaClO (50.0 mL).

Extracted with ethyl acetate (50.0 mL*3).

Washed with brine (50.0 mL*2) and dried with Na₂SO₄, filtered, and concentrated in vacuum.

The crude product was purified by silica gel chromatography (silica gel, Petroleum ether/Ethyl acetate=8/1, R_{f}=0.21) to obtain intermediate 2 (1.88 g, 8.20 mmol, 77.4% yield, 100% purity) as a yellow gum, confirmed by LCMS and ¹H NMR.

LCMS: RT=0.442 min, m/z=230.1 (M+H)+.

¹H NMR: (400 MHz, DMSO): δ 7.55-7.52 (m, 2H), 7.33-7.31 (m, 1H), 4.13-4.08 (m, 2H), 3.06-2.78 (m, 5H), 2.08-2.07 (m, 1H), 1.78-1.75 (m, 1H), 1.20 (t, J=7.2 Hz, 14.0 Hz, 3H).

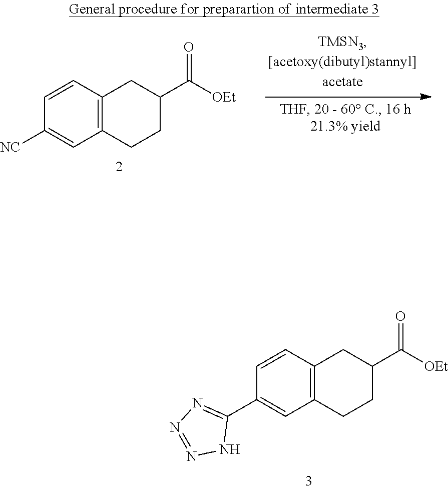 [SMILES: CCOC(=O)C1CCc2cc(C#N)ccc2C1 ; CCOC(=O)C1CCc2cc(-c3nnn[nH]3)ccc2C1]

Charged THF (20.0 mL) into a 100 mL stand-up flask (R1) at 20° C.

Charged intermediate 2 (1.88 g, 8.20 mmol, 1.00 eq) into R1 at 20° C.

Charged tetrabutylammonium-acetate (2.47 g, 8.20 mmol, 2.50 mL, 1.00 eq) into R1 at 20° C.

Charged TMSN₃ (3.78 g, 32.8 mmol, 4.31 mL, 4.00 eq) into R1 at 20° C.

Stirred the mixture at 60° C. for 16 h.

LCMS showed that intermediate 2 remained (RT=0.444 min), but intermediate 3 was found (RT=0.389 min).

Poured the mixture into water (80.0 mL), extracted with ethyl acetate (80.0 mL*3), combined the organic phases, washed with saturated brine (80.0 mL*2), dried with Na₂SO₄, filter, and concentrated in vacuum. The crude product was purified by silica gel chromatography (silica gel, Dichloromethane:Methanol=10/1, R_{f}=0.52) to obtain intermediate 3 (479 mg, 1.75 mmol, 21.3% yield, 99.6% purity) as a white solid, confirmed by LCMS.

LCMS: RT=0.389 min, m/z=273.1 (M+H)⁺.

LCMS: RT=0.389 min, m/z=273.1 (M+H)⁺.

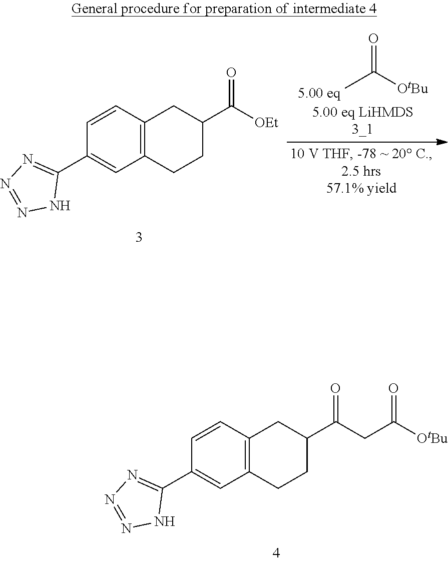

Charged intermediate 3_1 (1.12 g, 9.63 mmol, 1.29 mL, 5.00 eq) into a 50.0 mL stand-up flask (R1) at 20° C.

Charged THF (5.50 mL) into R1 at 20° C.

Cooled down to −78° C. and the mixture was degassed with N₂ for 3 times.

Dropwise LiHMDS (1 M, 9.63 mL, 5.00 eq) into R1 at −78° C.

Stirred the mixture under N₂ for 0.5 h under −78° C.

A solution of intermediate 3 (530 mg, 1.93 mmol, 1.00 eq) in THF (3.00 mL) was dropwise into R1 at −78° C.

Stirred the mixture at −78° C. for 2 h.

LCMS showed that intermediate 3 was consumed and intermediate 4 was found (RT=0.426 min).

Added the mixture dropwise into NH₄Cl (30.0 mL), extracted with ethyl acetate (15.0 mL*3), washed the combined organic layer with brine (30.0 mL), dried with Na₂SO₄, filtered and concentrated.

The crude product was purified by Prep-HPLC (column: Phenomenex Luna C18 200*40 mm*10 μm; mobile phase: [water (FA)-ACN]; gradient: 33%-63% B over 10 min) to obtain intermediate 4 (400 mg, 1.10 mmol, 57.1% yield, 94.2% purity) as white solid, confirmed by LCMS.

LCMS: RT=0.426 min, m/z=343.2 (M+H)⁺.

LCMS: RT=0.418 min, m/z=343.0 (M+H)⁺.

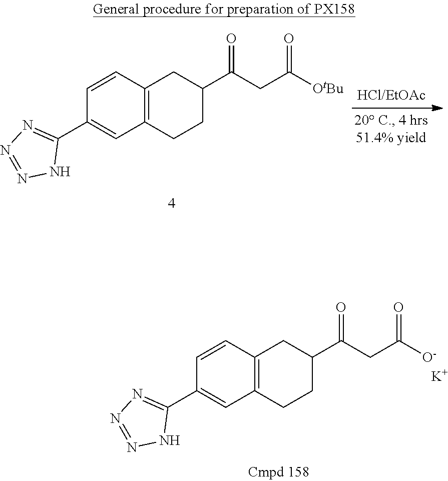 [SMILES: CC(C)(C)OC(=O)CC(=O)C1CCc2cc(-c3nnn[nH]3)ccc2C1 ; O=C([O-])CC(=O)C1CCc2cc(-c3nnn[nH]3)ccc2C1 ; [K+]]

Charged intermediate 4 (500 mg, 1.38 mmol, 1.00 eq) into R1 (10.0 mL flask) at 20° C.;

Charged HCl/EtOAc (4 M, 5.00 mL, 14.5 eq) into R1 at 20° C.;

Stirred the mixture at 20° C. under N₂ for 4 h.

LCMS showed that intermediate 4 was remained (RT=0.663 min), but desired mass was detected (RT=0.433 min).

The reaction mixture was concentrated under reduced pressure to give the residue. Add aq. K₂CO₃ (2 mL) into the mixture to get a solution.

The crude product was purified by Prep-HPLC (column: Waters xbridge 150*25 mm 10 μm; mobile phase: [water (NH4HCO3)-ACN]; gradient: 1%-20% B over 10 min) to obtain Cmpd 158 (210 mg, 708 μmol, 51.4% yield, 96.3% purity) as a white solid, confirmed by ¹H NMR, LCMS, HPLC, and DSC.

LCMS: RT=0.433 min, m/z=286.9 (M+H)⁺.

LCMS: RT=0.435 min, m/z=286.9 (M+H)⁺.

HPLC: RT=1.075 mins, 96.3% purity under 220 nm.

¹H NMR: (400 MHz, DMSO): δ 7.68-7.66 (m, 2H), 7.23 (d, J=7.60 Hz, 1H), 3.58-3.50 (m, 1H), 3.03-2.80 (m, 5H), 2.16-2.12 (m, 1H), 1.76-1.66 (m, 1H).

### Example 26—Preparation of Cmpd 174

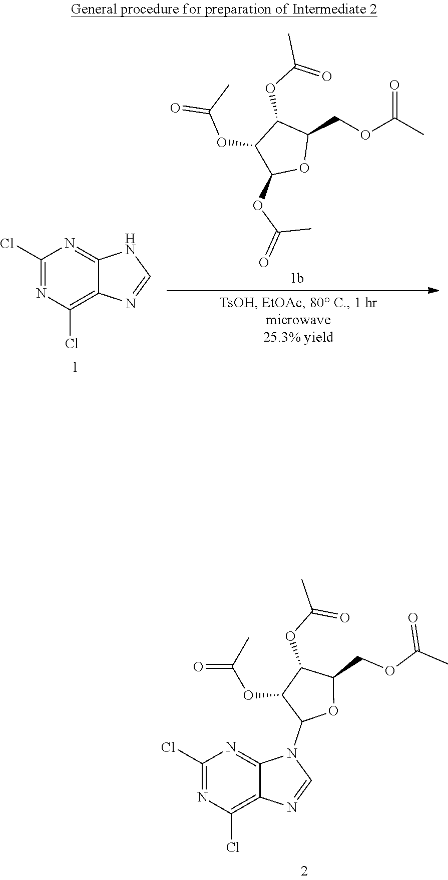 [SMILES: Clc1nc(Cl)c2nc[nH]c2n1 ; CC(=O)OC[C@H]1O[C@@H](OC(C)=O)[C@H](OC(C)=O)[C@@H]1OC(C)=O ; CC(=O)OC[C@H]1OC(n2cnc3c(Cl)nc(Cl)nc32)[C@H](OC(C)=O)[C@@H]1OC(C)=O]

Added Intermediate 1 (2.00 g, 10.6 mmol, 1.00 eq), Intermediate 1b (3.36 g, 10.6 mmol, 1.00 eq), TsOH (182 mg, 1.06 mmol, 0.10 eq) to EtOAc (20.0 mL).

Stirred at 80° C. for 1 h in a microwave reactor.

TLC (Petroleum ether:Ethyl acetate=2:1) showed Compound 1 (R_{f}=0.10) was consumed completely and a main spot (R_{f}=0.50) was formed.

The mixture was filtered and the filtrate was concentrated. The residue was purified by column chromatography (SiO₂, Petroleum ether:Ethyl acetate=100:1 to 2:1, R_{f}=0.50 (Petroleum ether:Ethyl acetate=2:1)) to obtain Intermediate 2 (1.20 g, 2.68 mmol, 25.3% yield), as a white solid.

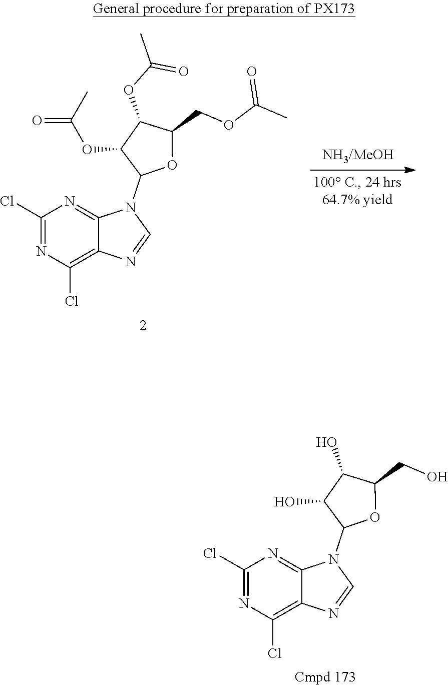 [SMILES: CC(=O)OC[C@H]1OC(n2cnc3c(Cl)nc(Cl)nc32)[C@H](OC(C)=O)[C@@H]1OC(C)=O ; OC[C@H]1OC(n2cnc3c(Cl)nc(Cl)nc32)[C@H](O)[C@@H]1O]

Intermediate 2 (1.20 g, 2.68 mmol, 1.00 eq) was dissolved in NH₃/MeOH (12.0 mL)

Stirred at 100° C. for 24 hrs.

TLC (Petroleum ether:Ethyl acetate=2:1) showed Intermediate 2 (R_{f}=0.50) was consumed completely and a main spot (R_{f}=0.10) was formed.

The mixture was concentrated in vacuum to get a residue. The residue was purified by reversed-phase HPLC (0.1% HCl condition) to obtain Cmpd 173 (557.91 mg, 1.74 mmol, 64.7% yield, 100% purity) as a white solid, confirmed by LCMS, HPLC, and ¹H NMR.

LCMS: RT=0.428 min, m/z (M+23)⁺=343.0.

HPLC: RT=1.440 mins, 100% purity under 220 nm.

¹H NMR: (400 MHz, DMSO-d6): δ 8.98 (s, 1H) 5.97 (d, J=5.2 Hz, 1H) 5.59 (d, J=6.0 Hz, 1H) 5.26 (d, J=5.6 Hz, 1H) 5.08 (t, J=5.2 Hz, 1H) 4.51 (dd, J=10.4, 5.2 Hz, 1H) 4.17 (dd, J=10.0, 4.8 Hz, 1H) 3.98 (dd, J=8.0, 4.0 Hz, 1H) 3.75-3.67 (m, 1H) 3.63-3.55 (m, 1H).

### Example 27—Preparation of Cmpd 174

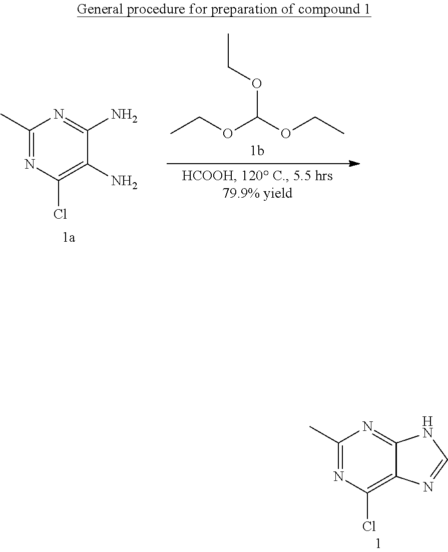

Added Intermediate 1a (4.00 g 25.2 mmol, 1.00 eq) to Intermediate 1b (60.0 mL) and HCOOH (1.00 mL).

Stirred at 120DC for 5.5 h under N₂.

LCMS showed Intermediate 1a was consumed and desired mass (RT=0.158 min) was detected.

The mixture was filtered, and the filtrate was concentrated to obtain Intermediate 1 (3.40 g, 20.1 mmol, 79.95 yield) as an off-white solid.

LCMS: RT=0.158 min, nm/z (M+H)⁺=168.9.

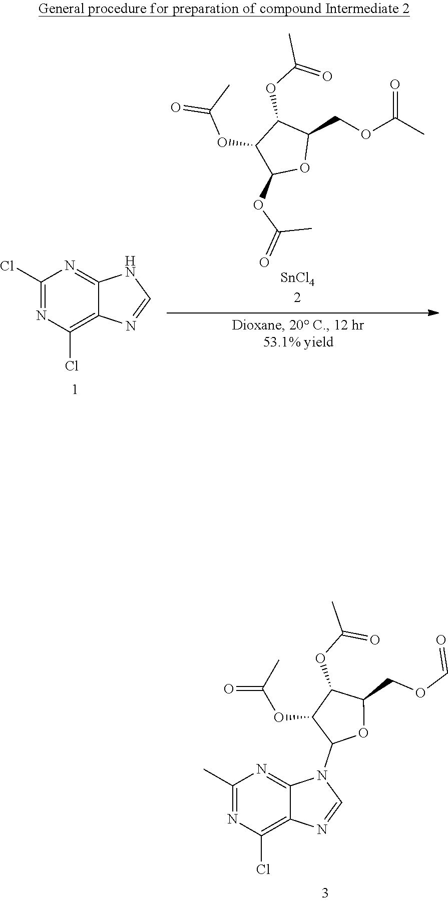 [SMILES: CC(=O)OC[C@H]1O[C@@H](OC(C)=O)[C@H](OC(C)=O)[C@@H]1OC(C)=O ; Clc1nc(Cl)c2nc[nH]c2n1 ; CC(=O)O[C@@H]1[C@@H](COC=O)OC(n2cnc3c(Cl)nc(C)nc32)[C@@H]1OC(C)=O]

Charged Intermediate 1 (500 mg, 2.97 mmol, 1.00 eq) into a 50 mL stand-up flask (R1) at 20′C.

Charged Intermediate 2 (944 mg, 2.97 mmol, 1.00 eq) into R1 at 20° C.

Charged Dioxane (15.0 mL) into R1 at 20° C.

Dropwise SnCl₄ (773 mg, 2.97 mmol, 347 μL, 1.00 eq) into R1 at 0° C.

Stirred the mixture at 20° C. for 12 h.

LCMS showed Intermediate 1 remained (RT=0.202 min), but Intermediate 3 was found (RT=0.371 min).

Poured the mixture into water (50.0 mL).

Extracted with EtOAc (50.0 mL*3).

The organic phase was washed with brine (50.0 mL*2), dried with Na₂SO₄, and filtered and concentrated in vacuum. The crude product was purified by prep-TLC (Ethyl acetate:Petroleum ether=2:1, R_{f}=0.4) to obtain Intermediate 3 (1.4 g, 3.15 mmol, 53.09% yield, 96% purity) as a yellow gum, confirmed by LCMS.

LCMS: RT=0.371 min, m/z=427.1 (M+H)⁺.

LCMS: RT=0.363 min, m/z=426.9 (M+H)⁺.

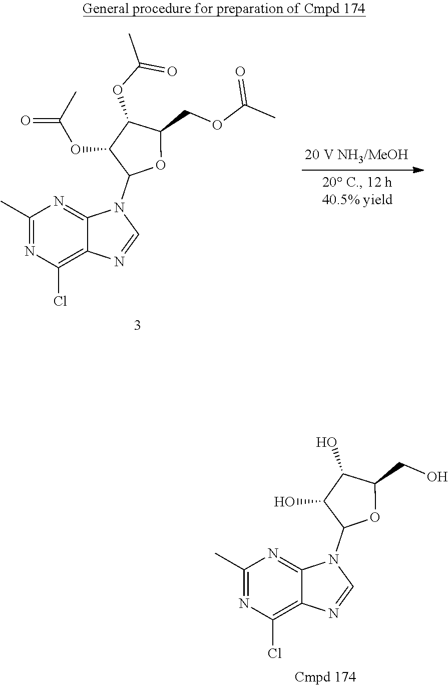

Charged Intermediate 3 (1.20 g, 2.70 mmol, 1.00 eq) into a 50 mL stand-up flask (R1) at 20° C.

Charged NH₃/MeOH (7 M, 23.0 mL, 59.8 eq) into R1 at 20° C.

Stirred the mixture at 20° C. for 12 h.

LCMS (EC13849-3-p1a4) showed Intermediate 3 was consumed, Cmpd 174 was found (RT=0.306 min), and then concentrated in vacuum.

The crude product was purified by prep-HPLC (column: Phenomenex luna C18 150*25 mm*10 μm; mobile phase: [water (FA)-ACN]; gradient: 0%-25% B over 10 min) to obtain Cmpd 174 (333 mg, 1.09 mmol, 40.5% yield, 98.6% purity) as a white solid, confirmed by LCMS, HPLC, and ¹H NMR.

LCMS: RT=0.306 min, m/z=301.1 (M+H)⁺.

LCMS: RT=0.294 min, m/z=301.1 (M+H)⁺.

HPLC: RT=1.272 mins, 98.6% purity under 220 nm.

¹H NMR (400 MHz, DMSO): δ 8.84 (s, 1H), 6.00 (d, J=5.6 Hz, 1H), 5.53 (d, J=6.0 Hz, 1H), 5.26 (d, J=5.2 Hz, 1H), 5.10-5.13 (m, 1H), 4.56-4.58 (m, 1H), 4.17-4.19 (m, 1H), 3.98-3.99 (m, 1H), 3.58-3.69 (m, 2H), 2.69 (s, 3H).

### Example 28—Preparation of Cmpd 175

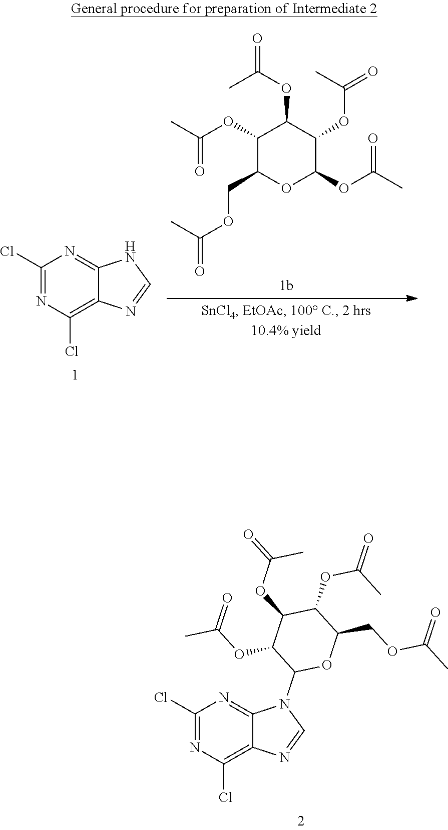 [SMILES: Clc1nc(Cl)c2nc[nH]c2n1 ; CC(=O)OC[C@@H]1O[C@H](OC(C)=O)[C@@H](OC(C)=O)[C@H](OC(C)=O)[C@H]1OC(C)=O ; CC(=O)OC[C@H]1OC(n2cnc3c(Cl)nc(Cl)nc32)[C@H](OC(C)=O)[C@@H](OC(C)=O)[C@@H]1OC(C)=O]

Equipped a 100 mL of three flask with stirrer, addition Nitrogen ball and thermometer.

Charged Intermediate 1b (14.4 g, 37.0 mmol, 1.00 eq) and EtOAc (70 mL) to the flask.

Warmed the flask to 100° C.

Charged Intermediate 1 (7.00 g, 37.0 mmol, 1.00 eq) to the flask at 100° C.

Charged SnCl₄ (192 mg, 740 μmol, 86.6 μL, 0.02 eq) to the flask at 100° C.

Stirred at 100° C. for 2 hrs under N₂.

LCMS (EC12863-2-P1B1) showed Compound 1 was consumed and desired mass (RT=0.396 min) was detected.

The mixture was filtered and the filtrate was concentrated to get a residue.

The residue was triturated with petroleum ether (200 mL) and ethyl acetate (10.0 mL) at 0° C. for 30 mins. The crude product was purified by reversed phase (0.1% HCl condition) to obtain Compound 2 (2.00 g, 3.85 mmol, 10.4% yield) as a yellow solid.

LCMS: EC12863-2-P1B1, RT=0.396 min, m/z (M+H)⁺=518.9

¹H NMR (400 MHz, DMSO): δ 9.08 (s, 1H), 6.36 (d, J=9.2 Hz, 1H), 5.79 (t, J=9.2 Hz, 1H), 5.66 (d, J=9.6 Hz, 1H), 5.24 (t, J=10.0 Hz, 1H), 4.47-4.41 (m, 1H), 4.11 (d, J=4.0 Hz, 2H), 2.04 (s, 3H), 1.99 (d, J=7.2 Hz, 6H), 1.72 (s, 3H).

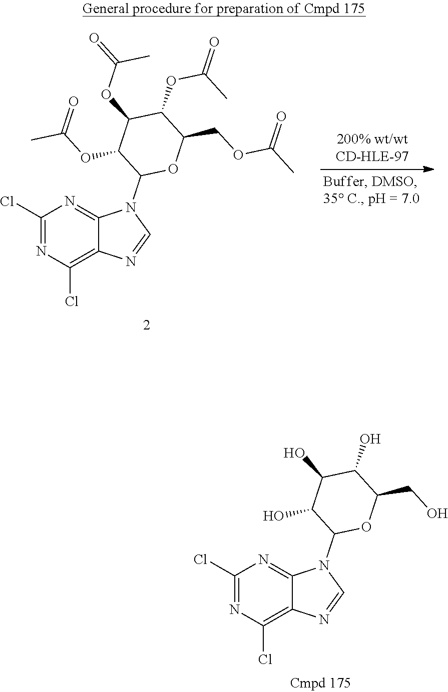 [SMILES: CC(=O)OC[C@H]1OC(n2cnc3c(Cl)nc(Cl)nc32)[C@H](OC(C)=O)[C@@H](OC(C)=O)[C@@H]1OC(C)=O ; OC[C@H]1OC(n2cnc3c(Cl)nc(Cl)nc32)[C@H](O)[C@@H](O)[C@@H]1O]

Charged CD-HLE-97 (0.80 g, 200% wt/wt) in Buffer (20.0 mL) (0.1 M solution of sodium phosphate pH=7.0) to reaction flask (R1).

Charged Intermediate 2 (400 mg, 770.28 μmol, 1.00 eq) in DMSO (2.0 mL) to R1;

Stirred mixture at 35° C. for 12 h.

LCMS (EC6017-222-P1A2) showed that the reactant was consumed completely, and product was detected (RT=0.685 min).

Poured the mixture into water (30.0 mL).

Extracted with EtOAc (30.0 mL*3).

The organic phase was washed with brine (30.0 mL*2), dried with Na₂SO₄, filtered, and concentrated in vacuum. The residue was purified by column chromatography (SiO₂, Ethyl acetate:Petroleum ether=3:1, R_{f}=0.30) to obtain Cmpd 175 (136.09 mg, 314.8 μmol, 70.5% purity) as a yellow solid, confirmed by LCMS, HPLC, and ¹H NMR.

LCMS: RT=0.196 min, m/z=188.8 (M+H)⁺.

HPLC: RT=1.193 mins, 70.5% purity under 220 nm.

¹H NMR (400 MHz, DMSO): δ 8.98 (s, 1H), 5.52 (d, J=9.6 Hz, 1H), 5.36-5.42 (m, 2H), 5.22 (d, J=5.6 Hz, 1H) 4.60-4.62 (m, 1H), 3.96-3.97 (m, 1H), 3.71-3.72 (m, 1H), 3.27-3.49 (m, 4H).

### Example 29—Preparation of Cmpd 178

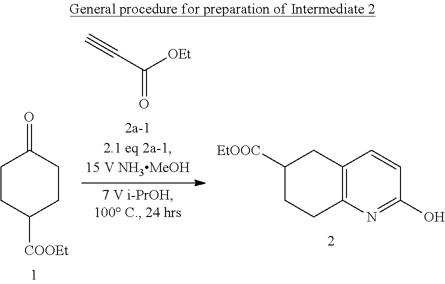

Charged Intermediate 1 (2.00 g, 11.7 mmol, 1.87 mL, 1.00 eq), i-PrOH (7.5 mL), Intermediate 2a-1 (2.42 g, 24.6 mmol, 2.42 mL, 2.1 eq), and NH₃/MeOH (7 M, 20.0 mL, 11.9 eq) into a 100 mL flask, at 20° C., and stirred for 24 h at 100° C. A sample was collected for TLC analysis, which indicated that Intermediate 1 remained, but one major new spot with larger polarity was detected (R_{f}=0.24). The mixture was concentrated by vacuum, the residue was purified by column chromatography (SiO₂, Petroleum ether/Ethyl acetate=5/1), and Intermediate 2 (1.80 g, 7.60 mmol, 16.1% yield, 93.4% purity) was obtained as a white solid.

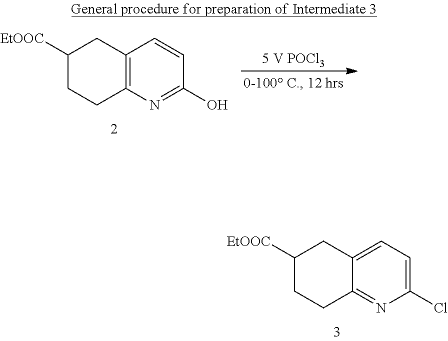 [SMILES: CCOC(=O)C1CCc2nc(O)ccc2C1 ; "5 V POCl3" ; CCOC(=O)C1CCc2nc(Cl)ccc2C1]

Charged Intermediate 2 (4.00 g, 18.0 mmol, 1.0 eq) and POCl₃ (32.9 g, 214 mmol, 20.0 mL, 11.8 eq) into a 100 mL flask, at 20° C., heat to 100° C., and reflux for 12 h. A sample was collected for LCMS analysis, which showed that Intermediate 2 was consumed and 62.7% of Intermediate 3 was detected (RT=0.411 min). The mixture was poured into ice water (15.0 mL) and extracted with EtOAc (15.0 mL*3). The combined organic layer was washed with brine (15.0 ml*3), dried with Na₂SO₄, filtered and concentrated. The residue was purified by column chromatography (SiO₂, Petroleum ether/Ethyl acetate=1/0 to 0/1) R_{f}=0.50, and Intermediate 3 (1.80 g, 7.30 mmol, 40.3% yield, 97.2% purity) was obtained as a white oil, confirmed by LCMS.

LCMS: RT=0.412 min, m/z=240.1 (M+H)⁺.

LCMS: RT=0.373 min, m/z=240.1 (M+H)⁺.

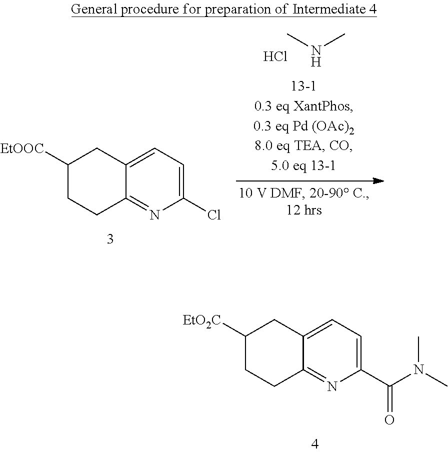

Loaded Intermediate 3 (1.5 g, 6.26 mmol, 1.00 eq), DMF (18 mL), Intermediate 13-1 (2.55 g, 31.29 mmol, 5.0 eq), Pd(OAc)₂ (421 mg, 1.88 mmol, 0.3 eq), XantPhos (1.09 g, 1.88 mmol, 0.3 eq), and TEA (5.07 g, 50.06 mmol, 6.97 mL, 8 eq) into a 100 mL flask (R1), at 20° C. The mixture was degassed with CO (175 mg, 6.26 mmol, 140 μL, 1.0 eq) for three times and stirred at 90° C. for 12 h. A sample was collected for LCMS analysis, which showed that Intermediate 3 was consumed and 11.8% of Intermediate 4 (RT=0.295 min) was detected.

The mixture was poured into aqueous NH₄Cl (15.0 mL) and extracted with EtOAc (15.0 mL*3). The combined organic layer was washed with brine (15.0 mL*3), dried with Na₂SO₄, filtered, and concentrated. The residue was purified by column chromatography by prep-TLC (SiO₂, Petroleum ether/Ethyl acetate=0:1) R_{f}=0.10, and Intermediate 4 (310 mg, 974 μmol, 15.5% yield, 86.9% purity) was obtained as a yellow oil, confirmed by LCMS.

LCMS: RT=0.295 min, m/z=277.1 (M+H)⁺.

LCMS: RT=0.291 min, m/z=277.1 (M+H)⁺.

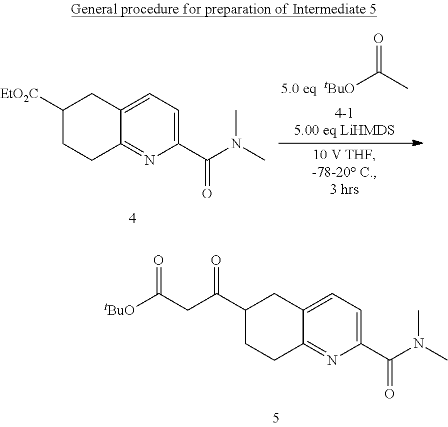

Loaded Intermediate 4-1 (546 mg, 4.70 mmol, 631 μL, 5.00 eq), THF (2.00 mL), and LiHMDS (2.5 M, 1.88 mL, 5.00 eq) into a flask (R1) at −78° C. The mixture was stirred at −78° C. for 1 h. In another flask (R2), at 20° C., Intermediate 4 (260 mg, 940 μmol, 1.00 eq) and THF (3.00 mL) were loaded. Then, R2 was loaded into R1, at −78° C., and the mixture was stirred for 2 h. A sample was collected for LCMS analysis, which showed that Intermediate 4 was consumed and 70.2% of Intermediate 5 (RT=0.351 min) was detected.

The mixture was poured into aqueous NH₄Cl (10.0 mL), extracted with EtOAc (10.0 mL*3), and the combined organic layer was washed with brine (10.0 mL*3), dried with Na₂SO₄, filtered and concentrated. The crude product was purified by prep. HPLC (Welch Xtimate C18 150*25 mm*5 um; mobile phase: [water (TFA)-ACN]; gradient: 25%-45% B over 10 min) to obtain Intermediate 5 (250 mg, crude) as a white oil.

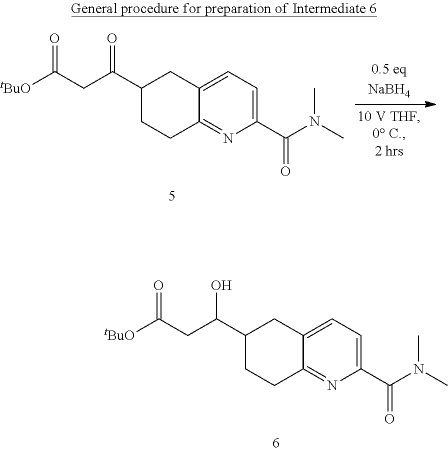 [SMILES: CN(C)C(=O)c1ccc2c(n1)CCC(C(=O)CC(=O)OC(C)(C)C)C2 ; CN(C)C(=O)c1ccc2c(n1)CCC(C(O)CC(=O)OC(C)(C)C)C2]

Charged Intermediate 5 (250 mg, 721 μmol, 1.0 eq) and THF (3 mL) into 50 ml flask at 15° C.

Purged three times with nitrogen.

Charged NaBH₄ (13.6 mg, 360 μmol, 0.5 eq) into flask at 0° C.

Stirred the mixture at 0° C. for 1 h.

LCMS showed that Intermediate 5 was consumed, and 35.8% of Intermediate 6 (RT=0.312 min) was detected.

Charged 2.00 mL brine into flask at 0° C.

Stirred the mixture at 0° C. for 1 h.

Poured the mixture into the water (5.00 mL), extracted with EtOAc (5.00 mL*3).

Washed the combined organic layer with brine (5.00 mL*3), dried with Na₂SO₄, filtered and concentrated. The product was purified by column chromatography by prep-TLC (SiO₂, Petroleum ether/Ethyl acetate=0:1; R_{f}=0.20) to obtain Intermediate 6 (50.0 mg, 116 μmol, 16.1% yield, 81.1% purity) as a white oil, confirmed by LCMS.

LCMS: RT=0.312 min, m/z=349.2 (M+H)⁺.

LCMS: RT=0.320 min, m/z=349.2 (M+H)⁺.

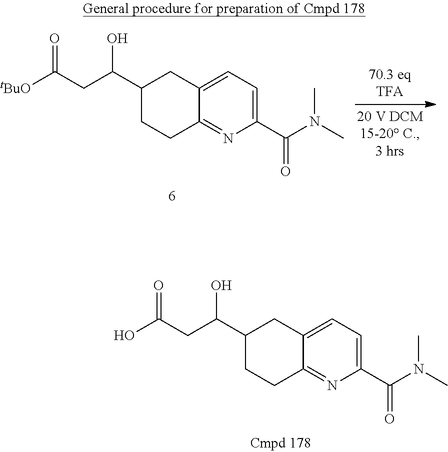 [SMILES: CN(C)C(=O)c1ccc2c(n1)CCC(C(O)CC(=O)OC(C)(C)C)C2 ; CN(C)C(=O)c1ccc2c(n1)CCC(C(O)CC(=O)O)C2]

Charged Intermediate 6 (50 mg, 143 μmol, 1.00 eq) and DCM (1.00 mL) into 50.0 ml flask at 15° C.

The reaction flask was replaced three times with nitrogen.

Charged TFA (1.15 g, 10.1 mmol, 749 μL, 70.3 eq) into flask at 20° C.

Stirred for 3 h at 20° C.

LCMS showed Intermediate 6 was consumed and 81.8% desired mass (RT=0.186 min) was detected.

The mixture was concentrated in vacuum.

The crude product was purified by prep. HPLC (Welch Xtimate C18 150*25 mm*5 um; mobile phase: [water (TFA)-ACN]; gradient: 0%-25% B over 10 min) to obtain Cmpd 178 (40 mg, 135 μmol, 94.6% yield, 99.3% purity) as a white oil, confirmed by LCMS, HPLC, and ¹H NMR.

LCMS: RT=0.186 min, m/z (M+H⁺)=293.1.

LCMS: RT=0.281 min, m/z (M+H⁺)=293.2.

HPLC: RT=1.091 min, 99.3% purity under 220 nm.

¹H NMR (400 MHz, DMSO): δ 7.68-7.63 (m, 1H), 7.33-7.31 (m 1H), 3.87-3.80 (m, 2H), 2.98 (s, 3H), 2.93 (s, 3H), 2.81-2.78 (m, 2H), 2.64-2.54 (m, 2H), 2.54-2.50 (m, 1H), 2.27-2.07 (m, 1H), 1.76-1.75 (m, 1H), 1.73-1.47 (m, 1H).

### Example 30—Preparation of Cmpd 180

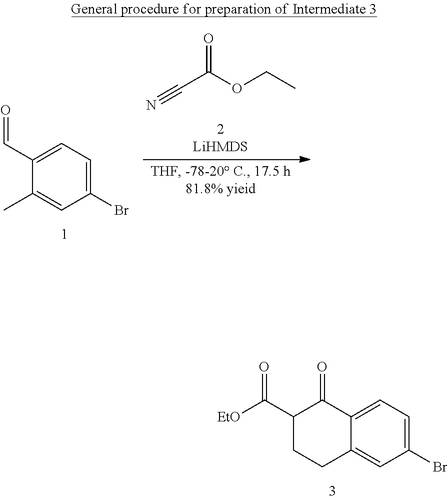

Charged Intermediate 1 (5.00 g, 22.2 mmol, 1.00 eq) and THF (75.0 mL) into R1 (500 mL flask) at 20° C. Cool down to −78° C. and degassed the mixture with N₂ for 3 times. Loaded LiHMDS (1 M, 66.6 mL, 3.00 eq) into R1 at −78° C. under N₂ and stirred the mixture at −78° C. under N₂ for 1.5 h. Loaded Intermediate 2 (6.60 g, 66.6 mmol, 6.53 mL, 3.00 eq) in THF (7.50 mL) to R1 at −78° C. under N₂ dropwise. Stirred the mixture at 20° C. under N₂ for 16 h.

LCMS (EC14042-18-p1a1) showed that Intermediate 1 was remained (RT=0.483 min) and Intermediate 3 was detected (RT=0.516 min).

Dropwise the mixture into NH₄Cl (300 mL), extracted with ethyl acetate (200 mL*3), washed the combined organic layer with brine (300 mL), dried with Na₂SO₄, filtered and concentrated. The combined crude product was purified by silica gel column chromatography (Petroleum ether:Ethyl acetate=1:0˜10:1 R_{f}=0.58) to obtain Intermediate 3 (5.60 g, 18.1 mmol, 81.8% yield, 96.5% purity) as yellow oil, confirmed by LCMS and ¹H NMR.

LCMS: RT=0.516 min, m/z=297.0 (M+H)⁺.

LCMS: RT=0.562 min, m/z=296.8 (M+H)⁺.

¹H NMR (400 MHz, DMSO): δ 7.48 (d, J=8.40 Hz, 1H), 7.35 (d, J=1.60 Hz, 1H), 7.13 (s, 1H), 3.90-3.82 (m, 2H), 2.08-2.02 (m, 2H), 1.87-1.80 (m, 1H), 1.71-1.62 (m, 1H), 1.59-1.47 (m, 1H), 0.91 (t, J=7.20 Hz, 14.0 Hz, 3H).

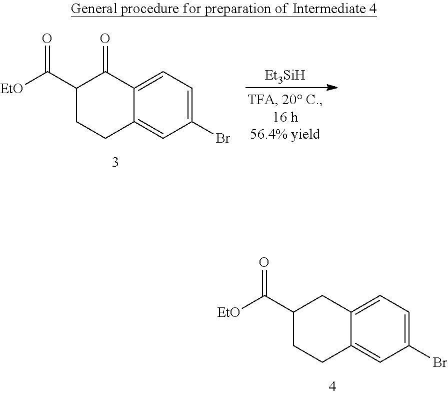 [SMILES: "Et3SiH" ; CCOC(=O)C1CCc2cc(Br)ccc2C1=O ; CCOC(=O)C1CCc2cc(Br)ccc2C1]

Charged Intermediate 3 (4.60 g, 15.4 mmol, 1.00 eq), TFA (35.0 mL), and Et₃SiH (10.0 g, 86.3 mmol, 13.8 mL, 5.58 eq) into R1 (100 mL flask) at 20° C. and stir the mixture at 20° C. for 16 h under N₂.

TLC analysis (plate 1, Petroleum ether:Ethyl acetate=10:1) showed that Intermediate 3 (R_{f}=0.52) was consumed and Intermediate 4 (R_{f}=0.45) was formed. The reaction mixture was concentrated under reduced pressure to give the residue. The crude product was purified by column chromatography (SiO₂, Petroleum ether:Ethyl acetate=1:0 to 5:1, R_{f}=0.65) to obtain Intermediate 4 (2.50 g, 8.74 mmol, 56.4% yield, 99.0% purity) as colorless oil, confirmed by LCMS and ¹H NMR.

LCMS: RT=0.482 min, m/z=283.0 (M+H)⁺.

¹H NMR (400 MHz, DMSO): δ 7.27-7.24 (m, 2H), 7.06 (d, J=8.00 Hz, 1H), 4.13-4.13 (m, 2H), 2.93-2.88 (m, 1H), 2.82-2.70 (m, 4H), 2.08-2.04 (m, 1H), 1.73-1.68 (m, 1H), 1.20 (t, J=6.80 Hz, 14.0 Hz, 3H).

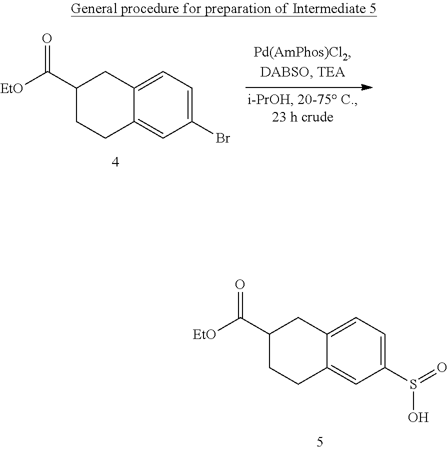 [SMILES: CCOC(=O)C1CCc2cc(Br)ccc2C1 ; CCOC(=O)C1CCc2cc(S(=O)O)ccc2C1]

Loaded Intermediate 4 (1.10 g, 3.85 mmol, 1.00 eq), i-PrOH (10.0 mL), DABSO (554 mg, 2.31 mmol, 0.60 eq), TEA (1.17 g, 11.5 mmol, 1.61 mL, 3.00 eq), and Pd(AmPhos)Cl₂ (272 mg, 384 μmol, 272 μL, 0.10 eq) into R1 (100 mL flask) at 20° C. and stirred the mixture at 75° C. for 23 h under N₂. LCMS analysis showed that Intermediate 4 was consumed and Intermediate 5 was detected (RT=0.417 min). The resulting product was concentrated in vacuum to obtain Intermediate 5 (1.03 g, crude) as yellow solid, confirmed by LCMS.

LCMS: RT=0.417 min, m/z=266.8 (M+H)⁺.

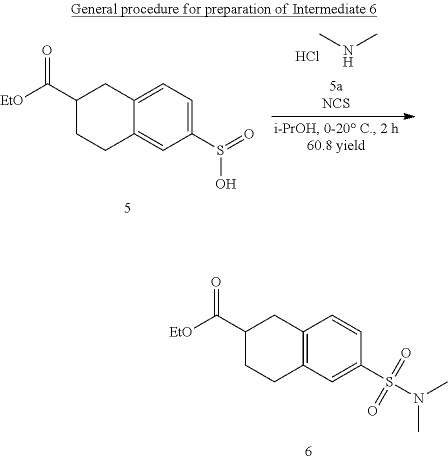

Loaded Intermediate 5 (1.03 g, 3.84 mmol, 1.00 eq), i-PrOH (11.0 mL), and Intermediate 5a (626 mg, 7.68 mmol, 2.00 eq) into R1 (10.0 mL flask) at 20° C. Cooled down the mixture to 0° C. and added NCS (1.03 g, 7.68 mmol, 2.00 eq) into R1. Stirred the mixture at 0˜20° C. for 2 h under N₂.

LCMS showed that Intermediate 5 was consumed and Intermediate 6 was detected (RT=0.436 min).

Poured the mixture to H₂O (60.0 mL), extracted with ethyl acetate (40.0 mL*3), washed the combined organic layer with brine (50.0 mL), dried with Na₂SO₄, filtered and concentrated.

The crude product was purified by column chromatography (SiO2, Petroleum ether:Ethyl acetate=1:0 to 3:1, R_{f}=0.30) to obtain Intermediate 6 (879 mg, 2.33 mmol, 60.8% yield, 82.7% purity) as white solid, confirmed by ¹H NMR, LCMS, and HPLC.

LCMS: RT=0.436 min, m/z=312.1 (M+H)⁺.

LCMS: RT=0.484 min, m/z=312.0 (M+H)⁺.

HPLC: RT=1.946 mins, 82.7% purity under 220 nm.

¹H NMR (400 MHz, DMSO): δ 7.45 (d, J=6.00 Hz, 1H), 7.39 (t, J=3.20 Hz, 6.4 1H), 4.16-4.08 (m, 2H), 3.09-2.81 (m, 5H), 2.59 (s, 6H), 2.14-2.10 (m, 1H), 1.81-1.73 (m, 1H), 1.20 (t, J=7.20 Hz, 14.4 Hz, 3H).

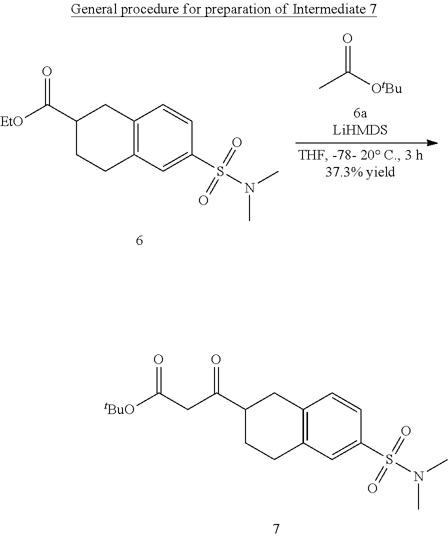

Loaded Intermediate 6a (1.36 g, 11.6 mmol, 1.57 mL, 5.00 eq) and THF (10.0 mL) into R1 (50.0 mL flask) at 20° C. Cooled down to −78° C. and degassed the mixture with N₂ for 3 times. Added LiHMDS (1 M, 11.6 mL, 5.00 eq) to R1 at −78° C. under N₂ and stirred the mixture at −78° C. under N₂ for 1 h.

A solution of Intermediate 6 (879 mg, 2.33 mmol, 1.00 eq) in THF (3.00 mL) was added dropwise into R1 at −78° C. The mixture was stirred at −78° C. under N₂ for 2 h.

LCMS (EC14042-63-p1a1) showed that Intermediate 6 was consumed and Intermediate 7 was detected (RT=0.458 min).

The mixture was poured into H₂O (30.0 mL), extracted with ethyl acetate (20.0 mL*3), the combined organic layer was washed with brine (40.0 mL), dried with Na₂SO₄, filtered and concentrated. The crude product was purified by column chromatography (SiO₂, Petroleum ether:Ethyl acetate=1:0 to 1:1, R_{f}=0.74) to obtain compound 7 (350 mg, 872 μmol, 37.3% yield, 95.1% purity) as white solid, confirmed by LCMS.

LCMS: RT=0.458 min, m/z=381.95 (M+H)⁺.

LCMS: RT=0.462 min, m/z=382.15 (M+H)⁺.

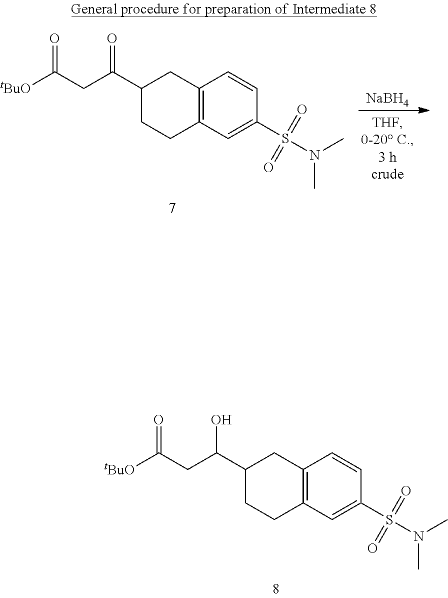 [SMILES: CN(C)S(=O)(=O)c1ccc2c(c1)CCC(C(=O)CC(=O)OC(C)(C)C)C2 ; "NaBH4" ; CN(C)S(=O)(=O)c1ccc2c(c1)CCC(C(O)CC(=O)OC(C)(C)C)C2]

Loaded Intermediate 7 (350 mg, 872 μmol, 1.00 eq) and THF (4.00 mL) into R1 (10.0 mL flask) at 20° C., and NaBH₄ (70.0 mg, 1.85 mmol, 2.12 eq) into R1 at 0-5° C. The mixture was stirred at 0-5° C. under N₂ for 3 h and a sample was collected for LCMS analysis, which showed that Intermediate 7 remained (RT=0.502) but Intermediate 8 was detected (RT=0.482 min).

Brine (5.00 mL) was added, and the resultant suspension was stirred at room temperature for 10 mins. The reaction mixture was diluted by the addition of ethyl acetate (10.0 mL) and distilled water (10.0 mL), and the layers were separated. The aqueous layer was extracted with ethyl acetate (10.0 mL*2). The combined organic layers were washed with brine (10.0 mL) and dried over Na₂SO₄ to obtain Intermediate 8 (390 mg, crude) as white solid, confirmed by LCMS.

LCMS: RT=0.482 min, m/z=384.1 (M+H)⁺.

LCMS: RT=0.450 min, m/z=384.1 (M+H)⁺.

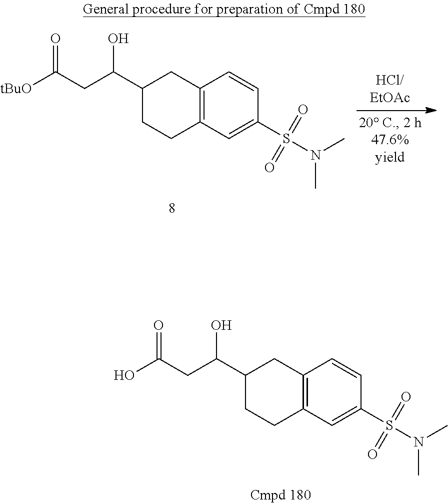 [SMILES: CN(C)S(=O)(=O)c1ccc2c(c1)CCC(C(O)CC(=O)OC(C)(C)C)C2 ; CN(C)S(=O)(=O)c1ccc2c(c1)CCC(C(O)CC(=O)O)C2]

Loaded Intermediate 8 (390 mg, 981 μmol, 1.00 eq) and HCl/EtOAc (2 M, 8.67 mL, 17.6 eq) into R1 (50.0 mL flask) at 20° C. Stir the mixture at 20° C. for 2 h.

LCMS showed that Intermediate 8 was consumed and Cmpd 180 (RT=0.336 min).

The reaction mixture was concentrated under reduced pressure to give the residue.

The crude product was purified by prep-HPLC (column: Welch Ultimate C18 150*25 mm*5 um; mobile phase: [water (FA)-ACN]; gradient: 16%-46% B over 10 min) to obtain Cmpd 180 (153 mg, 467 μmol, 47.6% yield, 100% purity) as white solid, confirmed by ¹H NMR, LCMS, and HPLC.

LCMS RT=0.336 min, m/z=328.0 (M+H)⁺.

LCMS: RT=0.332 min, m/z=328.1 (M+H)⁺.

HPLC: RT=1.329 mins, 100% purity under 220 nm.

¹H NMR (400 MHz, DMSO): δ 7.42 (d, J=6.40 Hz, 1H), 7.34 (t, J=8.4 Hz, 16.4 Hz, 1H), 3.87-3.72 (m, 1H), 2.95-2.62 (m, 4H), 2.58 (s, 6H), 2.48-2.46 (m, 1H), 2.33-2.25 (m, 1H), 2.03-1.70 (m, 2H), 1.53-1.32 (m, 1H).

### Example 31—Preparation of Cmpd 181

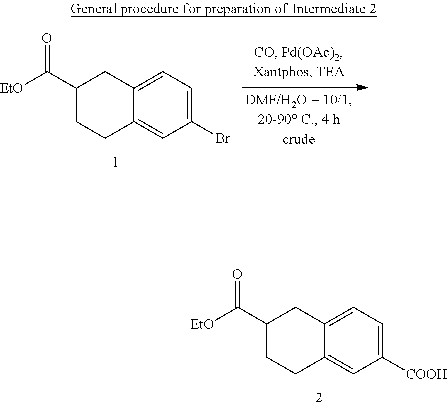 [SMILES: CCOC(=O)C1CCc2cc(Br)ccc2C1 ; CCOC(=O)C1CCc2cc(C(=O)O)ccc2C1]

Loaded Intermediate 1 (3.00 g, 10.5 mmol, 1.00 eq), DMF (30.0 mL) H₂O (3.00 mL), Pd(OAc)₂ (1.19 g, 5.30 mmol, 0.50 eq), TEA (3.22 g, 31.7 mmol, 4.42 mL, 3.00 eq), and Xantphos (3.07 g, 5.30 mmol, 0.50 eq) into a 100 mL stand-up flask (R1) at 20° C., degassed the mixture with CO (15 psi) for three times, and stirred at 90° C. for 4 h.

LCMS (EC13849-40-p1a1) showed Compound 1 was consumed and target mass was found (RT=0.532 min).

Filtered the mixture to get the liquid phase, added saturated Na₂CO₃ (80.0 mL) into the liquid to adjust the solution pH to 9˜10, extracted with ethyl acetate (50.0 mL*3), separated the mixed liquid phase to get the water phase, added 1M HCl (50.0 mL) into the water phase to adjust the solution pH to 2˜3, extracted with ethyl acetate (50.0 mL*3), washed the organic phase with brine (50.0 mL*2), dried with Na₂SO₄, filtered, and concentrated in vacuum to obtain Intermediate 2 (1.40 g, crude) as a yellow gum, confirmed by LCMS.

LCMS: RT=0.532 min, m/z=246.9 (M+H)⁺.

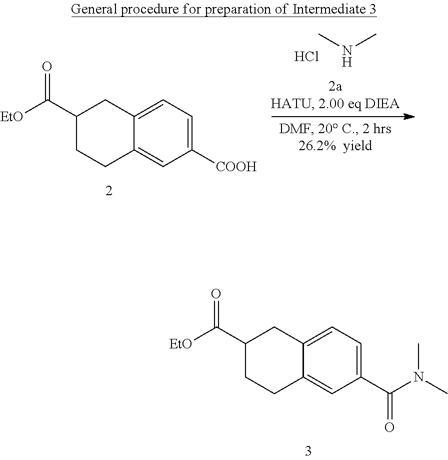

Loaded Intermediate 2 (1.40 g, 5.64 mmol, 1.00 eq), DMF (15.0 mL), Intermediate 2a (1.15 g, 14.1 mmol, 2.50 eq), DIEA (1.46 g, 11.2 mmol, 1.96 mL, 2.00 eq), and HATU (2.57 g, 6.77 mmol, 1.20 eq) into a 100 mL flask (R1) and stirred the mixture at 20° C. for 2 h under N₂.

LCMS (EC13849-43-p1a2) showed Compound 2 was consumed and target mass was found (RT=0.403 min).

Added water (30.0 mL) into R1 at 20° C., extracted with ethyl acetate (30.0 mL*3), washed with brine (30.0 mL*2), dried with Na₂SO₄, filtered and concentrated in vacuum.

The crude product was purified by prep-TLC (Dichloromethane:Methanol=10:1, R_{f}=0.86) and subsequently by prep-HPLC (column: Phenomenex luna C18 250*50 mm*15 um; mobile phase: [water (FA)-ACN]; gradient: 28%-58% B over 11 mins) to obtain Intermediate 3 (430 mg, 1.48 mmol, 26.2% yield, 94.9% purity) as a colorless oil, confirmed by LCMS, HPLC and ¹H NMR.

LCMS: RT=0.403 min, m/z=276.1 (M+H)⁺

LCMS: RT=0.398 min. m/z=276.1 (M+H)⁺

HPLC: RT=1.824 mins, 94.9% purity under 220 nm.

¹H NMR (400 MHz, DMSO): δ 7.17-7.10 (m, 3H), 4.14-4.07 (m, 2H), 2.96-2.88 (m, 8H), 2.82-2.78 (m, 3H), 2.12-2.08 (m, 1H), 1.78-1.75 (m, 1H), 1.20 (t, J=7.2 Hz, 14.0 Hz, 3H).

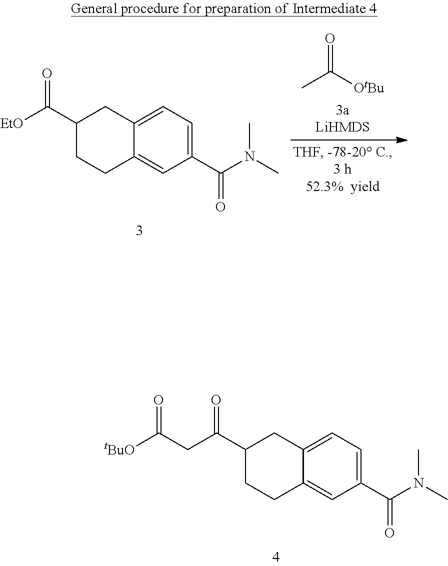

Loaded Intermediate 3a (1.35 g, 11.6 mmol, 1.56 mL, 5.00 eq) and THF (7.00 mL) into R1 (100 mL flask) at 20° C. Cooled down to −78° C. and degassed the mixture with N₂ for 3 times. Add LiHMDS (1 M, 11.6 mL, 5.00 eq) into R1 at −78° C. under N₂ and stirred the mixture at −78° C. under N₂ for 1 h. A solution of Intermediate 3 (640 mg, 2.32 mmol, 1.00 eq) in THF (3.00 mL) was added dropwise into R1 at −78° C. and the mixture was stirred at −78° C. under N₂ for 2 h.

LCMS (EC14042-64-p1a1) showed that Intermediate 3 was consumed and Intermediate 4 was detected (RT=0.428 min).

Poured the mixture to H₂O (20.0 mL), extracted with ethyl acetate (15.0 mL*3), washed the combined organic layer with brine (20.0 mL), dried with Na₂SO₄, filtered and concentrated.

The crude product was purified by column chromatography (SiO₂, Petroleum ether:Ethyl acetate=1:0 to 1:1, R_{f}=0.73) to obtain Intermediate 4 (500 mg, 1.22 mmol, 52.3% yield, 84.0% purity) as colorless oil, confirmed by LCMS.

LCMS: RT=0.428 min, m/z=346.0 (M+H)⁺.

LCMS: RT=0.436 min. m/z=346.1 (M+H)⁺.

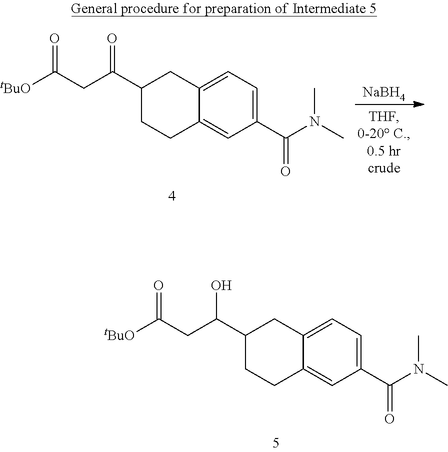 [SMILES: CN(C)C(=O)c1ccc2c(c1)CCC(C(=O)CC(=O)OC(C)(C)C)C2 ; "NaBH4" ; CN(C)C(=O)c1ccc2c(c1)CCC(C(O)CC(=O)OC(C)(C)C)C2]

Loaded Intermediate 4 (400 mg, 972 μmol, 1.00 eq) and THF (4.00 mL) into R1 (10.0 mL flask) at 20° C. Added NaBH₄ (30.0 mg, 793 μmol, 0.82 eq) to R1 at 0-5° C. and stirred the mixture at 0-5° C. under N₂ for 1 h.

LCMS analysis showed that Intermediate 4 was consumed and Intermediate 5 was detected (RT=0.438 min).

Brine (8.00 mL) was added and the resulting suspension was stirred at room temperature for 10 mins. The reaction mixture was diluted by the addition of ethyl acetate (5.00 mL) and distilled water (10.0 mL), and the layers were separated. The aqueous layer was extracted with ethyl acetate (10.0 mL*2). The combined organic layers were washed with brine (10.0 mL) and dried over Na₂SO₄ to obtain Intermediate 5 (450 mg, crude) as yellow oil, confirmed by LCMS.

LCMS: RT=0.438 min, m/z=348.3 (M+H)⁺.

LCMS: RT=0.423 min, m/z=348.2 (M+H)⁺.

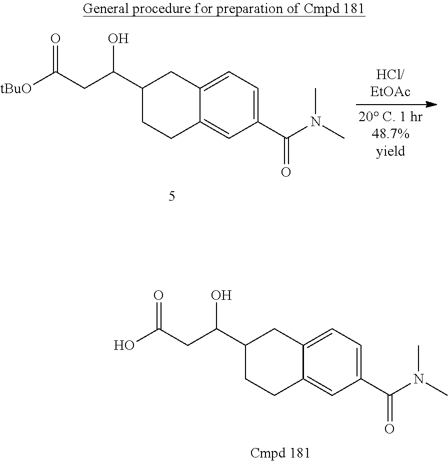 [SMILES: CN(C)C(=O)c1ccc2c(c1)CCC(C(O)CC(=O)OC(C)(C)C)C2 ; CN(C)C(=O)c1ccc2c(c1)CCC(C(O)CC(=O)O)C2]

Loaded Intermediate 5 (450 mg, 1.25 mmol, 1.00 eq) and HCl/EtOAc (4 M, 10.0 mL, 31.9 eq) into R1 (100 mL flask) and stirred the mixture at 20° C. under N₂ for 1 h.

LCMS show Intermediate 5 was remained (RT=0.439 min) and desired mass was detected (RT=0.308 min).

The reaction mixture was concentrated under reduced pressure to give the residue.

The crude product was purified by Prep-HPLC (column: Phenomenex luna C18 150*25 mm*10 um; mobile phase: [water (FA)-ACN]; gradient: 6%-36% B over 10 min) to obtain Cmpd 181 (180 mg, 610 μmol, 48.7% yield, 98.4% purity) as white solid, confirmed by ¹H NMR, LCMS, and HPLC.

LCMS: RT=0.308 min, m/z=292.2 (M+H)⁺.

LCMS: RT=0.309 min, m/z=292.2 (M+H)⁺.

HPLC: RT=1.115 mins, 98.4% purity under 220 nm.

¹H NMR (400 MHz, DMSO): δ 7.13-7.09 (m, 3H), 3.85-3.76 (m, 1H), 2.92 (s, 6H), 2.83-2.59 (m, 5H), 2.33-2.25 (m, 1H), 2.02-1.69 (m, 2H), 1.50-1.30 (m, 1H).

### Example 32—Preparation of Cmpd 184

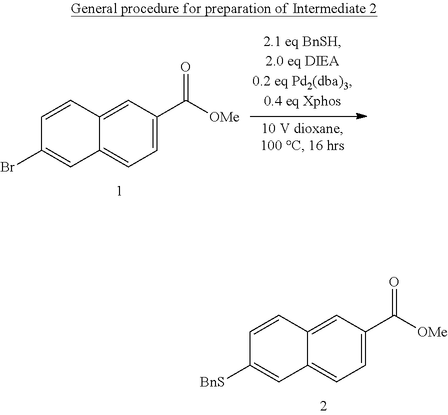

Loaded Intermediate 1 (10.0 g, 37.7 mmol, 1.0 eq) in dioxane (100 mL) into 500 mL flask (R1) at 25° C. Added DIEA (9.75 g, 75.4 mmol, 13.1 mL, 2.0 eq), Pd₂(dba)₃ (6.91 g, 7.54 mmol, 0.2 eq), Xphos (8.73 g, 15.0 mmol, 0.4 eq), and BnSH (9.84 g, 79.2 mmol, 9.30 mL, 2.1 eq) to R1, at 25° C., under N₂, and stirred the mixture at 100° C. for 16 h.

TLC analysis (Petroleum ether:Ethyl acetate=5/1) showed that Intermediate 1 was consumed and Intermediate 2 was found (R_{f}=0.55).

The mixture was quenched with water (100 mL), extracted with DCM (3*150 ml), and concentrated under vacuum. The crude product was purified by column chromatography (SiO₂, Petroleum ether:Ethyl acetate=5/1, R_{f}=0.55) to obtain Intermediate 2 (10.0 g, 27.1 mmol, 71.9% yield) was obtained as a yellow a solid, confirmed via ¹H NMR.

¹H NMR (400 MHz, DMSO): δ 8.57 (s, 1H), 8.06-8.03 (m, 1H), 7.96-7.91 (m, 3H), 7.56-7.53 (m, 1H), 7.46-7.44 (m, 2H), 7.33-7.28 (m, 3H), 4.43 (s, 2H), 3.90 (s, 3H).

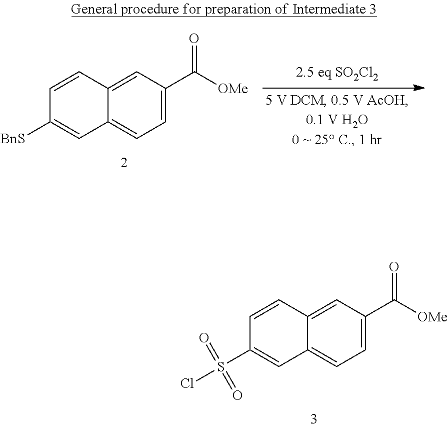

Loaded H₂O (0.90 mL) into 250 mL flask (R1) at 25° C. Added AcOH (4.50 mL), DCM (45.0 mL), and Intermediate 2 (9.00 g, 24.4 mmol, 1.0 eq), at 25° C., under N₂. Then, added SO₂Cl₂ (8.24 g, 61.0 mmol, 6.10 mL, 2.5 eq) into R1, at 0° C., under N₂, and stirred the mixture at 0° C. for 1 h under N₂.

TLC analysis (Petroleum ether:Ethyl acetate=5/1) showed that Intermediate 2 was consumed and Intermediate 3 was found (R_{f}=0.45).

The mixture was quenched with water (40 mL) and extracted with ethyl acetate (3*30 mL). The organic phase was separated and washed with brine (40 mL). The solution was dried over Na₂SO₄, filtered and then evaporated in vacuum to obtain Intermediate 3 (11.6 g, crude) a yellow solid.

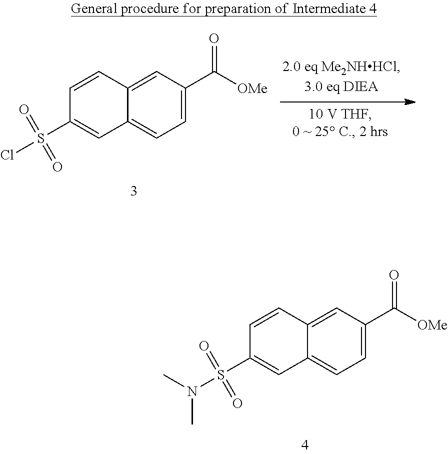 [SMILES: COC(=O)c1ccc2cc(S(=O)(=O)Cl)ccc2c1 ; COC(=O)c1ccc2cc(S(=O)(=O)N(C)C)ccc2c1]

Loaded Intermediate 3 (11.6 g, 40.8 mmol, 1.0 eq) into 250 mL flask (R1) at 25° C. Added THF (120 mL) into R1 at 25° C. under N₂. Added DIEA (15.8 g, 122 mmol, 21.3 mL, 3.0 eq) and Me₂NH·HCl (7.99 g, 98.0 mmol, 2.4 eq) into R1, at 0-5° C., under N₂, and stirred the mixture at 0-5° C. for 2 h under N₂.

LCMS (EC13398-54-P1A1) showed Intermediate 3 was consumed and the Intermediate 4 was found (RT=0.423 min).

The mixture was quenched with water (100 mL) and extracted with ethyl acetate (3*100 mL). The organic phase was separated and washed with brine (3*100 mL). The solution was dried over Na₂SO₄, filtered and then evaporated in vacuum. The crude product was purified by column chromatography (SiO₂, Petroleum ether:Ethyl acetate=5/1, R_{f}=0.20) to obtain Intermediate 4 (3.00 g, 10.2 mmol, 25.0% yield) was obtained as a white solid, confirmed by LCMS.

LCMS: RT=0.423 min, m/z=293.9 (M+H)⁺.

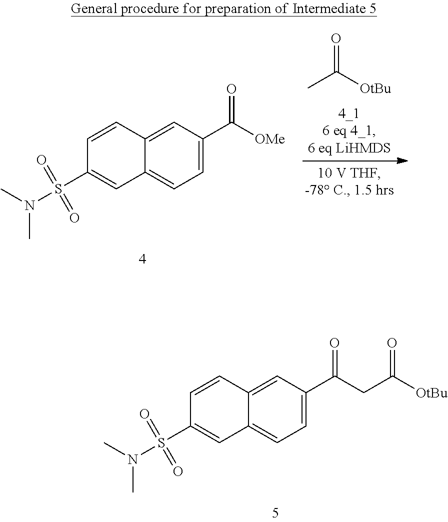

Loaded Intermediate 4_1 (7.60 g, 65.4 mmol, 8.78 mL, 6.0 eq) into 500 mL flask (R1) at 20° C. Added THF (32 mL) into R1 at 20° C. under N₂. Then, added LiHMDS (1.0 M, 65.4 mL, 6.0 eq) into R1 at −78˜−70° C. under N₂ and Stir for 0.5 h. Added Intermediate 4 (3.2 g, 10.9 mmol, 1.0 eq) into R1 at −78˜−70° C. under N₂, and stirred the mixture at −78˜−70° C. for 1 h under N₂.

LCMS showed that Intermediate 4 was consumed and Intermediate 5 was found (RT=0.738 min).

The mixture was quenched with aqueous NH₄Cl (50 mL) and extracted with ethyl acetate (3*30 mL). The organic phase was separated and washed with brine (3*30 mL). The solution was dried over Na₂SO₄, filtered and then evaporated in vacuum.

The crude product was purified by column chromatography (SiO₂, Petroleum ether:Ethyl acetate=3/1, R_{f}=0.50) to obtain Intermediate 5 (2.80 g, 7.42 mmol, 68.0% yield) was obtained as a yellow solid, confirmed by LCMS.

LCMS: RT=0.738 min. m/z=377.9 (M+H)⁺.

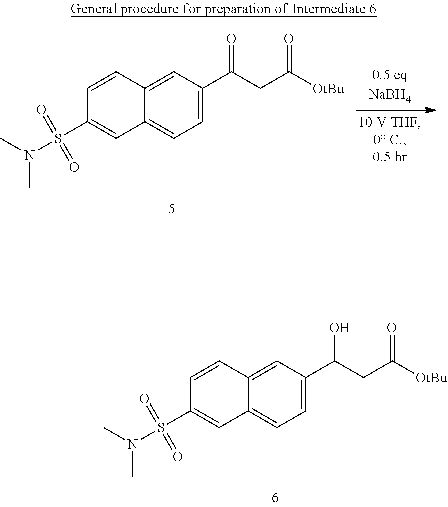 [SMILES: CN(C)S(=O)(=O)c1ccc2cc(C(=O)CC(=O)OC(C)(C)C)ccc2c1 ; CN(C)S(=O)(=O)c1ccc2cc(C(O)CC(=O)OC(C)(C)C)ccc2c1]

Loaded Intermediate 5 (1.00 g, 2.65 mmol, 1.0 eq) into 100 mL flask (R1) at 25° C. Added THF (10 mL) into R1 at 25° C. under N₂. Then, added NaBH₄ (50.1 mg, 1.32 mmol, 0.5 eq) into R1 at 0-5° C. under N₂, and stirred the mixture at 0-5° C. for 0.5 h under N₂.

LCMS (EC13398-101-P1A2) showed Intermediate 5 was consumed, and the Intermediate 6 was found (RT=0.436 min).

Brine (10 mL) was added and the resultant suspension was stirred at room temperature for 10 min. The reaction mixture was diluted by the addition of AcOEt (10 mL) and distilled water (10 mL) and extracted with ethyl acetate (2*8 mL). The organic phase was separated and washed with brine (10 mL). The solution was dried over Na₂SO₄, filtered and then evaporated in vacuum.

The crude product was purified by column chromatography (SiO₂, Petroleum ether:Ethyl acetate=3/1, R_{f}=0.30) to obtain Intermediate 6 (567 mg, 1.45 mmol, 54.71% yield, 97% purity) was obtained as a white solid, confirmed by LCMS.

LCMS: RT=0.436 min, m/z=380.3 (M+H)⁺.

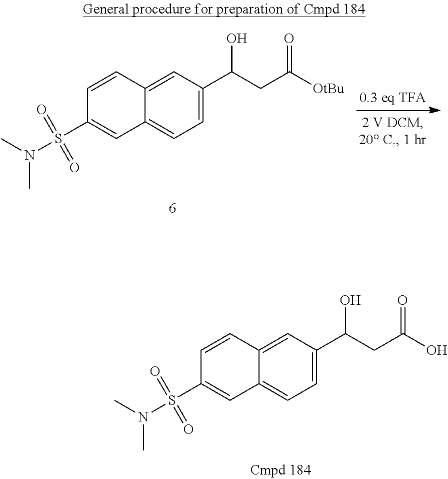 [SMILES: CN(C)S(=O)(=O)c1ccc2cc(C(O)CC(=O)OC(C)(C)C)ccc2c1 ; CN(C)S(=O)(=O)c1ccc2cc(C(O)CC(=O)O)ccc2c1]

Loaded Intermediate 6 (567 mg, 1.49 mmol, 1.0 eq), DCM (1 mL), and TFA (51.1 mg, 448 μmol, 33.30 μL, 0.3 eq) into 10 mL flask (R1) at 20° C. under N₂ and stirred the mixture at 20° C. for 1 h.

LCMS (EC13398-105-P1A2) showed that Intermediate 6 was consumed and Cmpd 184 was found (RT=0.320 min).

The mixture was evaporated in vacuum and the crude product was purified by prep-HPLC (column: Welch Xtimate C18 150*25 mm*5 μm; mobile phase: [water (TFA)-ACN]; gradient: 16%-46% B over 10 min) to obtain Cmpd 184 (375 mg, 1.16 mmol, 77.8% yield, 99.6% purity) as a white solid, confirmed via LCMS, HPLC, and ¹H NMR.

LCMS: RT=0.320 min, m/z=323.9 (M+H)⁺.

LCMS: RT=0.316 min, m/z=324.0 (M+H)⁺.

HPLC: RT=1.345 min, 99.6% purity under 220 nm.

¹H NMR: (400 MHz, DMSO): δ 8.40 (s, 1H), 8.19-8.13 (m, 2H), 8.02 (s, 1H), 7.76-7.68 (m, 2H), 5.17-5.13 (m, 1H), 2.68-2.61 (m, 8H).

### Example 33—Preparation of Cmpd 185

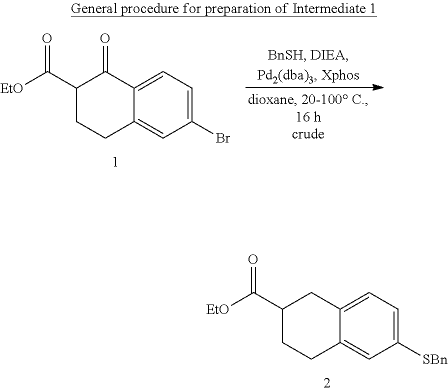

Loaded Intermediate 1 (1.50 g, 5.30 mmol, 1.00 eq), dioxane (15.0 mL), Pd₂(dba)₃ (970 mg, 1.06 mmol, 0.20 eq), XPhos (1.01 g, 2.12 mmol, 0.40 eq), DIEA (1.37 g, 10.5 mmol, 1.85 mL, 2.00 eq), and BnSH (1.58 g, 12.7 mmol, 1.49 mL, 2.40 eq) into a 100 mL stand-up flask (R1) at 20° C. and stirred at 100° C. for 16 h under N₂.

TLC analysis (plate 1, Petroleum ether:Ethyl acetate=8:1) showed that Intermediate 1 (R_{f}=0.75) was consumed and Intermediate 2 (R_{f}=0.60) formed.

The mixture was quenched with water and extracted with DCM (5.00 ml*3), washed the combined organic layer with brine (15.0 mL), dried with Na₂SO₄, filtered and concentrated.

The crude product on notebook page EC14042-3 (2.00 g) was combined to EC14042-4 for further purification. The combined crude product was purified by silica gel column chromatography (Petroleum ether:Ethyl acetate=1:0˜8:1 R_{f}=0.60) to obtain Compound 2 (3.50 g, crude) as a yellow oil, confirmed by ¹H NMR.

¹H NMR (400 MHz, DMSO): δ 7.25-7.18 (m, 5H), 7.10-7.01 (m, 3H), 4.17 (s, 2H), 4.13-4.05 (m, 2H), 2.95-2.87 (m, 2H), 2.69-2.75 (m, 3H), 2.08-2.03 (m, 1H), 1.75-1.65 (m, 1H), 1.19 (t, J=7.20 Hz, 14.4 Hz, 3H).

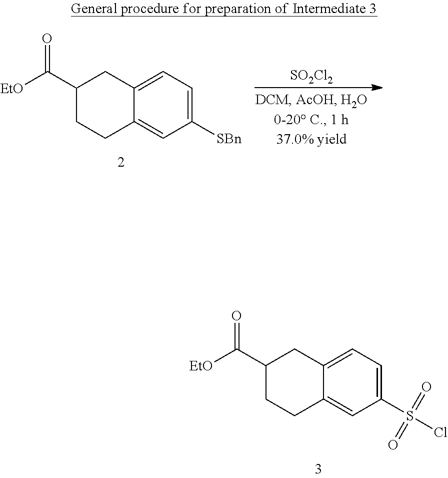

Loaded H₂O (1.20 mL) into R1 (100 mL flask) at 20° C. Added AcOH (5.00 mL), DCM (50.0 mL), and Intermediate 2 (2.50 g, 7.66 mmol, 1.00 eq) into R1 at 20° C. under N₂. Then, added SO₂Cl₂ (2.58 g, 19.1 mmol, 1.91 mL, 2.50 eq) into R1, at 0˜5° C., under N₂, and stirred mixture at 0˜5° C. for 1 h under N₂.

TLC analysis (plate 1, Petroleum ether:Ethyl acetate=8:1) showed that Intermediate 2 (R_{f}=0.53) was consumed and Intermediate 3 (R_{f}=0.40) was formed.

Poured the mixture to H₂O (100 mL), extracted with DCM (70.0 mL*3), washed the combined organic layer with brine (100 mL), dried with Na₂SO₄, filtered and concentrated.

The crude product was purified by column chromatography (SiO2, Petroleum ether:Ethyl acetate=1:0 to 5:1, R_{f}=0.55) to obtain Intermediate 3 (860 mg, 2.84 mmol, 37.0% yield) as a yellow oil, confirmed by ¹H NMR.

¹H NMR (400 MHz, DMSO): δ 7.31 (d, J=6.00 Hz, 2H), 7.04 (d, J=8.40 Hz, 1H), 4.13-4.07 (m, 2H), 2.96-2.70 (m, 5H), 2.10-2.06 (m, 1H), 1.79-1.70 (m, 1H).

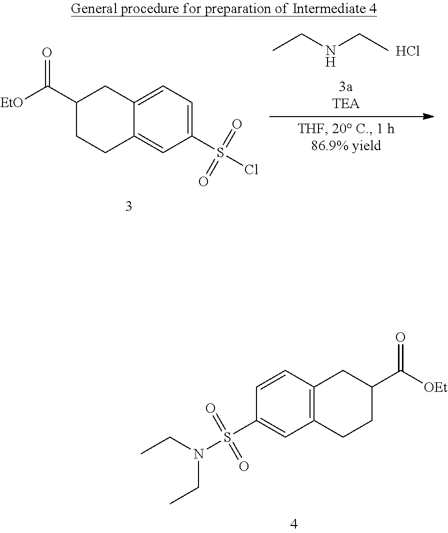

Loaded Intermediate 3 (780 mg, 2.58 mmol, 1.00 eq), Intermediate 3a (423 mg, 3.86 mmol, 1.50 eq), and THF (7.80 mL) into a 10.0 mL flask (R1) at 20° C. Adjusted the mixture pH to 7.0 with TEA (521 mg, 5.15 mmol, 717 μL, 2.00 eq), and stirred at 20° C. under N₂ for 1 h.

TLC analysis (plate 1, Petroleum ether:Ethyl acetate=3:1) showed that Intermediate 3 (R_{f}=0.68) was consumed and Intermediate 4 (R_{f}=0.55) was formed.

Poured the mixture to H₂O (15.0 mL), extracted with ethyl acetate (10.0 mL*3), washed the combined organic layer with brine (15.0 mL), dried with Na₂SO₄, filtered and concentrated.

The crude reaction mixture on notebook page EC14042-29 (100 mg) was combined to EC14042-30 for purification. The crude product was purified by prep-TLC (SiO₂, Petroleum ether:Ethyl acetate=3:1, R_{f}=0.55) to obtain compound 4 (760 mg, 2.24 mmol, 86.9% yield, 100% purity) as a yellow oil, confirmed by LCMS.

LCMS: RT=0.452 min, m/z=340.1 (M+H)⁺.

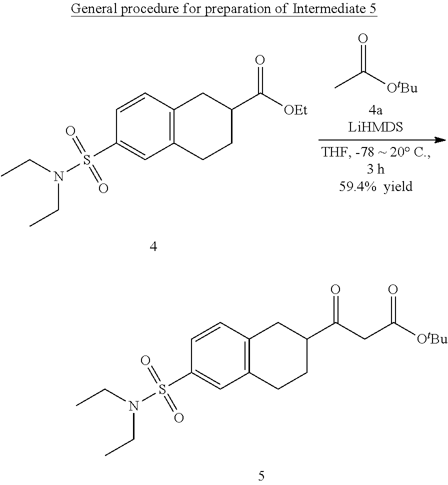

Loaded Intermediate 4a (941 mg, 8.10 mmol, 1.09 mL, 5.00 eq) and THF (6.00 mL) into R1 (100 mL flask) at 20° C. Cooled down the mixture to −78° C. and degassed with N₂ for 3 times. Added LiHMDS (1.00 M, 8.10 mL, 5.00 eq) into R1 at −78° C. under N₂ and stirred the mixture at −78° C. under N₂ for 1 h. Then, a solution of Intermediate 4 (550 mg, 1.62 mmol, 1.00 eq) in THF (2.00 mL) was added dropwise to R1 at −78° C. and stirred at −78° C. under N₂ for 2 h.

LCMS showed that Intermediate 4 was consumed and Intermediate 5 was detected (RT=0.502 min).

Poured the mixture to H₂O (50.0 mL), extracted with ethyl acetate (25.0 mL*3), washed the combined organic layer with brine (40.0 mL), dried with Na₂SO₄, filtered and concentrated.

The crude product was purified by prep-TLC (SiO₂, Petroleum ether:Ethyl acetate=1:1, R_{f}=0.75) to obtain Intermediate 5 (415 mg, 962 μmol, 59.4% yield, 94.9% purity) as a white solid, confirmed by ¹H NMR, LCMS, and HPLC.

LCMS: RT=0.502 min, m/z=410.0 (M+H)⁺.

LCMS: RT=0.557 min, m/z=410.1 (M+H)⁺.

HPLC: RT=2.341 mins, 94.9% purity under 220 nm.

¹H NMR (400 MHz, DMSO): δ 7.49 (d, J=7.60 Hz, 2H), 7.32 (d, J=7.60 Hz, 1H), 3.65 (s, 1H), 3.15-3.10 (m, 2H), 3.00-2.83 (m, 5H), 2.14-2.11 (m, 1H), 1.70-1.60 (m, 1H), 1.41 (s, 9H), 1.04 (t, J=7.20, 14.0 Hz, 6H).

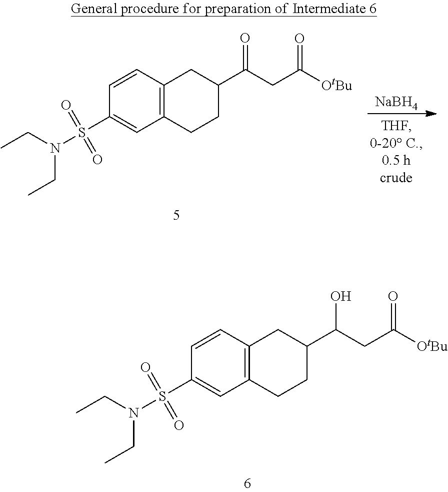 [SMILES: CCN(CC)S(=O)(=O)c1ccc2c(c1)CCC(C(=O)CC(=O)OC(C)(C)C)C2 ; "NaBH4" ; CCN(CC)S(=O)(=O)c1ccc2c(c1)CCC(C(O)CC(=O)OC(C)(C)C)C2]

Loaded Compound 5 (300 mg, 695 μmol, 1.00 eq) and THF (1.00 mL) into R1 (10.0 mL flask) at 20° C. Added NaBH₄ (13.1 mg, 347 μmol, 0.50 eq) at 0˜5° C. into R1 at 20° C. Stirred the mixture at 0˜5° C. under N₂ for 0.5 h.

LCMS (EC14042-61-P1A1) showed that Intermediate 5 remained (RT=0.535 min) but Intermediate 6 was found (RT=0.519 min).

Brine (5.00 mL) was added, and the resultant suspension was stirred at room temperature for 10 mins. The reaction mixture was diluted by the addition of ethyl acetate (10.0 mL) and distilled water (10.0 mL), and the layers were separated. The aqueous layer was extracted with ethyl acetate (10.0*3 mL). The combined organic layers were washed with brine (10.0 mL) and dried over Na₂SO₄ to obtain Intermediate 6 (260 mg, crude) as a yellow oil, confirmed by LCMS.

LCMS: RT=0.519 min, m/z=412.1 (M+H)⁺.

LCMS: RT=0.476 min. m/z=412.4 (M+H)⁺.

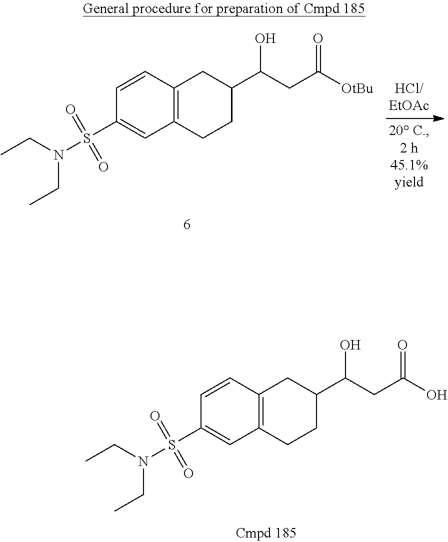 [SMILES: CCN(CC)S(=O)(=O)c1ccc2c(c1)CCC(C(O)CC(=O)OC(C)(C)C)C2 ; CCN(CC)S(=O)(=O)c1ccc2c(c1)CCC(C(O)CC(=O)O)C2]

Loaded Intermediate 6 (260 mg, 617 μmol, 1.00 eq) and HCl/EtOAc (4.00 M, 6.00 mL, 38.8 eq) into R1 (100 mL flask) and stirred the mixture at 20° C. under N₂ for 2 h.

LCMS (EC14042-66-p1a1) showed Intermediate 6 was consumed and Cmpd 185 was detected (RT=0.377 min).

The reaction mixture was concentrated under reduced pressure and the crude product was purified by Prep-HPLC (column: Phenomenex luna C18 150*25 mm*10 um; mobile phase: [water (FA)-ACN]; gradient: 23%-53% B over 10 min) to obtain Cmpd 185 (83.0 mg, 231 μmol, 37.4% yield, 99.0% purity) as white solid, confirmed by ¹H NMR, LCMS, and HPLC.

LCMS: RT=0.377 min, m/z=356.1 (M+H)⁺.

LCMS: RT=0.398 min, m/z=356.1 (M+H)⁺.

HPLC: RT=1.713 mins, 99.0% purity under 220 nm.

¹H NMR (400 MHz, DMSO): δ 7.47-7.45 (m, 2H), 7.28 (t, J=5.20 Hz, 15.6 Hz, 1H), 3.76-3.85 (m, 1H), 3.14-3.09 (m, 4H), 2.92-2.52 (m, 5H), 2.52-2.31 (m, 1H), 2.02-1.69 (m, 2H), 1.50-1.34 (m, 1H), 1.04 (t, J=7.20 Hz, 14.0 Hz, 6H).

### Example 34—In Vitro Assessment of Pannexin-1 Channel Blockade

The assessment of PANX1 blockade was assessed on HEK293 cells stably expressing human PANX1 channels, as previously described by Xu et al (2012). Briefly, wild type cells were seeded in six-well plates and 12-16 hours later, 60-70% confluent cells were transfected with the plasmid encoding hPANX1 using calcium phosphate. After 48 hours post-transfection, geneticin (G418), 0.8 mg/ml in fresh medium, was added to the cells to select for transfected clones and the medium was changed every other day. Once confluent, cells were transferred into flasks and maintained in medium containing G418 (0.8 mg/ml) for a total of 4 weeks from the transfection date.

To validate those cells as a tool to screen potential PANX1 inhibitors a number of known PANX1 inhibitors were assessed by whole cell patch clamp and the voltage/current diagrams compared to current literature. The pipette solution was 151 mM CaCl, 10 mM HEPES, 10 mM EGTA, 3 mM MgCl₂. Cells were maintained in normal extracellular solution (NES: 140 mM NaCl, 5 mM KCl, 2 mM CaCl₂, 1 mM MgCl₂, 10 mM D-glucose, 10 mM HEPES, pH 7.3). Voltage ramps of 1 s duration between −80 mV and +80 mV were applied at 5 s intervals. Opening of Panx1 channel was induced by hypotonic shock, by replacing the isotonic extracellular solution (310 mOsml/l) with hypotonic extracellular solution (198 mOsml/l).

The first run confirmed the functional expression of Panx1 on HEK293 cells (n=6) and also confirmed the known inhibitory effect of CBX (at 50 μM) and Spirolactone (at 3, 10, or 30 μM) on PANX1 (n=3).

TABLE 3
PANX1 blockade level by the compounds of the invention. Compounds
assigned * were those with lowest blockade potential, while
those assigned ***** were the most effective.

| | Inhibition @ + | Inhibition @ − |
|---|---|---|
| CMPD number | 80 mV | 80 mV |
| CMPD001 | ***** | ***** |
| CMPD004 | ***** | ***** |
| CMPD010 | *** | * |
| CMPD011 | ***** | ***** |
| CMPD017 | * | ***** |
| CMPD018 | * | * |
| CMPD019 | ***** | ***** |
| CMPD027 | * | ** |
| CMPD029 | ** | ** |
| CMPD038 | **** | ** |
| CMPD043 | *** | *** |
| CMPD054 | **** | *** |
| CMPD055 | **** | **** |
| CMPD062 | ** | — |
| CMPD064 | ** | * |
| CMPD065 | * | ** |
| CMPD066 | ** | * |
| CMPD068 | ** | * |
| CMPD069 | ** | * |
| CMPD070 | ** | *** |
| CMPD048 | ***** | ***** |
| CMPD056 | ** | — |
| CMPD057 | * | — |
| CMPD058 | *** | — |
| CMPD059 | ** | — |
| CMPD071 | *** | ** |
| CMPD158 | — | * |
| CMPD173 | — | **** |
| CMPD174 | — | * |
| CMPD175 | ** | **** |
| CMPD180 | — | ** |
| CMPD181 | — | ** |
| CMPD184 | — | ** |
| CMPD185 | — | **** |
| CBX | ***** | ***** |
| 10Panx | ***** | ***** |
| PBN | ***** | ***** |
| 15b by Crocetti | **** | —***** |
| et al (2021) | | |

### Example 35—In Vitro Assessment of Anti-Tumor Potential

The anti-tumor potential of the compounds of the invention was investigated over proliferation and tissue invasiveness capacity of MDA-MB-231 human epithelial breast cancer cells.

For the antiproliferative potential, MDA-MB-231 cells were seeded on 384-well plates, in an appropriate culture media, and treated with either one of the compounds of the invention (final concentration at 0.1, 1, 5, 10, 50, 100, 200, 1000 PM) or vehicle (DMSO). The treated cell cultures were maintained in a humid cell incubator at 37° C., under a 95% O₂/5% CO₂ atmosphere, for 7 days. The proliferation rate was automatically recorded by Incucyte S3 system. Representative results are shown in table 4.

TABLE 4
antiproliferative potential of the PANX1 blockers of the
invention over MDA-MB-231 breast cancers cells. Compounds
assigned * were below the 20th IC50 percentile, while those
assigned ***** were above the 80th IC50 percentile.

| Compounds | Potency |
|---|---|
| CMPD001 | ***** |
| CMPD004 | * |
| CMPD010 | ***** |
| CMPD011 | *** |
| CMPD017 | *** |
| CMPD019 | ***** |
| CMPD027 | ***** |
| CMPD029 | ***** |
| CMPD038 | ***** |
| CMPD043 | *** |
| CMPD048 | ***** |
| CMPD054 | **** |
| CMPD055 | * |
| CMPD059 | ***** |
| CMPD170 | **** |
| CMPD171 | ***** |
| CMPD173 | ***** |
| CMPD174 | ***** |
| CMPD175 | **** |
| CMPD180 | |
| CMPD181 | ***** |
| CMPD184 | **** |
| CMPD185 | *** |
| Target12 | **** |

To investigate whether the reduced proliferation was also associated with a concomitant reduction of protein expression of tumor invasiveness markers, MDA-MB-231 cells were seeded on 6-well cell culture plates in an appropriate culture medium and treated with the drug candidates of the invention at a final concentration of 200 μM. The cultures were maintained in a humid cell incubator at 37° C., under a 95% O₂/5% CO₂ atmosphere, for 7 days. At the end of the interval, the cells were collected for assessment of the expression level of proteins associated with breast cancer metastasis.

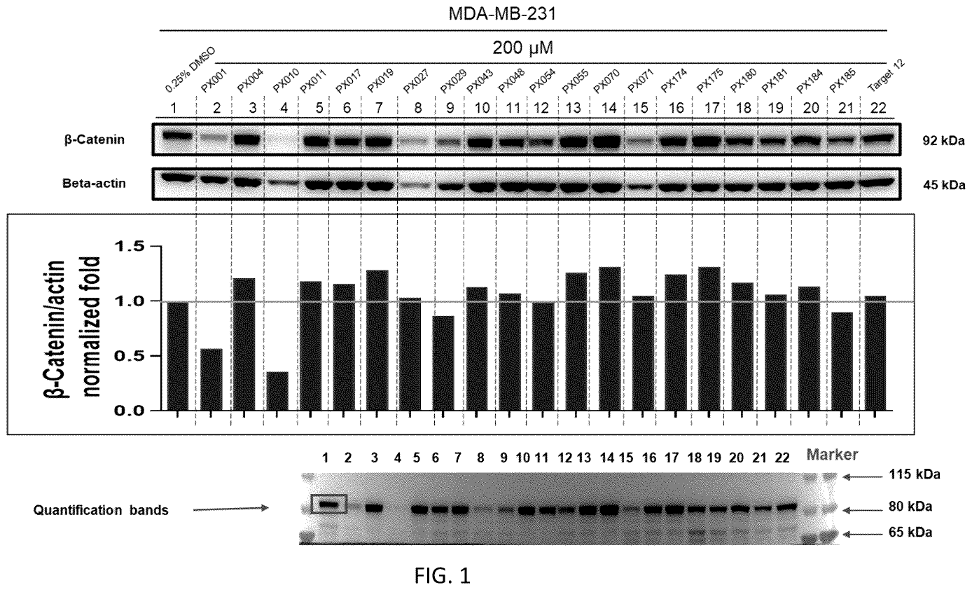

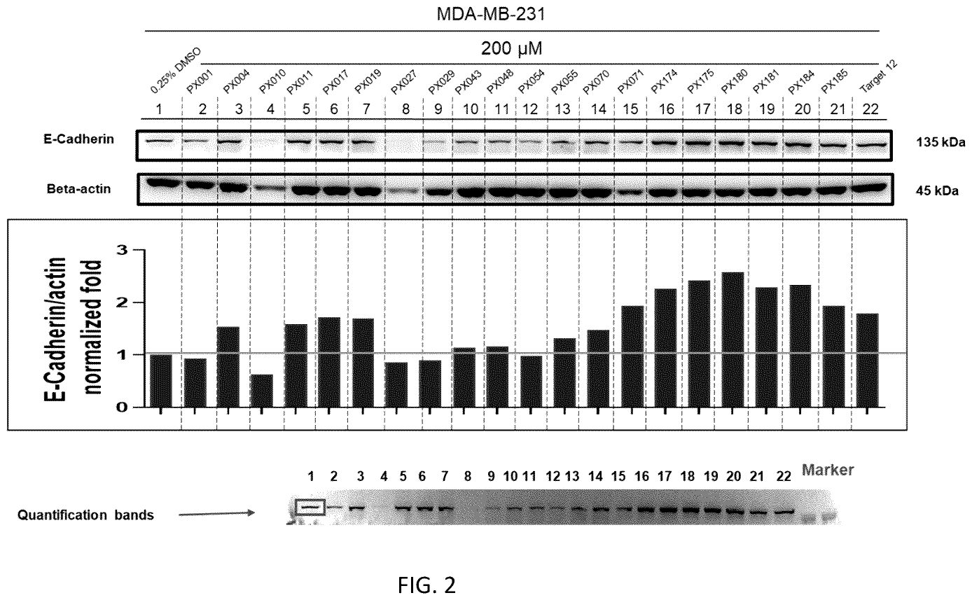

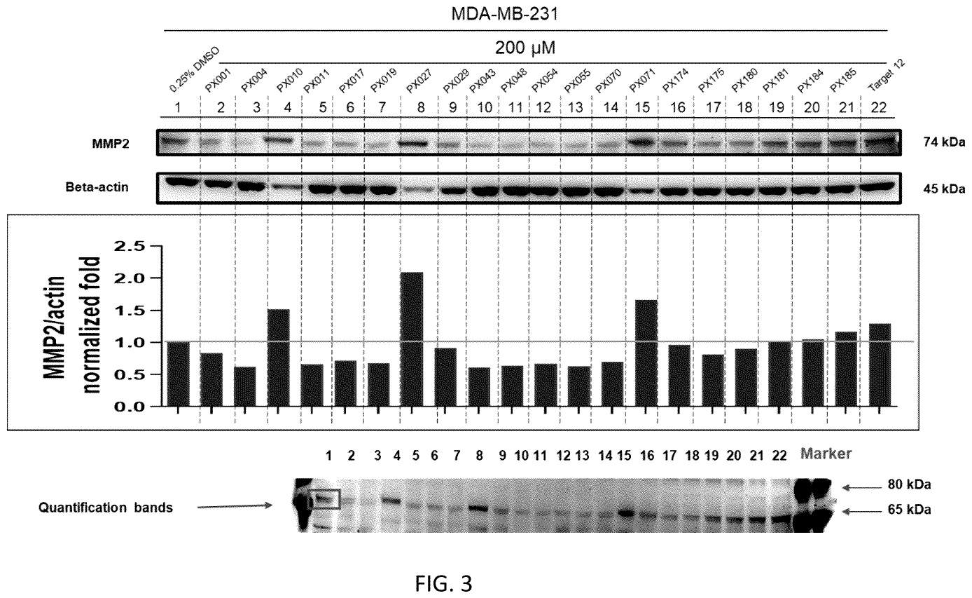

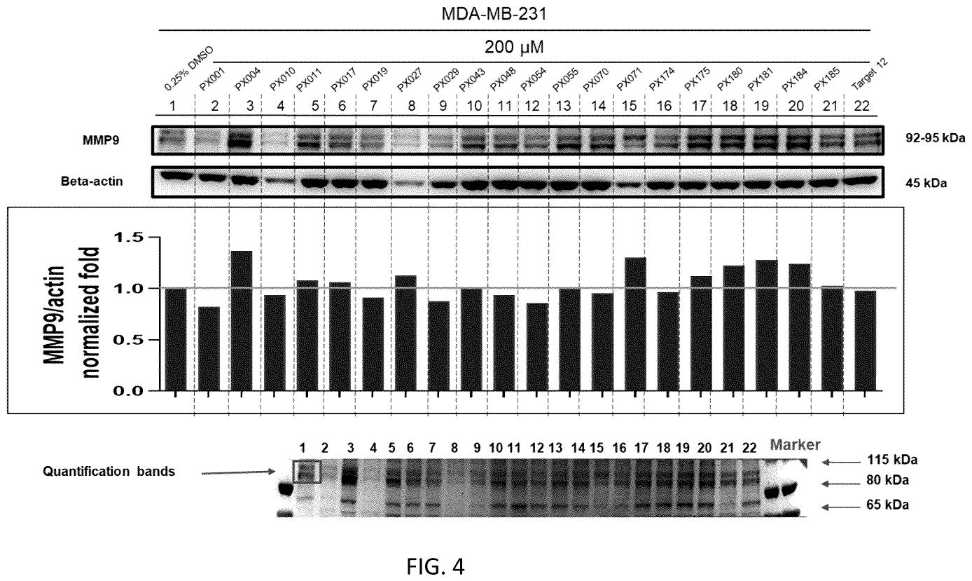

The expression level of E-Cadherin, Matrix Metalloproteinases (MMPs) 2 and 9, N-Cadherin, and β-Catenin was assessed by western blotting, in which the expression of β-Actin was taken as sample normalization standard. Several compounds were able to reduce the expression of β-Catenin (FIG. 1), E-Cadherin (FIG. 2), MMP2 (FIG. 3), and MMP 9 (FIG. 4).

Upon determining that the compounds of the invention could downregulate the protein expression of several metastasis-related proteins, the potential of those compounds to prevent a tumor metastasis was investigated in MDA-MB-231 in vitro.

Cells were allowed to migrate into an artificial matrix either in the presence or absence of the PANX1 blockers of the invention. The cells were seeded on a 24-well Transwell set containing Matrigel and an appropriate culture medium. Cells were treated with each test compound (at 10 μM, 200 μM, or 1000 μM final concentration) or vehicle (DMSO 0, 25%). The cell culture plates were maintained in a humid cell incubator at 37° C., under a 95% O₂/5% CO₂ atmosphere, for 48h. Then, the Matrigel layer was collected, fixed, and stained for cell counting.

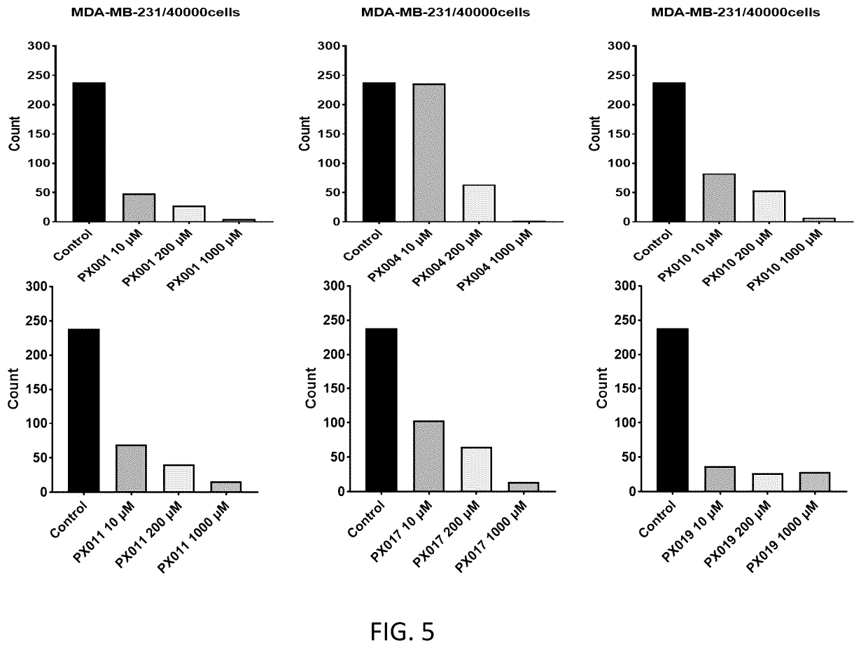

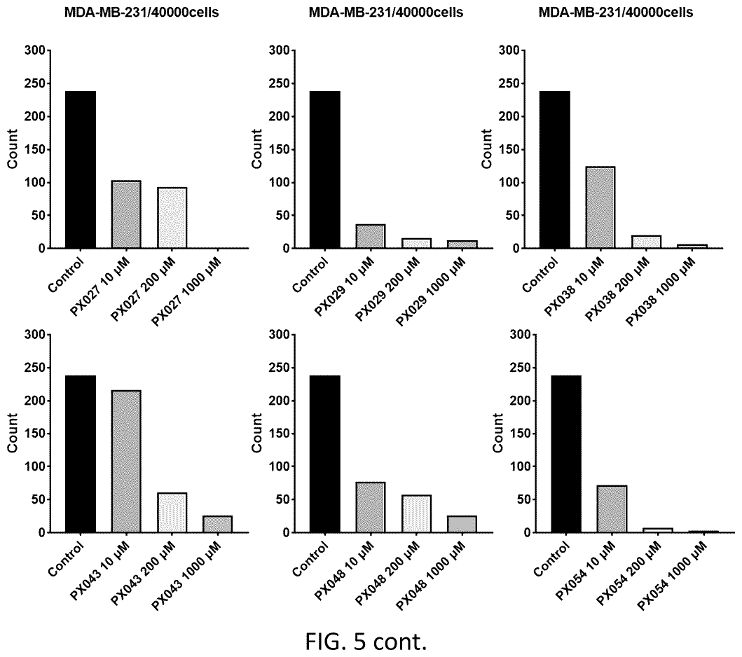

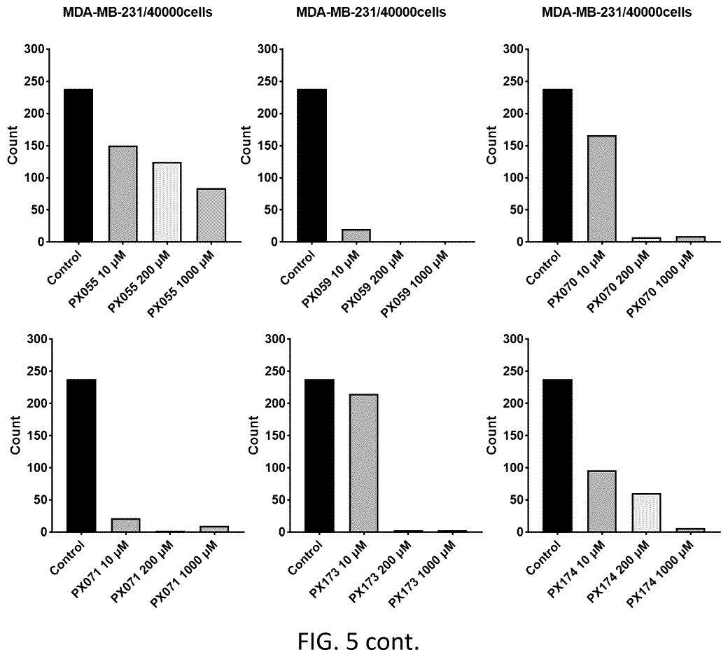

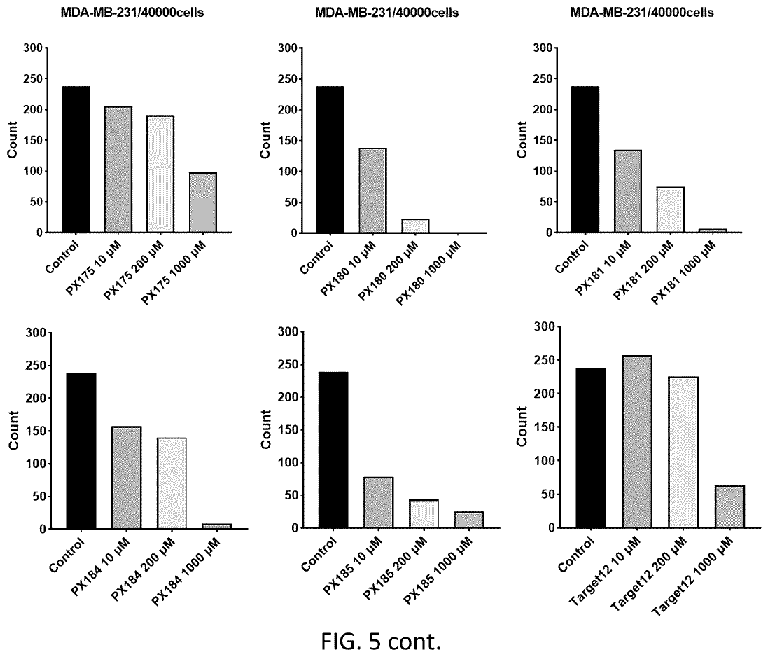

All the tested compounds were able to significantly prevent the invasion of the Matrigel support, thus showing a potential to reduce the tissue invasiveness potential of breast cancer cells (FIG. 5).

### Example 36—In Vivo Assessment of Analgesic Effect in a Rat Model of Neuropathic Pain

Neuropathy was induced by a modification of the model of neuropathic pain in rats, implemented by Decosterd and Wolf, (2000) which produces intense, prolonged, and robust changes in mechanical and thermal sensitivity that is closely related to various clinical features of neuropathic pain. In this model, the sural nerve was sectioned transversely. This model allows the generation of a neuropathic pain, preserving the locomotor activity, since the sural nerve contains almost no motor nerve fibers (Payronnard and Charron, 1982).

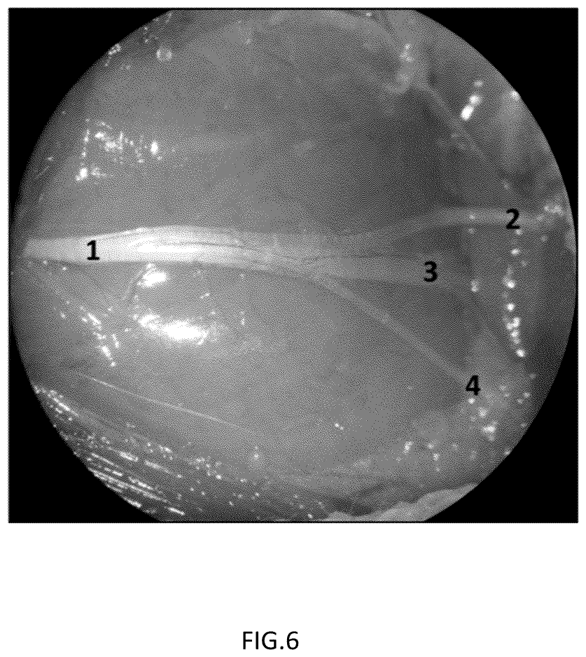

The animals were anesthetized with 400 mg/kg i.p. of Chloral hydrate 7% (w/v). After shaving the right hind leg at the level of the pelvic, origin of the sciatic nerve, a skin incision approximately 10 mm long was made. The subcutaneous tissue and the biceps femoris muscle were dissected to expose the sciatic nerve (FIG. 6, (1)). The course of the nerve was followed until its division into three branches: sural (4), peroneal (2) common and tibial (3). The sural nerve was cut 2 mm from its separation from the sciatic nerve, and the tissues that cover it were sutured in layers. The neuronal injury described above resulted in mechanical hindlimb hyperalgesia that persisted for at least 28 days.

### Schedule and Measurement of Pain

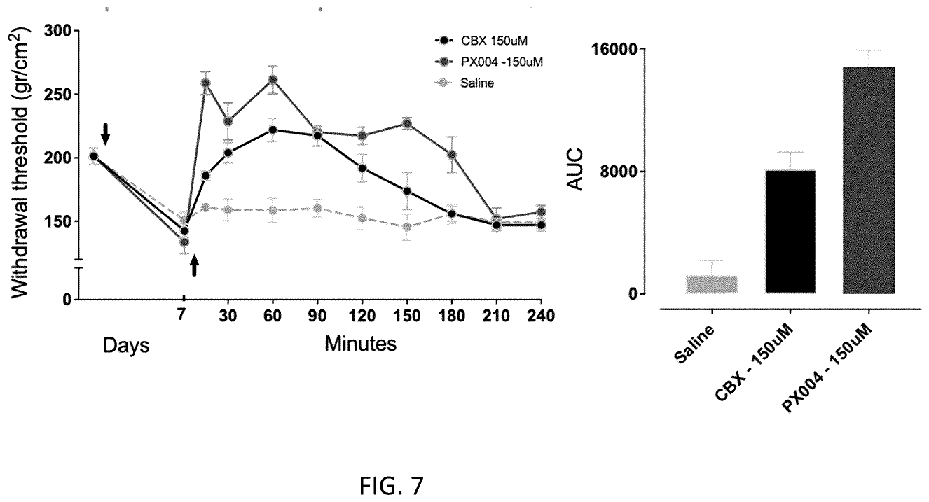

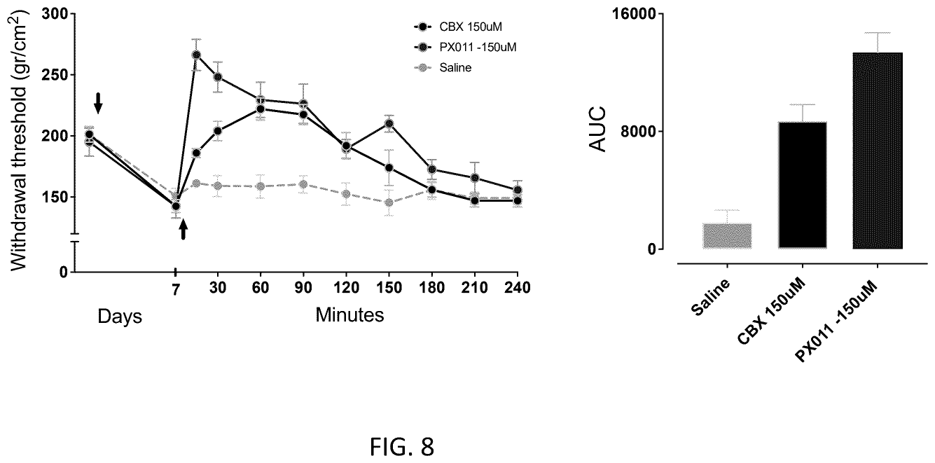

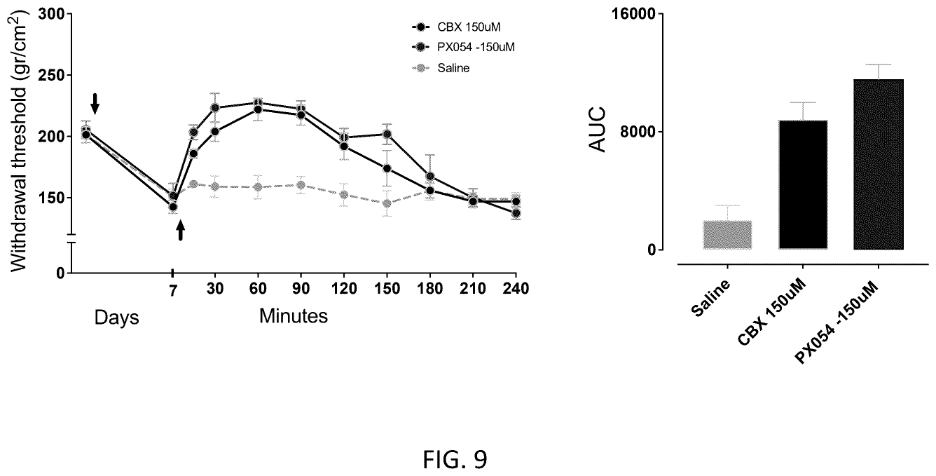

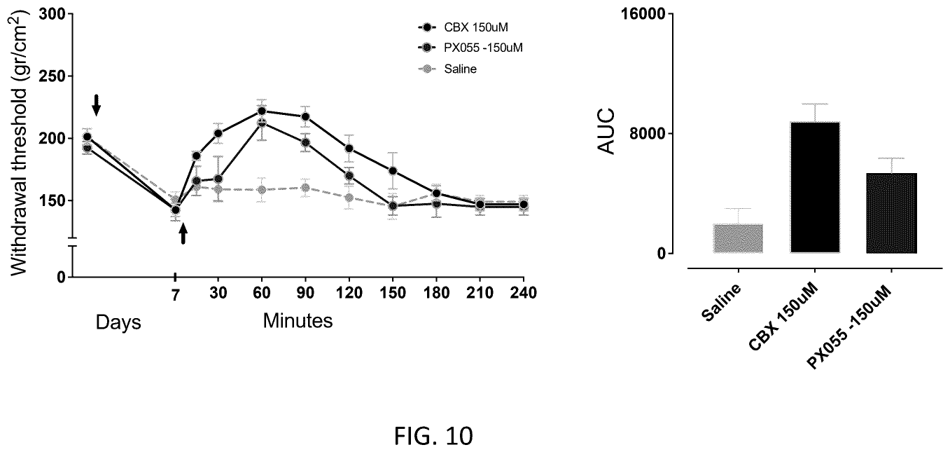

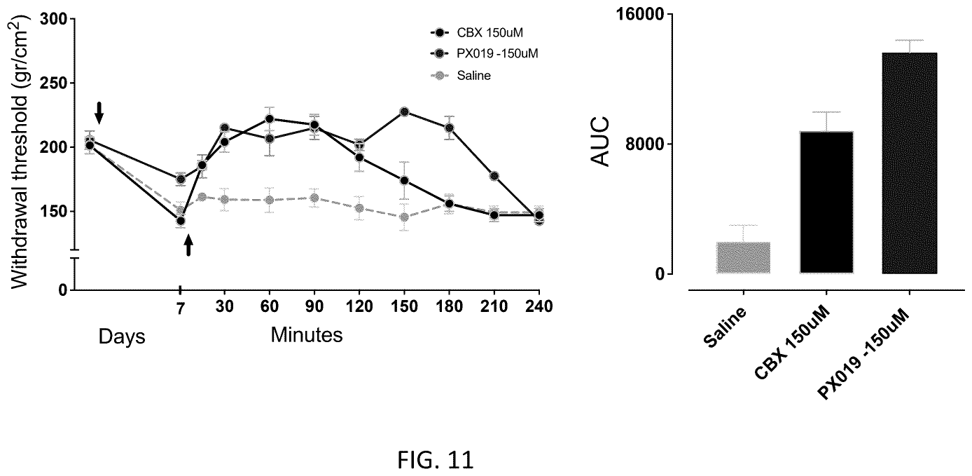

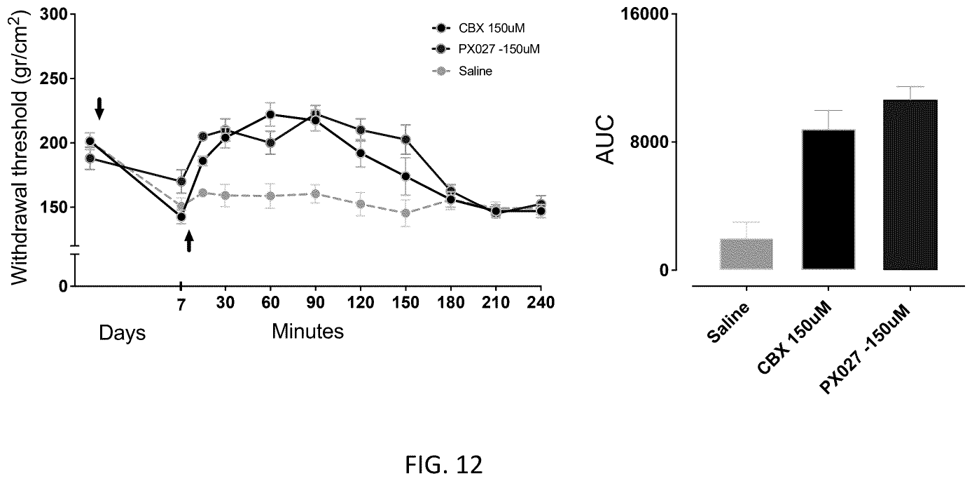

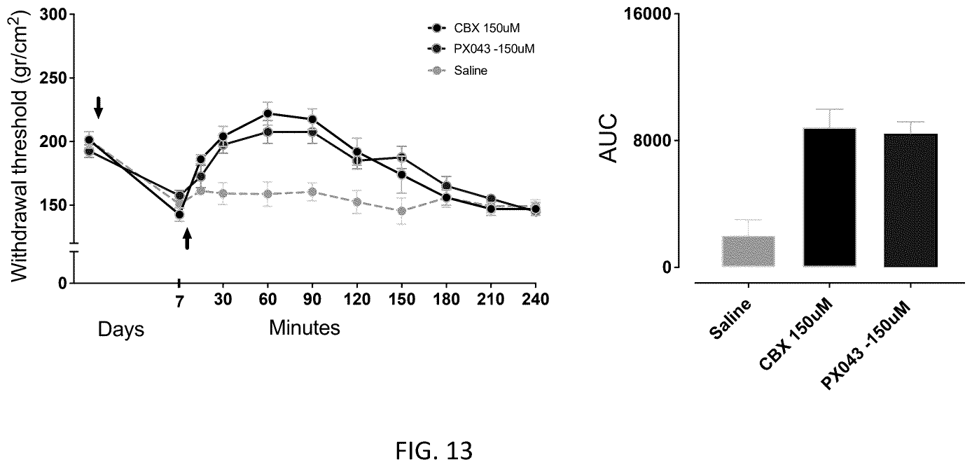

Seven days after surgical induction of neuropathic pain, the test compounds (CMPD004 (FIG. 7; PX004), CMPD011 (FIG. 8; PX011), CMPD19 (FIG. 11; PX019), CMPD027 (FIG. 12; PX027), CMPD043 (FIG. 13; PX043), CMPD054 (FIG. 9; PX054) and CMPD055 (FIG. 10; PX055)), and as comparator carbenoxolone (an unspecific PANX1 blocker) and saline, were injected intrathecally. Pain was measured by using the paw pressure test. Nociceptive behavior was quantified using the paw pressure test (Randall and Sellito, 1957), also known as the Randall Sellito test. Briefly explained, the rat's right hind paw was subjected to increasing pressure by means of an algesimeter (Ugo Basile, Italy) until paw withdrawal. The maximum pressure exerted on the paw was 480 g (cut-off), a pressure that does not induce injury in the rat's paw. This algesimetric test was performed in neuropathic rats before (day 0), and 7 days after surgery, to verify the generation of mechanical hyperalgesia. The effect of the drugs was studied 7 days after the surgery for induction of neuropathy. An algesimetric measurement was performed before the injection of the test compounds, 15 minutes and then every 30 minutes after injection, for a period of 4 hours.

In summary, all compounds showed significant and intense analgesic effect compared to control, in the paw pressure test. Although with different intensities, 5 of the tested compounds showed a better performance compared to carbenoxolone, an unspecific, potent pannexin 1 channel blocker. The other two showed equal or a worst performance (FIGS. 7-13).

### Example 37—In Vivo Assessment of Anti-Addictive Potential in a Rat Model of Opioid Addiction

Three groups of Male Sprague-Dawley rats received different treatments (Group 1: Cmpd 004 (PX004), Group 2: Cmpd 011 (PX011), Group 3: Saline, N=4) administered with an Alzet© mini-osmotic pump model 1007D (DUR-ECT Corporation, Cupertino, CA, USA). The osmotic pumps were loaded with 84 μL of the PX drugs, at a concentration of 150 μM, or saline (NaCl 0.9%) and were aseptically implanted in the subcutaneous tissue of the dorsal region of the anaesthetized rat (isoflurane 3%) through a small incision in the skin. This Alzet pump releases 0.5±0.1 μL/h of saline or the test molecules solution for 5 days. Simultaneously, during these 5 days, all the groups received ascending doses of morphine i.p. at 8-h intervals (day 1, 10 mg/kg; day 2, 20 mg/kg; day 3, 30 mg/kg; day 4, 40 mg/kg). On day 5, rats received a morning injection of morphine (45 mg/kg) and 2 h later, naloxone (2 mg/kg i.p., naloxone hydrochloride dihydrate, Sigma) to rapidly induce opiate withdrawal behavior.

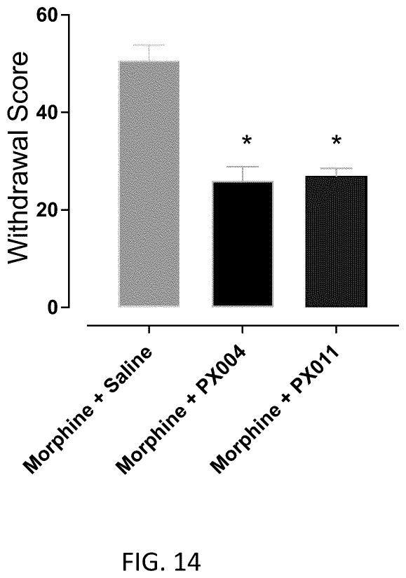

Signs of withdrawal were recorded as described by Ferrini et al. Jumping, teeth chattering, wet-dog shakes, headshakes and grooming behaviors were evaluated at 5-min intervals for a total test period of 30 min, and a standardized score of 0 to 3 will be assigned to each behavior. Allodynia, piloerection, salivation, ejaculation and tremors or twitching will also be evaluated, with 1 point given to the presence of the behavior during each 5-min interval. All signs were counted and compiled to yield a cumulative withdrawal score (FIG. 14).

### Example 38—In Vitro Assessment of ATP Release by Microglia and Astrocytes

Cortical microglia/astrocytes mechanically dissociated from decapitated postnatal P2-P4 rat pups (CD strain) were seeded on P75 flasks (1 brain per flask) and cultured at 37° C., 5% CO₂ for 10-20 days using DMEM media supplemented with 10% FBS and 1% pen/strep. On Day 10, microglia were collected by gently shaking and washing and purify by passing through 20 μm cell strainer and centrifugation.

Re-suspended microglia were counted and plated onto poly-D-lysine coated 96-well culture plate at a density of 80,000 cells/well. Dislodged the remaining astrocytes (enriched astrocytes) and plated onto poly-D-lysine coated 96-well culture plate at a density of 50,000 cells/well. Overnighted microglia and/or astrocytes were treated with the PANX-1 blockers of the invention (5 or 50 μM), vehicle or control (carbenoxolone, CBX) and subjected to lipopolysaccharide stimulation. Extracellular ATP was assessed by multilabel plate reader Envision (ultrasensitive luminescence) using CellTiter-Glo (Promega) kit.

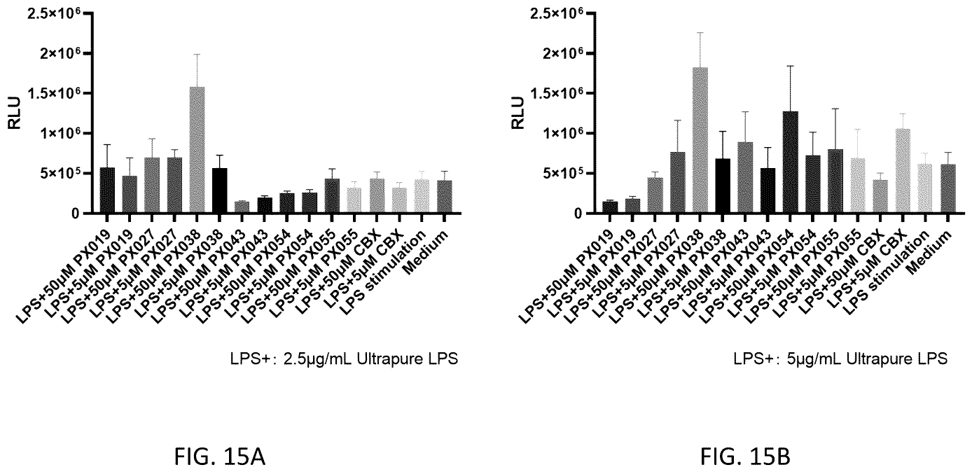

Proinflammatory LPS stimulation induced mild release of ATP by astrocytes (FIG. 15A) and microglial cells (FIG. 15B), which were prevented by compounds 19 (PX019), 43 (PX043), 53 (PX053), and 54 (PX054), confirming the biological activity of the compounds.

### REFERENCES

1. Navis K E, Fan C Y, Trang T, Thompson R J, Derksen D J. Pannexin 1 Channels as a Therapeutic Target: Structure, Inhibition, and Outlook. ACS Chem Neurosci. 2020 Aug. 5; 11(15):2163-2172.
2. Makarenkova H P, Shah S B, Shestopalov V I. The two faces of pannexins: new roles in inflammation and repair. J Inflamm Res. 2018 Jun. 21; 11:273-288.
3. Swayne L A, Boyce A K J. Regulation of Pannexin 1 Surface Expression by Extracellular ATP: Potential Implications for Nervous System Function in Health and Disease. Front Cell Neurosci. 2017 Aug. 8; 11:230.
4. Yeung A K, Patil C S, Jackson M F. Pannexin-1 in the CNS: Emerging concepts in health and disease. J Neurochem. 2020 September; 154(5):468-485.
5. Laird D W, Penuela S. Pannexin biology and emerging linkages to cancer. Trends Cancer. 2021 December; 7(12):1119-1131.
6. Locovei, S., Wang, J., & Dahl, G. (2006). Activation of pannexin 1 channels by ATP through P2Y receptors and by cytoplasmic calcium. FEBS letters, 580(1), 239-244.
7. Vanden Abeele, F., Bidaux, G., Gordienko, D., Beck, B., Panchin, Y. V., Baranova, A. V., . . . & Prevarskaya, N. (2006). Functional implications of calcium permeability of the channel formed by pannexin 1. The Journal of cell biology, 174(4), 535-546.
8. Yang, K., Xiao, Z., He, X., Weng, R., Zhao, X., & Sun, T. (2022). Mechanisms of Pannexin 1 (PANX1) Channel Mechanosensitivity and Its Pathological Roles. International Journal of Molecular Sciences, 23(3), 1523.
9. Pinheiro, A. R., Paramos-de-Carvalho, D., Certal, M., Costa, M. A., Costa, C., Magalhães-Cardoso, M. T., . . . & Correia-de-Sá, P. (2013). Histamine induces ATP release from human subcutaneous fibroblasts, via pannexin-1 hemichannels, leading to Ca2+ mobilization and cell proliferation. Journal of Biological Chemistry, 288(38), 27571-27583.
10. Jalaleddine, N., El-Hajjar, L., Dakik, H., Shaito, A., Saliba, J., Safi, R., . . . & El-Sabban, M. (2019). Pannexin1 is associated with enhanced epithelial-to-mesenchymal transition in human patient breast cancer tissues and in breast cancer cell lines. Cancers, 11(12), 1967.
11. Freeman, T. J., Sayedyahossein, S., Johnston, D., Sanchez-Pupo, R. E., O'Donnell, B., Huang, K., . . . & Penuela, S. (2019). Inhibition of pannexin 1 reduces the tumorigenic properties of human melanoma cells. Cancers, 11(1), 102.
12. Xu, X., Wang, J., Han, K., Li, S., Xu, F., & Yang, Y. (2018). Antimalarial drug mefloquine inhibits nuclear factor kappa B signaling and induces apoptosis in colorectal cancer cells. Cancer science, 109(4), 1220-1229.
13. Flores-Muñoz, C., Gómez, B., Mery, E., Mujica, P., Gajardo, I., Córdova, C., . . . & Ardiles, Á. O. (2020). Acute pannexin 1 blockade mitigates early synaptic plasticity defects in a mouse model of Alzheimer's disease. Frontiers in cellular neuroscience, 14,
46.
14. Hainz, N., Wolf, S., Tschernig, T., & Meier, C. (2016). Probenecid application prevents clinical symptoms and inflammation in experimental autoimmune encephalomyelitis. Inflammation, 39(1), 123-128.
15. D. Bravo et al., Pain, 2014 October; 155(10):2108-15. Pannexin 1: a novel participant in neuropathic pain signaling in the rat spinal cord.
16. Weaver, J., Arandjelovic, S., Brown, G. et al. Hematopoietic pannexin 1 function is critical for neuropathic pain. Sci Rep 7, 42550 (2017).
17. Mousseau M, Burma N E, Lee K Y, Leduc-Pessah H, Kwok C H T, Reid A R, O'Brien M, Sagalajev B, Stratton J A, Patrick N, Stemkowski P L, Biernaskie J, Zamponi G W, Salo P, McDougall J J, Prescott S A, Matyas J R, Trang T. Microglial pannexin-1 channel activation is a spinal determinant of joint pain. Sci Adv. 2018 Aug. 8; 4(8)
18. Gomez G I, Falcon R V, Maturana C J, Labra V C, Salgado N, Rojas C A, Oyarzun J E, Cerpa W, Quintanilla R A, Orellana J A. Heavy Alcohol Exposure Activates Astroglial Hemichannels and Pannexons in the Hippocampus of Adolescent Rats: Effects on Neuroinflammation and Astrocyte Arborization. Front Cell Neurosci. 2018 Dec. 4; 12:472.
19. Burma N E, Leduc-Pessah H, Trang T. Genetic deletion of microglial Panx1 attenuates morphine withdrawal, but not analgesic tolerance or hyperalgesia in mice. Channels (Austin). 2017 Sep. 3; 11(5):487-494. doi: 10.1080/19336950.2017.1359361. Epub 2017 Jul. 26. Erratum for: Addendum to: Burma N E, Bonin R P, Leduc-Pessah H, Baimel C, Cairncross Z F, Mousseau M, Shankara J V, Stemkowski P L, Baimoukhametova D, Bains J S, et al. Blocking microglial pannexin-1 channels alleviates morphine withdrawal in rodents. Nat Med 2017; 23:355-60.
20. Kim, J. E. and Kang, T. C. (2011) The P2X₇ receptor-pannexin-1 complex decreases muscarinic acetylcholine receptor-mediated seizure susceptibility in mice. J. Clin. Invest. 121, 2037-2047
21. Thompson, R. J., Jackson, M. F., Olah, M. E., Rungta, R. L., Hines, D. J., Beazely, M. A., MacDonald, J. F. and MacVicar, B. A. (2008) Activation of pannexin-1 hemichannels augments aberrant bursting in the hippocampus. Science 322, 1555-1559 CrossRef PubMed
22. Santiago, M. F., Veliskova, J., Patel, N. K., Lutz, S. E., Caille, D., Charollais, A., Meda, P. and Scemes, E. (2011) Targeting pannexin1 improves seizure outcome. PLoS ONE 6, e25178 CrossRef PubMed
23. Jiang, T., Long, H., Ma, Y., Long, L., Li, Y., Li, F., Zhou, P., Yuan, C. and Xiao, B. (2013) Altered expression of pannexin proteins in patients with temporal lobe epilepsy. Mol. Med. Rep. 8, 1801-1806
24. Shan Y, Ni Y, Gao Z. Pannexin-1 Channel Regulates ATP Release in Epilepsy. Neurochem Res. 2020 May; 45(5):965-971.
25. Aquilino M S, Whyte-Fagundes P, Zoidl G, Carlen P L. Pannexin-1 channels in epilepsy. Neurosci Lett. 2019 Mar. 16; 695:71-75.
26. Dossi E, Blauwblomme T, Moulard J, Chever O, Vasile F, Guinard E, Le Bert M, Couillin I, Pallud J, Capelle L, Huberfeld G, Rouach N. Pannexin-1 channels contribute to seizure generation in human epileptic brain tissue and in a mouse model of epilepsy. Sci Transl Med. 2018 May 30; 10(443)
27. Diaz E F, Labra V C, Alvear T F, Mellado L A, Inostroza C A, Oyarzún J E, Salgado N, Quintanilla R A, Orellana J A. Connexin 43 hemichannels and pannexin-1 channels contribute to the α-synuclein-induced dysfunction and death of astrocytes. Glia. 2019 August; 67(8):1598-1619.
28. Orellana J A, Froger N, Ezan P, Jiang J X, Bennett M V, Naus C C, Giaume C, Saez J C. ATP and glutamate released via astroglial connexin 43 hemichannels mediate neuronal death through activation of pannexin 1 hemichannels. J Neurochem. 2011 September; 118(5):826-40
29. Hainz N, Wolf S, Beck A, Wagenpfeil S, Tschernig T, Meier C. Probenecid arrests the progression of pronounced clinical symptoms in a mouse model of multiple sclerosis. Sci Rep. 2017 Dec. 8; 7(1):17214.
30. Lutz, S. E., Gonzalez-Fernandez, E., Ventura, J. C., Perez-Samartin, A., Tarassishin, L., Negoro, H., Patel, N. K., Suadicani, S. O., Lee, S. C., Matute, C. and Scemes, E. (2013) Contribution of pannexin1 to experimental autoimmune encephalomyelitis. PLoS ONE 8, e66657
31. Flores-Muñoz C, Gómez B, Mery E, Mujica P, Gajardo I, Córdova C, Lopez-Espindola D, Durán-Aniotz C, Hetz C, Muñoz P, Gonzalez-Jamett A M, Ardiles Á O. Acute Pannexin 1 Blockade Mitigates Early Synaptic Plasticity Defects in a Mouse Model of Alzheimer's Disease. Front Cell Neurosci. 2020 Mar. 19; 14:46.
32. Seo J H, Dalal M S, Calderon F, Contreras J E. Myeloid Pannexin-1 mediates acute leukocyte infiltration and leads to worse outcomes after brain trauma. J Neuroinflammation. 2020 Aug. 20; 17(1):245.
33. Bennett M V, Garré J M, Orellana J A, Bukauskas F F, Nedergaard M, Sáez J C. Connexin and pannexin hemichannels in inflammatory responses of glia and neurons. Brain Res. 2012 Dec. 3; 1487:3-15.
34. Karatas H, Erdener S E, Gursoy-Ozdemir Y, Lule S, Eren-Koçak E, Sen Z D, Dalkara T. Spreading depression triggers headache by activating neuronal Panx1 channels. Science. 2013 Mar. 1; 339(6123):1092-5. doi: 10.1126/science.1231897. Erratum in: Science. 2015 Oct. 2; 350(6256):aad5166. Erratum in: Science. 2015 Nov. 20; 350(6263):921.
35. Freitas-Andrade M, Bechberger J F, MacVicar B A, Viau V, Naus C C. Pannexin1 knockout and blockade reduces ischemic stroke injury in female, but not in male mice. Oncotarget. 2017 Jun. 6; 8(23):36973-36983.
36. Vultaggio-Poma V, Sarti A C, Di Virgilio F. Extracellular ATP: A Feasible Target for Cancer Therapy. Cells. 2020 Nov. 17; 9(11):2496.
37. Shi G, Liu C, Yang Y, Song L, Liu X, Wang C, Peng Z, Li H, Zhong L. Panx1 promotes invasion-metastasis cascade in hepatocellular carcinoma. J Cancer. 2019 Sep. 7; 10(23):5681-5688.
38. Bao L, Sun K, Zhang X. PANX1 is a potential prognostic biomarker associated with immune infiltration in pancreatic adenocarcinoma: A pan-cancer analysis. Channels (Austin). 2021 December; 15(1):680-696.
39. Boyd-Tressler A, Penuela S, Laird D W, Dubyak G R. Chemotherapeutic drugs induce ATP release via caspase-gated pannexin-1 channels and a caspase/pannexin-1-independent mechanism. J Biol Chem. 2014 Sep. 26; 289(39):27246-27263.
40. Fallon M T. Neuropathic pain in cancer. Br J Anaesth. 2013 July; 111(1):105-11.
41. Staff N P, Fehrenbacher J C, Caillaud M, Damaj M I, Segal R A, Rieger S. Pathogenesis of paclitaxel-induced peripheral neuropathy: A current review of in vitro and in vivo findings using rodent and human model systems. Exp Neurol. 2020 February; 324:113121.
42. Andelova K, Bacova B S, Sykora M, Hlivak P, Barancik M, Tribulova N. Mechanisms Underlying Antiarrhythmic Properties of Cardioprotective Agents Impacting Inflammation and Oxidative Stress. Int J Mol Sci. 2022 Jan. 26; 23(3):1416.
43. Andelova K, Egan Benova T, Szeiffova Bacova B, Sykora M, Prado N J, Diez E R, Hlivak P, Tribulova N. Cardiac Connexin-43 Hemichannels and Pannexin1 Channels: Provocative Antiarrhythmic Targets. Int J Mol Sci. 2020 Dec. 29; 22(1):260.
44. Sharma A K, Charles E J, Zhao Y, Narahari A K, Baderdinni P K, Good M E, Lorenz U M, Kron I L, Bayliss D A, Ravichandran K S, Isakson B E, Laubach V E. Pannexin-1 channels on endothelial cells mediate vascular inflammation during lung ischemia-reperfusion injury. Am J Physiol Lung Cell Mol Physiol. 2018 Aug. 1; 315(2):L301-L312.
45. Grimmer B, Krauszman A, Hu X, Kabir G, Connelly K A, Li M, Grune J, Madry C, Isakson B E, Kuebler W M. Pannexin 1-a novel regulator of acute hypoxic pulmonary vasoconstriction. Cardiovasc Res. 2021 Oct. 20:cvab326.
46. Good M E, Chiu Y H, Poon I K H, Jaffe I Z, Bayliss D A, Isakson B E, Ravichandran K S. Response by Good et al to Letter Regarding Article, “Pannexin-1 Channels as an Unexpected New Target of the Antihypertensive Drug Spironolactone”. Circ Res. 2018 May 25; 122(11)
47. Good M E, Chiu Y H, Poon I K H, Medina C B, Butcher J T, Mendu S K, DeLalio L J, Lohman A W, Leitinger N, Barrett E, Lorenz U M, Desai B N, Jaffe I Z, Bayliss D A, Isakson B E, Ravichandran K S. Pannexin 1 Channels as an Unexpected New Target of the Anti-Hypertensive Drug Spironolactone. Circ Res. 2018 Feb. 16; 122(4):606-615.
48. Larraflaga-Vera A, Marco-Bonilla M, Largo R, Herrero-Beaumont G, Mediero A, Cronstein B. ATP transporters in the joints. Purinergic Signal. 2021 December; 17(4):591-605.
49. Penuela S, Kelly J J, Churko J M, Barr K J, Berger A C, Laird D W. Panx1 regulates cellular properties of keratinocytes and dermal fibroblasts in skin development and wound healing. J Invest Dermatol. 2014 July; 134(7):2026-2035. doi:
50. Luu R, Valdebenito S, Scemes E, Cibelli A, Spray D C, Rovegno M, Tichauer J, Cottignies-Calamarte A, Rosenberg A, Capron C, Belouzard S, Dubuisson J, Annane D, de la Grandmaison G L, Cramer-Bordé E, Bomsel M, Eugenin E. Pannexin-1 channel opening is critical for COVID-19 pathogenesis. iScience. 2021 Dec. 17; 24(12):103478.
51. Ransford G A, Fregien N, Qiu F, Dahl G, Conner G E, Salathe M. Pannexin 1 contributes to ATP release in airway epithelia. Am J Respir Cell Mol Biol. 2009 November; 41(5):525-34.
52. Khan M, Huang Y A, Kuo C Y, Lin T, Lu C H, Chen L C, Kuo M L. Blocking pannexin1 reduces airway inflammation in a murine model of asthma. Am J Transl Res. 2020 Jul. 15; 12(7):4074-4083.
53. Crespo Yanguas, S., da Silva, T. C, Pereira, I. V. A. et al. Genetic ablation of pannexin1 counteracts liver fibrosis in a chemical, but not in a surgical mouse model. Arch Toxicol 92, 2607-2627 (2018).
54. Seref-Ferlengez Z, Maung S, Schaffler M B, Spray D C, Suadicani S O, Thi M M. P2X7R-Panx1 Complex Impairs Bone Mechanosignaling under High Glucose Levels Associated with Type-1 Diabetes. PLoS One. 2016 May 9; 11(5):e0155107.
55. Adamson S E, Meher A K, Chiu Y H, Sandilos J K, Oberholtzer N P, Walker N N, Hargett S R, Seaman S A, Peirce-Cottler S M, Isakson B E, McNamara C A, Keller S R, Harris T E, Bayliss D A, Leitinger N. Pannexin 1 is required for full activation of insulin-stimulated glucose uptake in adipocytes. Mol Metab. 2015 Jul. 3; 4(9):610-8.
56. Bartley C, Brun T, Oberhauser L, Grimaldi M, Molica F, Kwak B R, Bosco D, Chanson M, Maechler P. Chronic fructose renders pancreatic β-cells hyper-responsive to glucose-stimulated insulin secretion through extracellular ATP signaling. Am J Physiol Endocrinol Metab. 2019 Jul. 1; 317(1):E25-E41.
57. Tozzi M, Hansen J B, Novak I. Pannexin-1 mediated ATP release in adipocytes is sensitive to glucose and insulin and modulates lipolysis and macrophage migration. Acta Physiol (Oxf). 2020 February; 228(2):e13360.
58. Yang K, Xiao Z, He X, Weng R, Zhao X, Sun T. Mechanisms of Pannexin 1 (PANX1) Channel Mechanosensitivity and Its Pathological Roles. Int J Mol Sci. 2022 Jan. 28; 23(3):1523.
59. Dvoriantchikova G, Pronin A, Kurtenbach S, Toychiev A, Chou T H, Yee C W, Prindeville B, Tayou J, Porciatti V, Sagdullaev B T, Slepak V Z, Shestopalov V I. Pannexin 1 sustains the electrophysiological responsiveness of retinal ganglion cells. Sci Rep. 2018 Apr. 11; 8(1):5797.
60. Pronin A, Pham D, An W, Dvoriantchikova G, Reshetnikova G, Qiao J, Kozhekbaeva Z, Reiser A E, Slepak V Z, Shestopalov V I. Inflammasome Activation Induces Pyroptosis in the Retina Exposed to Ocular Hypertension Injury. Front Mol Neurosci. 2019 Mar. 13; 12:36.
61. Cui H, Liu Y, Qin L, Wang L, Huang Y. Increased membrane localization of pannexin1 in human corneal synaptosomes causes enhanced stimulated ATP release in chronic diabetes mellitus. Medicine (Baltimore). 2016 December; 95(49):
62. Ferrari D, Casciano F, Secchiero P, Reali E. Purinergic Signaling and Inflammasome Activation in Psoriasis Pathogenesis. Int J Mol Sci. 2021 Aug. 31; 22(17):9449. doi: 10.3390/ijms22179449. PMID: 34502368; PMCID: PMC8430580.
63. Garcia-Vega L, O'Shaughnessy E M, Jan A, Bartholomew C, Martin P E. Connexin 26 and 43 play a role in regulating proinflammatory events in the epidermis. J Cell Physiol. 2019 Feb. 2. doi: 10.1002/jcp.28206. Epub ahead of print. PMID: 30710344.
64. British pharmacopoeia. Vol. 1. London: Medicines and Healthcare products Regulatory Agency; 2018.
65. European pharmacopoeia. 9th ed, Strassbourg: Council of Europe: 2018.
66. United States Pharmacopoeia, 42, National Formulary 37, 2018.
67. REMINGTON, J. P., AND GENNARO, A. R. Remington's Pharmaceutical Sciences. Mack Publishing Co., 18th ed. 1990.
68. MARTINDALE, W. AND REYNOLDS, J. E. F. Martindale: The Extra Pharmacopoeia. London, The Pharmaceutical Press 31st ed, 1996.
69. HARRY, R. AND ROSEN, M. R. Harry's cosmeticology. Leonard Hill Books, 9th ed. 2015.
70. KANIKKANNAN, N. K., KANDIMALLA, K., LAMBA, S. S., SINGH, M. Structure-Activity Relationship of Chemical Penetration Enhancers in Transdermal Drug Delivery. Current Medicinal Chemistry, 7(6): 593-608. 2000.
71. JAVADZADEH Y., ADIBKIA K., HAMISHEKAR H. Transcutol® (Diethylene Glycol Monoethyl Ether.
72. A Potential Penetration Enhancer. In: Dragicevic N., Maibach H. (eds) Percutaneous Penetration Enhancers Chemical Methods in Penetration Enhancement. Springer, Berlin, Heidelberg, 2015.
73. WIECHERS, J. W. AND D E ZEEUW, R. A. Transdermal drug delivery: efficacy and potential applications of the penetration enhancer Azone. Drug Des Deliv. (2):87-100. 1990.
74. RAIMAN, J., KOLJONEN, M., HUIKKO, K., KOSTIANEN, R., HIRVONEN, J. Delivery and Stability of LHRH and Nafarelin in Human Skin: The Effect of Constant/Pulsed Iontophoresis. European Journal of Pharmaceutical Sciences: Official Journal of the European Federation for Pharmaceutical Sciences, 21(2-3): 371-77. 2004.
75. WANG, Y., TRAKUR, R., FAN, Q., MICHNIAK, B. Transdermal Iontophoresis: Combination Strategies to Improve Transdermal Iontophoretic Drug Delivery. European Journal of Pharmaceutics and Biopharmaceutics: Official Journal of Arbeitsgemeinschaft Fur Pharmazeutische Verfahrenstechnik, 60(2): 179-91. 2005.
76. PARK, E. J., WERNER, J., SMITH, N. B. Ultrasound Mediated Transdermal Insulin Delivery in Pigs Using a Lightweight Transducer. Pharmaceutical Research, 24(7): 1396-1401. 2007.
77. MELKAMU, G., WOHLRAB, J., NEUBERT, R. H. Dermal Delivery of Desmopressin Acetate Using Colloidal Carrier Systems. The Journal of Pharmacy and Pharmacology, 57(4):423-27. 2005.
78. MANOSROI, A., KHANRIN, P., LOHCHAROENKAL, W., WERNER, R. G., GOTZ, F., MANOSROI, W., MANOSROI, J. Transdermal Absorption Enhancement through Rat Skin of Gallidermin Loaded in Niosomes. International Journal of Pharmaceutics, 392(1-2): 304-10. 2010.
79. E L MAGHRABY, G. M., WILLIAMS, A. C., BARRY, B. W. Skin Delivery of Oestradiol from Deformable and Traditional Liposomes: Mechanistic Studies. The Journal of Pharmacy and Pharmacology, 51(10):1123-34. 1999.
80. DAYAN, N., and E. TOUITOU. Carriers for Skin Delivery of Trihexyphenidyl HCl: Ethosomes vs. Liposomes. Biomaterials, 21(18): 1879-85. 2000.
81. Crocetti L, Guerrini G, Puglioli S, Giovannoni M P, Di Cesare Mannelli L, Lucarini E, Ghelardini C, Wang J, Dahl G. Design and synthesis of the first indole-based blockers of Panx-1 channel. Eur J Med Chem. 2021 Nov. 5; 223:113650. doi: 10.1016/j.ejmech.2021.113650. Epub 2021 Jun. 19. PMID: 34174741.
82. Xu, X. J., Boumechache, M., Robinson, L. E., Marschall, V., Gorecki, D. C., Masin, M., & Murrell-Lagnado, R. D. (2012). Splice variants of the P2X7 receptor reveal differential agonist dependence and functional coupling with pannexin-1. Journal of Cell Science, 125(16), 3776-3789.

1. A compound of formula I or a pharmaceutically acceptable salt, hydrate, solvate, or polymorph thereof: 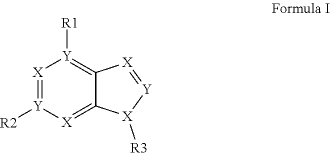
wherein,
X and Y are individually C, CH or N, or preferably wherein Y is C, or CH and X is N;
R¹, R² and R³ are independently hydrogen; lower alkyl, optionally substituted with hydroxy; lower cycloalkyl; lower alkenyl; lower alkoxy; lower alkynyl; phenyl; halogenated phenyl; halogen; a substituted or non-substituted aromatic or non-aromatic heterocycle, preferably diazine, pyrazole, diazole, triazole, or their alkyl derivatives; 2-hydroxyisopropyl; or have one of the following structures: 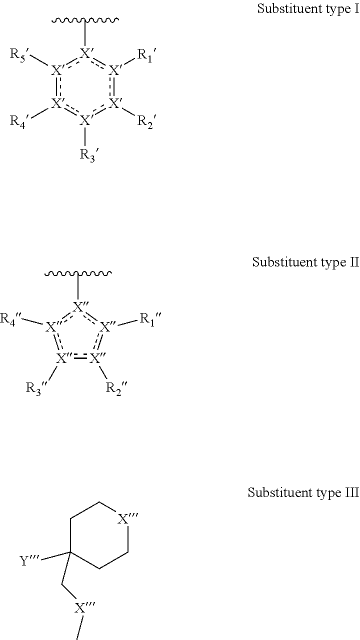
wherein,
X′ is C, CH, CH₂, N, S, or O, or preferably can be CH₂, N, or O;
×″ is independently C, CH, CH₂, N, S, or O, preferably CH₂, N, or O;
X′″ is NH, N-lower alkyl or O;
R1′-R5′ are independently absent, hydrogen, lower alkyl, lower cycloalkyl, halogen, OH, NH₂, hydroxy-lower alkyl, or NH-lower alkyl, O-lower alkyl, O—PO—OR₆′, O-Lower alkyl or alkyloxy-O—PO—O—R₆′, or O—PO—O-lower alkyl-O—PO—R₆′, preferably OH, NH₂, NH-lower alkyl, wherein vicinal R1′ and R5′ may form a 5-7 membered ring moiety;
R₆′ is H or O-lower alkyl;
R1″-R4″ are independently absent, hydrogen, lower alkyl, lower cycloalkyl, OH, NH₂, hydroxy-lower alkyl, NH-lower alkyl, O-lower alkyl, O—PO—OR₆′, O-lower alkyl or alkyloxy-O—PO—O—R₆′, or O—PO—O-lower alkyl-O—PO—R₆′, preferably OH, NH₂, NH-lower alkyl, or halogen;
Y′″ is halogen; and
wherein the bonds between adjacent ring substituents X′ or adjacent ring substituents X″ may constitute single or double bonds; or
a compound of formula I′ or a pharmaceutically acceptable salt, hydrate, solvate, or polymorph thereof: 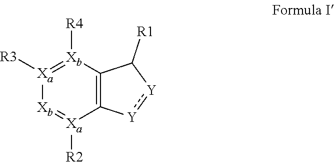
wherein,
Xₐ or X_{b} is C, CH or N, or preferably Xₐ is C or CH, and X_{b} is N; Y is CH, CH₂, C-lower alkyl, CH-lower alkyl, C-halogen, CH-halogen or C-dihalogen; Z is CH or N, preferably CH; wherein the bond between adjacent ring substituents Y may constitute a single or double bond;
R¹, R², R³, and R⁴ are independently absent, hydrogen; lower alkyl; lower cycloalkyl; lower alkenyl; lower alkoxy; lower alkynyl; phenyl; halogenated phenyl; halogen; a substituted or non-substituted aromatic or non-aromatic heterocycle, preferably triazole, or diazine; 2-hydroxyisopropyl, or have one of the following structures: 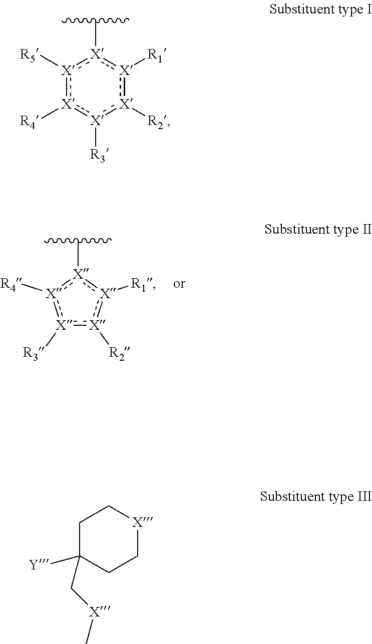
wherein,
X′ is C, CH, CH₂, N, S or O, preferably CH₂, N, or O;
X″ is C, CH, CH₂, N, S or O, preferably CH₂, N, or O;
X′″ is NH, N-lower alkyl or O;
R1′-R5′ are independently absent, or are H, lower alkyl, lower cycloalkyl, OH, NH₂, NH-lower alkyl, halogen, O-lower alkyl, O—PO—OR₆′, O-Lower alkyl or alkyloxy-O—PO—O—R₆′, or O—PO—O-lower alkyl-O—PO—R₆′, preferably OH, NH₂, NH-lower alkyl, wherein vicinal R1′ and R5′ may form a 5-7 membered ring moiety;
R₆′ is H or O-lower alkyl;
R1″-R4″ are independently absent, or are hydrogen, lower alkyl, lower cycloalkyl, or halogen;
Y′″ is halogen, and
wherein the bonds between adjacent ring substituents X′ or adjacent ring substituents X″ may constitute single or double bonds, forming thereby a non-aromatic or aromatic ring.

2. A compound according to claim 1 selected from the group consisting of: 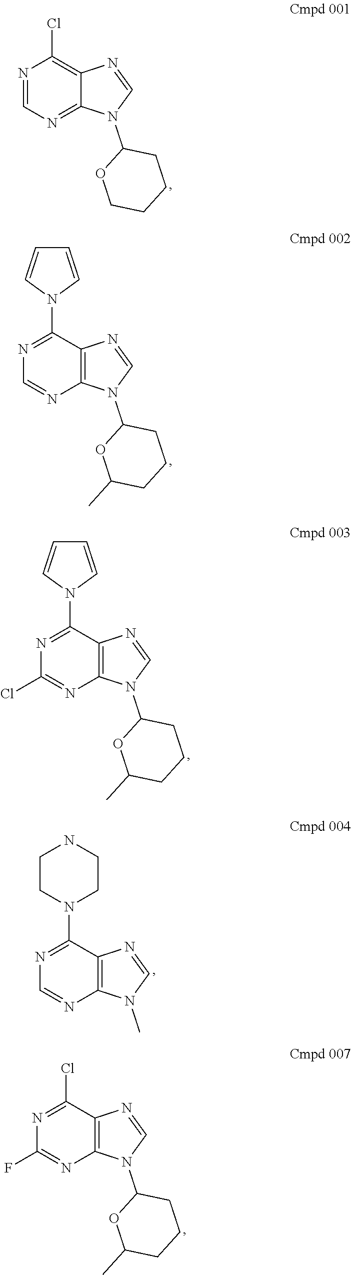 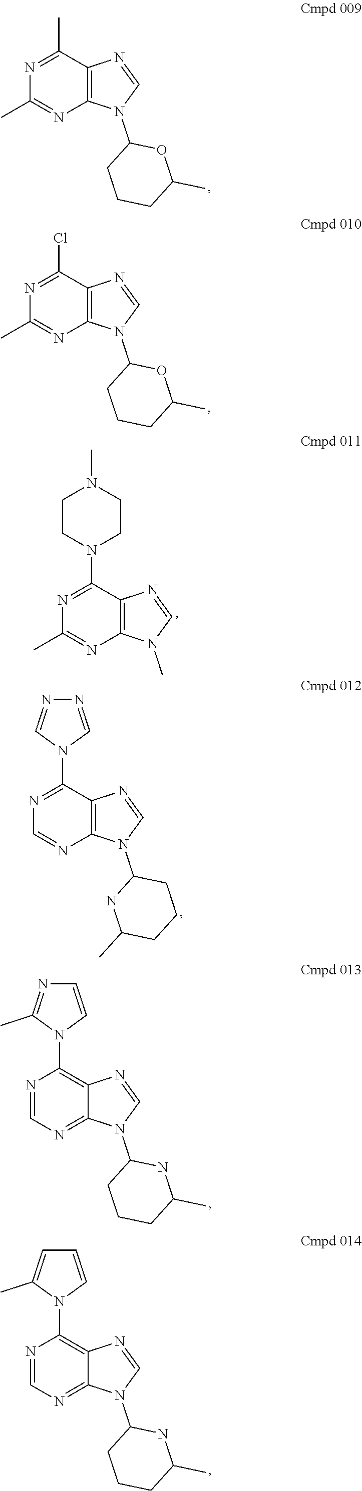 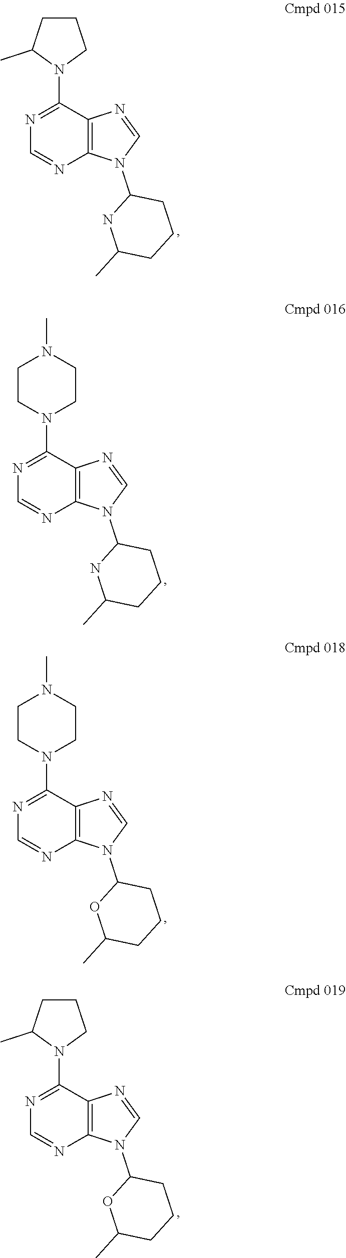 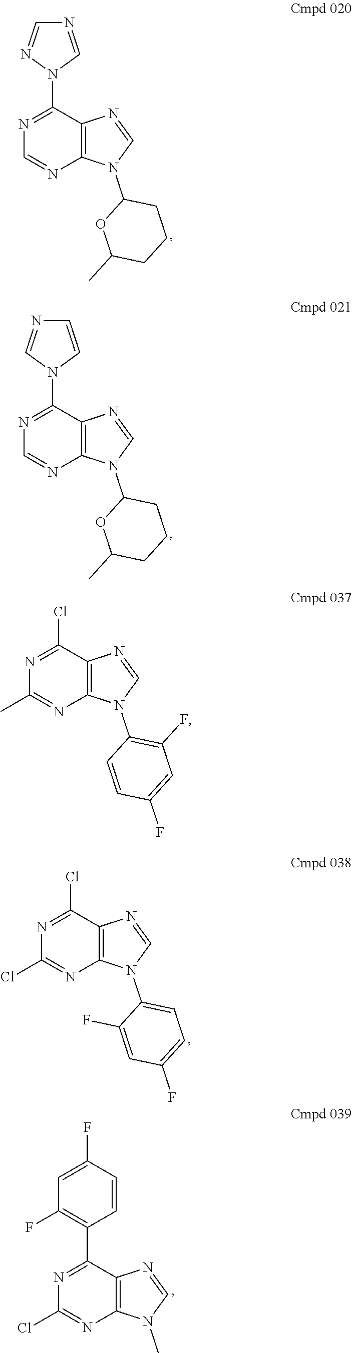 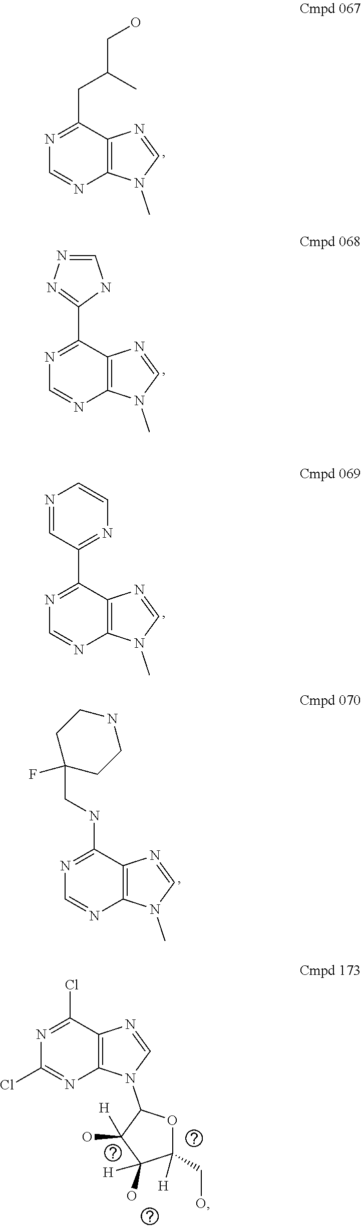 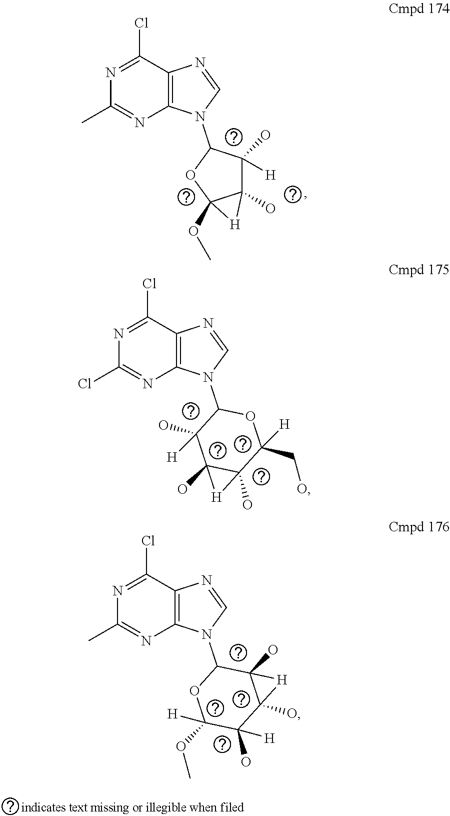
and a pharmaceutically acceptable salt, hydrate, solvate, or polymorph thereof.

3. (canceled)

4. A compound according to claim 1 selected from the group consisting of: 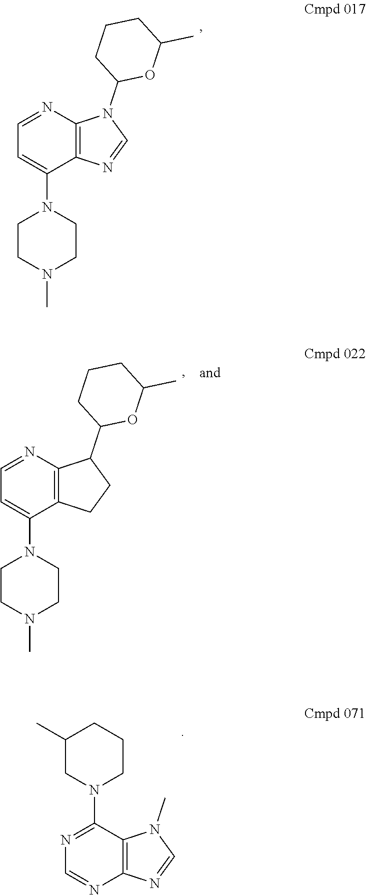
and a pharmaceutically acceptable salt, hydrate, solvate, or polymorph thereof.

5. A compound of formula II or a pharmaceutically acceptable salt, hydrate, solvate, or polymorph thereof: 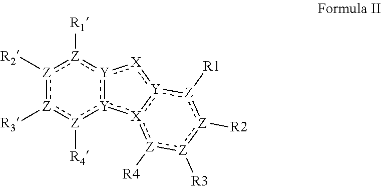
wherein,
X is N; Y is C; and Z is N, C or CH; wherein bonds between adjacent ring substituents X, Y and/or Z constitute single or double bonds;
R¹-R⁴ are independently absent when Z is N, or,
one or more of R¹-R⁴ or R¹′-R⁴′ are lower to medium chain oxo- or keto fatty acids, optionally substituted with oxo or lower alkyl; H; halogen; lower alkyl; lower cycloalkyl; or COOH; or
one or more of R¹-R⁴ or R¹′-R⁴′ comprise the substituent of Formula X: 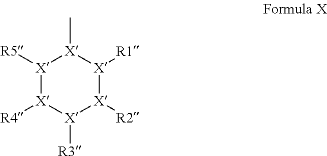
wherein,
X′ is CH, CO, N or O, preferably wherein X′ comprises up to two N or O; R¹″-R⁵″ are independently absent, or are H; lower alkyl; lower cycloalkyl; halogen; NH₂; NH-lower alkyl; hydroxy; oxo; COOH, SO₂NH₂; SO₂NH-loweralkyl; a substituted or non-substituted aromatic or non-aromatic heterocycle, preferably tetrazole; or comprises the substituent of Formula XI: 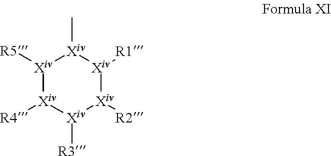
wherein,
X′″ is CH, CH₂, CO, N or O, or preferably wherein X^{iv} comprises up to two N or O; R¹′″-R⁵′″ are independently absent, H, halogen, NH₂, lower alkyl, lower cycloalkyl, NH-lower alkyl, hydroxy, COOH, SO₂NH₂, SO₂NH-loweralkyl; a substituted or non-substituted aromatic or non-aromatic heterocycle, preferably tetrazole; or preferably wherein only one of R¹′″-R⁵′″ comprises a non-substituted aromatic or non-aromatic heterocycle; wherein the bonds between adjacent ring substituents X^{iv} may constitute single or double bonds, forming thereby a non-aromatic or aromatic ring; or
one or more of R¹-R⁴ comprise the substituent of formula XII: 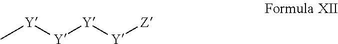
wherein,
Y′ is absent or can each be, independently, CH₂, CH₂(CH₃); CO, SO, SO₂, CHOH or NH; and Z′ is COOH; SO₂NH₂; a substituted or non-substituted aromatic or non-aromatic heterocycle, preferably tetrazole; forming a C₃-C₈ linear or branched chain; or
one or more of R₁-R₄ comprise the substituent of formula XIII: 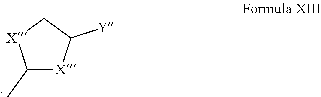
wherein,
Y″ is COOH, SO₂NH₂, or tetrazole; and X′″ is N or O; or
a compound of formula III, or a pharmaceutically acceptable salt, hydrate, solvate, or polymorph thereof; 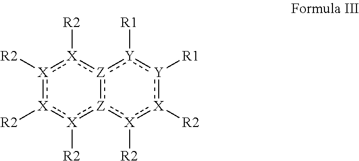
wherein,
each Z is independently selected from CH or C; each X is independently selected from CH, C, CH₂, O, N, S, SO, or SO₂; each Y is independently selected from N, C, CH, CH₂, CO, SO, S, or N-alkyl; wherein the bonds between adjacent ring substituents X, Z and/or Y constitute single or double bonds;
each R¹ is either absent or is H, halogen, O, NH₂, N-lower cycloalkyl, a phenyl group, alkyl group, optionally substituted with one or both of oxo and carboxy, OH, SO₂N(CH₃)₂, C(O)N(CH₃)₂, a five membered heterocycle, preferably tetrazole, —CO—(CH₂)ₙ—COOH, wherein n is 0-4, wherein R₁ optionally may form together with neighboring substituents, an aromatic or aliphatic 5, 6, or 7-membered ring system, which may contain an heteroatom, optionally substituted with one or more COOH, carboxamide, alkoxy, oxo, halogen, or triazole groups, or R, optionally comprises the substituent of Formula XIV: 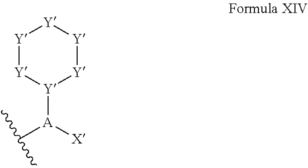
wherein,
X′ is absent or is O, N, or N-lower cycloalkyl; A is CH or may be a direct bond when X′ is absent; Y′ is O, N, N-lower cycloalkyl, CH₂, CH-lower alkyl, or CH—OH, or preferably X′ is absent or is O and the Y′-ring contains 1 or 2 N or O atoms; wherein the bonds between adjacent substituents Y′ and other atoms in the ring constitute single or double bonds, thereby forming a non-aromatic or aromatic ring; and
R² is absent, or is halogen; H; lower alkyl, optionally substituted with carboxy or oxo; hydroxy; alkoxy, COOH; SO₂NH₂; SO₂NH—O—CH₃, SO₂NH—C(O)—CH₃, SO₂N(CH₃)₂, SO₂-lower alkyl; C(O)N(CH₃)₂, a substituted or non-substituted aromatic or non-aromatic heterocycle, preferably triazole or tetrazole; or comprises the substituent of formula XV; 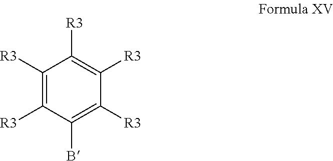
wherein,
B′ is attachment side, wherein B′ may comprise a linker or a direct bond, wherein the linker is —N—; and R³ is independently H, COOH, SO₂NH₂, SO₂NH-lower alkyl, halogen, or a substituted or non-substituted aromatic or non-aromatic heterocycle, preferably triazole or tetrazole; or
a compound of formula IV or a pharmaceutically acceptable salt, hydrate, solvate, or polymorph thereof: 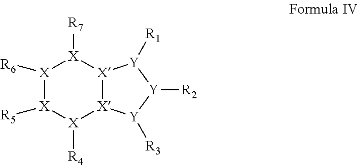
wherein,
each X is independently selected from CH or C;
X′ is C;
Each Y is independently selected from N, CH, C, SO₂ or CO;
R¹ and R² are each independently H, COOH O—CH₃, lower cycloalkyl, or join together to form a five- or six-membered heterocyclic ring, which is optionally substituted;
R³-R⁷ is each independently H, COOH O—CH₃, lower cycloalkyl, or comprises the substituent of formula XVI: 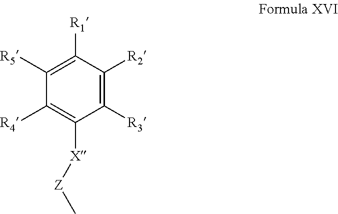
wherein,
Z is —N—CO—N or lower alkyl-CO—N;
X″ is lower alkyl or partly halogenated lower alkyl; and
R1′-R5′ are independently H; CHF2; COOH SO2NH2; NH—CH₂—COOH; lower alkyl, lower alkyl-COOH, lower alkyl-CO—,lower alkyl-COOH, —O—CO—CH₃, OCH3, N—CO—N—O-lower alkyl, N—CO—N—O—CF₂, —SO₂—N (lower alkyl)₂, lower alkyl-O-lower alkyl-COOH, SO₂—N-lower alkyl-phenylcarboxylic acid; or a substituted or non-substituted aromatic or non-aromatic heterocycle, preferably tetrazole, which may optionally be connected to the bicyclic ring of formula IV with a lower alkyl linker; or
a compound of formula V or a pharmaceutically acceptable salt, hydrate, solvate, or polymorph thereof: 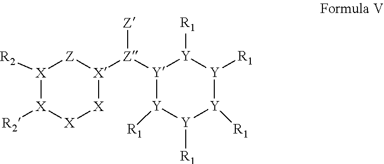
wherein,
X is CH₂, C, or CH; X′ is either C or CH; Y is C or CH; Y′ is C; Z is independently N, O, or N-lower alkyl; Z′ is O; R₂ is H, halogen, lower alkyl, ketobutyric acid, cyclohexanonecarboxylic acid or hydroxymethyl(cyclohexenone); wherein the bonds between adjacent ring substituents X, X′ and/or Z constitute single or double bonds, and wherein the bonds between adjacent Y and/or Y′ constitute single or double bonds; Z″ can be CH or N, or lower alkyl, lower cycloalkyl, alkyl ether or cycloalkyl; and R₁ can be H, lower alkyl, halogen, O-lower alkyl, COOH, N—CO—N—O-lower alkyl or cycloalkyl, partly halogenated lower alkyl or cycloalkyl, —CO—CH₃, -lower alkyl-O-phenyl carboxylic acid with a lower alkyl substituent, or 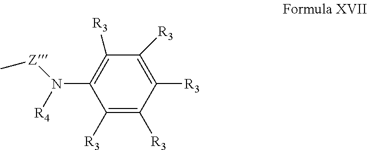
wherein,
Z′″ is lower alkyl or cycloalkyl, R₃ is each independently H, COOH NH—CO—NH—OCH₃ or partly halogenated NH—CO—NH—CH₃, O-lower alkyl, CO—CH₃ or lower alkyl, and R₄ is lower alkyl; or
a compound of formula VI or a pharmaceutically acceptable salt, hydrate, solvate, or polymorph thereof: 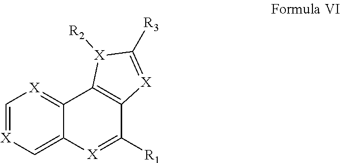
wherein,
independently X is N or CH; R¹ is NH₂, NH-lower alkyl, OH, or O-lower cycloalkyl; R² and R³ can be H, lower alkyl, SO₂NH₂ or comprise the substituent of formula XVIII: 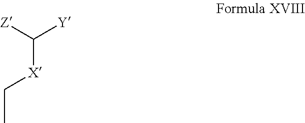
wherein,
X′ is a direct bond, NH, N—CH₃ or CH₂; Y′ is H or lower alkyl; and Z′ is H, lower alkyl, COOH, SO₂NH₂, SO₂NH—CH₃, or a substituted or non-substituted aromatic or non-aromatic heterocycle, preferably tetrazole, wherein X′ is N—CH₃ when both Y′ and Z′ are H; or
a compound of formula VII or a pharmaceutically acceptable salt, hydrate, solvate, or polymorph thereof: 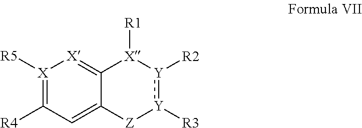
wherein,
X is N or C, X′ is CH or N; X″ is N, or CH; Y is CH or C; Z is CH₂— or C═O; wherein the bond between adjacent ring substituents Y constitute a single or double bond;
R¹ comprises a substituent of formula XIX: 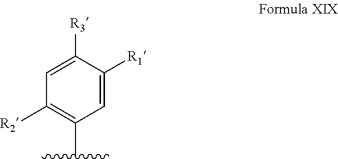
wherein,
R1′-R3′ can be independently halogen, carboxylic acid, SO₂NH₂, SO₂—NH-lower alkyl, COOH, or NO₂, or a substituted or non-substituted aromatic or non-aromatic heterocycle, preferably tetrazole; or
R1 can comprise a substituent of formula XX: 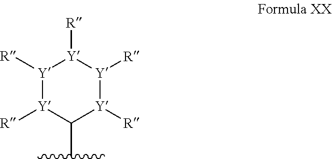
wherein,
Y′ is N, CH, or C; R″ is independently H, halogen, dihalogen or lower alkyl;
R² is H, lower cycloalkyl or a substituted or non-substituted aromatic or non-aromatic heterocycle, preferably 1,3-diazolidine;
R³ is halogen, carboxylic acid, SO₂NH₂, or SO₂—NH-lower alkyl, or a substituted or non-substituted aromatic or non-aromatic heterocycle, preferably tetrazole;
R⁴ is H, halogen, or low alkyl ketoacid, preferably ketopropionic acid;
R⁵ is absent if X is N or CH; if X is C, R5 is a substituted or non-substituted aromatic or non-aromatic heterocycle, preferably pyrrolidine, a piperidine, pyrazine, or a bicyclic structure of formula XXI: 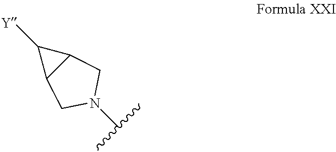
wherein
Y″ is N, N-lower alkyl, or COOH; or
a compound of formula VIII or a pharmaceutically acceptable salt, hydrate, solvate, or polymorph thereof: 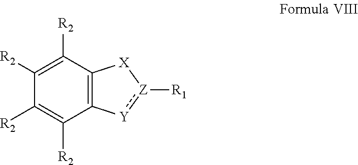
wherein
X is SO₇, NR₄ or O; Z is C or N; Y is N, CR₃ or CH; R₁-R₄ are each independently H, oxo, halogen, —(CH₂)ₙ—COOH, wherein n=0 to 6, —NH—C(O)—NH—CH₃, —NH—C(O)—NH—CHF₂, alkyl or alkoxy, optionally substituted with COOH or oxo, or wherein one or more of R₁-R₄ comprise a substituent of Formula XXII: 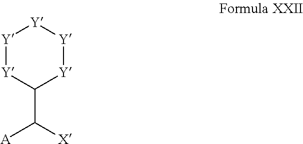
wherein,
A is attachment side, wherein the substituent of Formula XXII is attached directly or through a linker L to the compound of formula VIII, wherein L is alkyl, —CH₂—C(O)—NH—, or —NH—C(O)—NH—;
X′ is H, O, N, or N-lower cycloalkyl;
Y′ is O, N, N-lower cycloalkyl, CH₂, CR₅, CHR₅, wherein R₅ is alkyl, hydroxy, alkoxy, halogen, wherein the alkyl is optionally substituted by one or more of COOH oxo, alkoxy, halogen, or hydroxylamine, wherein adjacent Y′ are bonded with a single or double bond, and wherein two R₅ attached to two adjacent Y′ are optionally joined to form an aromatic or non-aromatic five- or six-membered ring; preferably X′ is O and the Y′-ring contains 1 or 2 N or O atoms.

6. A compound according to claim 5, selected from the group consisting of: 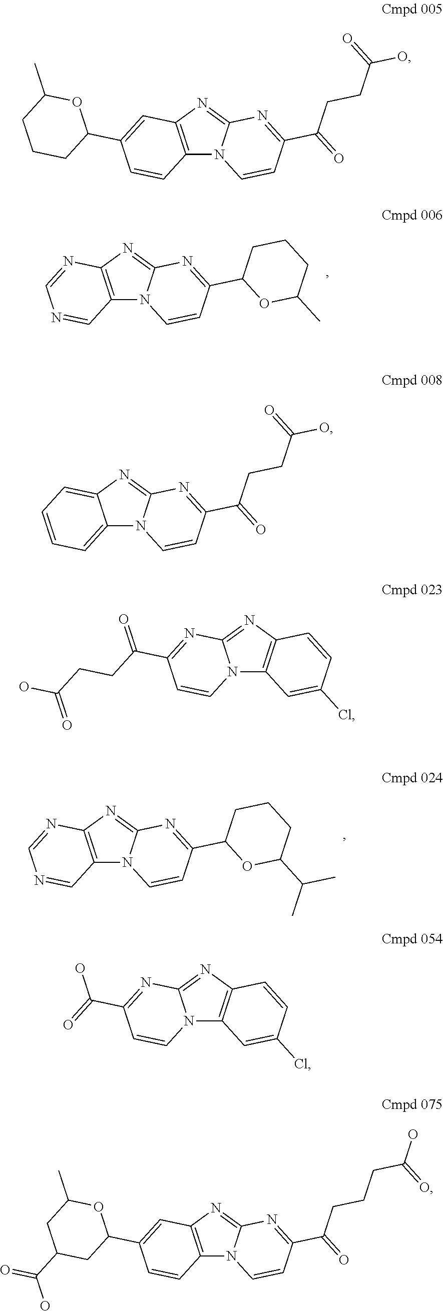 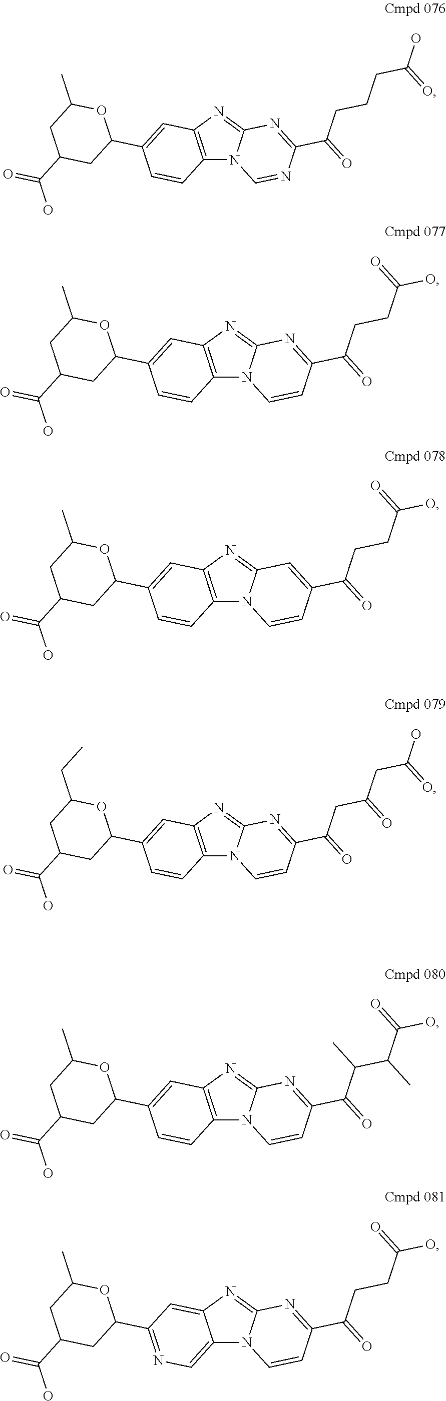 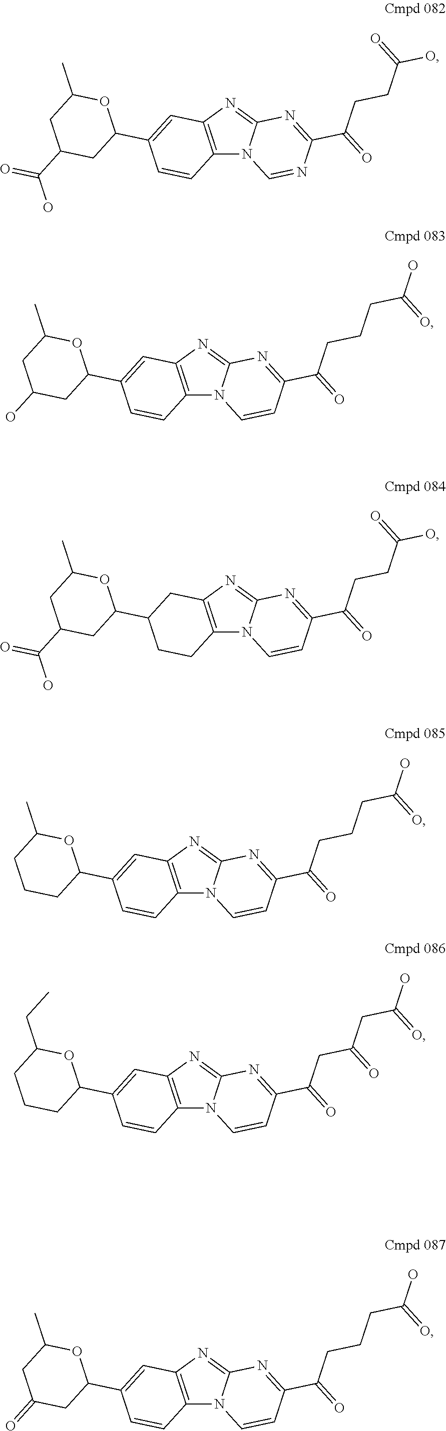 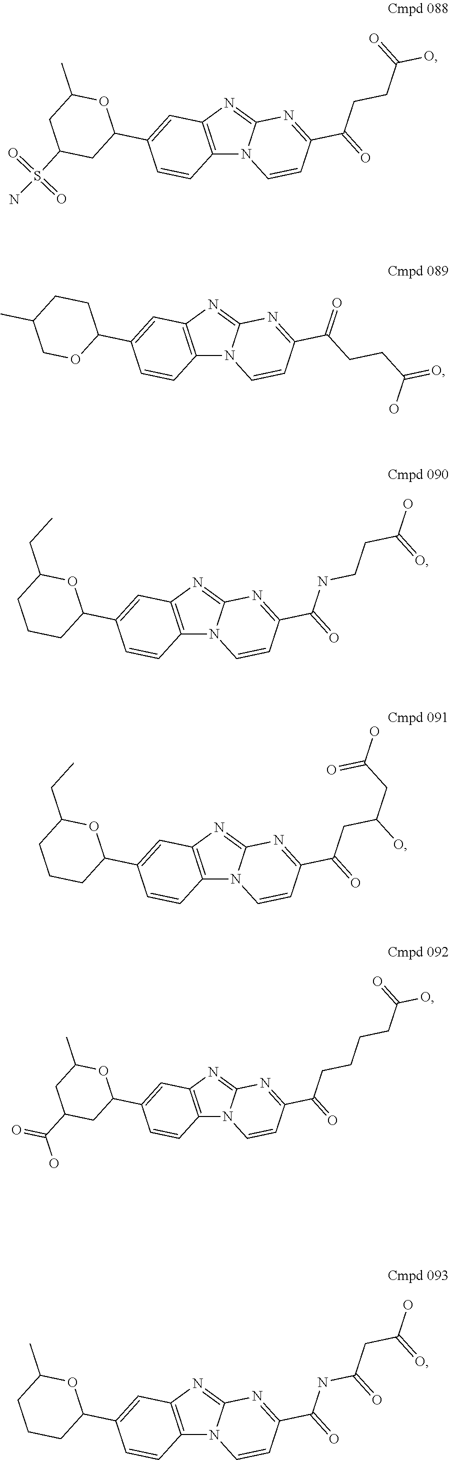 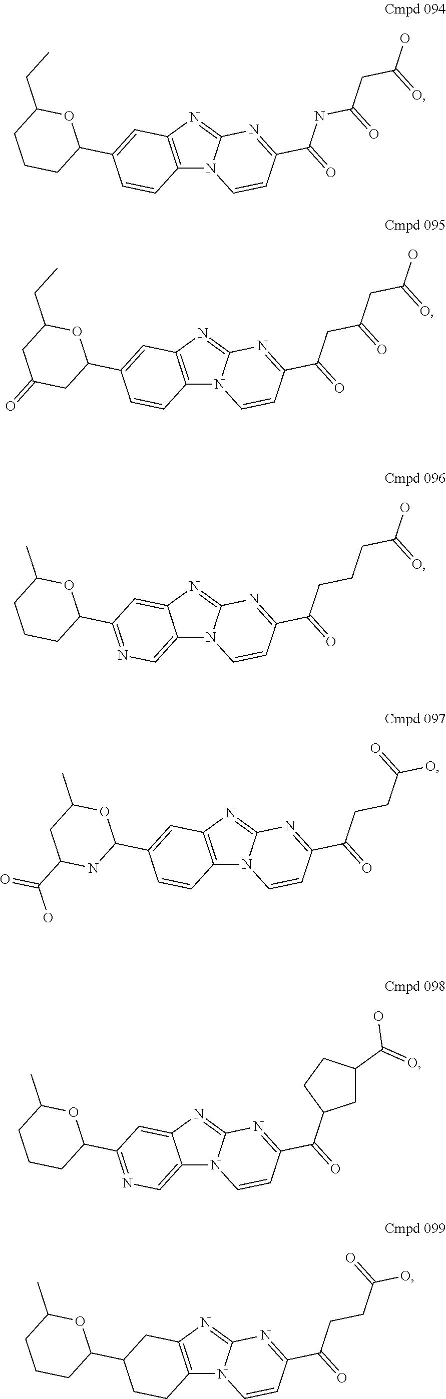 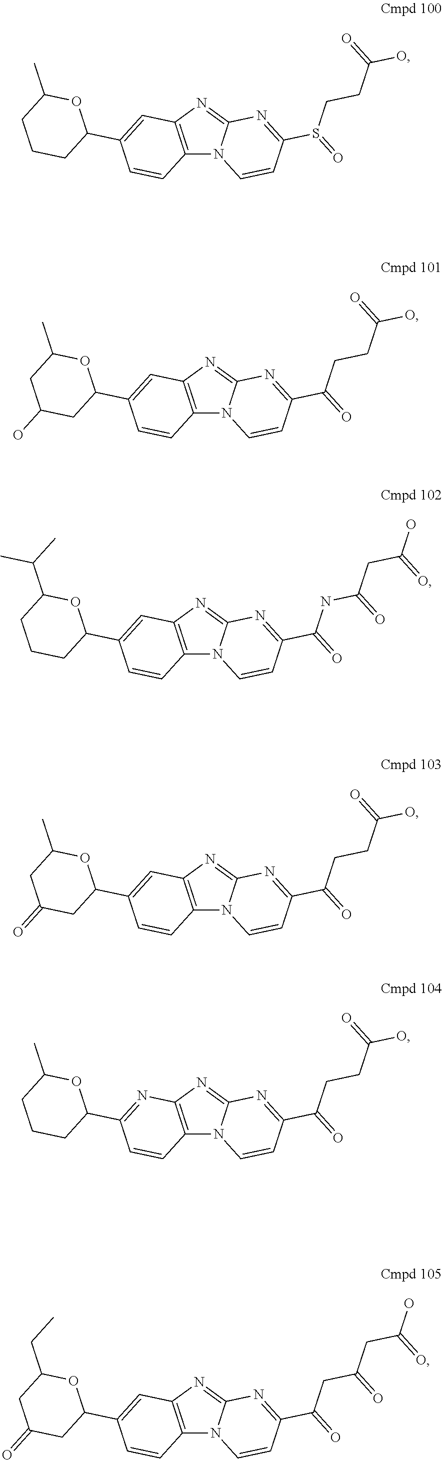 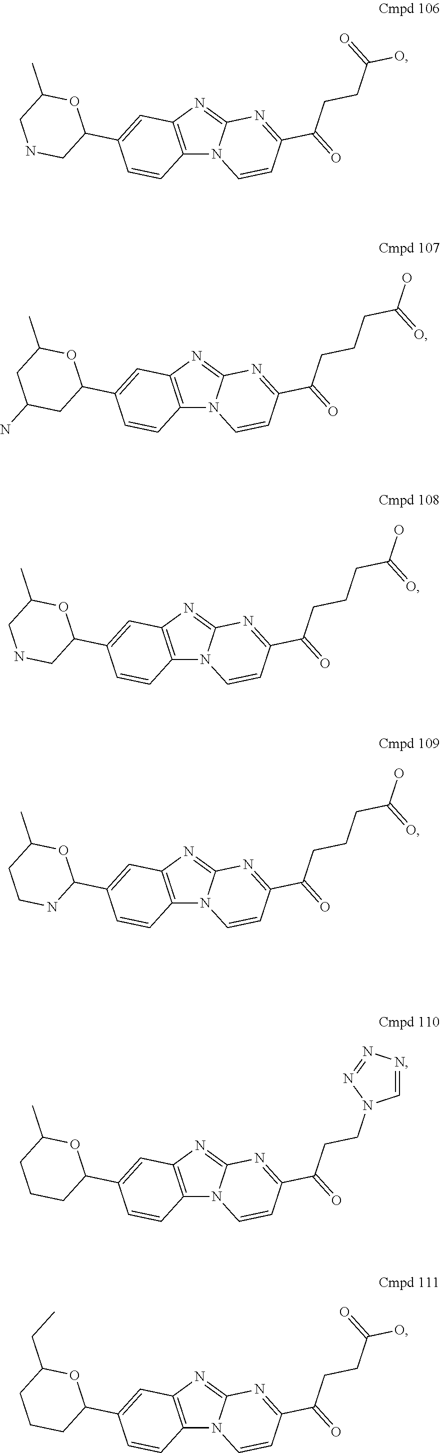 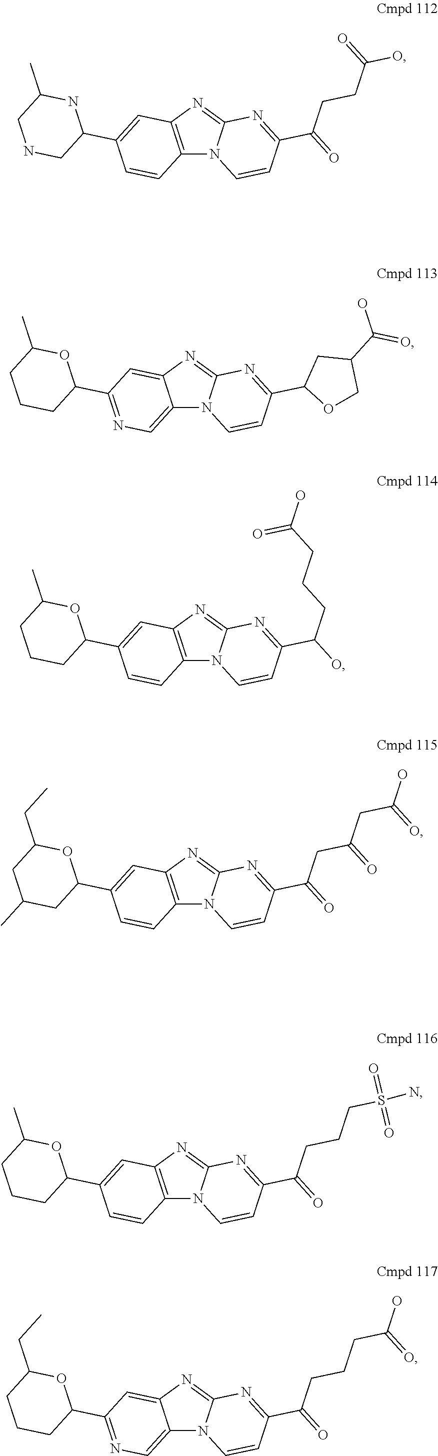 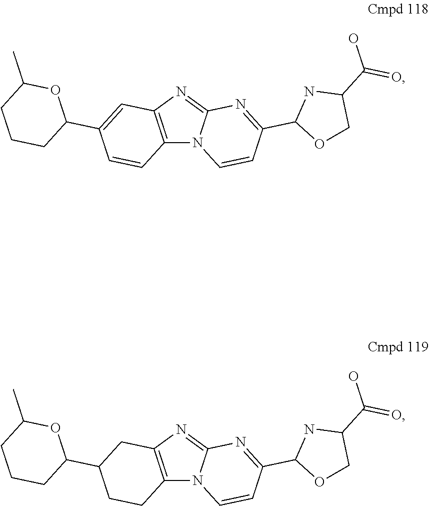 [SMILES: CC1CCCC(c2ccc3c(c2)nc2nc(C4NC(C(=O)O)CO4)ccn23)O1 ; CC1CCCC(C2CCc3c(nc4nc(C5NC(C(=O)O)CO5)ccn34)C2)O1]
and a pharmaceutically acceptable salt, hydrate, solvate, or polymorph thereof.

7. (canceled)

8. A compound according to claim 5 selected from the group consisting of: 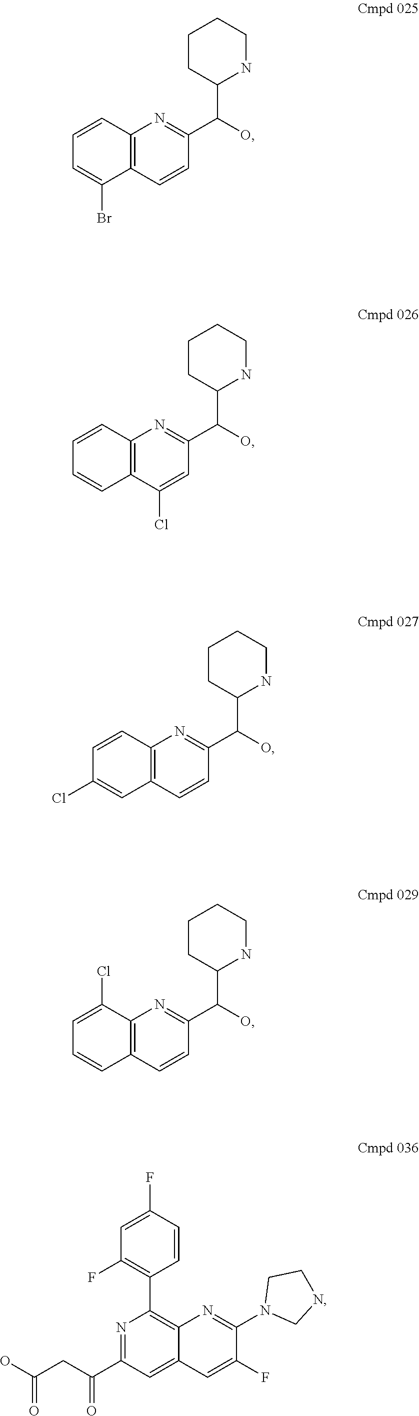 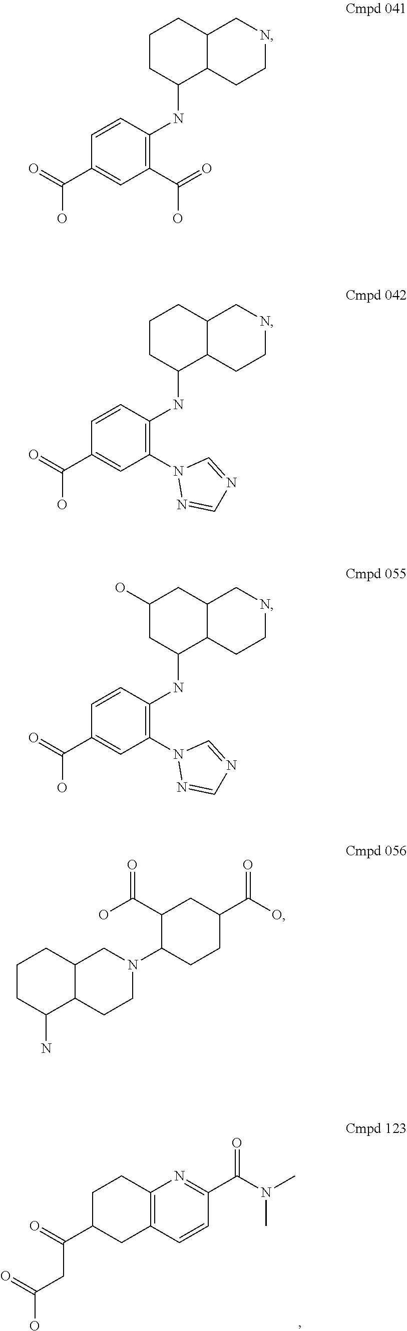 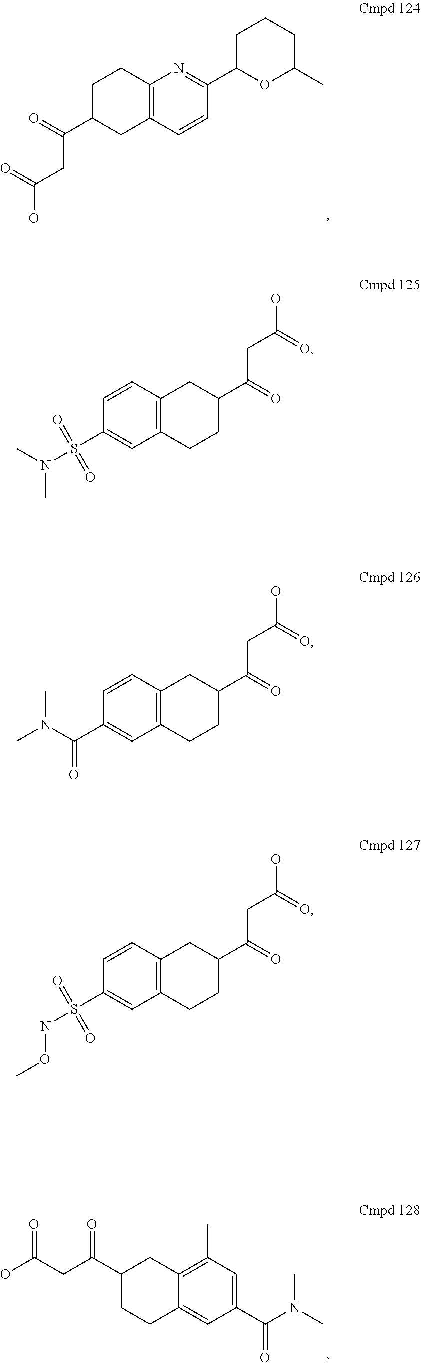 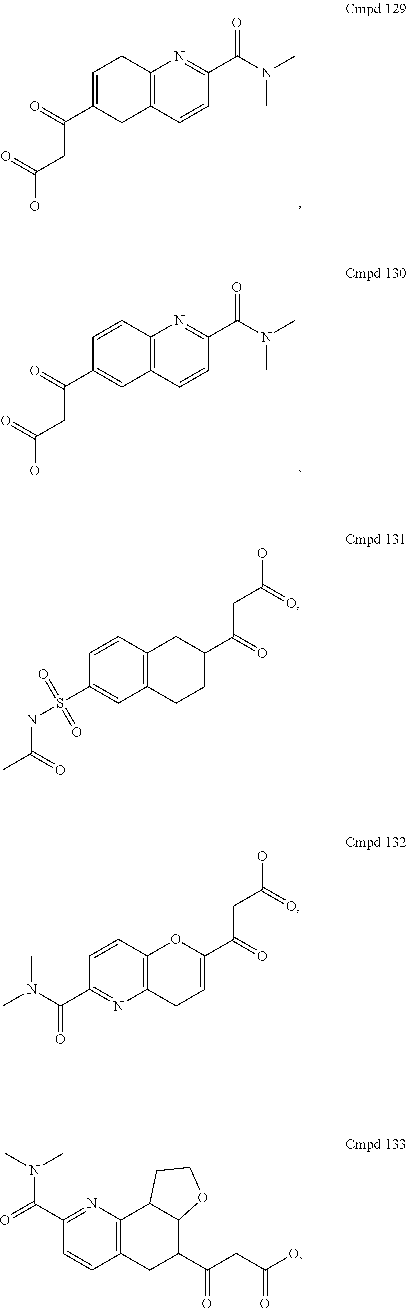 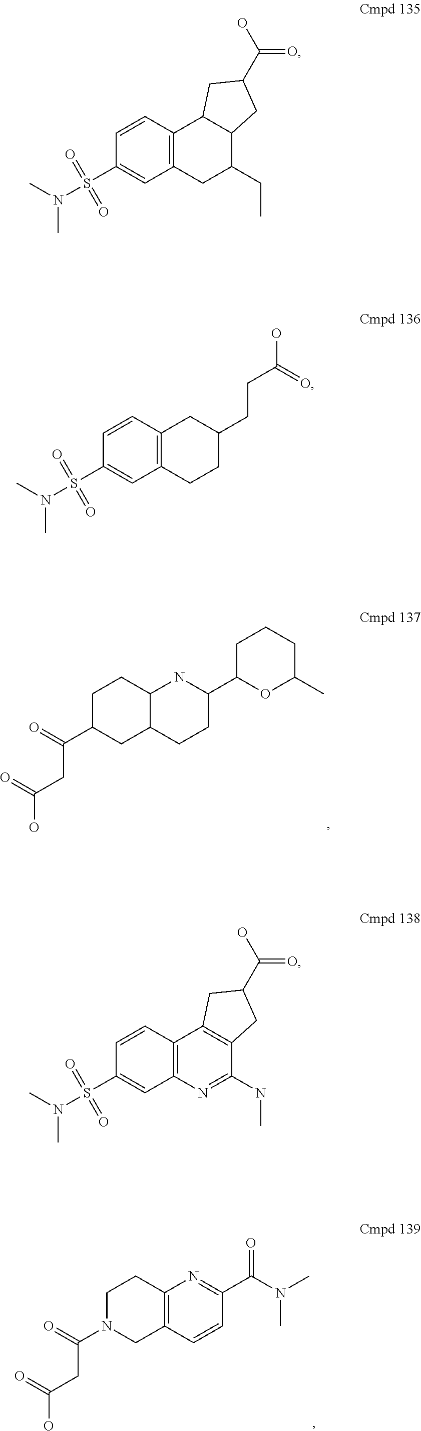 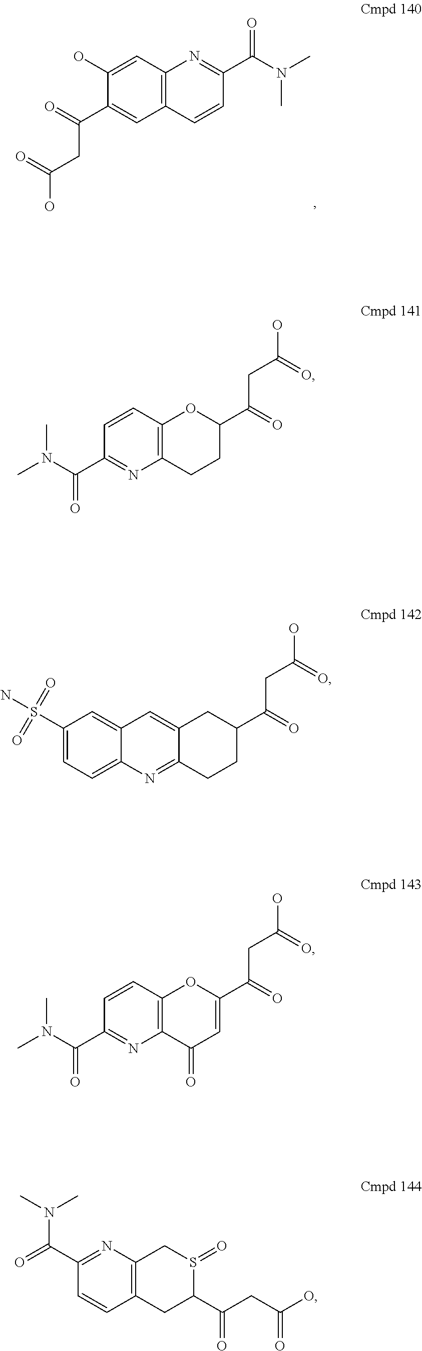 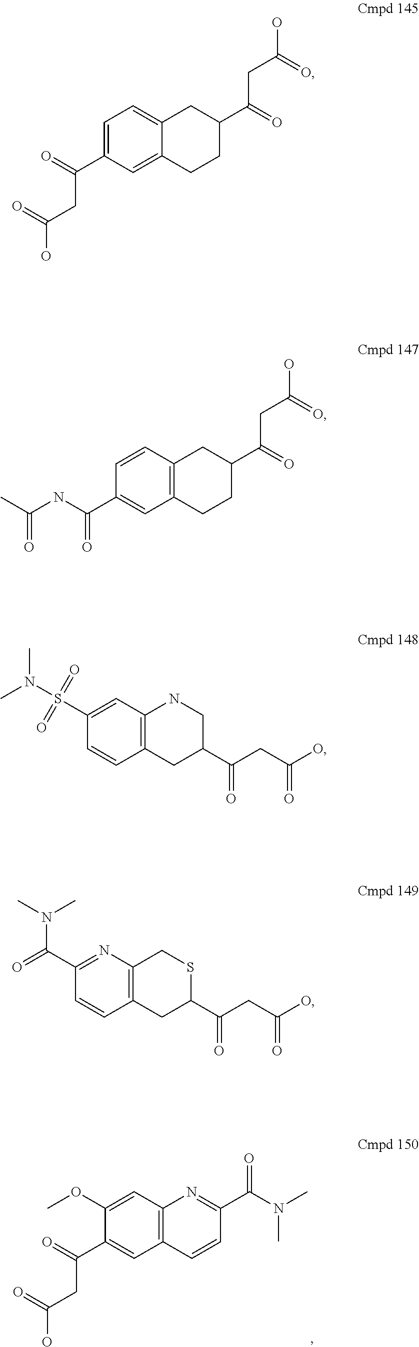 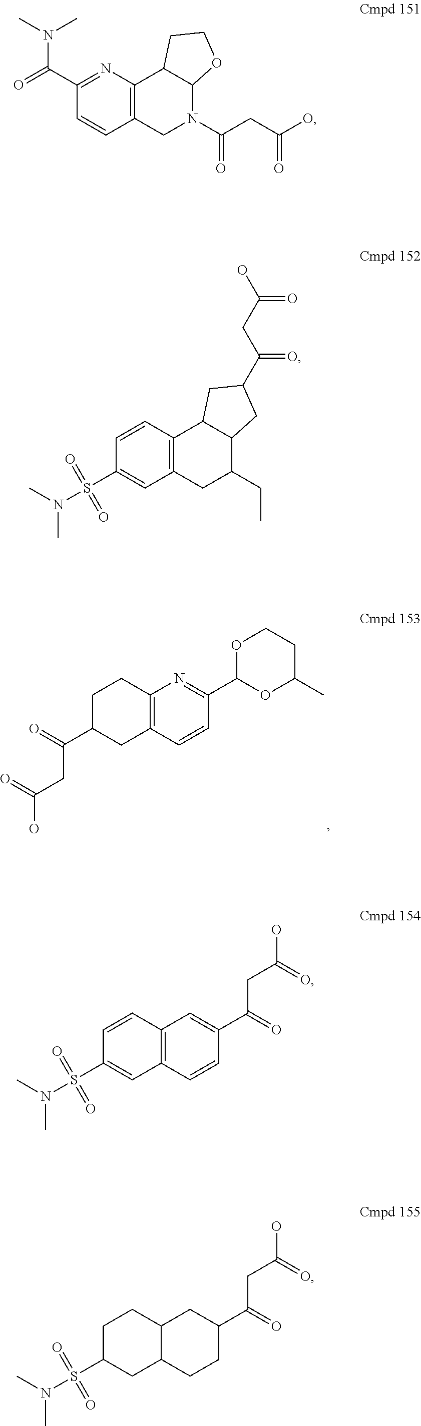 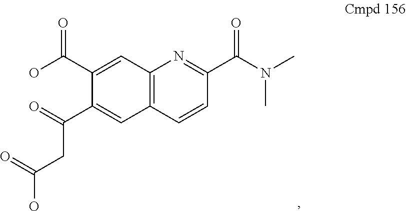 [SMILES: CN(C)C(=O)c1ccc2cc(C(=O)CC(=O)O)c(C(=O)O)cc2n1] 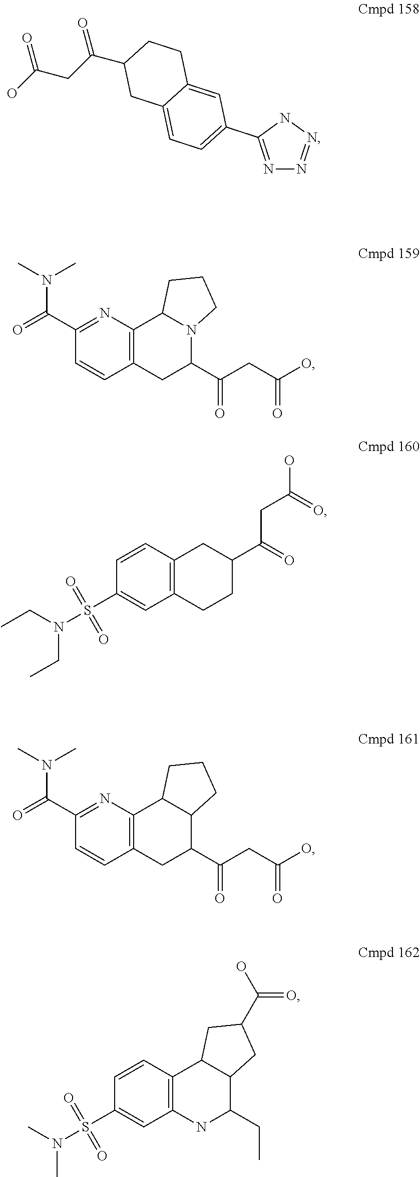 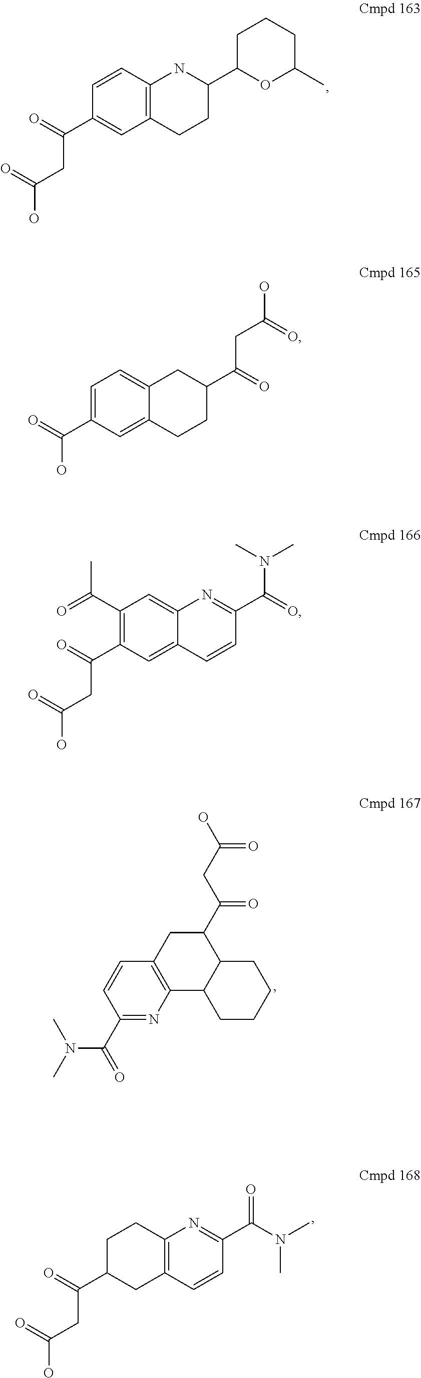 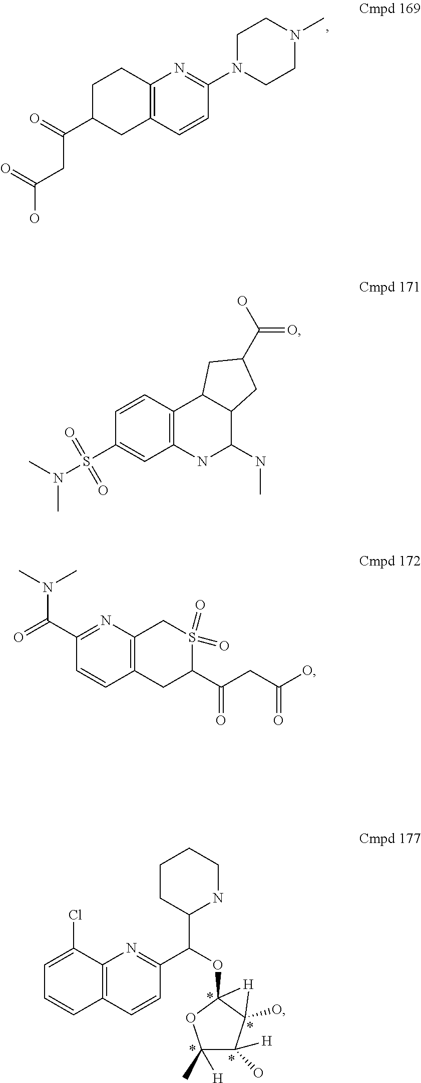 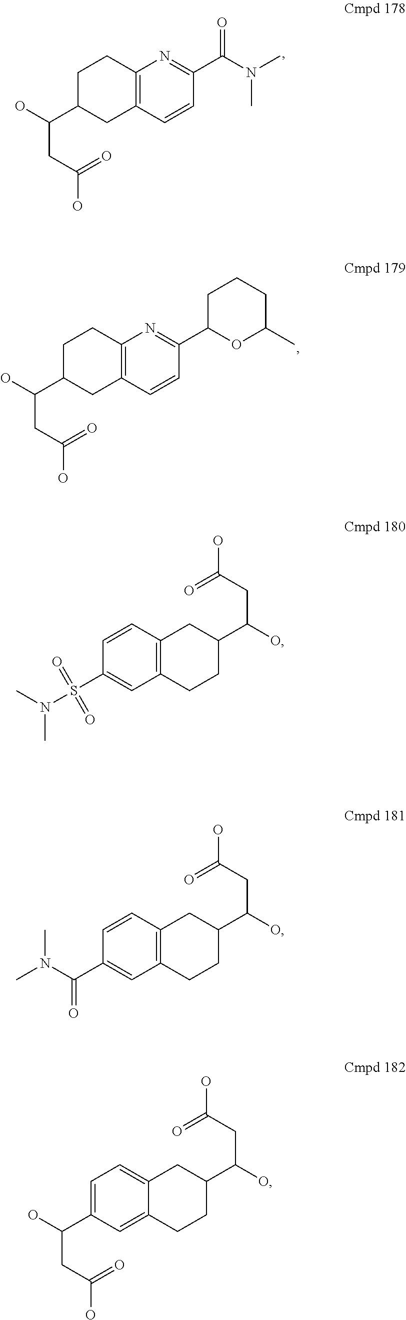 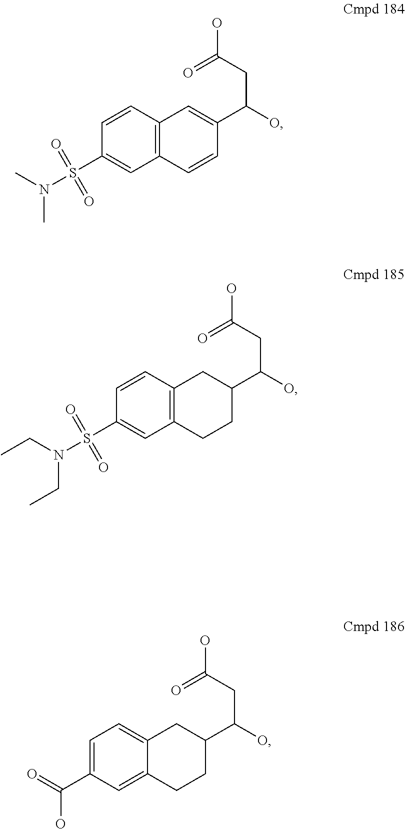
and a pharmaceutically acceptable salt, hydrate, solvate, or polymorph thereof.

9. (canceled)

10. A compound according to claim 5 selected from the group consisting of: 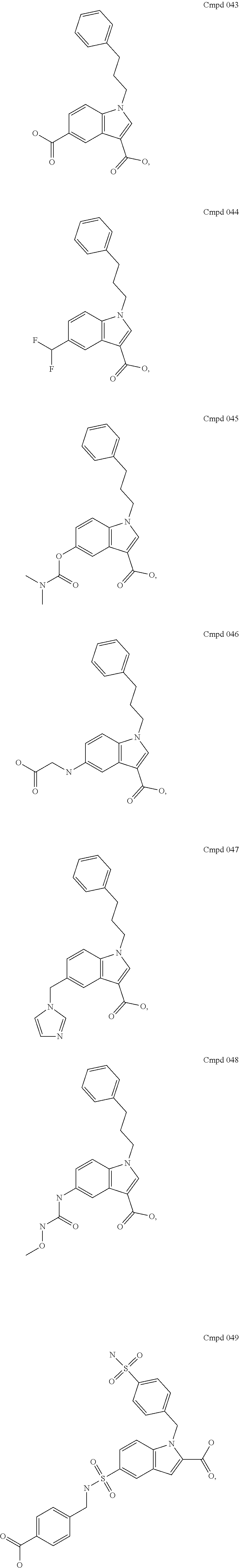 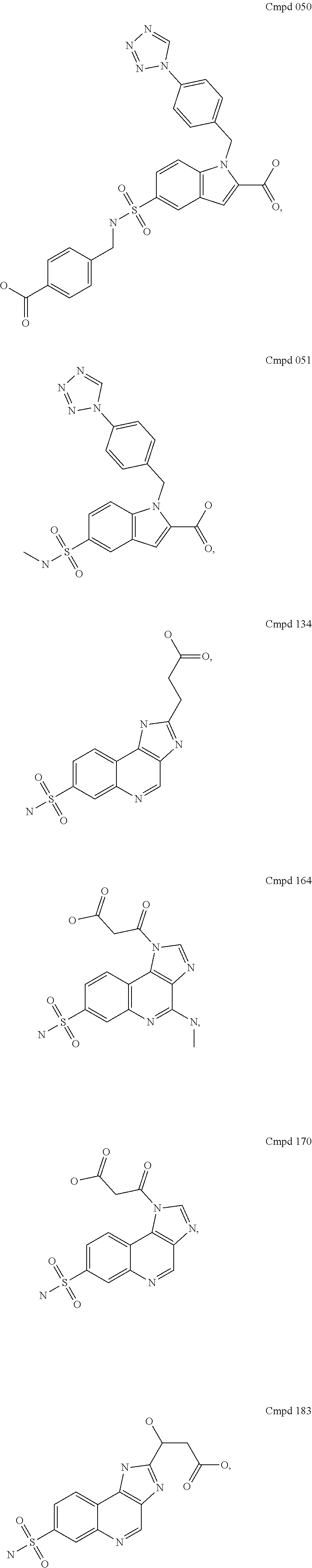
and a pharmaceutically acceptable salt, hydrate, solvate, or polymorph thereof.

11. (canceled)

12. A compound according to claim 5 selected from the group consisting of: 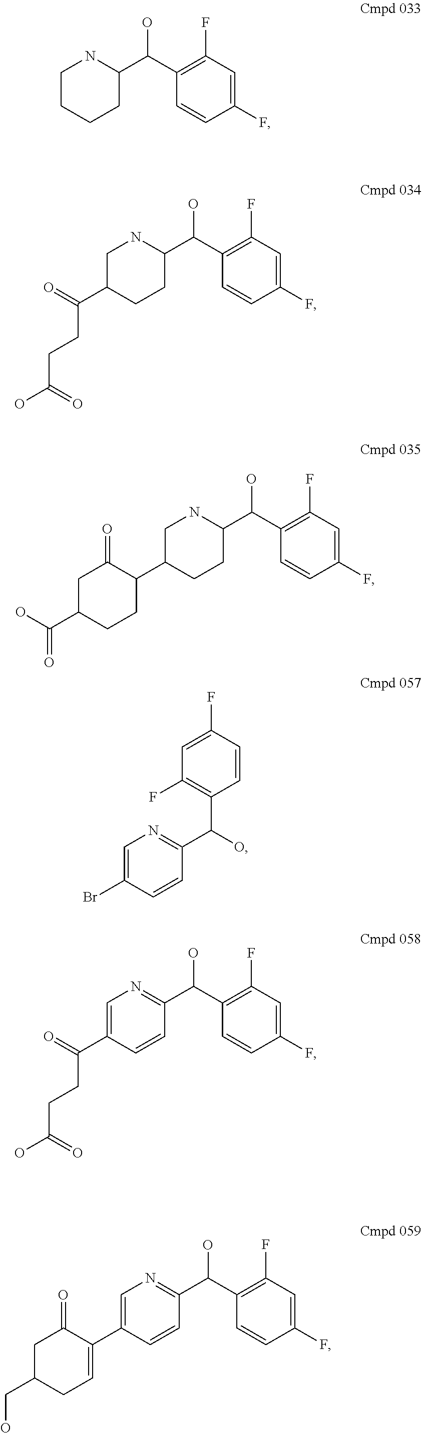
and a pharmaceutically acceptable salt, hydrate, solvate, or polymorph thereof.

13. (canceled)

14. A compound according to claim 5 selected from the group consisting of: 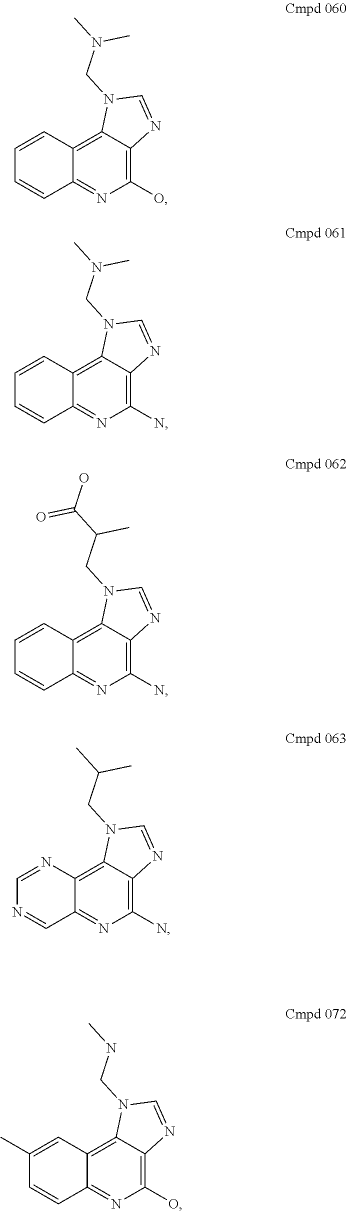 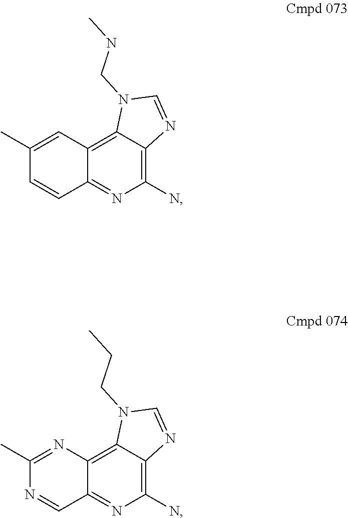
and a pharmaceutically acceptable salt, hydrate, solvate, or polymorph thereof.

15. (canceled)

16. A compound according to claim 5, selected from the group consisting of: 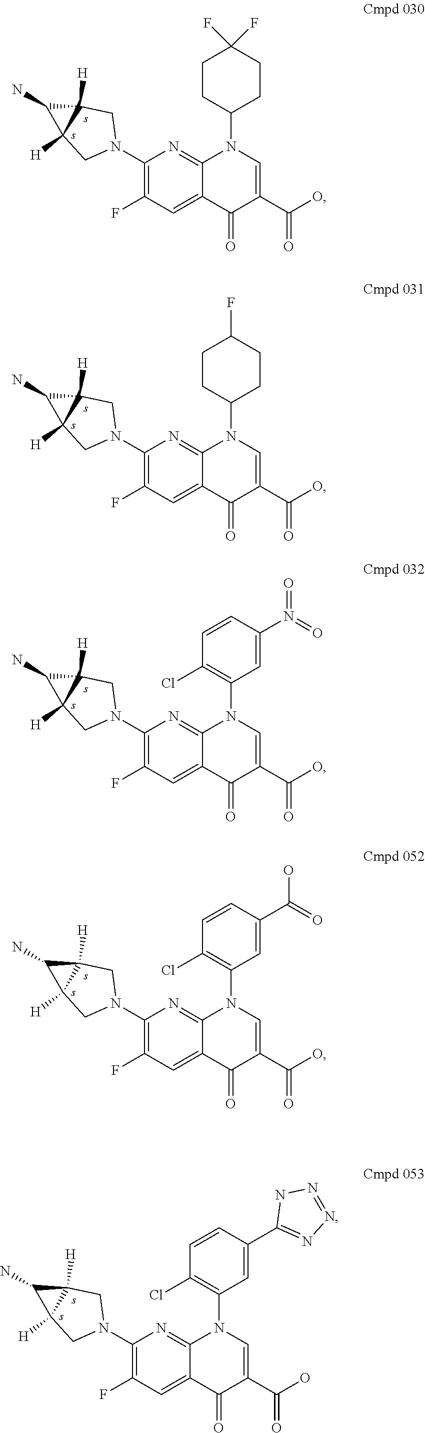
and a pharmaceutically acceptable salt, hydrate, solvate, or polymorph thereof.

17. (canceled)

18. A compound according to claim 1, selected from the group consisting of: 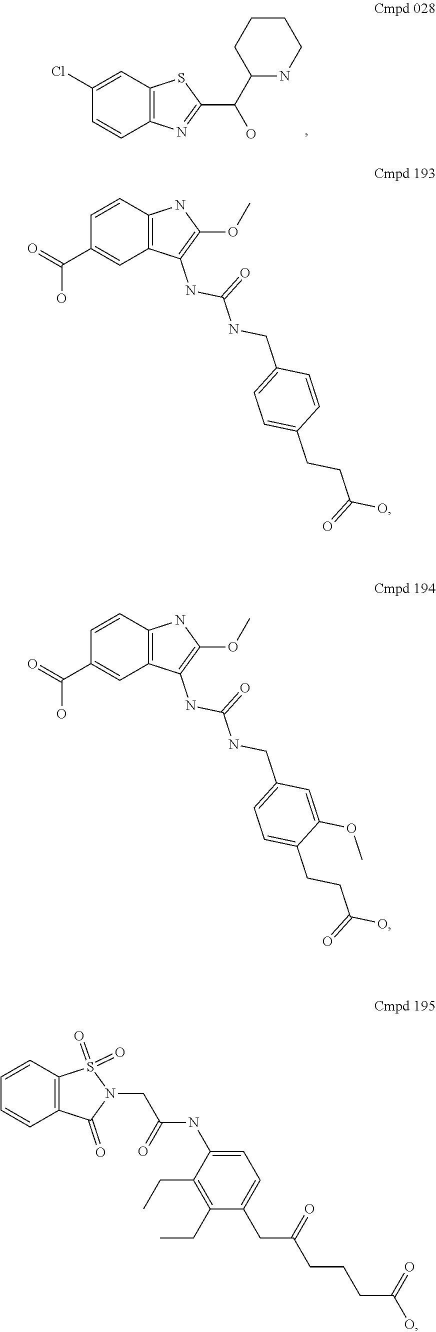 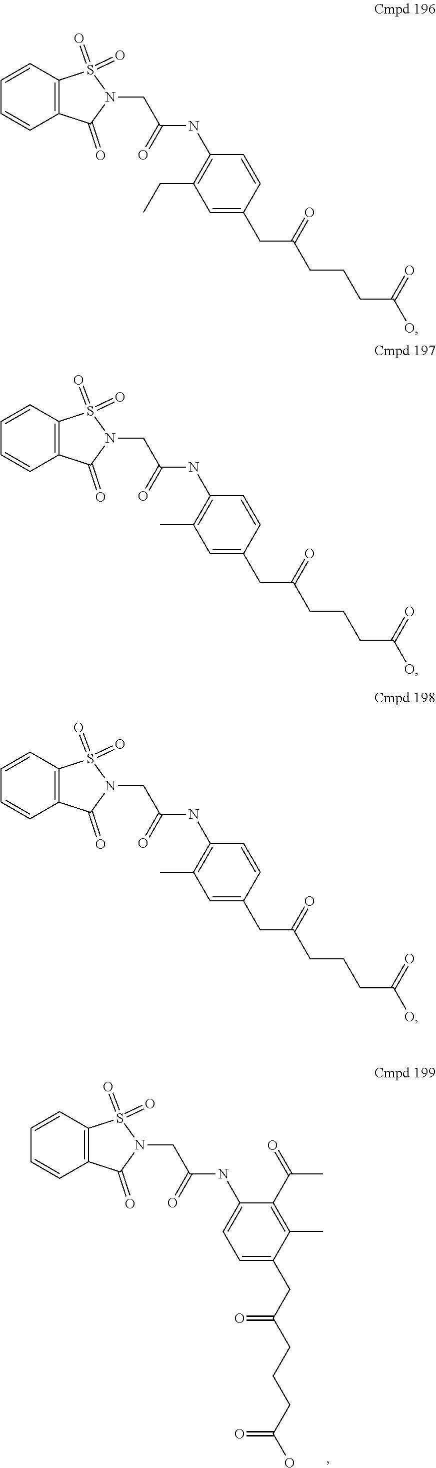 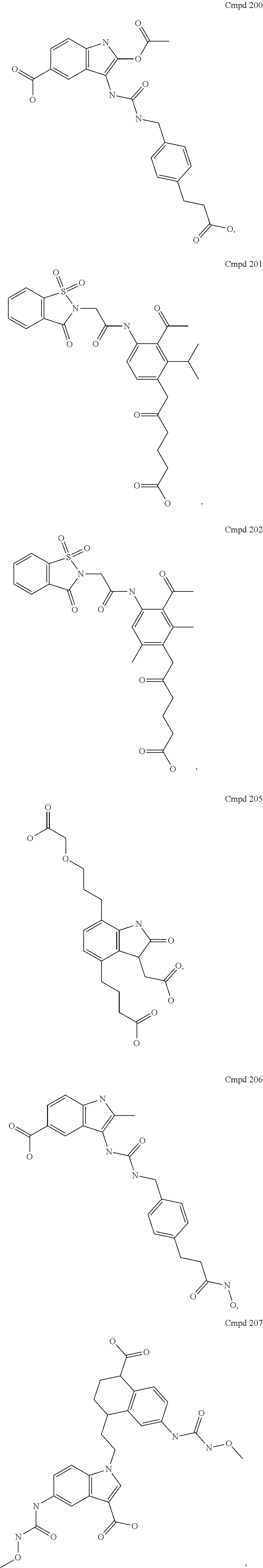 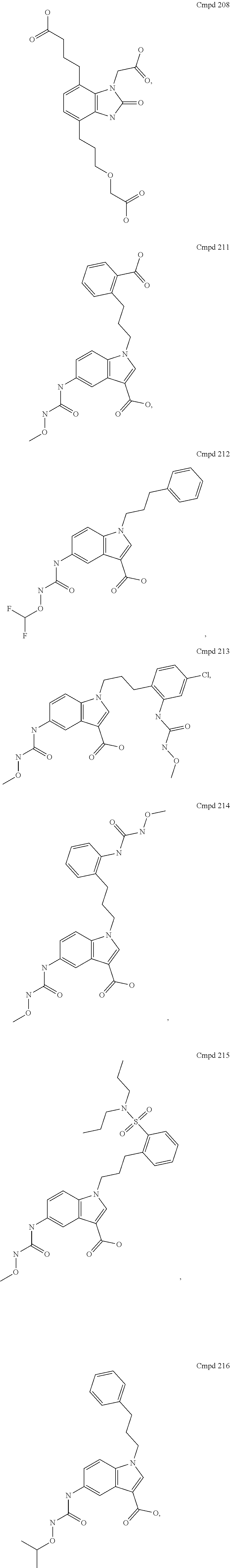 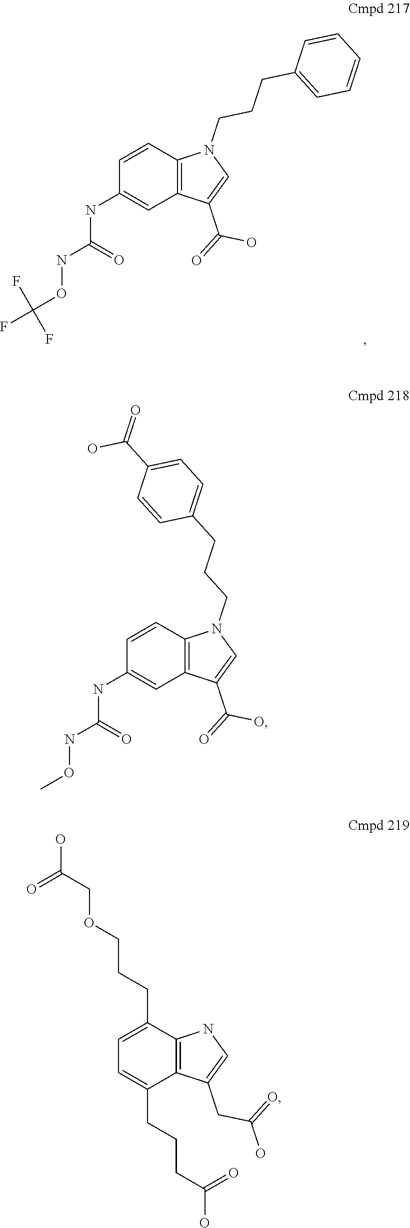
and a pharmaceutically acceptable salt, hydrate, solvate, or polymorph thereof.

19. A pharmaceutical composition comprising a compound of formula I, I′, II, III, IV, V, VI, VII, or VIII or a salt or solvate thereof, and one or more pharmaceutically acceptable excipients.

20. A method of treating a disease or disorder related to PANX1 abnormal signaling comprising administering a compound of formula I, I′, II, III, IV, V, VI, VII, or VIII, or a salt or solvate thereof, to a subject in need thereof.

21. A method according to claim 20, wherein the disease or disorder is selected from the group comprising chronic pain, chemotherapy-associated pain; addiction, particularly opioid addiction; epilepsy; Parkinson's Disease; Alzheimer's Disease; multiple sclerosis; traumatic brain injury; migraine; stroke; cancer, particularly melanoma, hepatocellular carcinoma, breast cancer, colorectal cancer, pancreatic cancer, and leukemia; cardiovascular diseases, particularly arrythmia, vascular inflammation, elevated blood pressure, and pulmonary arterial hypertension; inflammatory diseases, particularly joint inflammation and wound healing inflammatory disorders; pulmonary diseases, particularly Covid-19, asthma, and primary and secondary ciliary dyskinesia; fibrosis; diabetes; eye diseases; and skin diseases.

22. A method according to claim 20, wherein the disease or disorder is chronic pain.

23. A method according to claim 20, wherein the disease or disorder is opioid addiction.
